(19) United States
(12) Patent Application Publication
(10) Pub. No.: US 2023/0210834 A1
(43) Pub. Date: Jul. 6, 2023

(54) RNA METHYLTRANSFERASE INHIBITOR, SCREENING METHOD THEREFOR, ANTI-CANCER AGENT EFFICACY ASSESSMENT MARKER, AND KIT FOR EFFECTIVELY PREDICTING FTSJ1 INHIBITOR

(71) Applicants: National University Corporation Okayama University, Okayama (JP); General Incorporated Association Pharma Valley Project Supporting Organization, Shizuoka (JP); National University Corporation Kumamoto University, Kumamoto (JP)

(72) Inventors: Atsushi FUJIMURA, Okayama (JP); Kengo INOUE, Shizuoka (JP); Takayuki ANDO, Shizuoka (JP); Yoshinobu ISHIKAWA, Shizuoka (JP); Kazuhito TOMIZAWA, Kumamoto (JP)

(73) Assignees: National University Corporation Okayama University, Okayama (JP); General Incorporated Association Pharma Valley Project Supporting Organization, Shizuoka (JP); National University Corporation Kumamoto University, Kumamoto (JP)

(21) Appl. No.: 17/638,540

(22) PCT Filed: Aug. 26, 2020
(86) PCT No.: PCT/JP2020/032130

§ 371 Date: Feb. 25, 2022

(30) Foreign Application Priority Data
Aug. 26, 2019 (JP) 2019-153832

(51) Int. Cl. A61K 31/4545; G01N 33/50; C12Q 1/6886; A61P 35/00; A61K 31/343; A61K 31/451; A61K 31/18; A61K 31/445; A61K 31/415; A61K 31/4166; A61K 31/47

(52) U.S. Cl. CPC A61K 31/4545; G01N 33/5011; C12Q 1/6886; A61P 35/00; A61K 31/343; A61K 31/451; A61K 31/18; A61K 31/445; A61K 31/415; A61K 31/4166; A61K 31/47; C12Q 2600/158; C12Q 2600/106; C12Q 2600/156

(57) ABSTRACT

An RNA methyltransferase inhibitor comprising sulfonamide-based compounds and/or pyrazoline-based compounds is provided

### TECHNICAL FIELD

The present invention relates to an RNA methyltransferase inhibitor, a screening method for the inhibitor, a marker for determining the efficacy of anti-cancer agents, and a kit for predicting the efficacy of FTSJ1 inhibitors.

### BACKGROUND ART

In the field of anti-cancer agents, for example, anti-cancer agents with a mechanism of action associated with cell cycle activities have conventionally been proposed from the standpoint of inhibiting the proliferation of cancer cells and thereby inhibiting the hypertrophy etc. of tumor tissue.

However, many cancer cells have an extremely long cell cycle or are in the quiescent phase of the cell cycle. On these cancer cells, conventional anti-cancer agents with a mechanism of action associated with cell cycle activities cannot efficiently exert their anticancer effect.

### SUMMARY OF INVENTION

### Technical Problem

With the recent development of technology for nucleic acid analysis, it is becoming elucidated that translation forms based on specific nucleic acid modifications play an important role in maintaining the functions of cells and viruses.

For cancer cells, for example, translation forms specific to cancer cells are becoming elucidated, and it is becoming clear that such translation forms play a major role in various phenomena, such as survival, proliferation, and metastasis of cancer cells, and maintenance of stem cell properties. Accordingly, enzymes involved in the translation function of cells, such as RNA methyltransferases, are attracting attention.

In view of the above circumstances, an object of the present invention is to provide an RNA methyltransferase inhibitor and a screening method for the inhibitor.

### Solution to Problem

The present inventors have conducted extensive research to solve the above problems, and consequently found that at least one compound selected from the group consisting of sulfonamide-based compounds represented by formula (1) below and pyrazoline compounds represented by formula (2) has a desired RNA methyltransferase inhibitory effect. The present invention has been completed based on the above findings.

More specifically, the present invention provides the following RNA methyltransferase inhibitor, novel sulfonamide-based compound, screening method, marker for determining the efficacy of anti-cancer agents, and kit for predicting the efficacy of FTSJ1 inhibitors.

### Item 1.

An RNA methyltransferase inhibitor comprising at least one compound selected from the group consisting of sulfonamide-based compounds represented by the following formula (1) and pyrazoline-based compounds represented by the following formula (2):

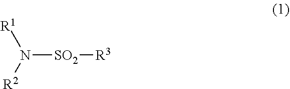

wherein R¹ represents any of the following groups (1-1) to (1-5):
(1-1) an optionally substituted nitrogen-containing heterocyclic group,
(1-2) optionally substituted cycloalkyl,
(1-3) optionally substituted alkyl,
(1-4) pyrazolylamino, and
(1-5) phenyl;
R² represents (2-1) hydrogen or (2-2) alkyl; and
R³ represents any of the following groups (3-1) to (3-9):
(3-1) phenyl,
(3-2) naphthyl,
(3-3) a nitrogen- or sulfur-containing heterocyclic group,
(3-4) dihydrocarbostyril,
(3-5) tetrahydronaphthyl,
(3-6) indanyl,
(3-7) benzoxolyl,
(3-8) benzothiadiazolyl, and
(3-9) dihydrobenzodioxepinyl;
wherein each group shown in (3-1) to (3-9) further optionally has one or more substituents, or R¹ and R², taken together with the nitrogen atom to which they are attached, optionally form a ring; and

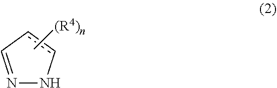

wherein n represents an integer of 2 to 4, and R⁴ is the same or different, and represents any of the following groups (4-1) to (4-34):
(4-1) phenyl,
(4-2) phenyl sulfonyl,
(4-3) alkyl carbonyl,
(4-4) aminothiocarbonyl,
(4-5) benzodioxolyl,
(4-6) alkyl sulfonyl,
(4-7) adamantylcarbonyl,
(4-8) benzopyrazyl,
(4-9) phenylcarbonyl,
(4-10) naphthyl,
(4-11) furylcarbonyl,
(4-12) thienylcarbonyl,
(4-13) quinazolyl,
(4-14) quinoxalyl,
(4-15) hydroxyl,
(4-16) alkenyl,
(4-17) thiazolyl,
(4-18) cycloalkylcarbonyl,
(4-19) aminocarbonyl,
(4-20) furyl,
(4-21) thienyl,
(4-22) pyridyl,
(4-23) cycloalkenyl,
(4-24) alkyl,
(4-25) pyrazolyl,
(4-26) quinolyl,
(4-27) alkenylcarbonyl,
(4-28) benzopyranyl,
(4-29) benzopyrimidyl,
(4-30) pyrrolidinoalkylcarbonyl,
(4-31) quinolylcarbonyl,
(4-32) alkoxy carbonyl,
(4-33) morpholino,
(4-34) pyrrolidinocarbonyl alkoxy, and
(4-35) benzodioxy-6-yl;
wherein each group shown in (4-1) to (4-35) further optionally has one or more substituents; the bond between the carbon atom at 4-position and the carbon atom at 5-position in the pyrazole skeleton is a single bond or a double bond, or two adjacent carbon atoms constituting the pyrazoline ring are optionally bonded to each other to form a ring, or the nitrogen atom constituting the pyrazoline ring and the carbon atom adjacent to the nitrogen atom are optionally bonded to each other to form a ring.

### Item 2.

The RNA methyltransferase inhibitor according to Item 1, wherein the one or more substituents on the nitrogen-containing heterocyclic group shown in (1-1) above are at least one member selected from the group consisting of alkyl, hydroxyl cyclopropyl, phenylthiopropylcarbonyl, phenyl sulfonyl, alkyl sulfonyl, thienyl sulfonyl, alkyl carbonyl, alkoxy carbonyl, phenyl sulfonylamino, aminocarbonylalkyl, pyrazolylcarbonyl, cyclopropylcarbonyl, piperidyl sulfonyl, and morpholinosulfonyl.

### Item 3.

The RNA methyltransferase inhibitor according to Item 1, wherein the number of substituents on the nitrogen-containing heterocyclic group shown in (1-1) above is 1 to 5.

### Item 4.

The RNA methyltransferase inhibitor according to Item 2 or 3, wherein the number of carbon atoms in the alkyl moiety and the alkoxy moiety constituting the alkyl, alkyl sulfonyl, alkyl carbonyl, alkoxy carbonyl, and aminocarbonylalkyl on the nitrogen-containing heterocyclic group shown in (1-1) above is 1 to 4.

### Item 5.

The RNA methyltransferase inhibitor according to any one of Items 2 to 4, wherein the phenyl sulfonyl on the nitrogen-containing heterocyclic group shown in (1-1) above further has at least one substituent selected from the group consisting of halogen, alkyl, fluoroalkyl, alkoxy, and nitro.

### Item 6.

The RNA methyltransferase inhibitor according to Item 1, wherein the cycloalkyl shown in (1-2) above has a carbon number of 3 to 6.

### Item 7.

The RNA methyltransferase inhibitor according to Item 1, wherein the cycloalkyl shown in (1-2) above has a carbon number of 5 or 6.

### Item 8.

The RNA methyltransferase inhibitor according to Item 1, wherein the alkyl shown in (1-3) above is optionally substituted C₁₋₆ linear alkyl.

### Item 9.

The RNA methyltransferase inhibitor according to any one of Items 1 to 8, wherein the one or more substituents on the cycloalkyl shown in (1-2) above and the one or more substituents on the alkyl shown in (1-3) above are each at least one member selected from the group consisting of phenyl, biphenyl, cycloalkyl, cycloalkenyl, nitrogen-containing heterocyclic groups, and hydroxyl.

### Item 10.

The RNA methyltransferase inhibitor according to any one of Items 1 to 9, wherein the phenyl, biphenyl, cycloalkyl, cycloalkenyl, nitrogen-containing heterocyclic group, or hydroxyl present on the alkyl shown in (1-3) further has C₁₋₅ alkyl as a substituent.

### Item 11.

The RNA methyltransferase inhibitor according to Item 1, wherein the one or more substituents on each group shown in (3-1) to (3-9) above are at least one member selected from the group consisting of alkyl, alkoxy, halogen, carboxyl, amino, nitro, phenyl, and cycloalkyl.

### Item 12.

The RNA methyltransferase inhibitor according to Item 1, wherein the number of substituents on each group shown in (3-1) to (3-9) above is 1 to 5.

### Item 13.

The RNA methyltransferase inhibitor according to Item 1, wherein the alkyl and the alkoxy on the phenyl shown in (3-1) above each have a carbon number of 1 to 5, and the cycloalkyl has a carbon number of 3 to 7.

### Item 14.

The RNA methyltransferase inhibitor according to Item 1, wherein the number of substituents on each group shown in (4-1) to (4-35) above is 1 to 6.

### Item 15.

The RNA methyltransferase inhibitor according to Item 1, wherein the one or more substituents on each group shown in (4-1) to (4-35) above are further at least one member selected from the group consisting of linear or branched alkyl, cycloalkyl, alkoxy, alkylamino, phenyl, phenylalkyl, phenylalkenyl, halogen, nitro, carboxy, furyl, dihydroxyphenyl, biphenylyl, alkyl carbonyl, oxo-substituted quinolyl, benzofuranyl, thienyl, trialkylamino, oxo, and pyridyl.

### Item 16.

The RNA transferase inhibitor according to Item 1, wherein the one or more substituents on the phenyl shown in (4-1) above are at least one member selected from the group consisting of halogen, alkyl, haloalkyl, alkoxy, hydroxyl, alkylsulfonylamino, nitro, amino, carboxyl, and phenyl.

### Item 17.

The RNA methyltransferase inhibitor according to Item 1, wherein the one or more substituents on the alkyl carbonyl shown in (4-3) above are at least one member selected from the group consisting of phenylalkylamino, triazolylthio, phenoxy, oxadiazolylthio, esters, piperazinyl, carboxyl, pyrimidinylthio, quinazolyloxy, morpholinocarbonyl, morpholino, benzotriazolyl, pyrazolyl carbonyl, pyrimidyl, pyrrolidino, piperidino, tetrahydroimidazolyl, halogen, naphthyloxy, alkoxy, imidazolyl, tetrazolylthio, alkylamino, pyridyl, tetrazolyl, benzodioxonyloxy, aminocarbonyl, piperazinyl, phenylalkylthio, alkylcarbonyloxy, benzotriazolylthio, pyridazinyl, pyrrolylcarbonyloxy, piperidino, dihydrothiazolylthio, benzopyrazyl, thienopyridinoxy, thienopyrimidinylthio, cyclopentathienopyrimidinyl, thiadiazolylthio, azepinylthio, dioxoloquinolinyl, diazaspirononanyl, imidazolidinyl, triazolylthio, dihydropyridazinyl, and 1,3-diazaspiroundecanyl.

### Item 18.

The RNA methyltransferase inhibitor according to Item 1, wherein each group on the alkyl carbonyl shown in (4-3) above further optionally has 1 to 6 substituents.

### Item 19.

The RNA methyltransferase inhibitor according to Item 1, wherein each group on the alkyl carbonyl shown in (4-3) above has at least one substituent selected from the group consisting of linear, branched, or cyclic alkyl, alkoxy, alkoxyphenyl, amino, carbamoyl, carbamoylalkyl, thienyl, furyl, tetrazolyl, alkyl carbonyl, halogen, phenyl, furanyl, alkylpyrrolidinyl, thiophenyl, furylcarbonyl, oxo, trifluoroalkyl, hydroxyl, thienylalkyl, alkylaminosulfonyl, hydroxyalkyl, furanylcarbonyl, benzylthio, nonanyl, bicyclononanyl, alkylthiadiazolyl, and alkylisoxazolyl.

### Item 20.

The RNA methyltransferase inhibitor according to Item 1, wherein in formula (2) above, the ring formed by the bonding of the two adjacent carbon atoms constituting the pyrazoline ring is a cyclohexane ring.

### Item 21.

The RNA methyltransferase inhibitor according to Item 20, wherein the cyclohexane ring has optionally substituted vinyl.

### Item 22.

The RNA methyltransferase inhibitor according to Item 21, wherein the one or more substituents on the vinyl are at least one member selected from the group consisting of phenyl, benzoxonyl, furyl, thienyl, and a cyclopentane ring.

### Item 23.

The RNA methyltransferase inhibitor according to Item 1, wherein in formula (2) above, the nitrogen atom constituting the pyrazoline ring and the carbon atom adjacent to the nitrogen atom are bonded to each other to form an optionally substituted cyclohexane ring.

### Item 24.

The RNA methyltransferase inhibitor according to Item 1, wherein in formula (2) above, the nitrogen atom constituting the pyrazoline ring and the carbon atom adjacent to the nitrogen atom are bonded to each other to form an optionally substituted cyclohexane ring, and the one or more substituents on the cyclohexane ring are at least one member selected from the group consisting of halogen, alkyl, alkoxy, optionally substituted (bi)phenyl, alkylphenyl, alkoxyphenyl, pyridyl, alkoxyphenyl, nitrophenyl, (di)fluorophenyl, (di)chlorophenyl, and spiro rings.

### Item 25.

The RNA methyltransferase inhibitor according to Item 1, wherein in formula (2) above, the nitrogen-containing heterocyclic group on the nitrogen atom constituting the pyrazoline ring, and the hydroxyphenyl on the carbon atom adjacent to the nitrogen atom constituting the pyrazoline ring on the pyrazoline ring are bonded to each other to form a ring.

### Item 26.

The RNA transferase inhibitor according to any one of Items 1 to 25, for use in the treatment of cancer.

### Item 27.

A sulfonamide-based compound represented by the following formula (1a):

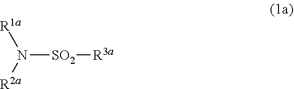

wherein R^{1a} represents optionally substituted piperidyl, optionally substituted pyridyl, optionally substituted pyrazolyl, cyclohexyl, optionally substituted C₁₋₅ linear alkyl, optionally substituted pyrazolylamino, or optionally substituted phenylamino; R^{2a} represents hydrogen or methyl; and R^{3a} represents optionally substituted phenyl.

### Item 28.

The sulfonamide-based compound according to Item 27, wherein the number of substituents on the optionally substituted piperidyl, optionally substituted pyridyl, optionally substituted pyrazolyl, optionally substituted cyclohexyl, optionally substituted C₁₋₅ linear alkyl, optionally substituted pyrazolylamino, or optionally substituted phenylamino represented by R^{1a} is 1 to 5.

### Item 29.

The sulfonamide-based compound according to Item 27, wherein the one or more substituents on the piperidyl represented by Ria are at least one member selected from the group consisting of methyl and hydroxyl.

### Item 30.

The sulfonamide-based compound according to Item 27, wherein the one or more substituents on the C₁₋₅ linear alkyl represented by Ria are at least one member selected from the group consisting of cycloalkyl, cycloalkenyl, nitrogen-containing heterocyclic groups, and hydroxyl.

### Item 31.

The sulfonamide-based compound according to Item 27, wherein the one or more substituents on the C₁₋₅ linear alkyl represented by R^{1a} are at least one member selected from the group consisting of cyclohexyl, cyclohexenyl, piperidyl, and hydroxyl.

### Item 32.

The sulfonamide-based compound according to Item 27, wherein the number of substituents on the phenyl represented by R^{3a} is 1 to 3.

### Item 33.

The sulfonamide-based compound according to Item 27, wherein the one or more substituents on the phenyl represented by R^{3a} are at least one member selected from the group consisting of C₁₋₆ alkyl, C₁₃ alkoxy, phenyl, halogen, and carboxyl.

### Item 34.

The sulfonamide-based compound according to Item 27, wherein the one or more substituents on the phenyl represented by R³ are at least one member selected from the group consisting of methyl, isopropyl, tert-butyl, tert-pentyl, methoxy, phenyl, cyclopropyl, chlorine, and carboxyl.

### Item 35.

A screening method for RNA transferase inhibitors, comprising the step of measuring RNA methylation inhibitory effects of a test substance against cells or viruses.

### Item 36.

The method according to Item 35, wherein the RNA methylation inhibitory effects are based on FTSJ inhibition.

### Item 37.

The method according to Item 35, wherein the FTSJ is FTSJ1.

### Item 38.

The method according to any one of Items 35 to 37, wherein the RNA methylation inhibitory effects are measured by a reporter assay using a sequence in which a translation regulatory region is added to a reporter region,

wherein

the translation regulatory region comprises a sequence formed by bonding of at least one member selected from the group consisting of glutamine, phenylalanine, tryptophan, methionine, and leucine.

### Item 39.

The method according to Item 38, wherein the translation regulatory region comprises a sequence in which 5 to 50 of at least one member selected from the group consisting of glutamine, phenylalanine, tryptophan, methionine, and leucine are continuously bonded.

### Item 40.

The method according to Item 38, wherein the translation regulatory region comprises polyglutamine, polyphenylalanine, polytryptophan, polymethionine, or polyleucine respectively comprising continuously bonded 5 to 50 glutamines, phenylalanines, tryptophans, methionines, or leucines.

### Item 41.

The method according to Item 38, wherein the translation regulatory region is any of SEQ ID No: 1 to 12.

### Item 42.

The method according to any one of Items 35 to 41, further comprising a reporter assay using a sequence comprising the transcription factor binding region and a reporter region represented by SEQ ID No: 13.

### Item 43.

A screening method for FTSJ1 inhibitors, comprising, in this order, the step of adding a methyl group donor to a test substance to obtain a reaction product; and the step of measuring FTSJ1 activity of the test substance using the reaction product.

### Item 44.

The method according to Item 43, wherein the methyl group donor is S-adenosylmethionine (SAM).

### Item 45.

The method according to Item 44, wherein the FTSJ1 activity is measured by a luciferase assay.

### Item 46.

A method for predicting the efficacy of an FTSJ1 inhibitor against a cancer, or a method for predicting prognosis after use of an FTSJ1 inhibitor against cancer, comprising step A of measuring the FTSJ1 expression level in a sample.

### Item 47.

The method according to Item 46, wherein step A is performed by an immunological method or genetic method.

### Item 48.

The method according to Item 46 or 47, wherein the sample is taken from a patient.

### Item 49.

The method according to any one of Items 46 to 48, further comprising step B for determining the efficacy of an FTSJ1 inhibitor against a cancer, or step B for determining prognosis of cancer pathology of the patient, based on the FTSJ1 expression level obtained in step A.

### Item 50.

The method according to any one of Items 46 to 49, wherein the cancer is at least one member selected from the group consisting of glioblastoma (malignant brain tumor), pancreatic cancer, acute myeloid leukaemia, lung cancer, liver cancer, kidney cancer, gastric cancer, and breast cancer.

### Item 51.

A marker for determining efficacy of an anti-cancer agent, comprising an FTSJ1 inhibitor sensitivity-related gene marker or FTSJ1 inhibitor resistance-related gene marker.

### Item 52.

The marker according to Item 51, wherein the FTSJ1 inhibitor sensitivity-related gene marker or FTSJ1 inhibitor resistance-related gene marker is an FTSJ1 modified nucleic acid RNA.

### Item 53.

The marker according to Item 50, wherein the FTSJ1 inhibitor resistance-related gene marker is at least one member selected from the group consisting of AHNAK nucleoprotein 2 (AHNAK2, SEQ ID No: 14), extended synaptotagmin 1 (ESYT1, SEQ ID No: 15), SLIT-ROBO Rho GTPase activating protein 1 (SRGAP1, SEQ ID No: 16), ras homolog family member F, filopodia associated (RHOF, SEQ ID No: 17), microRNA 4746 (MIR4746, SEQ ID No: 18), UBX domain protein 6 (UBXN6, SEQ ID No: 19), cytochrome c oxidase assembly factor COX16 (COX16, SEQ ID No: 20), ferritin heavy chain 1 (FTH1, SEQ ID No: 21), lysophosphatidic acid receptor 1 (LPAR1, SEQ ID No: 22), ankyrin repeat domain 29 (ANKRD29, SEQ ID No: 23), twist family bHLH transcription factor 2 (TWIST2, SEQ ID No: 24), JNK1/MAPK8 associated membrane protein (JKAMP, SEQ ID No: 25), protein kinase AMP-activated catalytic subunit alpha 2 (PRKAA2, SEQ ID No: 26), cleavage stimulation factor subunit 2 tau variant (CSTF2T, SEQ ID No: 27), thrombospondin type 1 domain containing 4 (THSD4, SEQ ID No: 28), membrane associated guanylate kinase, WW and PDZ domain containing 1 (MAGI1, SEQ ID No: 29), ubiquitin conjugating enzyme E2 L3 (UBE2L3, SEQ ID No: 30), glycosylphosphatidylinositol specific phospholipase D1 (GPLD1, SEQ ID No: 31), FRY like transcription coactivator (FRYL, SEQ ID No: 32), and myosin IXA (MYO9A, SEQ ID No: 33).

### Item 54.

The marker according to Item 50, wherein the FTSJ1 inhibitor sensitivity-related gene marker is at least one member selected from the group consisting of RNA binding motif protein 15 (RBM15, SEQ ID No: 34), nuclear autoantigenic sperm protein (NASP, SEQ ID No: 35), pre-mRNA processing factor 38A (PRPF38A, SEQ ID No: 36), chromosome 1 open reading frame 50 (C1orf50, SEQ ID No: 37), peroxisomal biogenesis factor 16 (PEX16, SEQ ID No: 38), zinc finger protein 213 (ZNF213, SEQ ID No: 39), fem-1 homolog B (FEM1B, SEQ ID No: 40), regulatory factor X associated protein (RFXAP, SEQ ID No: 41), Sin3A associated protein 18 (SAP18, SEQ ID No: 42), alanyl-tRNA synthetase 2, mitochondrial (AARS2, SEQ ID No: 43), regulator of chromosome condensation 2 (RCC2, SEQ ID No: 44), tyrosyl-tRNA synthetase 1 (YARS1, SEQ ID No: 45), RNA binding motif protein 10 (RBM10, SEQ ID No: 46), ribosomal protein L5 (RPL5, SEQ ID No: 47), zinc finger HIT-type containing 2 (ZNHIT2, SEQ ID No: 48), oxidative stress induced growth inhibitor family member 2 (OSGIN2, SEQ ID No: 49), egl-9 family hypoxia inducible factor 3 (EGLN3, SEQ ID No: 50), tRNA phosphotransferase 1 (TRPTI, SEQ ID No: 51), CRACD like (CRACDL, SEQ ID No: 52), capping actin protein, gelsolin like (CAPG, SEQ ID No: 53), RAB11 family interacting protein 3 (RAB11FIP3, SEQ ID No: 54), calcium homeostasis modulator family member 5 (CALHM5, SEQ ID No: 55), BICD cargo adaptor 1 (BICD1, SEQ ID No: 56), and FtsJ RNA 2′-O-Methyltransferase 1 (FTSJ1, SEQ ID No: 57).

### Item 55.

A kit for predicting efficacy of an FTSJ1 inhibitor, comprising the marker according to any one of Items 51 to 54.

### Advantageous Effects of Invention

The present invention provides an RNA methyltransferase inhibitor, a screening method thereof, a marker for determining the efficacy of anti-cancer agents, and a kit for predicting the efficacy of FTSJ1 inhibitors.

### BRIEF DESCRIPTION OF DRAWINGS

FIG. 1 shows the animal experiment results with regard to an RNA methyltransferase inhibitor of the present invention.

FIG. 2 shows the animal experiment results with regard to an RNA methyltransferase inhibitor of the present invention.

FIG. 3 shows the results of FTSJ1 inhibitory activity detected according to the screening method of the present invention.

FIG. 4 shows the results obtained by evaluating FTSJ1 inhibitor sensitive-related genetic markers.

### DESCRIPTION OF EMBODIMENTS

### 1. RNA Methyltransferase Inhibitor

The RNA methyltransferase inhibitor of the present invention contains a compound represented by the following formula (1) and/or a pyrazoline-based compound represented by the following formula (2):

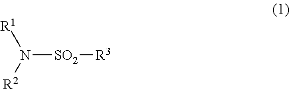

wherein R¹ represents any of the following groups (1-1) to (1-5):
(1-1) an optionally substituted nitrogen-containing heterocyclic group,
(1-2) optionally substituted cycloalkyl,
(1-3) optionally substituted alkyl,
(1-4) pyrazolylamino, and
(1-5) phenyl;
R² represents (2-1) hydrogen or (2-2) alkyl; and
R³ represents any of the following groups (3-1) to (3-9):
(3-1) phenyl,
(3-2) naphthyl,
(3-3) a nitrogen- or sulfur-containing heterocyclic group,
(3-4) dihydrocarbostyril,
(3-5) tetrahydronaphthyl,
(3-6) indanyl,
(3-7) benzoxolyl,
(3-8) benzothiadiazolyl, and
(3-9) dihydrobenzodioxepinyl;
wherein each group shown in (3-1) to (3-9) further optionally has one or more substituents, or R¹ and R², taken together with a nitrogen atom to which they are attached, optionally form a ring; and

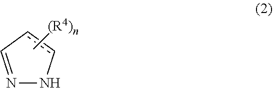

wherein n represents an integer of 2 to 4, and R⁴ is the same or different, and represents any of the following groups (4-1) to (4-35):
(4-1) phenyl,
(4-2) phenyl sulfonyl,
(4-3) alkyl carbonyl,
(4-4) aminothiocarbonyl,
(4-5) benzodioxolyl,
(4-6) alkyl sulfonyl,
(4-7) adamantylcarbonyl,
(4-8) benzopyrazyl,
(4-9) phenylcarbonyl,
(4-10) naphthyl,
(4-11) furylcarbonyl,
(4-12) thienylcarbonyl,
(4-13) quinazolyl,
(4-14) quinoxalyl,
(4-15) hydroxyl,
(4-16) alkenyl,
(4-17) thiazolyl,
(4-18) cycloalkylcarbonyl,
(4-19) aminocarbonyl,
(4-20) furyl,
(4-21) thienyl,
(4-22) pyridyl,
(4-23) cycloalkenyl,
(4-24) alkyl,
(4-25) pyrazolyl,
(4-26) quinolyl,
(4-27) alkenylcarbonyl,
(4-28) benzopyranyl,
(4-29) benzopyrimidyl,
(4-30) pyrrolidinoalkylcarbonyl,
(4-31) quinolylcarbonyl,
(4-32) alkoxy carbonyl,
(4-33) morpholino,
(4-34) pyrrolidinocarbonyl alkoxy, and
(4-35) benzodioxy-6-yl;
wherein each group shown in (4-1) to (4-35) further optionally has one or more substituents; the bond between the nitrogen atom at 2-position and the carbon atom at 3-position in the pyrazole skeleton is a single bond or a double bond, or two adjacent carbon atoms constituting the pyrazoline ring are optionally bonded to each other to form a ring, or the nitrogen atom constituting the pyrazoline ring and the carbon atom adjacent to the nitrogen atom are optionally bonded to each other to form a ring.

The present inventors found that by binding the compounds represented by formulae (1) and (2), competitively with S-adenosylmethionine (hereinafter also referred to simply as SAM), to a region (hereinafter also referred to simply as the SAM binding region) to which SAM in FTSJ, which is a tRNA methylation modification enzyme, binds, an RNA methylation modification reaction can be inhibited.

The present inventors have also found that the compounds represented by formulae (1) and (2) have anti-tumor effects based on effects of inhibiting RNA methylation modification, and are useful as cancer therapeutic agents. The cancer therapeutic agents defined in the present specification include not only what are generally called anti-cancer agents, but also cancer metastasis inhibitors.

The compounds represented by formulae (1) and (2) are described in detail below.

In this specification, examples of the alkyl include C₁₋₆ alkyl, such as methyl, ethyl, n-propyl, isopropyl, n-butyl, isobutyl, sec-butyl, tert-butyl, n-pentyl, and n-hexyl.

In this specification, examples of the alkoxy include C₁₋₆ alkoxy, such as methoxy, ethoxy, n-propyloxy, isopropyloxy, n-butyloxy, isobutyloxy, sec-butyloxy, tert-butyloxy, n-pentyloxy, and n-hexyloxy.

In this specification, examples of the cycloalkyl include C₃₋₈ cycloalkyl, such as cyclopropyl, cyclobutyl, cyclopentyl, cyclohexyl, and cycloheptyl.

In this specification, examples of the cycloalkenyl include C₃₋₈ cycloalkenyl, such as cyclopropenyl, cyclobutenyl, cyclopentenyl, cyclohexenyl, and cycloheptenyl.

In formula (1), examples of the nitrogen-containing heterocyclic represented by R¹ include piperidyl, pyridyl, and pyrazolyl. The nitrogen-containing heterocyclic group is preferably piperidyl.

Examples of the substituent on the nitrogen-containing heterocyclic group include alkyl, hydroxyl, cyclopropyl, phenylthiopropylcarbonyl, phenyl sulfonyl, alkyl sulfonyl, thienyl sulfonyl, alkyl carbonyl, alkoxy carbonyl, phenylsulfonylamino, aminocarbonylalkyl, pyrazolylcarbonyl, cyclopropylcarbonyl, piperidyl sulfonyl, and morpholinosulfonyl. The substituent on the nitrogen-containing heterocyclic group is preferably alkyl, and more preferably isopropyl. The number of substituents is 1 to 5, and preferably 1 to 4.

The number of carbon atoms in the alkyl moiety and alkoxy moiety that constitute the alkyl, alkyl sulfonyl, alkyl carbonyl, alkoxy carbonyl, and aminocarbonyl alkyl on the nitrogen-containing heterocyclic group shown in (1-1) above is 1 to 4.

The phenyl sulfonyl on the nitrogen-containing heterocyclic group shown in (1-1) above further contains at least one substituent selected from the group consisting of halogen, alkyl, fluoroalkyl, alkoxy, and nitro.

In formula (1), examples of the cycloalkyl represented by R¹ include C₃₋₈ cycloalkyl, such as cyclopropyl, cyclobutyl, cyclopentyl, cyclohexyl, and cycloheptyl. The cycloalkyl preferably has 3 to 6 carbon atoms. The cycloalkyl is more preferably cyclopentyl or cyclohexyl.

In formula (1), examples of the alkyl represented by R¹ include C₁₋₆ alkyl, such as methyl, ethyl, n-propyl, isopropyl, n-butyl, isobutyl, sec-butyl, tert-butyl, n-pentyl, and n-hexyl. The alkyl is preferably methyl, ethyl, and isopropyl.

The substituent on the cycloalkyl shown in (1-2) above, and the substituent on the alkyl shown in (1-3) above are each at least one member selected from the group consisting of phenyl, biphenyl, cycloalkyl, cycloalkenyl, nitrogen-containing heterocyclic (e.g., piperidyl, pyridyl, and pyrazolyl), and hydroxyl.

The phenyl, biphenyl, cycloalkyl, cycloalkenyl, nitrogen-containing heterocyclic, or hydroxyl present on the alkyl shown in (1-3) above may further contain C₁₋₅ alkyl as a substituent.

The substituent on each group shown in (3-1) to (3-9) is at least one member selected from the group consisting of alkyl, alkoxy, halogen, carboxyl, amino, nitro, phenyl, and cycloalkyl. The number of substituents on each group shown in (3-1) to (3-9) above is 1 to 5, and preferably 1 to 3.

The number of carbon atoms in the alkyl and alkoxy on the phenyl shown in (3-1) above is 1 to 5, and the number of carbon atoms in the cycloalkyl is 3 to 7. The number of carbon atoms in the alkyl and alkoxy is preferably 1 to 3.

Examples of the nitrogen- or sulfur-containing heterocyclic group shown in (3-3) above include pyrrolyl, piperidyl, quinolyl, and thienyl.

R³ is preferably (3-1) phenyl or (3-2) naphthyl. The phenyl is preferably substituted with one to three C₁₋₅ alkyl groups, and is more preferably substituted with three isopropyl groups.

In formula (2), the number of substituents on each group shown in (4-1) to (4-35) above defined by R⁴ is 1 to 6, and preferably 1 to 3.

The substituent on each group shown in (4-1) to (4-35) above is at least one member selected from the group consisting of linear or branched alkyl, cycloalkyl, alkoxy, alkylamino, phenyl, phenylalkyl, phenylalkenyl, halogen, nitro, carboxy, furyl, dihydroxyphenyl, biphenylyl, alkyl carbonyl, oxo-substituted quinolyl, benzofuranyl, thienyl, trialkylamino, oxo, and pyridyl.

The substituent on the phenyl shown in (4-1) above is preferably at least one member selected from the group consisting of halogen, alkyl, haloalkyl, alkoxy, hydroxyl, alkylsulfonylamino, nitro, amino, carboxyl, and phenyl.

The substituent on the alkyl carbonyl shown in (4-3) above is at least one member selected from the group consisting of phenylalkylamino, triazolylthio, phenoxy, oxadiazolylthio, esters, piperazinyl, carboxyl, pyrimidinylthio, quinazolyloxy, morpholinocarbonyl, morpholino, benzotriazolyl, pyrazolyl carbonyl, pyrimidyl, pyrrolidino, piperidino, tetrahydroimidazolyl, halogen, naphthyloxy, alkoxy, imidazolyl, tetrazolylthio, alkylamino, pyridyl, tetrazolyl, benzodioxonyloxy, aminocarbonyl, piperazinyl, phenylalkylthio, alkylcarbonyloxy, benzotriazolylthio, pyridazinyl, pyrrolylcarbonyloxy, piperidino, dihydrothiazolylthio, benzopyrazyl, thienopyridinoxy, thienopyrimidinylthio, cyclopentathienopyrimidinyl, thiadiazolylthio, azepinylthio, dioxoloquinolinyl, diazaspirononanyl, imidazolidinyl, triazolylthio, dihydropyridazinyl, and 1,3-diazaspiroundecanyl.

Each group on the alkyl carbonyl shown in (4-3) above may further have 1 to 6 substituents, and preferably 1 to 3 substituents.

Each group on the alkyl carbonyl shown in (4-3) above has at least one substituent selected from the group consisting of linear, branched, or cyclic alkyl, alkoxy, alkoxyphenyl, amino, carbamoyl, carbamoylalkyl, thienyl, furyl, tetrazolyl, alkyl carbonyl, halogen, phenyl, furanyl, alkylpyrrolidinyl, thiophenyl, furylcarbonyl, oxo, trifluoroalkyl, hydroxyl, thienylalkyl, alkylaminosulfonyl, hydroxyalkyl, furanylcarbonyl, benzylthio, nonanyl, bicyclononanyl, alkylthiadiazolyl, and alkylisoxazolyl.

In formula (2) above, the ring formed by the bonding of the two adjacent carbon atoms constituting the pyrazoline ring is, for example, a cyclohexane ring. The cyclohexane ring preferably has optionally substituted vinyl.

The substituent on the vinyl is at least one member selected from the group consisting of phenyl, benzoxonyl, furyl, thienyl, and a cyclopentane ring.

In formula (2) above, it is preferable that the nitrogen atom constituting the pyrazoline ring and the carbon atom adjacent to the nitrogen atom are bonded to each other to form an optionally substituted cyclohexane ring. The substituent on the cyclohexane ring is at least one member selected from the group consisting of halogen, alkyl, alkoxy, optionally substituted (bi)phenyl, alkylphenyl, alkoxyphenyl, pyridyl, alkoxyphenyl, nitrophenyl, (di)fluorophenyl, (di)chlorophenyl, and spiro rings.

In formula (2), the nitrogen-containing heterocyclic group on the nitrogen atom constituting the pyrazoline ring, and the hydroxyphenyl on the carbon atom adjacent to the nitrogen atom constituting the pyrazoline ring on the pyrazoline ring are optionally bonded to each other to form a ring.

Of the groups shown in (4-1) to (4-35) above, the (4-1) phenyl, (4-2) phenyl sulfonyl, (4-3) alkyl carbonyl, (4-4) aminothiocarbonyl, (4-6) alkyl sulfonyl, (4-11) furylcarbonyl, (4-12) thienylcarbonyl, (4-20) furyl, (4-21) thienyl, (4-22) pyridyl, (4-25) pyrazolyl, or (4-35) benzodioxy-6-yl is preferred.

Of the groups shown in (4-1) to (4-35) above, the (4-1) phenyl, (4-2) phenyl sulfonyl, (4-3) alkyl carbonyl, (4-4) aminothiocarbonyl, (4-6) alkyl sulfonyl, (4-11) furylcarbonyl, (4-12) thienylcarbonyl, (4-20) furyl, (4-21) thienyl, (4-22) pyridyl, (4-25) pyrazolyl, or (4-35) benzodioxy-6-yl is preferred.

Of the groups shown in (4-1) to (4-35) above, the (4-1) phenyl, (4-3) alkyl carbonyl, (4-6) alkyl sulfonyl, or (4-35) benzodioxy-6-yl is more preferred.

Of the groups shown in (4-1) to (4-35) above, the (4-1) phenyl and (4-35) benzodioxy-6-yl are particularly preferred.

In formula (2), examples of the substituent on the phenyl shown in (4-1) include halogen such as bromine, alkoxy such as methoxy, and hydroxyl; and preferable example include hydroxyl. The number of substituents on the phenyl is 1 to 5, preferably 1 to 3, and more preferably 1.

When the RNA methyltransferase inhibitor of the present invention is used as a cancer therapeutic agent, it may further contain a pharmaceutically acceptable carrier in addition to the above compounds. Examples of the pharmaceutically acceptable carrier include usually employed diluents and excipients, such as fillers, extenders, binders, wetting agents, disintegrants, surfactants, and lubricants. The RNA methyltransferase inhibitor of the present invention may be prepared in the form of common pharmaceutical preparations, such as tablets, flash-melt tablets, pills, sprays, solutions, suspensions, emulsions, granules, capsules, suppositories, injections (solutions, suspensions, etc.), troches, nasal sprays, and transdermal patches. The RNA methyltransferase inhibitor can be used in various cancers without any particular limitation.

The RNA methyltransferase inhibitor of the present invention can be administered by any method, and administered by a method according to the form of the preparation, the patient's age and sex, and other conditions (degree of disease). For example, tablets, pills, solutions, suspensions, emulsions, granules, and capsules are administered orally. Injections are intravenously administered singly or as mixed with usual injection transfusions, such as glucose solutions or amino acid solutions; or singly administered intramuscularly, intracutaneously, subcutaneously or intraperitoneally. Suppositories are administered intrarectally.

### 2. Sulfonamide-Based Compound

The present invention also includes an invention relating to a novel sulfonamide-based compound. The sulfonamide-based compound is represented by the following formula (1a).

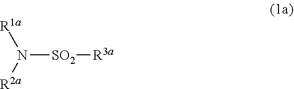

In formula (1a), Ria represents optionally substituted piperidyl, optionally substituted pyridyl, optionally substituted pyrazolyl, cyclohexyl, optionally substituted C₁₋₅ linear alkyl, optionally substituted pyrazolylamino, or optionally substituted phenylamino. R^{2a} represents hydrogen or methyl. R^{3a} represents optionally substituted phenyl.

R^{1a} is optionally substituted piperidyl, cyclohexyl, or C₁₋₅ linear alkyl. R^{2a} represents hydrogen or methyl.

In formula (1a), the substituent on the piperidyl represented by R^{1a} is preferably trifluoromethyl-substituted pyridyl.

In formula (1a), the substituent on the pyridyl represented by R^{1a} is preferably difluorophenyloxy.

In formula (1a), the substituent on the pyrazolyl represented by R^{1a} is preferably trifluoromethyl-substituted phenyl.

In formula (1a), the substituent on the C₁₋₅ linear alkyl represented by R^{1a} is preferably carbonylamino or piperidyl.

In formula (1a), the phenyl represented by R^{3a} is preferably substituted with three C₁₋₅ alkyl groups (preferably isopropyl).

The compound represented by formula (1a) above can be obtained by the method described in the Production Examples below or by an equivalent method.

### 3. Screening Method

Furthermore, the present invention includes an invention relating to a screening method for anti-cancer agents. In the screening method of the present invention, the RNA methylation inhibitory effects of test substances are measured by using cells or viruses.

The cells to be used are preferably cancer cells. The type of cancer is not particularly limited. Specific examples include pharyngeal cancer (e.g., lip cancer, gingival cancer, tongue cancer, oral cancer, oral floor cancer, and salivary gland cancer), gastrointestinal cancer (e.g., esophageal cancer, gastric cancer, appendiceal cancer, colon cancer, and rectal cancer), lung cancer, liver cancer, hepatocellular carcinoma, cholangiocarcinoma, bone cancer, articular cartilage cancer, malignant melanoma of the skin, spinocellular carcinoma, other skin cancers, mesothelioma, breast cancer, uterine cancer (e.g., cervical cancer, and endometrial cancer), ovarian cancer, prostate cancer, bladder cancer, brain tumor, thyroid cancer, non-Hodgkin's lymphoma, lymphocytic leukaemia, sarcoma, and cancers of metastatic tissue in which the aforementioned cancers are the primary tumors.

Specific cancer cells are not particularly limited, and cancer cells known in the cancer types mentioned above can be used. Of these, cancer stem cells are preferably used.

As the test substance, a low-molecular-weight compound is preferably used to measure methylation inhibition effects on the above cells or viruses, thus selecting a test substance with a desired level of inhibition.

In one preferred embodiment in the screening method of the present invention, examples of the method for measuring RNA methylation inhibitory effects include a method for measuring the activity of an enzyme that modifies RNA methylation, i.e., RNA methyltransferase. Wide variety of known RNA methyltransferases can be used. Specific examples include enzymes belonging to the ALKBH family and the Mettle family.

Of FTSJ, since FTSJ1 is considered to be a poor cancer prognostic factor, measuring the FTSJ1 inhibitory activity of the test substance in screening for anti-cancer agents is preferred.

The more specific method for measuring the activity of RNA methyltransferase is not particularly limited, and wide variety of known methods can be used. Examples include ELISA, RIA, immunoprecipitation, bisulfite, quantitative PCR, and reporter assay. Of these, the reporter assay is preferred because easy and accurate measurement is possible.

It is predicted that FTSJ1 performs 2′-O-methylatation of nucleotides at positions 32 and 34 of the tRNA corresponding to each of the polyglutamine (Q), phenylalanine (F), methionine (M), and asparagine (N) codons in mammalian cells.

In the reporter assay, the sequence of the translation regulatory region to which the reporter region is bonded preferably contains a sequence formed by the bonding of at least one member selected from the group consisting of glutamine, phenylalanine, tryptophan, methionine, and leucine.

The translation regulatory region is preferably a repeated sequence of each of the above five amino acids, i.e., polyglutamine, polyphenylalanine, polytryptophan, polymethionine, or polyleucine; or may be a sequence randomly containing these five amino acids. Further, the translation regulatory region may contain other amino acids to the extent that the function is not inhibited.

The size of amino acid tandem repeat is preferably 3 or more, more preferably 5 or more, and even more preferably 8 or more. The upper limit of the number of repeats is not particularly limited, and it can be set to 50.

Specific examples of such a translation regulatory region include sequences represented by SEQ ID Nos. 1 to 12. Examples include the sequence of SEQ ID No: 1 or 2 as polyglutamine, the sequence of SEQ ID No: 3 or 4 as polyphenylalanine, the sequence of SEQ ID No: 5 as polytryptophan, the sequence of SEQ ID No: 6 as polymethionine, and the sequences of SEQ ID No: 7 to 12 as polyleucine.

Furthermore, as a result of the research of the present inventors, YAP/TAZ is predicted to be a protein responsible for the nature of cancer stem cells, and whose protein synthesis is promoted by FTSJ1. Accordingly, in addition to the reporter assay in which the translation regulatory region is added to the reporter, it is also preferable to perform a reporter assay in which expression is regulated in the transcription factor binding region (GTIIC) that contains the sequence represented by SEQ ID No: 13, 3 to 15 times repeatedly (another sequence may be contained between each of the repeating units). In addition, sphere-formation assay and/or mass spectrometry are preferably performed.

Wide variety of known reporter genes can be used in the reporter assay without any particular limitation. Specific examples include a β-galactosidase gene, chloramphenicol acetyltransferase gene derived from bacterial transposon, and luciferase gene derived from Lucida cruciata. Of these, the luciferase gene derived from Lucida cruciata is preferably used because of its superior detection sensitivity.

Wide variety of known methods can be used as a method for linking the transcriptional regulatory region to the reporter gene, without any particular limitation. Specifically, a method in which the purified transcription sequence region is cleaved by a suitable limitation enzyme to link to the reporter gene, can be used.

As a vector for inserting the linked sequence, wide variety of known vectors for reporter assays, such as plasmids, shuttle vectors, and helper plasmids, can be used.

The method for transfecting the vector into cells is not particularly limited, and wide variety of known methods can be used. Examples include an electroporation method, spheroplast method, and lithium acetate method.

The luminescence intensity of the vector-transfected cells is measured using a luminometer according to a usual method. In the screening method of the present invention, it is preferable to measure the luminescence of solvents such as DMSO as a control group (blank) to calculate the assay value (%) of the test substance relative to the control group.

In the reporter assay in which the translation regulatory region containing a sequence formed by the bonding of at least one member selected from the group consisting of glutamine, phenylalanine, tryptophan, methionine, and leucine is added to a reporter, those having an assay value of preferably 100 or less, more preferably 80 or less, and even more preferably 40 or less can be selected by screening.

In the reporter assay in which the transcriptional regulatory region (GTIIC) represented by SEQ ID No: 13 is added to a reporter, those having an assay value of preferably 100 or less, more preferably 80 or less, and even more preferably 40 or less can be selected by screening.

Other embodiments include a screening method comprising the step of adding a methyl group donor to the test substance to obtain a reaction product, and the step of measuring the test substance using the reaction product.

Preferable examples of the methyl group donor include that can become a precursor of ATP as described below by desorbing the methyl group from the methyl group donor. Specific examples include S-adenosylmethionine (hereinafter simply referred to as “SAM”).

In the step of measuring the RNA methylation inhibitory effects against cells or viruses, ELISA, RIA, immunoprecipitation, bisulfite, quantitative PCR, reporter assay, and luciferase assay can be used.

In particular, when SAM is used as a methyl group donor in the step of adding the methyl group donor to the test substance, FTSJ1 contained in the test substance converts SAM into S-adenosylhomocysteine (hereinafter also referred to simply as “SAH”). Then, a reaction with a reagent that converts the obtained SAH into adenosine diphosphate (hereinafter also referred to simply as “ADP”) is performed to further add a predetermined reagent to ADP, thus obtaining adenosine triphosphate (hereinafter also referred to simply as “ATP”). By incorporating the obtained ATP into an assay system such as a luciferase assay, the activity of FTSJ1 in the test substance can be directly evaluated, which ensures highly accurate evaluation results.

### 4. Method for Predicting Efficacy of FTSJ1 Inhibitor Against Cancer, or Method for Predicting Prognosis

The present invention also comprises a method for predicting the efficacy of an FTSJ1 inhibitor against cancer, and a method for predicting the prognosis of cancer after the use of an FSTJ1 inhibitor. In the present specification, “prognosis” is defined as the medical outlook of a patient after chemotherapy.

The efficacy of the FTSJ1 inhibitor refers to the effect of the FTSJ1 inhibitor on cancer pathology. In other words, the method for predicting the efficacy of an FTSJ1 inhibitor according to the present invention includes the concept of the method for predicting the sensitivity to an FTSJ1 inhibitor of a cancer patient, or the concept of the method for predicting the sensitivity to an FTSJ1 inhibitor of cancer tissues or cells collected from a cancer patient. The method for predicting the efficacy of an FTSJ1 inhibitor according to the present invention includes the concept of the method for predicting the resistance to an FTSJ1 inhibitor of a cancer patient, or cancer tissues or cancer cells collected from a cancer patient.

The method for predicting the efficacy of an FTSJ1 inhibitor against cancer, or method for predicting prognosis after the use of an FTSJ1 inhibitor against cancer according to the present invention comprises step A of measuring the FTSJ1 expression level in a sample.

Cancer tissues or cancer cells derived from living organisms (including humans and animals) can be used as a sample. Specifically, cancer tissues or cancer cells collected from patients (cancer patients) can be used.

The FTSJ1 expression level can be measured by a wide variety of known methods without limitation. The immunological method and the genetic method can both be preferably used.

There is no particular limitation on the immunological method, and examples include ELISA, inmunostaining, flow cytometry, and immunoblotting.

There is no particular limitation on the genetic method, and examples include western blotting and RT-PCR.

The method for predicting the efficacy of an FTSJ1 inhibitor against cancer, or method for predicting prognosis after the use of an FTSJ1 inhibitor against cancer according to the present invention further comprises, after step A, step B for determining the efficacy of the FTSJ1 inhibitor against cancer, or for determining the prognosis of cancer pathology of the patient, based on the FTSJ1 expression level obtained in step A.

In particular, in step B, setting the predetermined cutoff value of the FTSJ1 expression level in the sample obtained in step A is preferred. For example, in predicting the efficacy of an FTSJ1 inhibitor, a sample whose expression level obtained in step A is above the cut-off value is predicted to have high FTSJ1 efficacy, whereas a sample with an expression level below the cut-off value is predicted to have low FTSJ1 efficacy.

Similarly, in the method for predicting the cancer pathogenesis of the patient after the use of an FTSJ1 inhibitor as well, for a sample whose expression level obtained in step A is above a predetermined cut-off value, patient prognosis is predicted to be good after the use of the FTSJ1 inhibitor; whereas for a sample with an expression level below the cut-off value, patient prognosis is predicted to be poor after the use of an FTSJ1 inhibitor.

The method for predicting the efficacy of an FTSJ1 inhibitor against cancer, or method for predicting the prognosis of cancer after the use of an FTSJ1 inhibitor according to the present invention can be widely used in known cancers, without any particular limitation. Specific examples include glioblastoma (malignant brain tumor), pancreatic cancer, acute myeloid leukemia, lung cancer, liver cancer, kidney cancer, gastric cancer, and breast cancer.

### 5. Marker for Determining Efficacy of Anti-Cancer Agent

The present invention also comprises the invention relating to a marker for determining the efficacy of an anti-cancer agent. The marker is a gene marker: an FTSJ1 inhibitor sensitivity-related gene marker and an FTSJ1 inhibitor resistance-related gene marker.

The sensitivity to an FTSJ1 inhibitor of the patient can be determined by whether these markers are detected from samples (tissues or cells) collected from patients (including humans and animals). The detection of an FTSJ1 inhibitor sensitivity-related gene marker from a sample suggests that the FTSJ1 inhibitor is effective for the patient in chemotherapy. On the other hand, the detection of an FTSJ1 inhibitor resistance-related gene marker from a sample suggests that the FTSJ1 inhibitor is not effective for the patient in chemotherapy.

These markers for determining the efficacy of an anti-cancer agent relating to FTSJ1 (FTSJ1 inhibitor sensitivity-related gene marker or FTSJ1 inhibitor resistance-related gene marker) are preferably FTSJ1-modified nucleic acid RNAs.

The FTSJ1 inhibitor resistance-related gene marker is preferably at least one member selected from the group consisting of AHNAK2 (SEQ ID No: 14), ESYT1 (SEQ ID No: 15), SRGAP1 (SEQ ID No: 16), RHOF (SEQ ID No: 17), MIR4746 (SEQ ID No: 18), UBXN6 (SEQ ID No: 19), COX16 (SEQ ID No: 20), FTH1 (SEQ ID No: 21), LPAR1 (SEQ ID No: 22), ANKRD29 (SEQ ID No: 23), TWIST2 (SEQ ID No: 24), JKAMP (SEQ ID No: 25), PRKAA2 (SEQ ID No: 26), CSTF2T (SEQ ID No: 27), THSD4 (SEQ ID No: 28), MAGI1 (SEQ ID No: 29), UBE2L3 (SEQ ID No: 30), GPLD1 (SEQ ID No: 31), FRYL (SEQ ID No: 32), and MYO9A (SEQ ID No: 33).

The FTSJ1 inhibitor sensitive-related gene marker is preferably at least one member selected from the group consisting of RBM15, SEQ ID No: 34), NASP (SEQ ID No: 35), PRPF38A (SEQ ID No: 36), C1orf50 (SEQ ID No: 37), PEX16 (SEQ ID No: 38), ZNF213 (SEQ ID No: 39), FEM1B (SEQ ID No: 40), RFXAP (SEQ ID No: 41), SAP18 (SEQ ID No: 42), AARS2 (SEQ ID No: 43), RCC2 (SEQ ID No: 44), YARS1 (SEQ ID No: 45), RBM10 (SEQ ID No: 46), RPL5 (SEQ ID No: 47), ZNHIT2 (SEQ ID No: 48), OSGIN2 (SEQ ID No: 49), EGLN3 (SEQ ID No: 50), TRPTI (SEQ ID No: 51), CRACDL (SEQ ID No: 52), CAPG (SEQ ID No: 53), RAB11FIP3 (SEQ ID No: 54), CALHM5 (SEQ ID No: 55), BICD1 (SEQ ID No: 56), and FTSJ1 (SEQ ID No: 57).

### 6. Kit for Predicting Efficacy of FTSJ1 Inhibitors

The present invention also includes a kit comprising the marker for determining the efficacy of anti-cancer agents. If at least one of the above FTSJ1 inhibitor resistance-related gene markers is detected in the sample, the FTSJ1 inhibitor is determined to not be effective in the chemotherapy of patients.

In contrast, if at least one of the FTSJ1 inhibitor sensitivity-related gene markers is detected in the sample, the FTSJ1 inhibitor is determined to be effective in the chemotherapy of patients.

The kit is not particularly limited as long as it uses a mechanism of detecting the gene marker in a sample. In an embodiment, for example, cDNA is obtained from a sample, and amplified by PCR to detect the gene marker. In this case, the kit of the present invention preferably contains a primer for each genetic marker for performing PCR.

The embodiments of the present invention are explained above; however, the present invention is not limited thereto. The present invention can be performed in various forms as long as these forms do not depart from the gist of the present invention.

### EXAMPLES

The embodiments of the present invention are described in more detail based on Examples. However, the present invention is not limited to these Examples.

### Experimental Examples

### Screening

Human gastric cancer cell line NUGC3 was cultured in a DMEM medium (high glucose with L-glutamine and phenol red, produced by FUJIFILM Wako Pure Chemical Corporation) containing 10% fetal bovine serum (produced by Thermo Fisher Scientific Inc.) and penicillin-streptomycin (produced by FUJIFILM Wako Pure Chemical Corporation) (this medium is simply referred to below as “DMEM+10% FBS+1×P/S”). Then, the NUGC3 cells were seeded in a 60-mm culture dish (produced by BioLite) so that the cells were 80% confluent after 24 hours. Subsequently, 5 μg of a reporter plasmid and 15 μL of a lipofection reagent (transIT-LT1, produced by Mirus Bio LLC) were mixed in 500 μL of Opti-MEM medium (produced by Thermo Fisher Scientific, Inc.) to form a complex, which was added to the medium in which the NUGC3 cells were cultured in the 60-mm culture dish. For use as the reporter plasmid, 5 μg of a polyglutamine luciferase reporter was introduced (in the polyglutamine luciferase reporter, a Renilla luciferase expressed in the IRES was located as an internal standard downstream of the sequence of a firefly luciferase sequence to which polyglutamine was added). Alternatively, a YAP/TAZ activity reporter was used by simultaneously introducing 3 μg of 8×GTIIC plasmid and 2 μg of a Renilla luciferase reporter as an internal standard. After another 24 hours, the cells were exfoliated with a 0.05 w/v % trypsin-0.53 mmol/l EDTA-4Na solution (produced by FUJIFILM Wako Pure Chemical Corporation), and then seeded in a 96-well plate (produced by BioLite) so that the cells were 80% confluent after 24 hours. Twenty-four hours after seeding in the 96-well plate, the medium in each well was replaced with 100 μL of a medium (DMEM+10% FBS+an antibiotic) containing 10 μM or 5 μM of a compound for evaluation. After another 24 hours, the medium containing the compound was removed, and the 96-well plate was transferred on ice. Each well was washed with 100 μL of phosphate buffer (PBS, produced by FUJIFILM Wako Pure Chemical Corporation). Thereafter, 20 μL of Passive Lysis Buffer (1×) contained in a Dual-Luciferase Reporter Assay System (produced by Promega Corporation) was added. The plate was then gently shaken for 15 minutes at room temperature to lyse the cells. After confirming that the cells were lysed, 10 μL of the lysate was transferred from each well to each corresponding well of a white 96-well plate (produced by Greiner Bio-one). To each well of the white 96-well plate was added 100 μL of a mixture of a Luciferase Assay Buffer II and Luciferase Assay Substrate contained in the Dual-Luciferase Reporter Assay System (produced by Promega Corporation), whereby the luminescence of the firefly luciferase was induced, and the luminescence intensity was detected with a microluminometer (produced by Berthold Japan K.K.). Subsequently, 100 μL of a mixture of Stop & Glo Buffer and Stop & Glo Substrate contained in the Dual-Luciferase Reporter Assay System (produced by Promega Corporation) was added, whereby the luminescence of firefly luciferase was quenched while the luminescence of Renilla luciferase was induced, and the luminescence intensity was detected with a microluminometer (produced by Berthold Japan K.K.) in a manner similar to the above. The ratio of the luminescence intensity of the firefly luciferase and the luminescence intensity of the Renilla luciferase was calculated, and the luminescence intensity (%) of the wells containing each compound was calculated with the luminescence intensity of the well containing DMSO (dimethyl sulfoxide, used as a solvent for the compound liquids) taken as 100%. Table 1 below shows the results of inhibition of each compound. Unless otherwise specified, the measured values in the table represent the results obtained by evaluation with the addition of 10 μM of each compound for evaluation.

TABLE 1

| Structure | Chemical No. | Polyglutamine | 8×GTIIC |
|---|---|---|---|
| 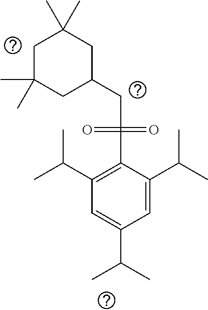 | PVZF2001 | 19.01 (10 μM) 41.05 (5 μM) | 20.02 (10 μM) 40.38 (5 μM) |
| 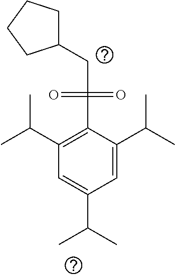 | PVZF2002 | 36.92 | 38.2 |
| 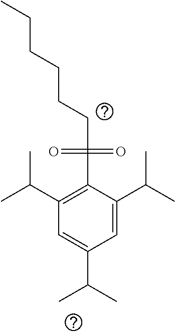 | PVZF2003 | 41.74 | 75.26 |
| 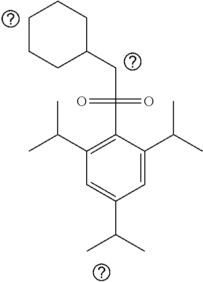 | PVZF2005 | 63.28 | 43.24 |
| 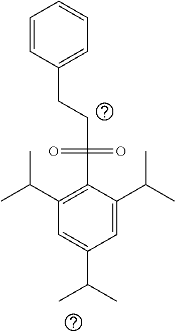 | PVZF2006 | 41.3 | 58.43 |
| 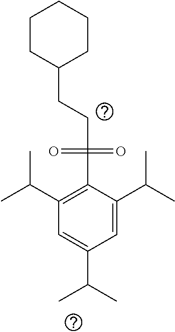 | PVZF2007 | 87.24 | 155.8 |
| 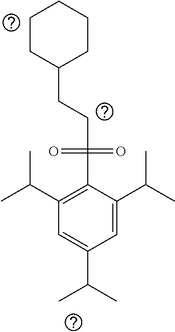 | PVZF2008 | 64.94 | 70.12 |
| 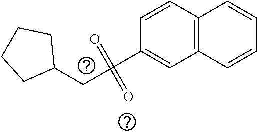 | PVZF2010 | 33.13 | 24.49 |
| 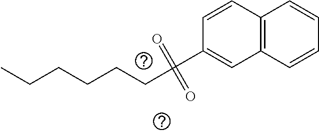 | PVZF2011 | 28.85 | 36.2 |
| 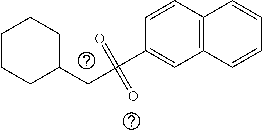 | PVZF2012 | 33.76 | 34.08 |
| 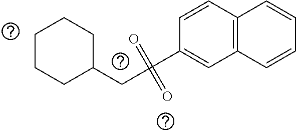 | PVZF2013 | 99.66 | 94.64 |
| 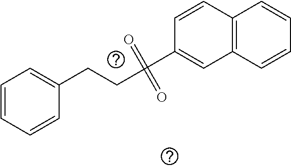 | PVZF2014 | 40.32 | 59.08 |
| 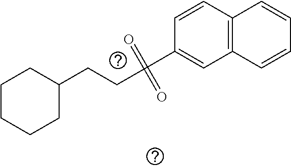 | PVZF2015 | 51.29 | 51.25 |
| 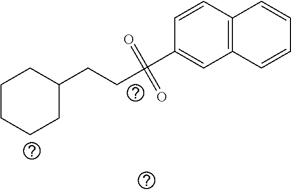 | PVZF2016 | 96.2 | 94.97 |
| 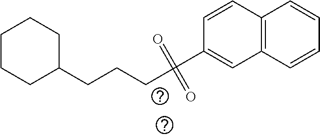 | PVZF2017 | 66.66 | 58.91 |
| 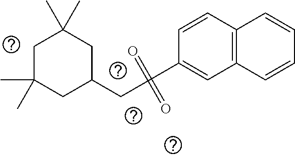 | PVZF2018 | 61.19 | 73.29 |
| 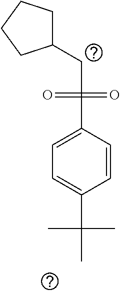 | PVZF2019 | 31.45 | 36.82 |
| 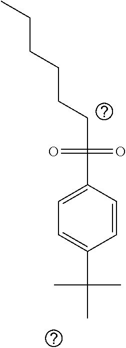 | PVZF2020 | 36.76 | 45.04 |
| 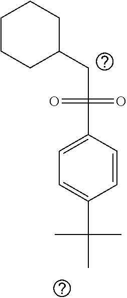 | PVZF2021 | 32.95 | 44.55 |
| 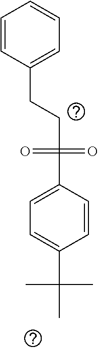 | PVZF2023 | 58.32 | 66.96 |
| 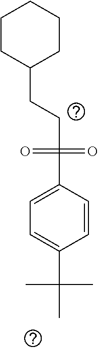 | PVZF2024 | 63.4 | 55.45 |
| 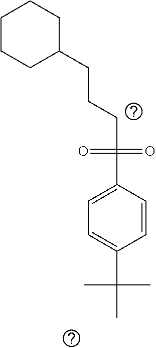 | PVZF2026 | 31.89 | 34.4 |
| 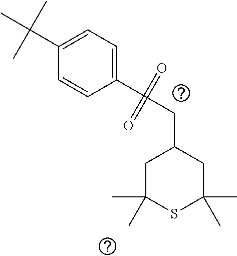 | PVZF2027 | 54.28 | 64.18 |
| 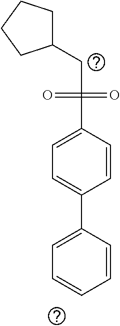 | PVZF2028 | 42.32 | 40.24 |
| 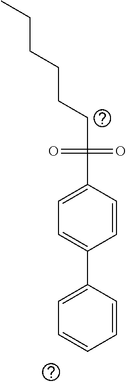 | PVZF2029 | 49.5 | 49.28 |
| 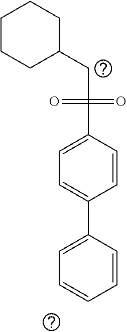 | PVZF2030 | 50.36 | 49.29 |
| 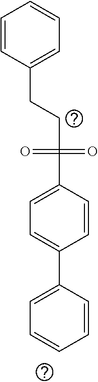 | PVZF2032 | 73.08 | 76.99 |
| 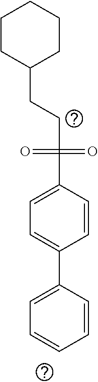 | PVZF2033 | 67.7 | 69.36 |
| 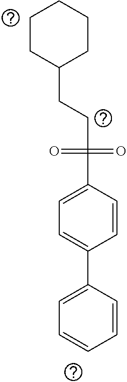 | PVZF2034 | 73.32 | 67.59 |
| 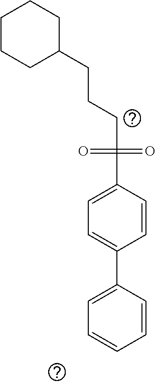 | PVZF2035 | 58.85 | 71.12 |
| 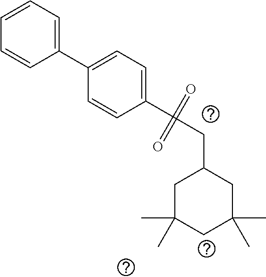 | PVZF2036 | 79.76 | 70.46 |
| 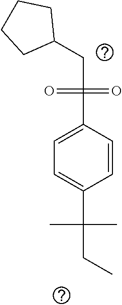 | PVZF2037 | 51.06 | 43.28 |
| 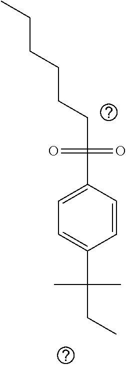 | PVZF2038 | 58.2 | 56.84 |
| 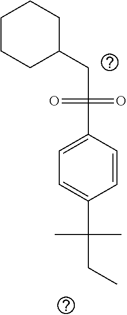 | PVZF2039 | 53.6 | 60 |
| 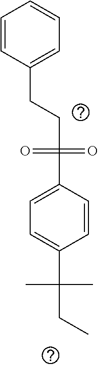 | PVZF2041 | 78.87 | 79.61 |
| 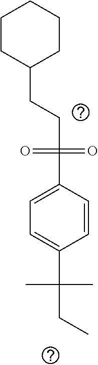 | PVZF2042 | 34.02 | 35.98 |
| 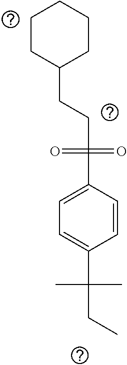 | PVZF2043 | 79.21 | 107.9 |
| 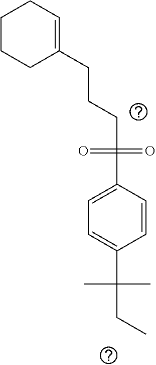 | PVZF2044 | 45.42 | 55.05 |
| 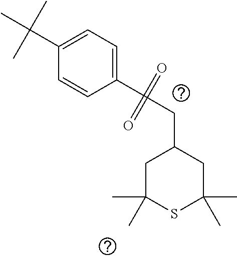 | PVZF2045 | 65.16 | 66.26 |
| 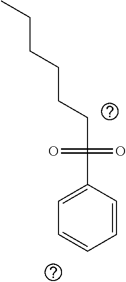 | PVZF2047 | 48.95 | 63.56 |
| 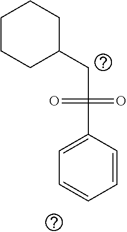 | PVZF2048 | 73.33 | 69.86 |
| 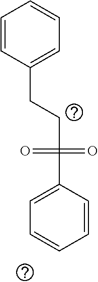 | PVZF2050 | 49.43 | 39.57 |
| 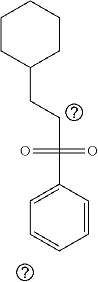 | PVZF2051 | 38.06 | 35.1 |
| 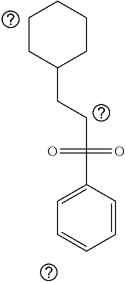 | PVZF2052 | 87.34 | 92.55 |
| 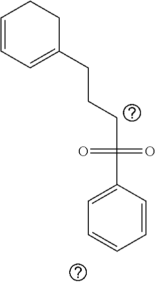 | PVZF2053 | 49.63 | 52.43 |
| 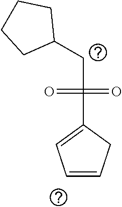 | PVZF2055 | 67.66 | 73.97 |
| 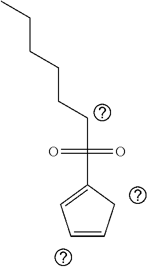 | PVZF2056 | 53.21 | 57.21 |
| 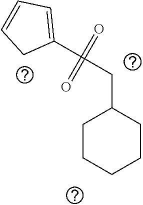 | PVZF2057 | 74.93 | 68.03 |
| 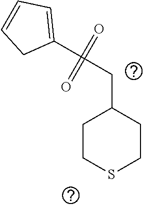 | PVZF2058 | 84.3 | 87.86 |
| 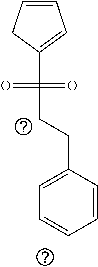 | PVZF2059 | 47.64 | 52.27 |
| 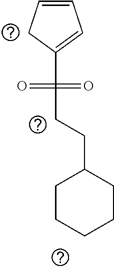 | PVZF2060 | 38.67 | 47.23 |
| 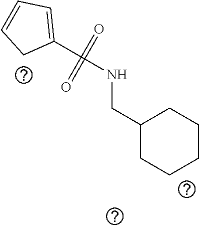 | PVZF2061 | 98.03 | 111 |
| 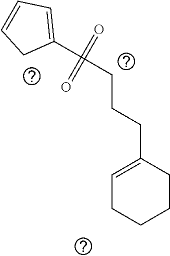 | PVZF2062 | 56.54 | 69.32 |
| 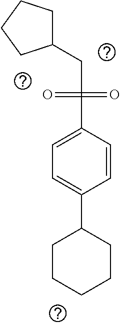 | PVZF2064 | 64 | 75.47 |
| 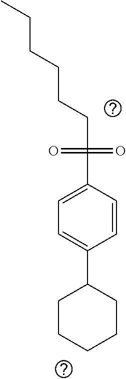 | PVZF2065 | 60.68 | 70.27 |
| 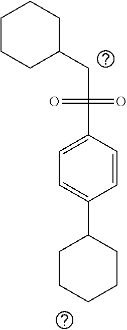 | PVZF2066 | 35.48 | 31.11 |
| 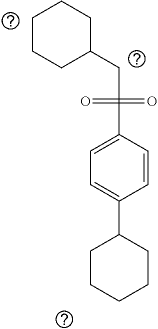 | PVZF2067 | 96.94 | 88.84 |
| 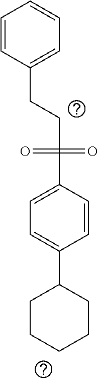 | PVZF2068 | 53.21 | 74.24 |
| 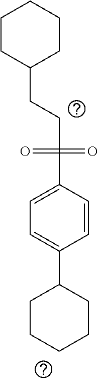 | PVZF2069 | 46.92 | 60.88 |
| 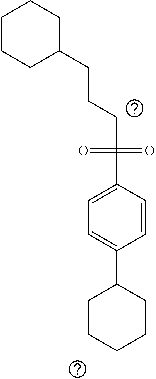 | PVZF2071 | 63.89 | 70.27 |
| 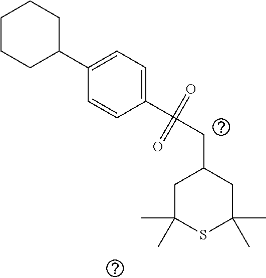 | PVZF2072 | 80.1 | 83.58 |
| 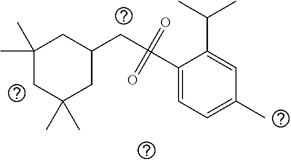 | PVZF2074 | 51.3 | 67.6 |
| 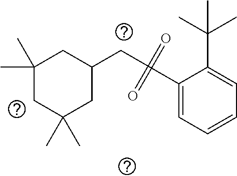 | PVZF2075 | 67.3 | 64.7 |
| 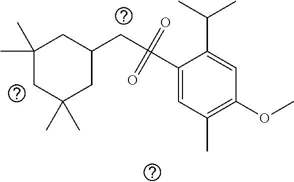 | PVZF2076 | 46.5 | 66.6 |
| 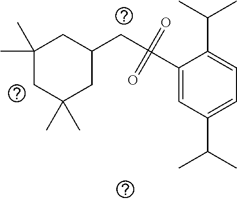 | PVZF2077 | 39.9 | 61.9 |
| 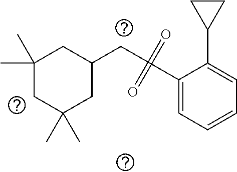 | PVZF2078 | 82.7 | 107.8 |
| 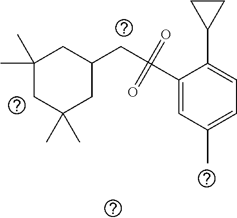 | PVZF2079 | 74.5 | 103.7 |
| 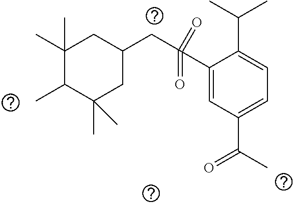 | PVZF2081 | 80.7 | 82.6 |
| 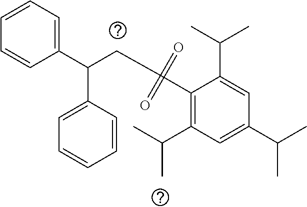 | PVZF2082 | 52.7 | 70.4 |
| 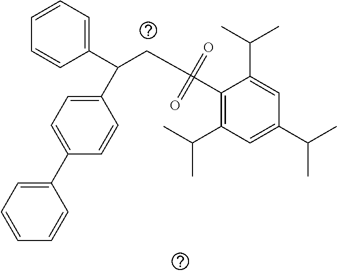 | PVZF2083 | 69 | 108.4 |
| 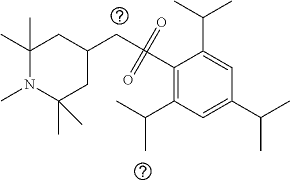 | PVZF2085 | 90.6 | 124.9 |
| 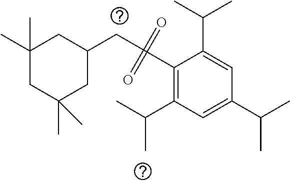 | PVZF2086 | 71.4 | 108.5 |
| 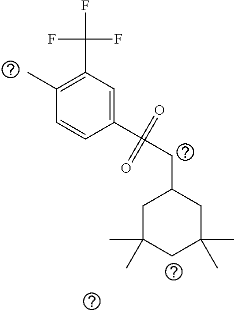 | SA001 | 83.34 | 74.98 |
| 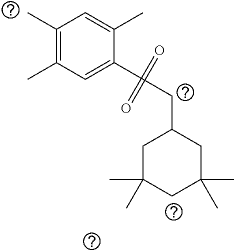 | SA002 | 49.58 | 47.03 |
| 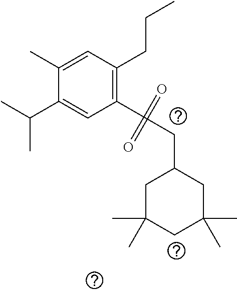 | SA003 | 37.79 | 46.84 |
| 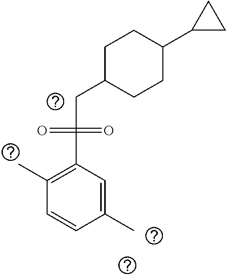 | SA004 | 51.07 | 52.64 |
| 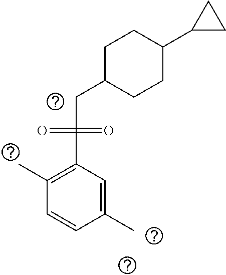 | SA005 | 76.08 | 90.78 |
| 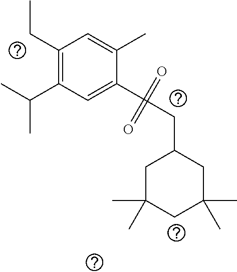 | SA006 | 37.54 | 40.21 |
| 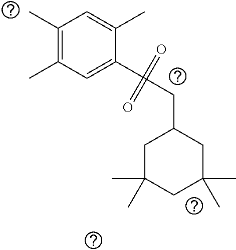 | SA007 | 55.29 | 56.96 |
| 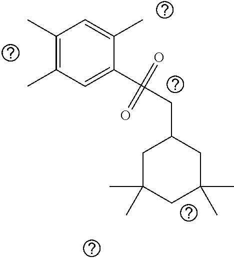 | SA008 | 51.03 | 57.06 |
| 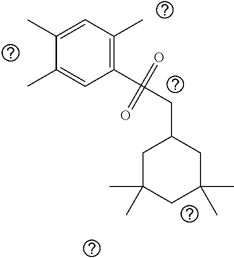 | SA009 | 73.02 | 80.29 |
| 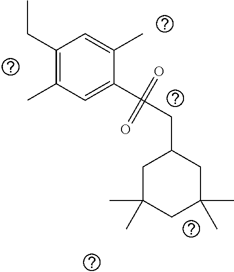 | SA010 | 59.61 | 64.43 |
| 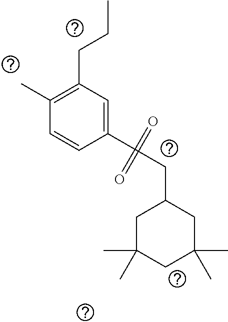 | SA011 | 60.35 | 67.54 |
| 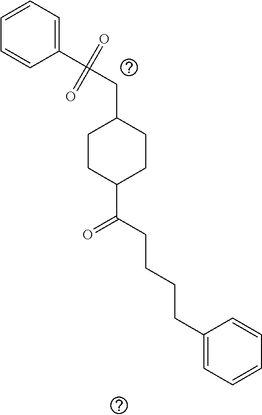 | SA012 | 86.48 | 91.92 |
| 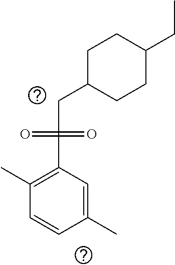 | SA013 | 77.67 | 101.6 |
| 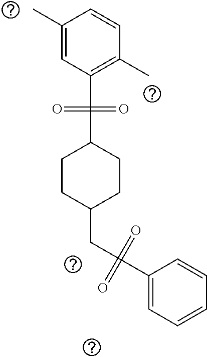 | SA014 | 72.95 | 93.69 |
| 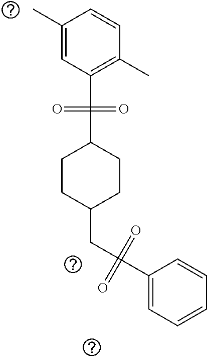 | SA015 | 60.36 | 69.21 |
| 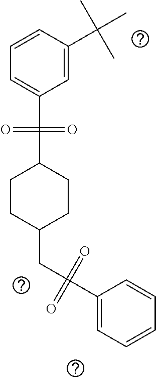 | SA016 | 43.01 | 53.02 |
| 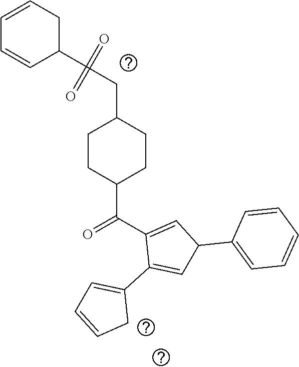 | SA017 | 50.98 | 79.52 |
| 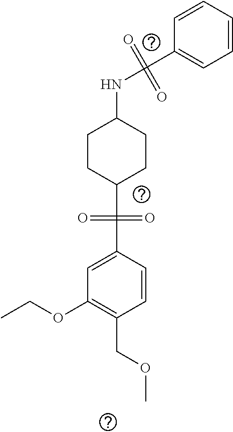 | SA018 | 78.85 | 74.98 |
| 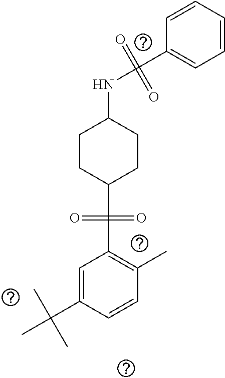 | SA019 | 55.62 | 66.45 |
| 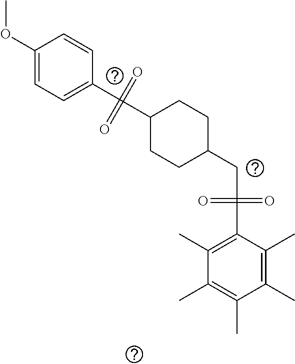 | SA020 | 27.71 | 36.19 |
| 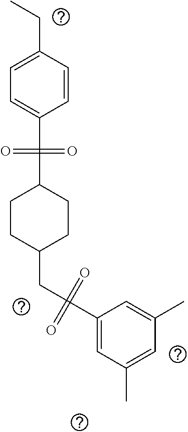 | SA021 | 44.6 | 70.64 |
| 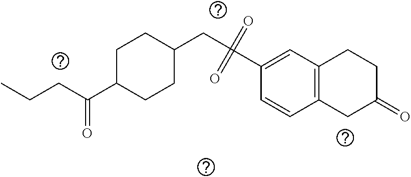 | SA022 | 102.2 | 66.88 |
| 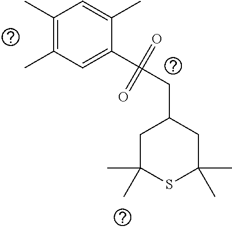 | SA023 | 63.47 | 87.74 |
| 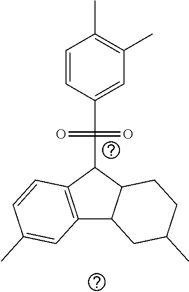 | SA024 | 59.36 | 65.63 |
| 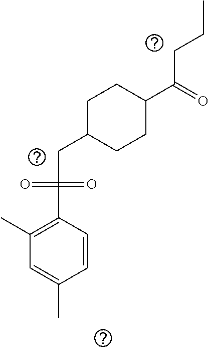 | SA025 | 49.45 | 46.27 |
| 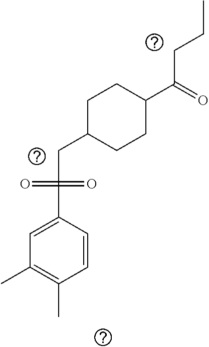 | SA026 | 63.06 | 46.21 |
| 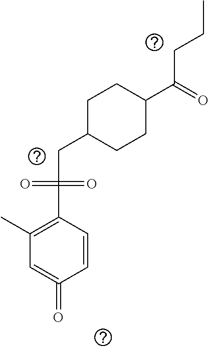 | SA027 | 95.84 | 30.08 |
| 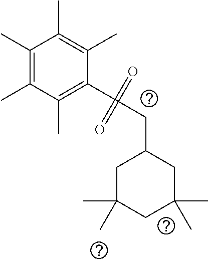 | SA028 | 60.03 | 59.49 |
| 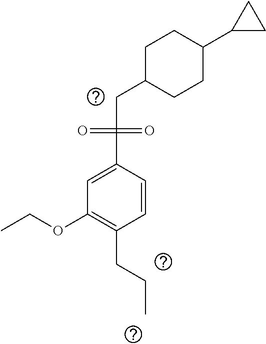 | SA029 | 58.64 | 65.65 |
| 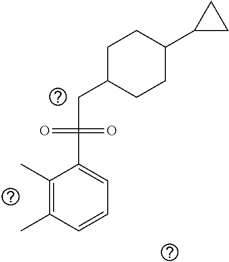 | SA030 | 86.26 | 73.56 |
| 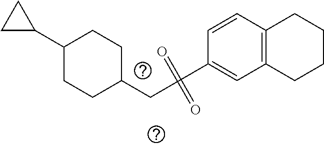 | SA031 | 62.23 | 76.14 |
| 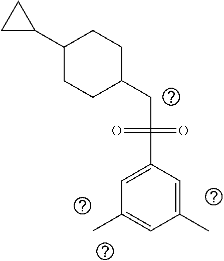 | SA032 | 80.29 | 88.64 |
| 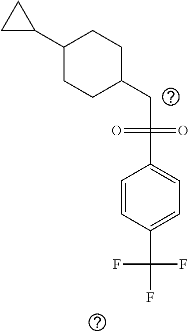 | SA033 | 96.01 | 96.16 |
| 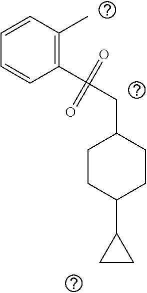 | SA034 | 77.96 | 87.42 |
| 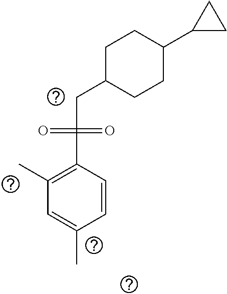 | SA035 | 98.96 | 116.6 |
| 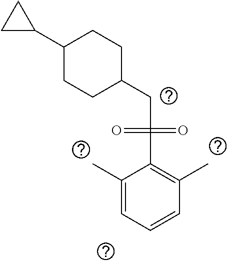 | SA036 | 71.57 | 57.59 |
| 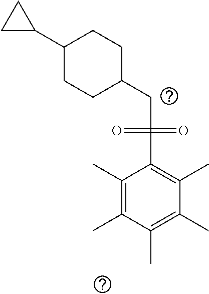 | SA037 | 42.93 | 35.53 |
| 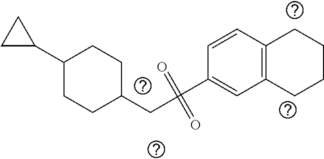 | SA038 | 69.77 | 94.4 |
| 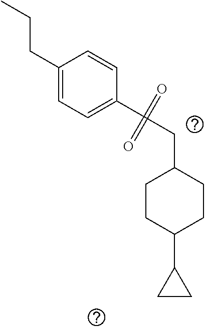 | SA039 | 97.44 | 56.65 |
| 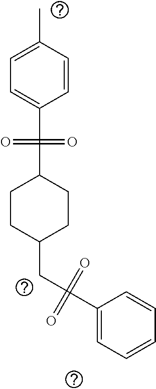 | SA040 | 44.87 | 51.54 |
| 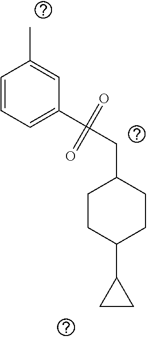 | SA041 | 88.82 | 87.17 |
| 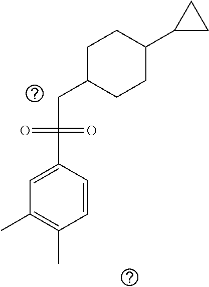 | SA042 | 94.62 | 100.9 |
| 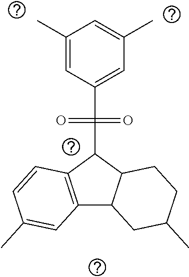 | SA043 | 90.09 | 124.5 |
| 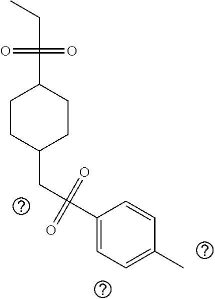 | SA044 | 87.25 | 63.33 |
| 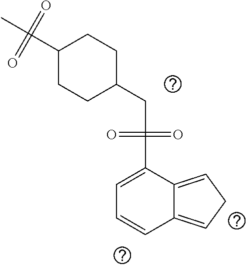 | SA045 | 111 | 75.33 |
| 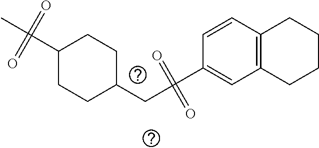 | SA046 | 75.23 | 83.81 |
| 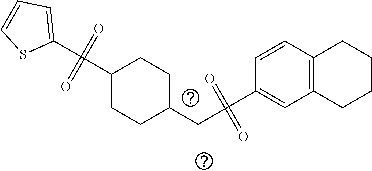 | SA047 | 47.4 | 56.12 |
| 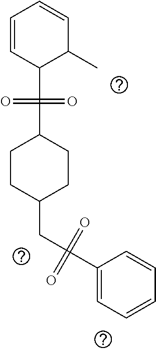 | SA048 | 35.33 | 43.56 |
| 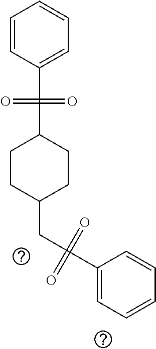 | SA049 | 39.58 | 43.7 |
| 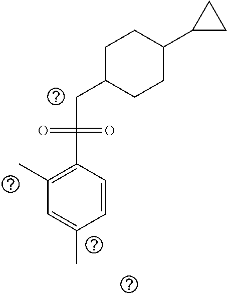 | SA050 | 60.42 | 92.26 |
| 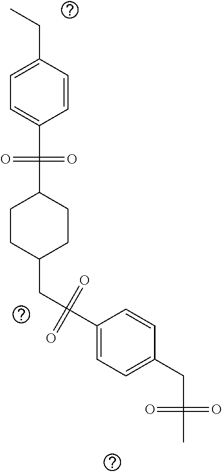 | SA051 | 42.46 | 37.26 |
| 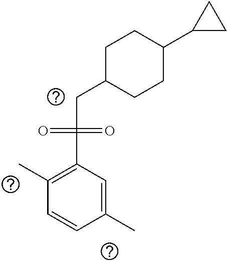 | SA052 | 47.69 | 54.65 |
| 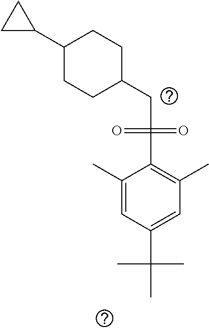 | SA053 | 35.39 | 55.02 |
| 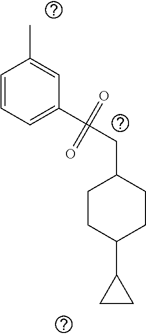 | SA054 | 109.1 | 83.34 |
| 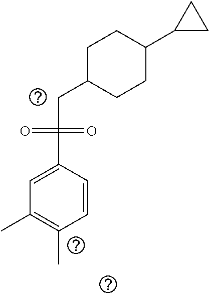 | SA055 | 81.09 | 82.18 |
| 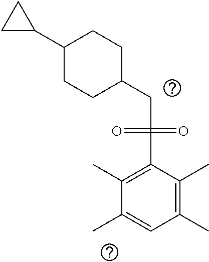 | SA056 | 83.19 | 102.1 |
| 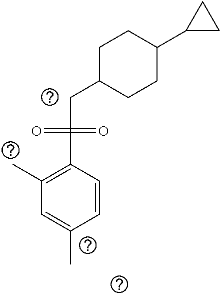 | SA057 | 79.6 | 73.76 |
| 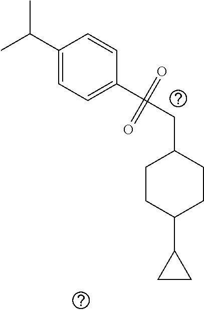 | SA058 | 76.81 | 35.45 |
| 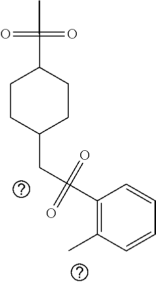 | SA059 | 100.9 | 88.99 |
| 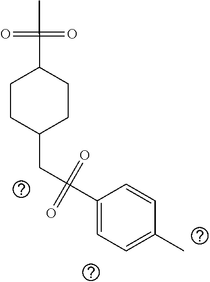 | SA060 | 92.41 | 90.52 |
| 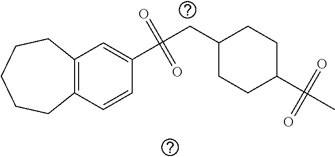 | SA061 | 66.4 | 96.27 |
| 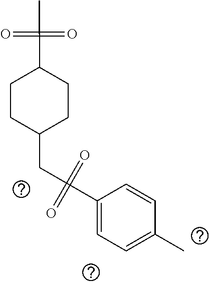 | SA062 | 93.21 | 100.9 |
| 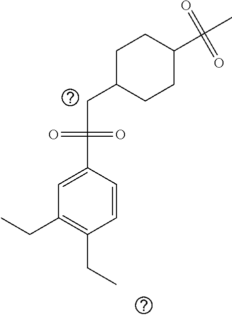 | SA063 | 78.75 | 93.35 |
| 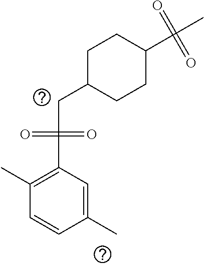 | SA064 | 54.39 | 64.48 |
| 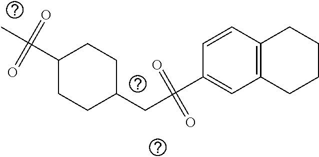 | SA065 | 47.58 | 39.43 |
| 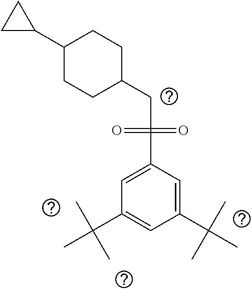 | SA066 | 66.16 | 64.4 |
| 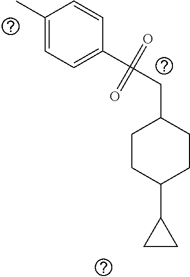 | SA067 | 75.3 | 75.49 |
| 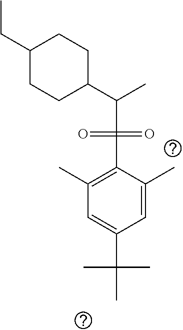 | SA068 | 81.58 | 110.2 |
| 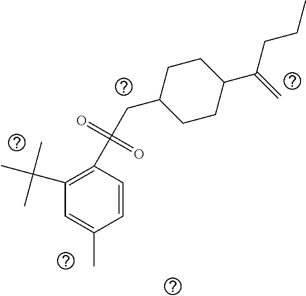 | SA069 | 76.83 | 67.61 |
| 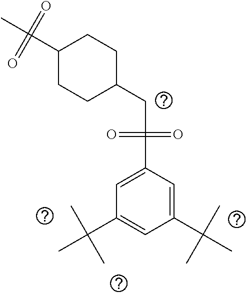 | SA070 | 59.04 | 109.7 |
| 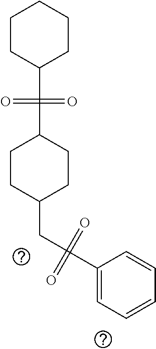 | SA071 | 61.72 | 138.5 |
| 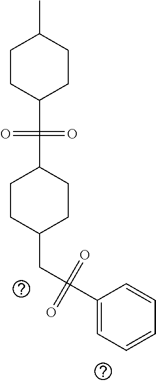 | SA072 | 73.98 | 68.16 |
| 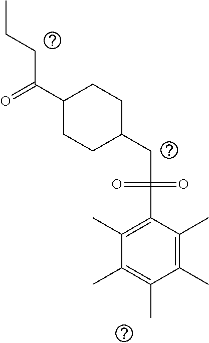 | SA073 | 60.47 | 52.33 |
| 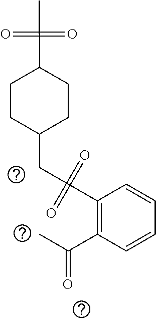 | SA074 | 92.5 | 88.44 |
| 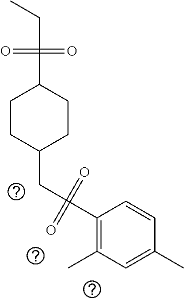 | SA075 | 110.7 | 95.02 |
| 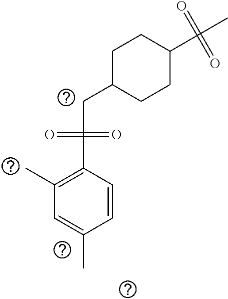 | SA076 | 98.03 | 64.29 |
| 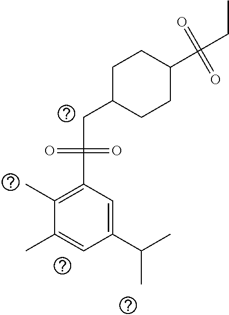 | SA077 | 65.09 | 66.59 |
| 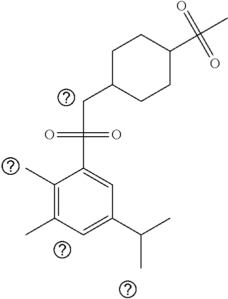 | SA078 | 87.12 | 95.87 |
| 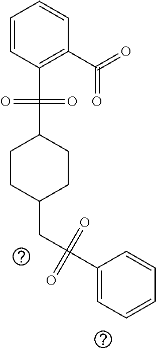 | SA079 | 50.51 | 61.75 |
| 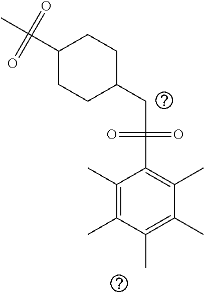 | SA080 | 55.01 | 62.45 |
| 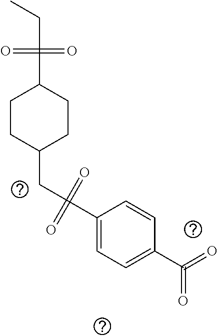 | SA081 | 77.49 | 104.4 |
| 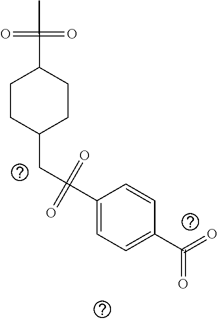 | SA082 | 51.66 | 95.64 |
| 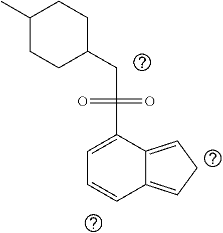 | SA083 | 93.28 | 74.52 |
| 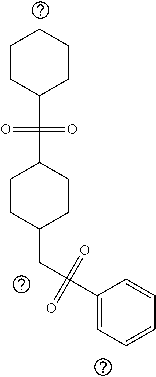 | SA084 | 108.6 | 91.76 |
| 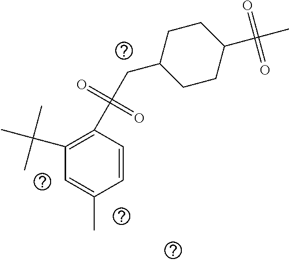 | SA085 | 86 | 83.09 |
| 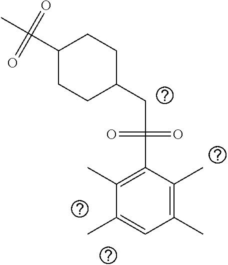 | SA086 | 33.07 | 40.75 |
| 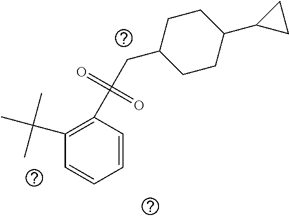 | SA087 | 78.43 | 59.57 |
| 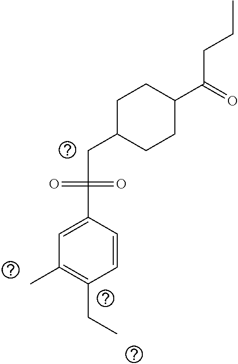 | SA088 | 65.61 | 47.6 |
| 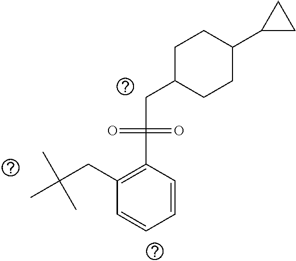 | SA089 | 56.61 | 50.1 |
| 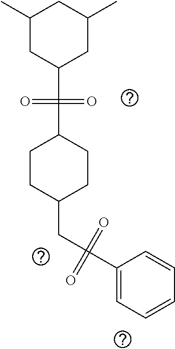 | SA090 | 84.73 | 64.02 |
| 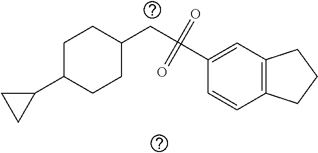 | SA091 | 88.36 | 101.3 |
| 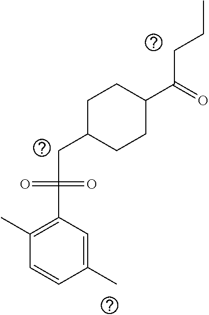 | SA092 | 67.66 | 73.04 |
| 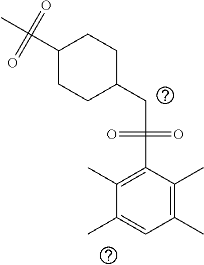 | SA093 | 38.43 | 39.06 |
| 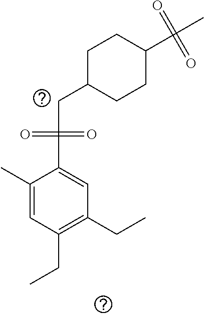 | SA094 | 65.61 | 56.61 |
| 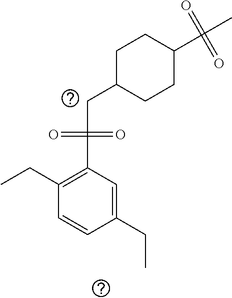 | SA095 | 44.97 | 67.3 |
| 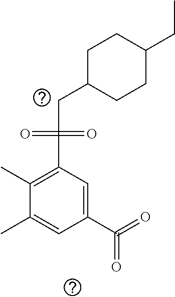 | SA096 | 81.13 | 102.2 |
| structure | Chemical No. | [symbol] | 8×GTIIC |
| 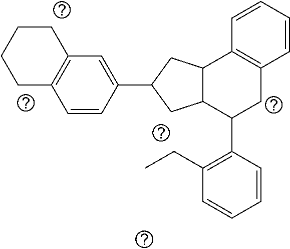 [SMILES: CCc1ccccc1C1Cc2ccccc2C2CC(c3ccc4c(c3)CCCC4)CC12] | PZ001 | 19.18 | 29.63 |
| 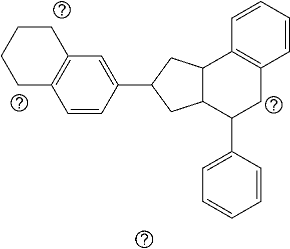 [SMILES: c1ccc(C2Cc3ccccc3C3CC(c4ccc5c(c4)CCCC5)CC23)cc1] | PZ002 | 37.71 | 64.05 |
| 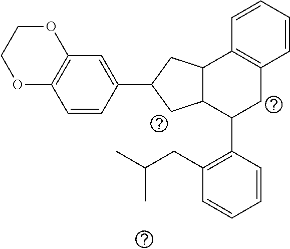 [SMILES: CC(C)Cc1ccccc1C1Cc2ccccc2C2CC(c3ccc4c(c3)OCCO4)CC12] | PZ003 | 23.24 | 43.64 |
| 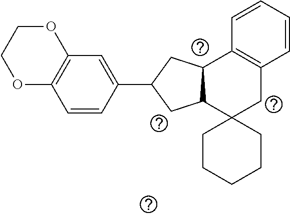 | PZ004 | 35.59 | 54.69 |
| 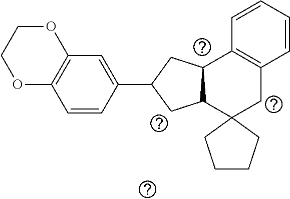 | PZ005 | 24.83 | 33.54 |
| 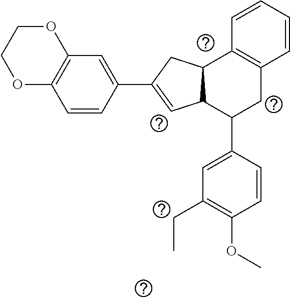 | PZ006 | 18.37 | 30.88 |
| 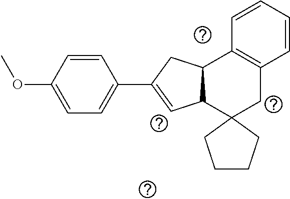 | PZ007 | 82.36 | 129 |
| 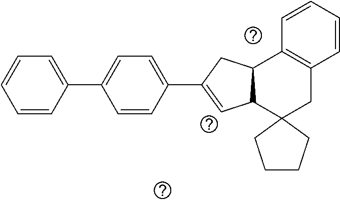 | PZ008 | 79.23 | 99.52 |
| 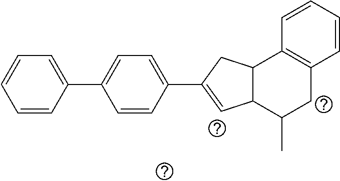 [SMILES: CC1Cc2ccccc2C2CC(c3ccc(-c4ccccc4)cc3)=CC12] | PZ009 | 66.76 | 105.3 |
| 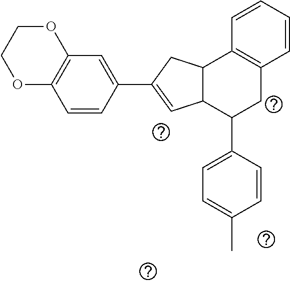 [SMILES: Cc1ccc(C2Cc3ccccc3C3CC(c4ccc5c(c4)OCCO5)=CC23)cc1] | PZ010 | 65.58 | 83.2 |
| 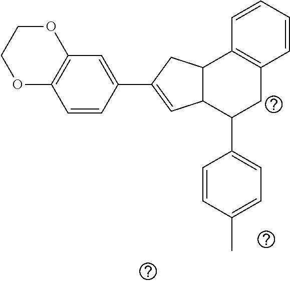 [SMILES: Cc1ccc(C2Cc3ccccc3C3CC(c4ccc5c(c4)OCCO5)=CC23)cc1] | PZ011 | 78.31 | 103.7 |
| 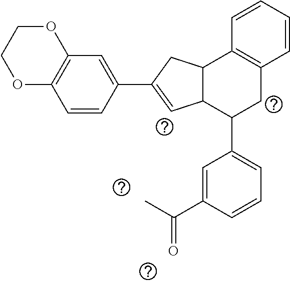 [SMILES: CC(=O)c1cccc(C2Cc3ccccc3C3CC(c4ccc5c(c4)OCCO5)=CC23)c1] | PZ012 | 93.87 | 150.8 |
| 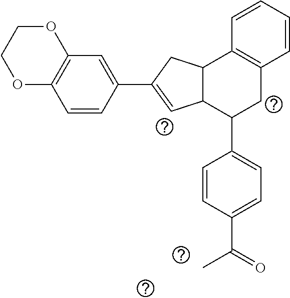 [SMILES: CC(=O)c1ccc(C2Cc3ccccc3C3CC(c4ccc5c(c4)OCCO5)=CC23)cc1] | PZ013 | 66.01 | 100 |
| 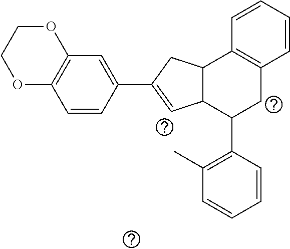 [SMILES: Cc1ccccc1C1Cc2ccccc2C2CC(c3ccc4c(c3)OCCO4)=CC12] | PZ014 | 70.81 | 105.5 |
| 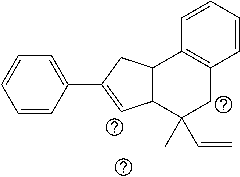 [SMILES: C=CC1(C)Cc2ccccc2C2CC(c3ccccc3)=CC21] | PZ015 | 52.43 | 107.7 |
| 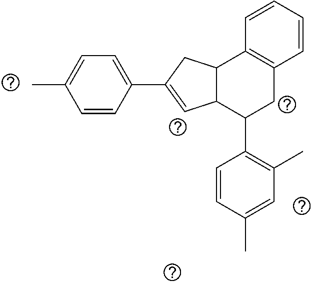 | PZ016 | 82.72 | 103.6 |
| 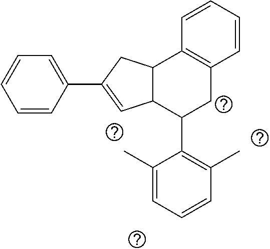 [SMILES: Cc1cccc(C)c1C1Cc2ccccc2C2CC(c3ccccc3)=CC21] | PZ017 | 95.99 | 116.7 |
| 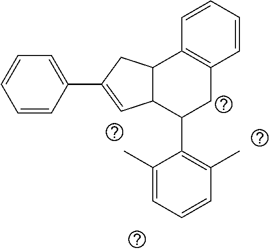 [SMILES: Cc1cccc(C)c1C1Cc2ccccc2C2CC(c3ccccc3)=CC21] | PZ018 | 74.68 | 112 |
| 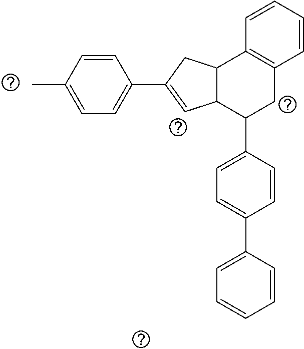 [SMILES: Cc1ccc(C2=CC3C(c4ccc(-c5ccccc5)cc4)Cc4ccccc4C3C2)cc1] | PZ019 | 90.41 | 152.3 |
| 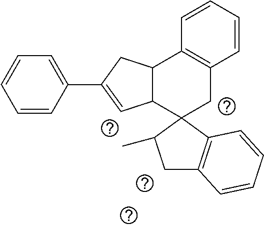 [SMILES: CC1Cc2ccccc2C12Cc1ccccc1C1CC(c3ccccc3)=CC12] | PZ020 | 37.56 | 71.35 |
| 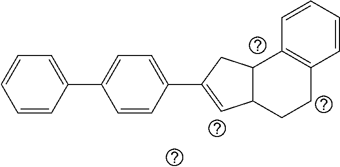 [SMILES: C1=C(c2ccc(-c3ccccc3)cc2)CC2c3ccccc3CCC12] | PZ021 | 96.99 | 128 |
| 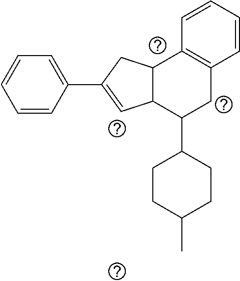 [SMILES: CC1CCC(C2Cc3ccccc3C3CC(c4ccccc4)=CC32)CC1] | PZ022 | 93.11 | 121.4 |
| 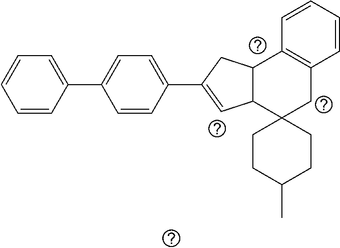 [SMILES: CC1CCC2(CC1)Cc1ccccc1C1CC(c3ccc(-c4ccccc4)cc3)=CC12] | PZ023 | 42.63 | 59.03 |
| 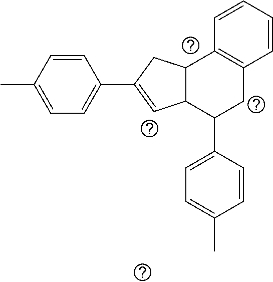 [SMILES: Cc1ccc(C2=CC3C(c4ccc(C)cc4)Cc4ccccc4C3C2)cc1] | PZ024 | 74.04 | 102.5 |
| 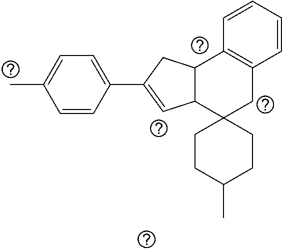 [SMILES: Cc1ccc(C2=CC3C(C2)c2ccccc2CC32CCC(C)CC2)cc1] | PZ025 | 86.63 | 125.1 |
| 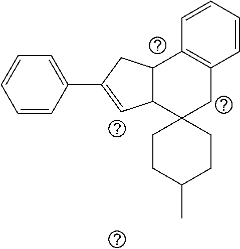 [SMILES: CC1CCC2(CC1)Cc1ccccc1C1CC(c3ccccc3)=CC12] | PZ026 | 72.2 | 36.55 |
| 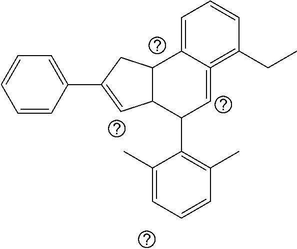 | PZ027 | 75.97 | 120.1 |
| 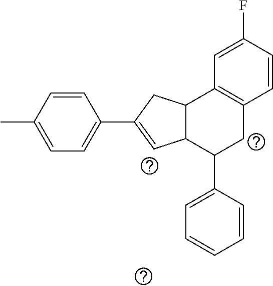 [SMILES: Cc1ccc(C2=CC3C(c4ccccc4)Cc4ccc(F)cc4C3C2)cc1] | PZ028 | 51.11 | 67.71 |
| 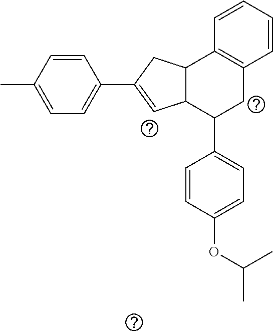 [SMILES: Cc1ccc(C2=CC3C(c4ccc(OC(C)C)cc4)Cc4ccccc4C3C2)cc1] | PZ029 | 63.67 | 105.6 |
| 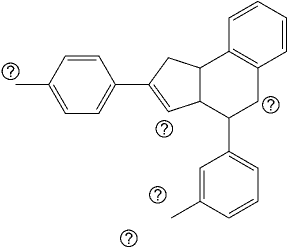 [SMILES: Cc1ccc(C2=CC3C(c4cccc(C)c4)Cc4ccccc4C3C2)cc1] | PZ030 | 76.11 | 82.09 |
| 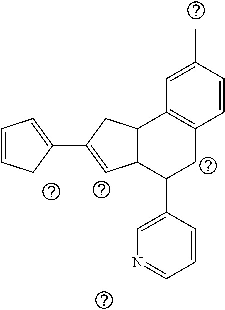 [SMILES: Cc1ccc2c(c1)C1CC(C3=CC=CC3)=CC1C(c1cccnc1)C2] | PZ031 | 54 | 61.92 |
| 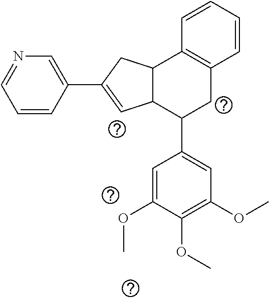 [SMILES: COc1cc(C2Cc3ccccc3C3CC(c4cccnc4)=CC23)cc(OC)c1OC] | PZ032 | 87.58 | 76.29 |
| 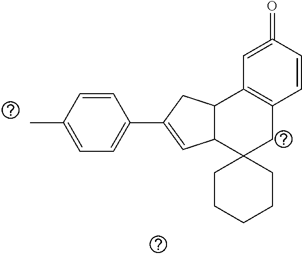 | PZ033 | 43.62 | 64.98 |
| 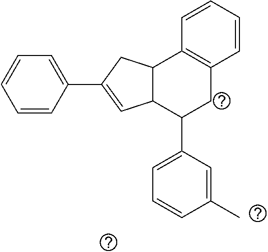 [SMILES: Cc1cccc(C2Cc3ccccc3C3CC(c4ccccc4)=CC23)c1] | PZ034 | 53.73 | 105.4 |
| 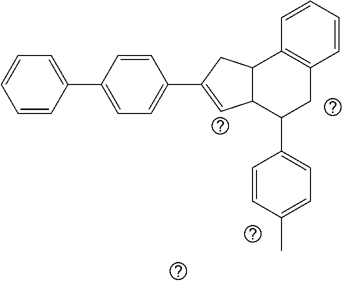 [SMILES: Cc1ccc(C2Cc3ccccc3C3CC(c4ccc(-c5ccccc5)cc4)=CC23)cc1] | PZ035 | 61.23 | 91.89 |
| 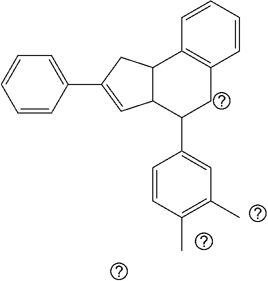 [SMILES: Cc1ccc(C2Cc3ccccc3C3CC(c4ccccc4)=CC23)cc1C] | PZ036 | 55.74 | 61.13 |
| 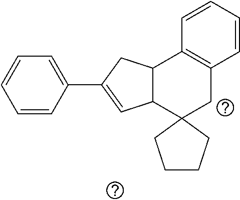 [SMILES: C1=C(c2ccccc2)CC2c3ccccc3CC3(CCCC3)C12] | PZ037 | 76.51 | 115.1 |
| 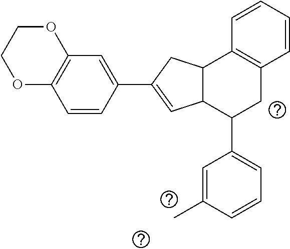 [SMILES: Cc1cccc(C2Cc3ccccc3C3CC(c4ccc5c(c4)OCCO5)=CC23)c1] | PZ038 | 67.66 | 90.72 |
| 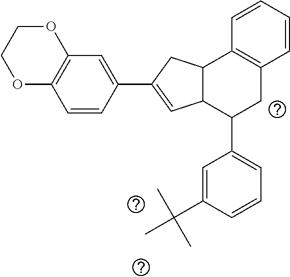 [SMILES: CC(C)(C)c1cccc(C2Cc3ccccc3C3CC(c4ccc5c(c4)OCCO5)=CC23)c1] | PZ039 | 58.55 | 110.5 |
| 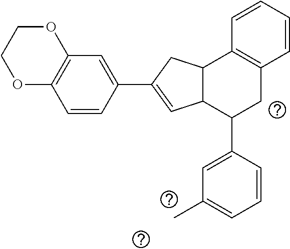 [SMILES: Cc1cccc(C2Cc3ccccc3C3CC(c4ccc5c(c4)OCCO5)=CC23)c1] | PZ040 | 84.86 | 67.19 |
| 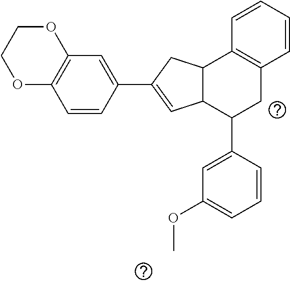 [SMILES: COc1cccc(C2Cc3ccccc3C3CC(c4ccc5c(c4)OCCO5)=CC23)c1] | PZ041 | 57.79 | 105.5 |
| 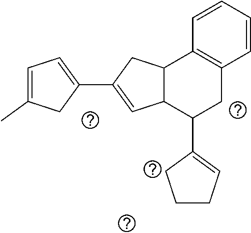 [SMILES: CC1=CC=C(C2=CC3C(C4=CCCC4)Cc4ccccc4C3C2)C1] | PZ042 | 93.02 | 145 |
| 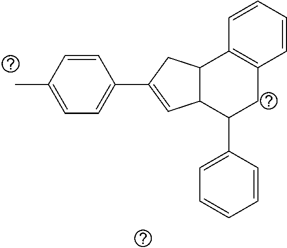 [SMILES: Cc1ccc(C2=CC3C(c4ccccc4)Cc4ccccc4C3C2)cc1] | PZ043 | 70.79 | 106.6 |
| 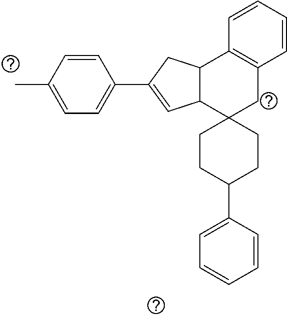 [SMILES: Cc1ccc(C2=CC3C(C2)c2ccccc2CC32CCC(c3ccccc3)CC2)cc1] | PZ044 | 54.5 | 77.6 |
| 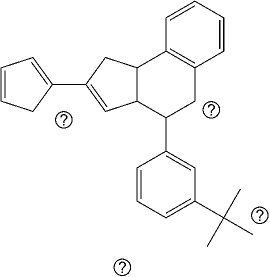 [SMILES: CC(C)(C)c1cccc(C2Cc3ccccc3C3CC(C4=CC=CC4)=CC23)c1] | PZ045 | 36.58 | 66.83 |
| 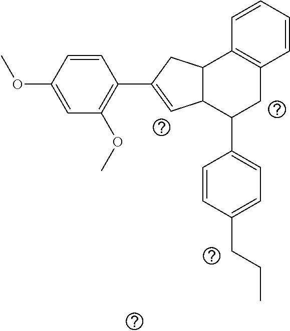 [SMILES: CCCc1ccc(C2Cc3ccccc3C3CC(c4ccc(OC)cc4OC)=CC23)cc1] | PZ046 | 38.57 | 61.21 |
| 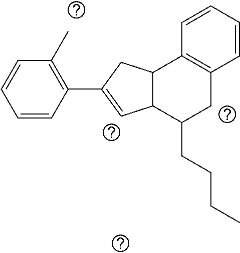 [SMILES: CCCCC1Cc2ccccc2C2CC(c3ccccc3C)=CC12] | PZ047 | 77.8 | 137.7 |
| 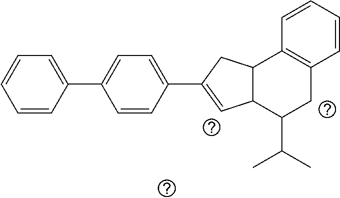 [SMILES: CC(C)C1Cc2ccccc2C2CC(c3ccc(-c4ccccc4)cc3)=CC21] | PZ048 | 56.46 | 109.3 |
| 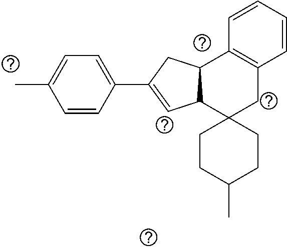 | PZ049 | 64.99 | 112.3 |
| 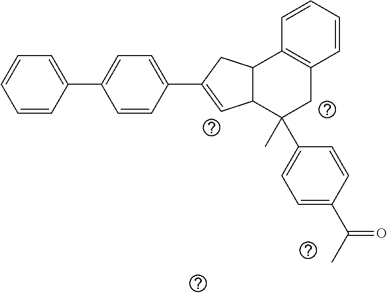 [SMILES: CC(=O)c1ccc(C2(C)Cc3ccccc3C3CC(c4ccc(-c5ccccc5)cc4)=CC32)cc1] | PZ050 | 53.1 | 54.17 |
| 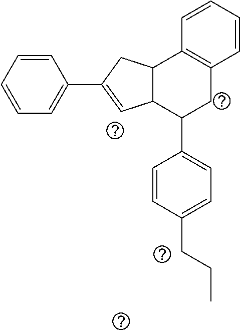 [SMILES: CCCc1ccc(C2Cc3ccccc3C3CC(c4ccccc4)=CC23)cc1] | PZ051 | 65.92 | 100.2 |
| 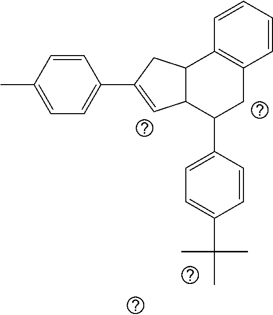 [SMILES: Cc1ccc(C2=CC3C(c4ccc(C(C)(C)C)cc4)Cc4ccccc4C3C2)cc1] | PZ052 | 90.33 | 129.3 |
| 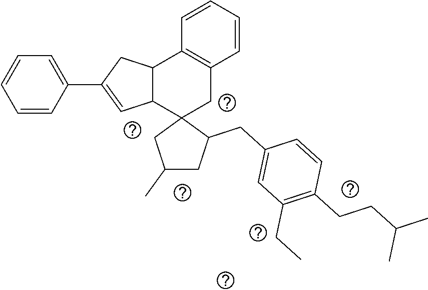 [SMILES: CCc1cc(CC2CC(C)CC23Cc2ccccc2C2CC(c4ccccc4)=CC23)ccc1CCC(C)C] | PZ053 | 52.69 | 55.2 |
| 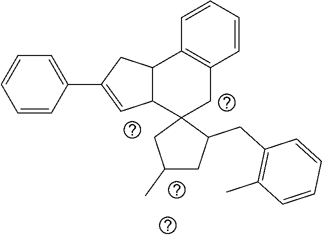 [SMILES: Cc1ccccc1CC1CC(C)CC12Cc1ccccc1C1CC(c3ccccc3)=CC12] | PZ054 | 88.06 | 186.3 |
| 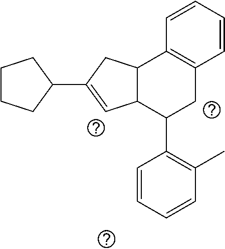 [SMILES: Cc1ccccc1C1Cc2ccccc2C2CC(C3CCCC3)=CC12] | PZ055 | 60.21 | 112.7 |
| 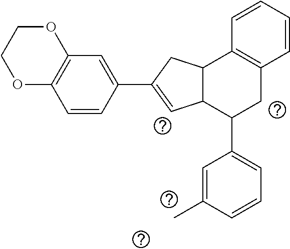 [SMILES: Cc1cccc(C2Cc3ccccc3C3CC(c4ccc5c(c4)OCCO5)=CC23)c1] | PZ056 | 61.76 | 90.61 |
| 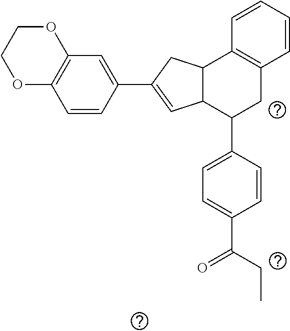 [SMILES: CCC(=O)c1ccc(C2Cc3ccccc3C3CC(c4ccc5c(c4)OCCO5)=CC23)cc1] | PVZF0001 | 166 | 99.6 |
| 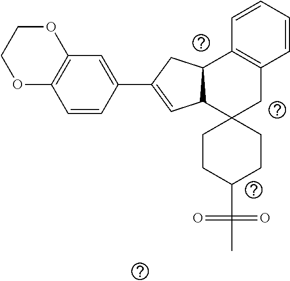 | PVZF0002 | 71 | 67.6 |
| 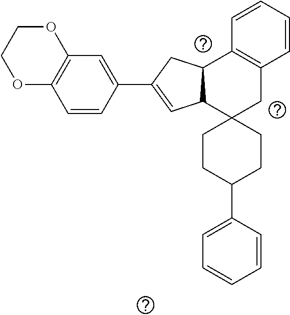 | PVZF0005 | 125 | [symbol] .8 |
| 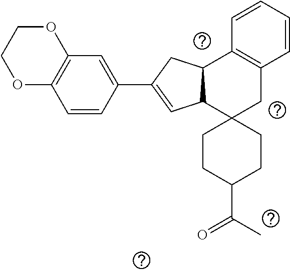 | PVZF0003 | 87 | 119 |
| 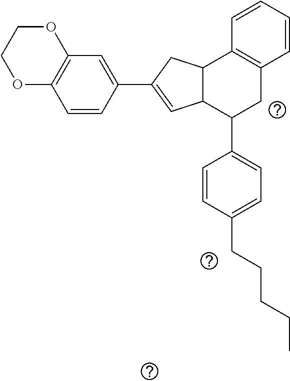 [SMILES: CCCCCc1ccc(C2Cc3ccccc3C3CC(c4ccc5c(c4)OCCO5)=CC23)cc1] | PVZF00 [symbol] | 92 | 144 |
| 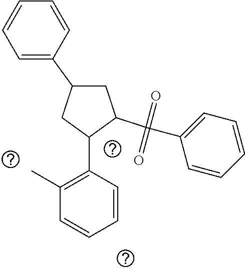 | PZ057 | 44.75 | 48.07 |
| 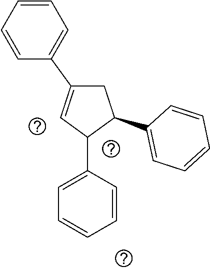 [SMILES: C1=C(c2ccccc2)C[C@@H](c2ccccc2)C1c1ccccc1] | PZ058 | 92.72 | 55.65 |
| 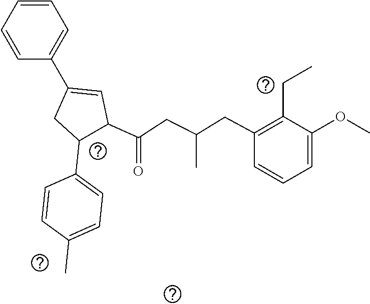 [SMILES: CCc1c(CC(C)CC(=O)C2C=C(c3ccccc3)CC2c2ccc(C)cc2)cccc1OC] | PZ059 | 79.39 | 79.35 |
| 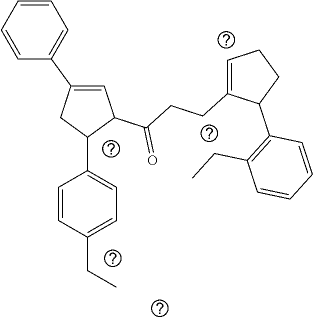 [SMILES: CCc1ccc(C2CC(c3ccccc3)=CC2C(=O)CCC2=CCCC2c2ccccc2CC)cc1] | PZ060 | 53.18 | 42.7 |
| 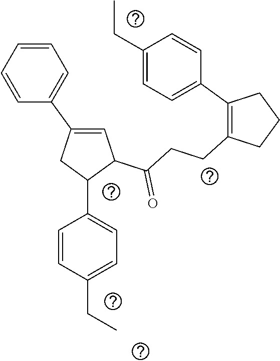 [SMILES: CCc1ccc(C2=C(CCC(=O)C3C=C(c4ccccc4)CC3c3ccc(CC)cc3)CCC2)cc1] | PZ061 | 75.2 | 31.1 |
| 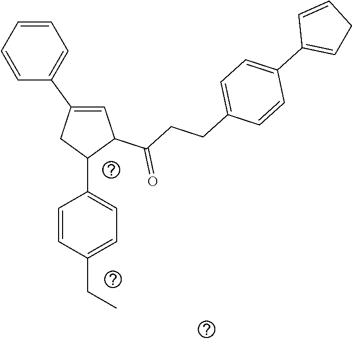 [SMILES: CCc1ccc(C2CC(c3ccccc3)=CC2C(=O)CCc2ccc(C3=CCC=C3)cc2)cc1] | PZ062 | 126.3 | 48.43 |
| 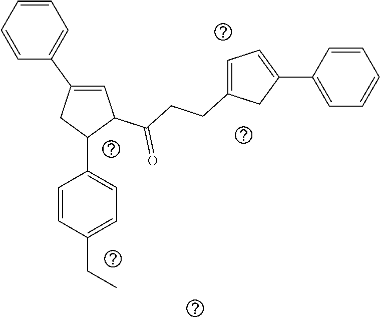 [SMILES: CCc1ccc(C2CC(c3ccccc3)=CC2C(=O)CCC2=CC=C(c3ccccc3)C2)cc1] | PZ063 | 91.76 | 58.96 |
| 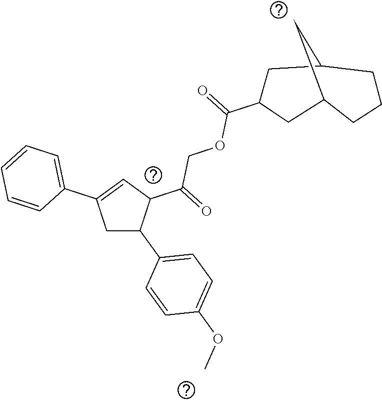 [SMILES: COc1ccc(C2CC(c3ccccc3)=CC2C(=O)COC(=O)C2CC3CCCC(C3)C2)cc1] | PZ064 | 97.1 | 72.85 |
| 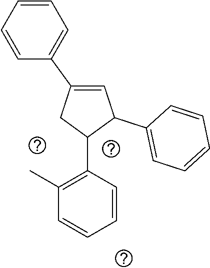 [SMILES: Cc1ccccc1C1CC(c2ccccc2)=CC1c1ccccc1] | PZ065 | 98.53 | 117.3 |
| 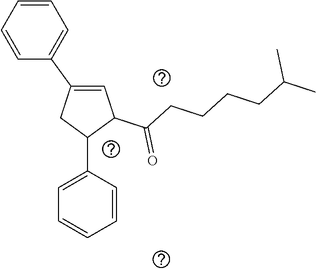 [SMILES: CC(C)CCCCC(=O)C1C=C(c2ccccc2)CC1c1ccccc1] | PZ066 | 110.6 | 97.54 |
| 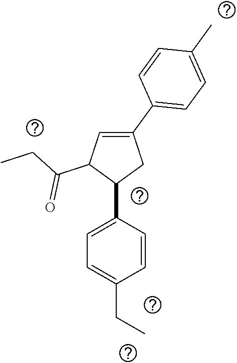 [SMILES: CCC(=O)C1C=C(c2ccc(C)cc2)C[C@H]1c1ccc(CC)cc1] | PZ067 | 109.1 | 85.81 |
| 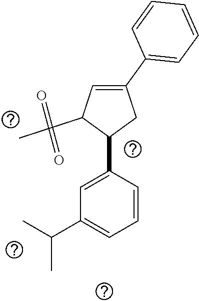 | PZ068 | 62.93 | 69.86 |
| 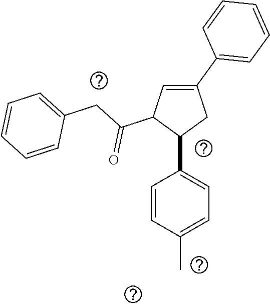 [SMILES: Cc1ccc([C@@H]2CC(c3ccccc3)=CC2C(=O)Cc2ccccc2)cc1] | PZ069 | 70.4 | 145.7 |
| 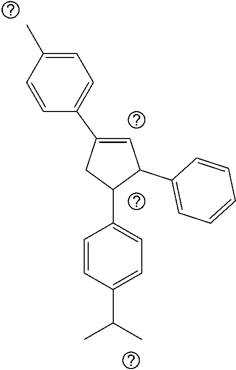 [SMILES: Cc1ccc(C2=CC(c3ccccc3)C(c3ccc(C(C)C)cc3)C2)cc1] | PZ070 | 70.39 | 95.42 |
| 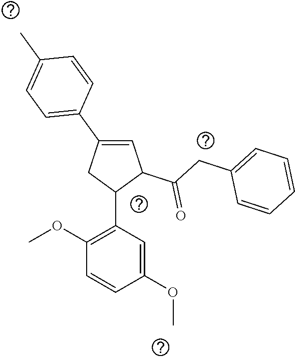 [SMILES: COc1ccc(OC)c(C2CC(c3ccc(C)cc3)=CC2C(=O)Cc2ccccc2)c1] | PZ071 | 73.6 | 111.5 |
| 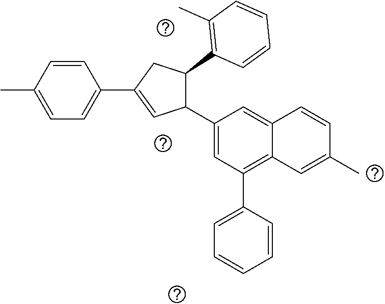 | PZ072 | 80.05 | 110.6 |
| 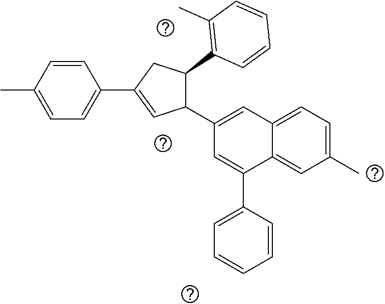 | PZ073 | 73.31 | 119.9 |
| 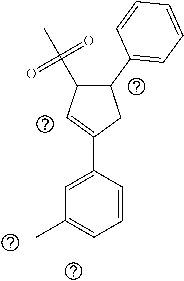 | PZ074 | 71.16 | 108.2 |
| 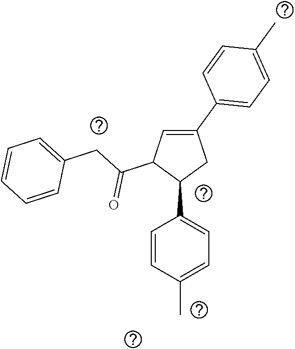 [SMILES: Cc1ccc(C2=CC(C(=O)Cc3ccccc3)[C@H](c3ccc(C)cc3)C2)cc1] | PZ075 | 62.91 | 75.95 |
| 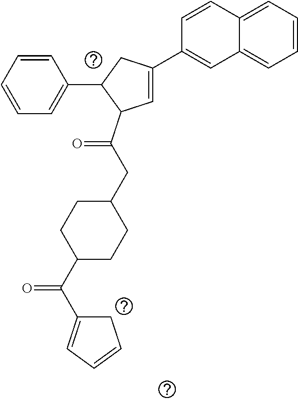 [SMILES: O=C(C1=CC=CC1)C1CCC(CC(=O)C2C=C(c3ccc4ccccc4c3)CC2c2ccccc2)CC1] | PZ076 | 73.13 | 96.21 |
| 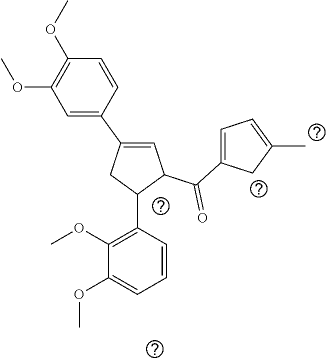 | PZ077 | 76.72 | 59.71 |
| 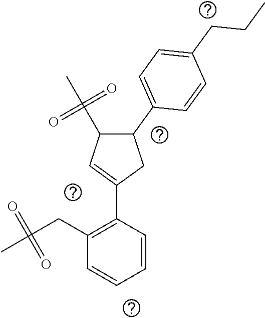 | PZ078 | 83.74 | 139.2 |
| 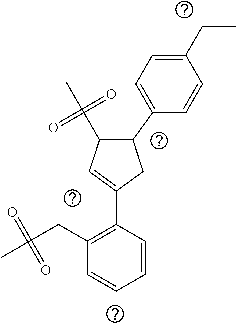 | PZ079 | 53.62 | 82.76 |
| 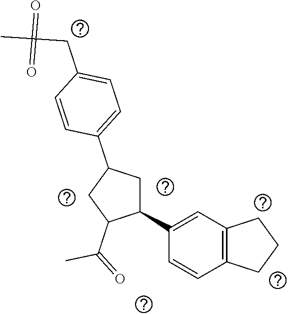 | PZ080 | 67.2 | 157.4 |
| 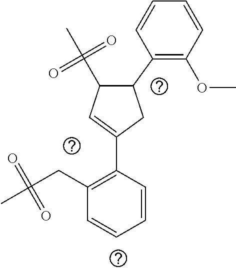 | PZ081 | 70.55 | 77.69 |
| 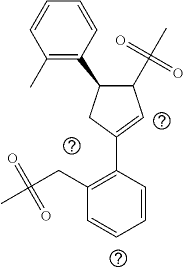 | PZ082 | 89.08 | 111.1 |
| 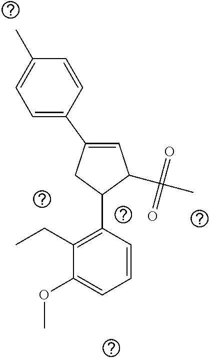 | PZ083 | 25.29 | 62.24 |
| 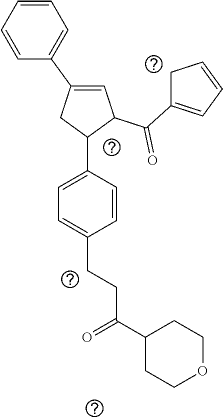 [SMILES: O=C(CCc1ccc(C2CC(c3ccccc3)=CC2C(=O)C2=CC=CC2)cc1)C1CCOCC1] | PZ084 | 70.56 | 91.66 |
| 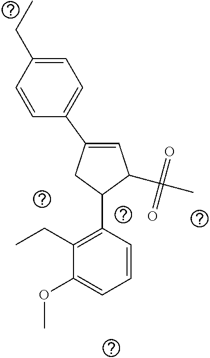 | PZ085 | 32.52 | 41.51 |
| 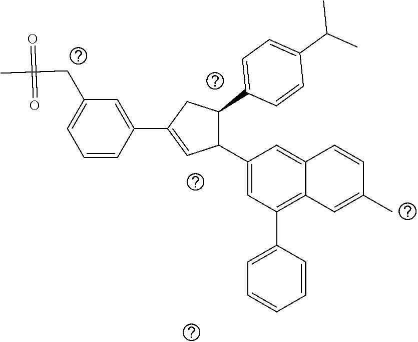 | PZ086 | 87.09 | 104.4 |
| 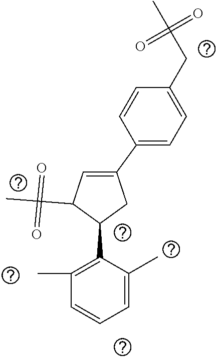 | PZ087 | 53.16 | 65.72 |
| 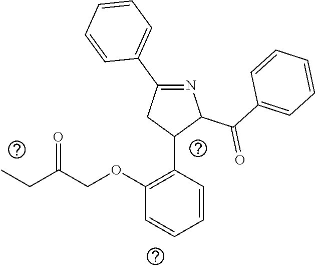 [SMILES: CCC(=O)COc1ccccc1C1CC(c2ccccc2)=NC1C(=O)c1ccccc1] | PZ088 | 99.13 | 110.6 |
| 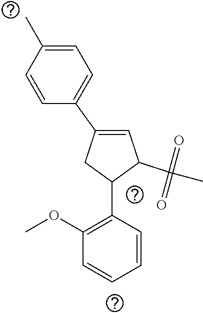 | PZ089 | 44.33 | 53.54 |
| 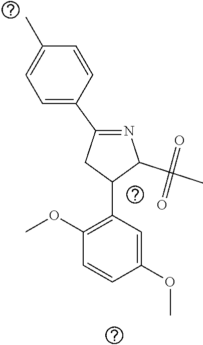 | PZ090 | 48.49 | 58.15 |
| 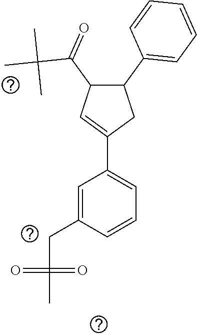 | PZ091 | 97.11 | 93.83 |
| 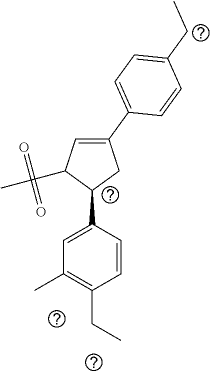 | PZ092 | 67.61 | 150.5 |
| 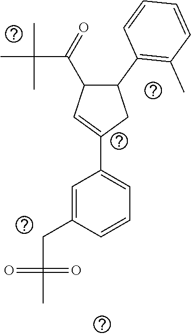 | PZ093 | 80.69 | 90.44 |
| 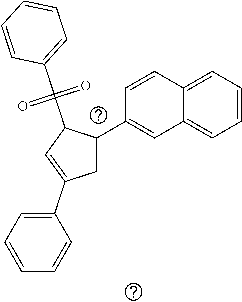 | PZ094 | 62.14 | 77.23 |
| 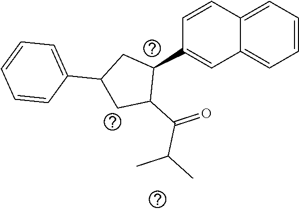 [SMILES: CC(C)C(=O)C1CC(c2ccccc2)C[C@H]1c1ccc2ccccc2c1] | PZ095 | 97.81 | 116 |
| 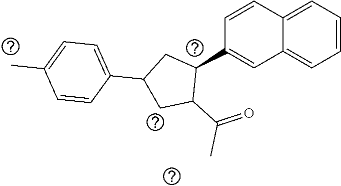 [SMILES: CC(=O)C1CC(c2ccc(C)cc2)C[C@H]1c1ccc2ccccc2c1] | PZ096 | 85.6 | 103 |
| 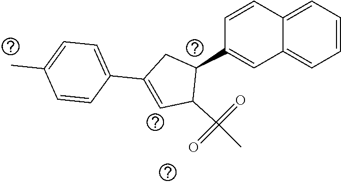 | PZ097 | 43.99 | 71.58 |
| 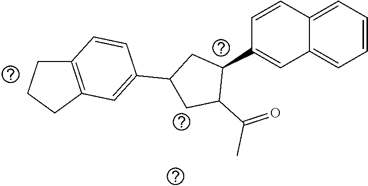 [SMILES: CC(=O)C1CC(c2ccc3c(c2)CCC3)C[C@H]1c1ccc2ccccc2c1] | PZ098 | 85.52 | 73.3 |
| 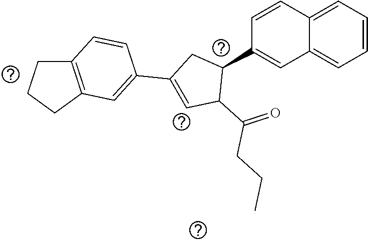 [SMILES: CCCC(=O)C1C=C(c2ccc3c(c2)CCC3)C[C@H]1c1ccc2ccccc2c1] | PZ099 | 85.48 | 76.46 |
| 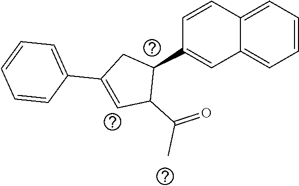 [SMILES: CC(=O)C1C=C(c2ccccc2)C[C@H]1c1ccc2ccccc2c1] | PZ100 | 63.42 | 88.84 |
| 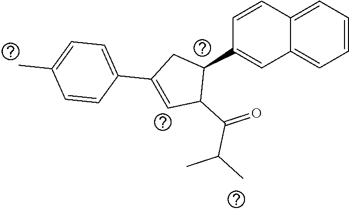 [SMILES: Cc1ccc(C2=CC(C(=O)C(C)C)[C@H](c3ccc4ccccc4c3)C2)cc1] | PZ101 | 34.12 | 110.9 |
| 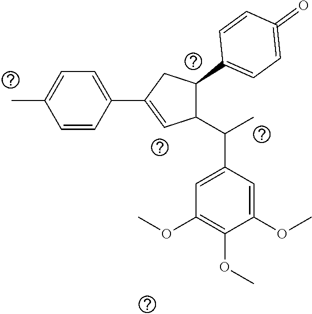 | PZ102 | 102.7 | 79.86 |
| 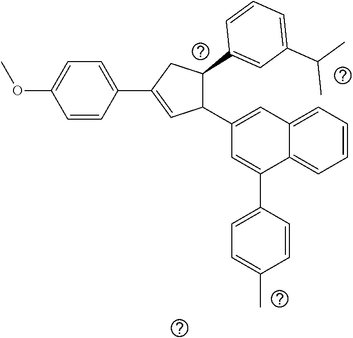 [SMILES: COc1ccc(C2=CC(c3cc(-c4ccc(C)cc4)c4ccccc4c3)[C@H](c3cccc(C(C)C)c3)C2)cc1] | PZ103 | 94.98 | 124.2 |
| 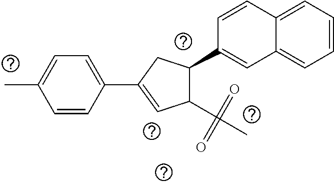 | PZ104 | 60.23 | 119.3 |
| 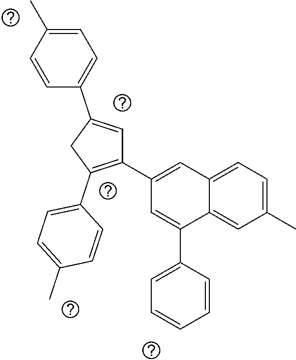 [SMILES: Cc1ccc(C2=CC(c3cc(-c4ccccc4)c4cc(C)ccc4c3)=C(c3ccc(C)cc3)C2)cc1] | PZ105 | 97.55 | 117.8 |
| 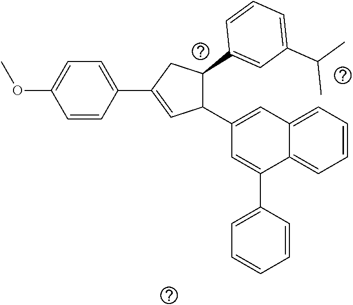 [SMILES: COc1ccc(C2=CC(c3cc(-c4ccccc4)c4ccccc4c3)[C@H](c3cccc(C(C)C)c3)C2)cc1] | PZ106 | 96.91 | 121.7 |
| 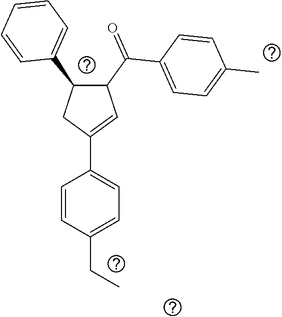 [SMILES: CCc1ccc(C2=CC(C(=O)c3ccc(C)cc3)[C@H](c3ccccc3)C2)cc1] | PZ107 | 110.3 | 83.13 |
| 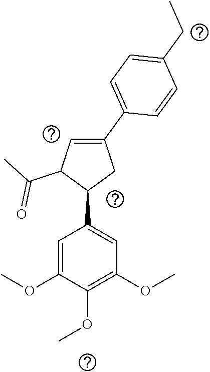 [SMILES: CCc1ccc(C2=CC(C(C)=O)[C@H](c3cc(OC)c(OC)c(OC)c3)C2)cc1] | PZ108 | 85.97 | 69.96 |
| 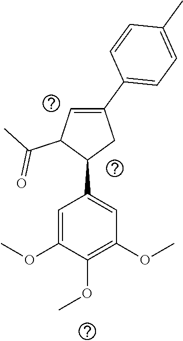 | PZ109 | 91.93 | 92.75 |
| 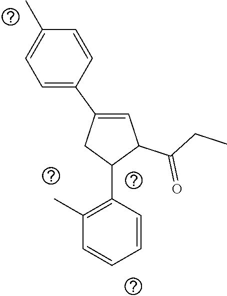 [SMILES: CCC(=O)C1C=C(c2ccc(C)cc2)CC1c1ccccc1C] | PZ110 | 98.3 | 99.95 |
| 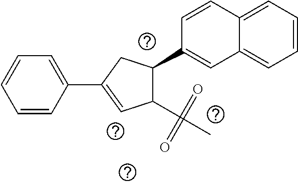 | PZ111 | 66.51 | 98.65 |
| 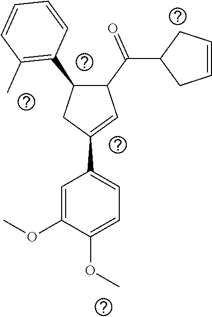 | PZ112 | 140 | 73.73 |
| 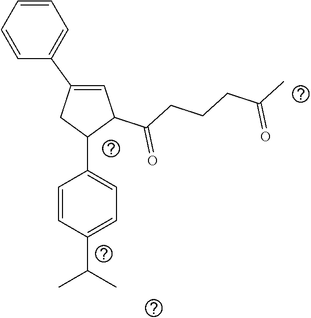 [SMILES: CC(=O)CCCC(=O)C1C=C(c2ccccc2)CC1c1ccc(C(C)C)cc1] | PZ113 | 92.96 | 85.84 |
| 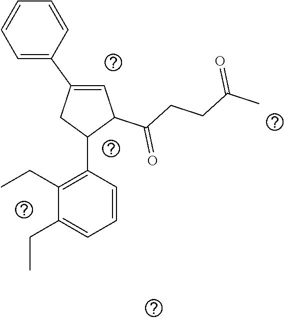 [SMILES: CCc1cccc(C2CC(c3ccccc3)=CC2C(=O)CCC(C)=O)c1CC] | PZ114 | 90.25 | 84.92 |
| 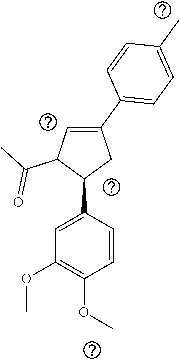 [SMILES: COc1ccc([C@@H]2CC(c3ccc(C)cc3)=CC2C(C)=O)cc1OC] | PZ115 | 111.1 | 93.63 |
| 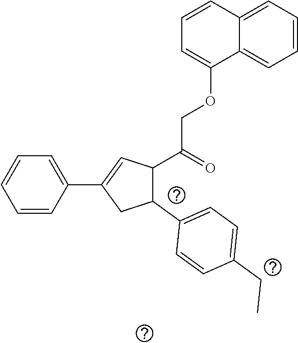 [SMILES: CCc1ccc(C2CC(c3ccccc3)=CC2C(=O)COc2cccc3ccccc23)cc1] | PZ116 | 70.63 | 26.92 |
| 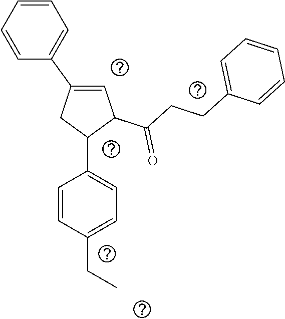 [SMILES: CCc1ccc(C2CC(c3ccccc3)=CC2C(=O)CCc2ccccc2)cc1] | PZ117 | 86.98 | 43.96 |
| 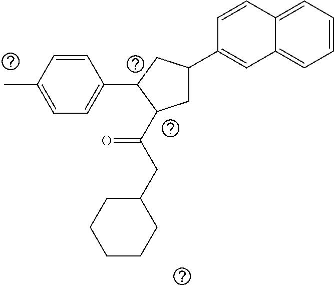 [SMILES: Cc1ccc(C2CC(c3ccc4ccccc4c3)CC2C(=O)CC2CCCCC2)cc1] | PZ118 | 65.96 | 111.6 |
| 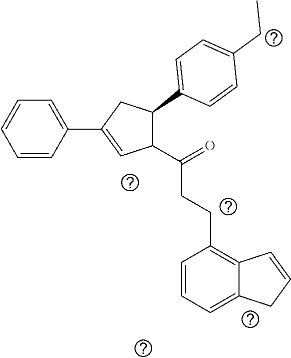 [SMILES: CCc1ccc([C@@H]2CC(c3ccccc3)=CC2C(=O)CCc2cccc3c2C=CC3)cc1] | PZ119 | 95.24 | 37.57 |
| 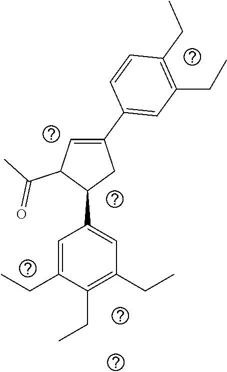 [SMILES: CCc1ccc(C2=CC(C(C)=O)[C@H](c3cc(CC)c(CC)c(CC)c3)C2)cc1CC] | PZ120 | 130.3 | 75.28 |
| 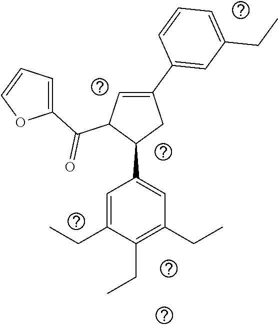 [SMILES: CCc1cccc(C2=CC(C(=O)c3ccco3)[C@H](c3cc(CC)c(CC)c(CC)c3)C2)c1] | PZ121 | 79.03 | 48.9 |
| 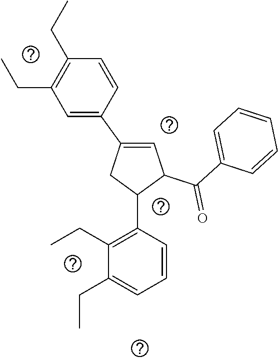 [SMILES: CCc1ccc(C2=CC(C(=O)c3ccccc3)C(c3cccc(CC)c3CC)C2)cc1CC] | PZ122 | 91.68 | 66.81 |
| 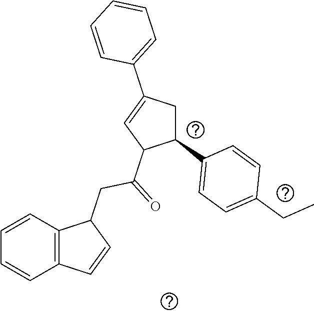 [SMILES: CCc1ccc([C@@H]2CC(c3ccccc3)=CC2C(=O)CC2C=Cc3ccccc32)cc1] | PZ123 | 84.65 | 64.86 |
| 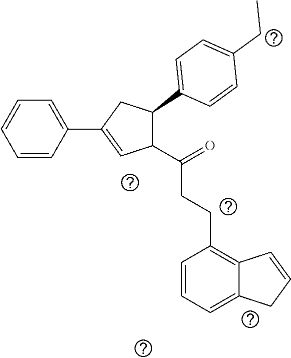 [SMILES: CCc1ccc([C@@H]2CC(c3ccccc3)=CC2C(=O)CCc2cccc3c2C=CC3)cc1] | PZ124 | 96.69 | 36.72 |
| 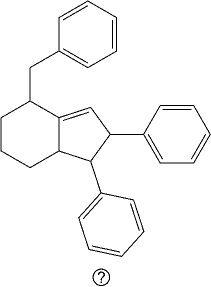 [SMILES: C1=C2C(Cc3ccccc3)CCCC2C(c2ccccc2)C1c1ccccc1] | PZ125 | 92.06 | 113.2 |
| 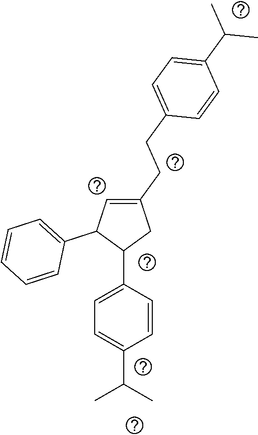 [SMILES: CC(C)c1ccc(CCC2=CC(c3ccccc3)C(c3ccc(C(C)C)cc3)C2)cc1] | PZ126 | 97.63 | 92.65 |
| 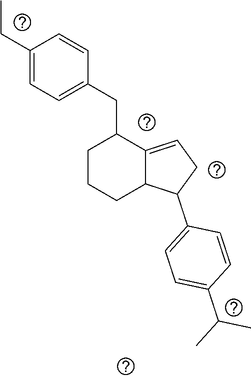 [SMILES: CCc1ccc(CC2CCCC3C2=CCC3c2ccc(C(C)C)cc2)cc1] | PZ127 | 94.66 | 76.86 |
| 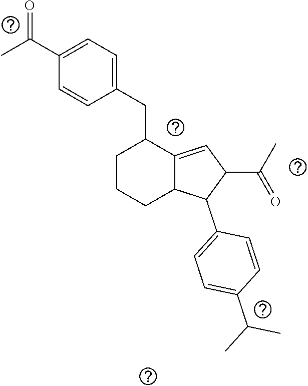 [SMILES: CC(=O)c1ccc(CC2CCCC3C2=CC(C(C)=O)C3c2ccc(C(C)C)cc2)cc1] | PZ128 | 120.2 | 98.26 |
| 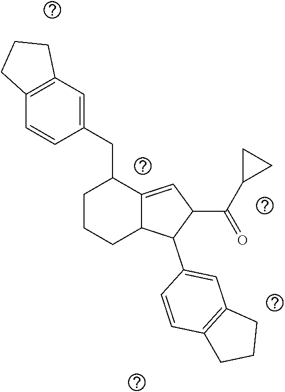 [SMILES: O=C(C1CC1)C1C=C2C(Cc3ccc4c(c3)CCC4)CCCC2C1c1ccc2c(c1)CCC2] | PZ129 | 91.43 | 69.92 |
| 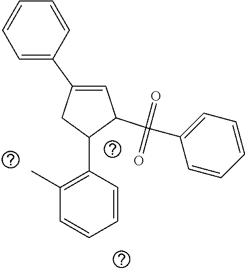 | PZ130 | 44.75 | 46.07 |
| 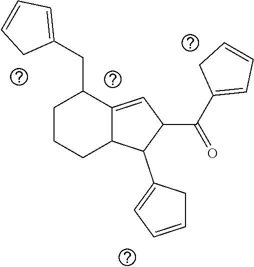 [SMILES: O=C(C1=CC=CC1)C1C=C2C(CC3=CC=CC3)CCCC2C1C1=CC=CC1] | PZ131 | 118.3 | 64.77 |
| 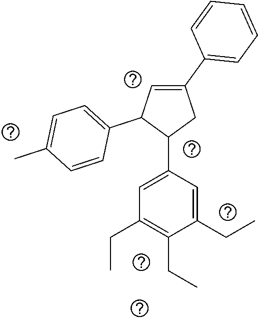 [SMILES: CCc1cc(C2CC(c3ccccc3)=CC2c2ccc(C)cc2)cc(CC)c1CC] | PZ132 | 97.1 | 50.5 |
| 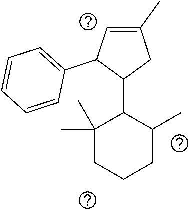 [SMILES: CC1=CC(c2ccccc2)C(C2C(C)CCCC2(C)C)C1] | PZ133 | 101.1 | 94.34 |
| 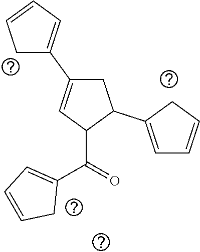 [SMILES: O=C(C1=CC=CC1)C1C=C(C2=CC=CC2)CC1C1=CC=CC1] | PZ134 | 108.4 | 91.43 |
| 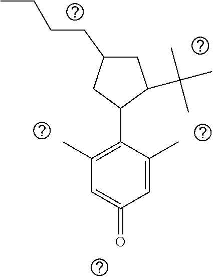 | PZ135 | 79.78 | 91.44 |
| 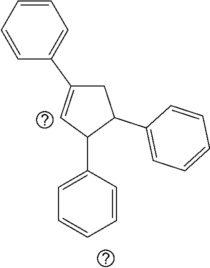 [SMILES: C1=C(c2ccccc2)CC(c2ccccc2)C1c1ccccc1] | PZ136 | 92.72 | 35.69 |
| 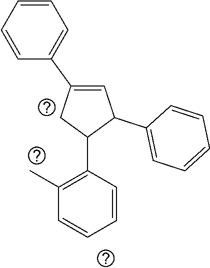 [SMILES: Cc1ccccc1C1CC(c2ccccc2)=CC1c1ccccc1] | PZ137 | 56.63 | 117.3 |
| 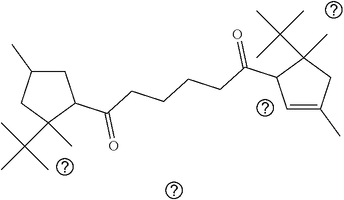 [SMILES: CC1=CC(C(=O)CCCCC(=O)C2CC(C)CC2(C)C(C)(C)C)C(C)(C(C)(C)C)C1] | PZ138 | 103.5 | 99.47 |
| 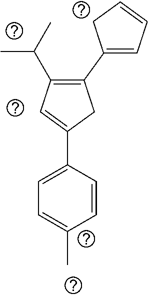 [SMILES: Cc1ccc(C2=CC(C(C)C)=C(C3=CC=CC3)C2)cc1] | PZ139 | 114.3 | 88.75 |
| 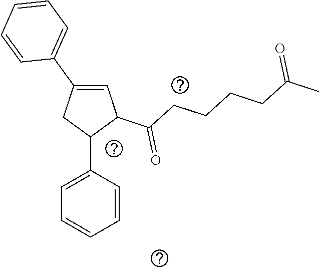 [SMILES: CC(=O)CCCCC(=O)C1C=C(c2ccccc2)CC1c1ccccc1] | PZ140 | 110.5 | 97.54 |
| 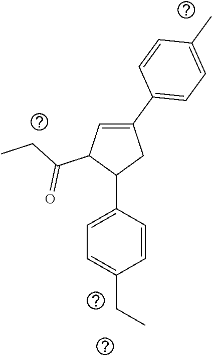 [SMILES: CCC(=O)C1C=C(c2ccc(C)cc2)CC1c1ccc(CC)cc1] | PZ141 | 109.1 | 35.31 |
| 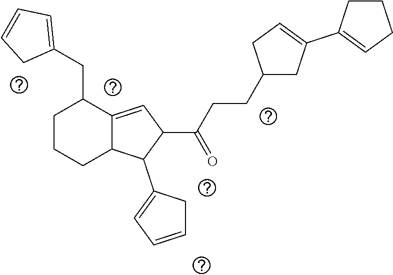 [SMILES: O=C(CCC1CC=C(C2=CCCC2)C1)C1C=C2C(CC3=CC=CC3)CCCC2C1C1=CC=CC1] | PZ142 | 74.27 | 48.38 |
| 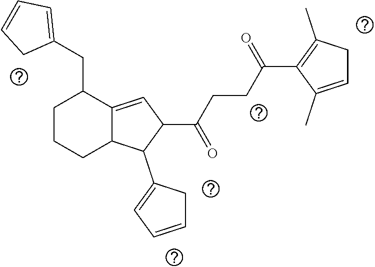 [SMILES: CC1=CCC(C)=C1C(=O)CCC(=O)C1C=C2C(CC3=CC=CC3)CCCC2C1C1=CC=CC1] | PZ143 | 95.76 | 80.04 |
| 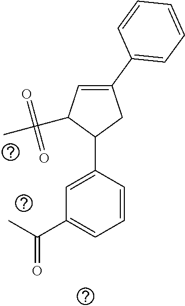 | PZ144 | 62.93 | 69.86 |
| 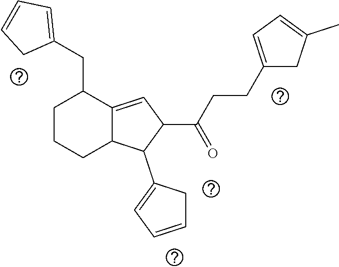 [SMILES: CC1=CC=C(CCC(=O)C2C=C3C(CC4=CC=CC4)CCCC3C2C2=CC=CC2)C1] | PZ145 | 80.69 | 62.71 |
| 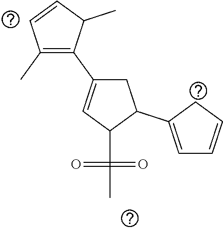 | PZ146 | 106.6 | 93.55 |
| 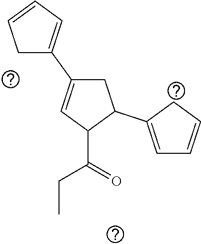 [SMILES: CCC(=O)C1C=C(C2=CC=CC2)CC1C1=CC=CC1] | PZ147 | 92.27 | 83.97 |
| 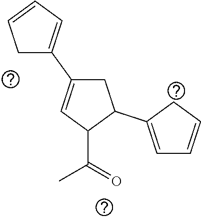 [SMILES: CC(=O)C1C=C(C2=CC=CC2)CC1C1=CC=CC1] | PZ148 | 85.33 | 87.36 |
| 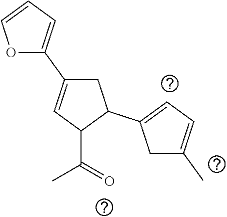 [SMILES: CC(=O)C1C=C(c2ccco2)CC1C1=CC=C(C)C1] | PZ149 | 98.35 | 102.6 |
| 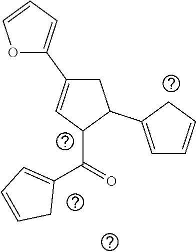 [SMILES: O=C(C1=CC=CC1)C1C=C(c2ccco2)CC1C1=CC=CC1] | PZ150 | 111.9 | 92.09 |
| 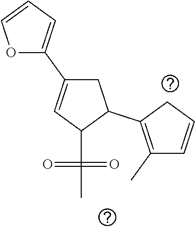 | PZ151 | 100.6 | 92.31 |
| 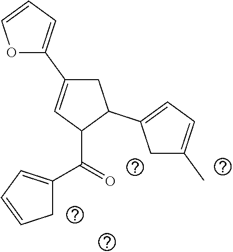 [SMILES: CC1=CC=C(C2CC(c3ccco3)=CC2C(=O)C2=CC=CC2)C1] | PZ152 | 30.35 | 78.18 |
| 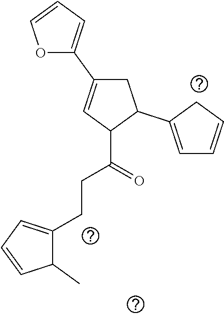 [SMILES: CC1C=CC=C1CCC(=O)C1C=C(c2ccco2)CC1C1=CC=CC1] | PZ153 | 85.98 | 71.39 |
| 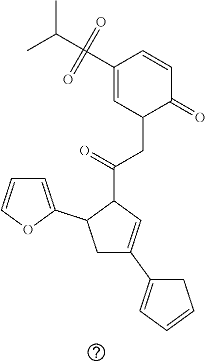 | PZ154 | 95.22 | 84.94 |
| 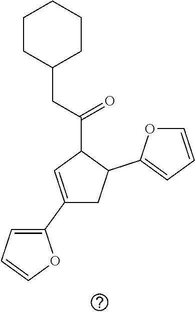 [SMILES: O=C(CC1CCCCC1)C1C=C(c2ccco2)CC1c1ccco1] | PZ155 | 101.4 | 88.93 |
| 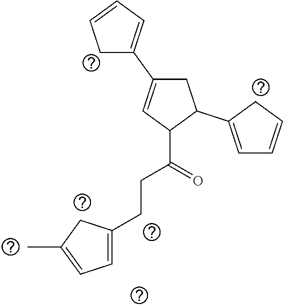 [SMILES: CC1=CC=C(CCC(=O)C2C=C(C3=CC=CC3)CC2C2=CC=CC2)C1] | PZ156 | 91.75 | 89.15 |
| 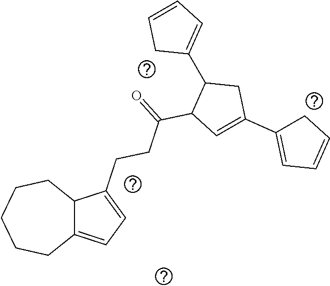 [SMILES: O=C(CCC1=CC=C2CCCCCC21)C1C=C(C2=CC=CC2)CC1C1=CC=CC1] | PZ157 | 82.42 | [symbol] |
| 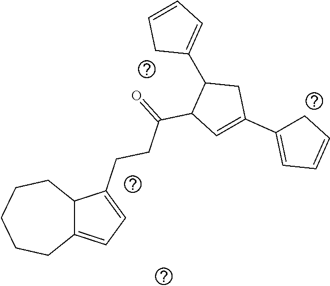 [SMILES: O=C(CCC1=CC=C2CCCCCC21)C1C=C(C2=CC=CC2)CC1C1=CC=CC1] | PZ158 | 102.3 | 82.46 |
| 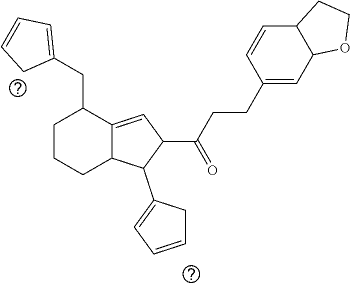 [SMILES: O=C(CCC1=CC2OCCC2C=C1)C1C=C2C(CC3=CC=CC3)CCCC2C1C1=CC=CC1] | PZ159 | 76.87 | 77.47 |
| 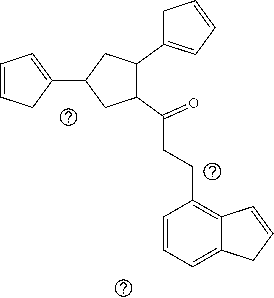 [SMILES: O=C(CCc1cccc2c1C=CC2)C1CC(C2=CC=CC2)CC1C1=CC=CC1] | PZ160 | 119.1 | 49.1 |
| 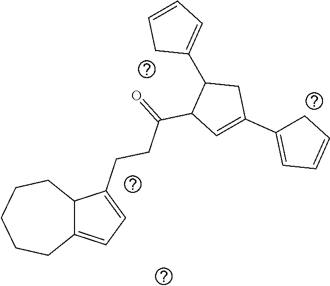 [SMILES: O=C(CCC1=CC=C2CCCCCC21)C1C=C(C2=CC=CC2)CC1C1=CC=CC1] | PZ161 | 96.87 | 101.5 |
| 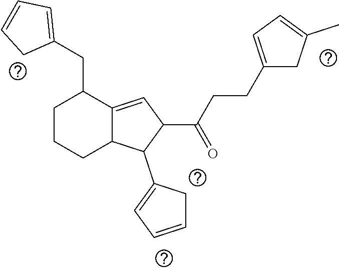 [SMILES: CC1=CC=C(CCC(=O)C2C=C3C(CC4=CC=CC4)CCCC3C2C2=CC=CC2)C1] | PZ162 | 60.32 | 74.77 |
| 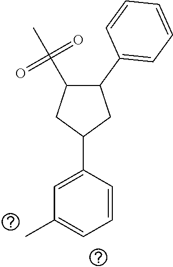 | PZ163 | 71.16 | 103.2 |
| 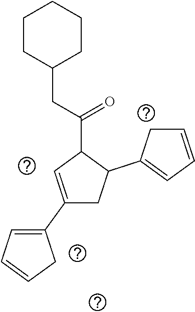 [SMILES: O=C(CC1CCCCC1)C1C=C(C2=CC=CC2)CC1C1=CC=CC1] | PZ164 | 79.01 | 82.49 |
| 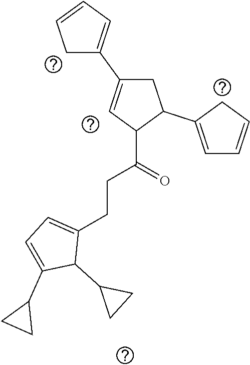 [SMILES: O=C(CCC1=CC=C(C2CC2)C1C1CC1)C1C=C(C2=CC=CC2)CC1C1=CC=CC1] | PZ165 | 69.14 | 78.23 |
| 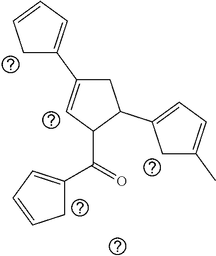 [SMILES: CC1=CC=C(C2CC(C3=CC=CC3)=CC2C(=O)C2=CC=CC2)C1] | PZ166 | 69.17 | 110.5 |
| 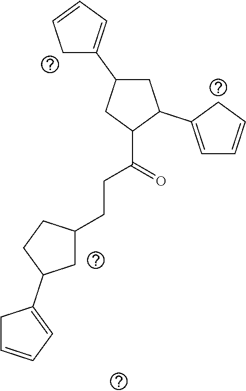 [SMILES: O=C(CCC1CCC(C2=CC=CC2)C1)C1CC(C2=CC=CC2)CC1C1=CC=CC1] | PZ167 | 62.35 | 86.14 |
| 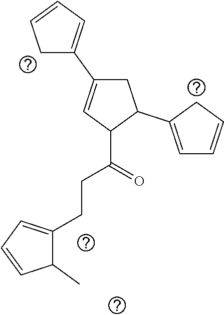 [SMILES: CC1C=CC=C1CCC(=O)C1C=C(C2=CC=CC2)CC1C1=CC=CC1] | PZ168 | 80.74 | 96.93 |
| 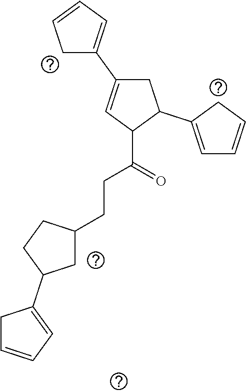 [SMILES: O=C(CCC1CCC(C2=CC=CC2)C1)C1C=C(C2=CC=CC2)CC1C1=CC=CC1] | PZ169 | 96.58 | 68.75 |
| 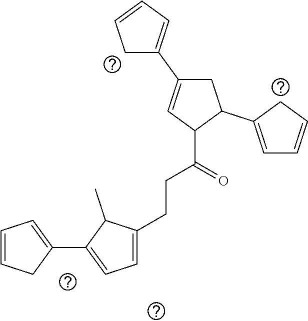 [SMILES: CC1C(CCC(=O)C2C=C(C3=CC=CC3)CC2C2=CC=CC2)=CC=C1C1=CC=CC1] | PZ170 | 46.16 | 66.93 |
| 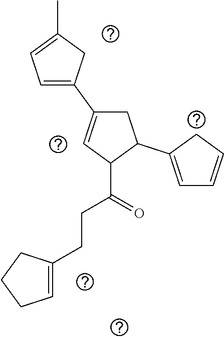 [SMILES: CC1=CC=C(C2=CC(C(=O)CCC3=CCCC3)C(C3=CC=CC3)C2)C1] | PZ171 | 68.74 | 60.48 |
| 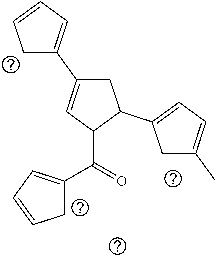 [SMILES: CC1=CC=C(C2CC(C3=CC=CC3)=CC2C(=O)C2=CC=CC2)C1] | PZ172 | 69.38 | 114.3 |
| 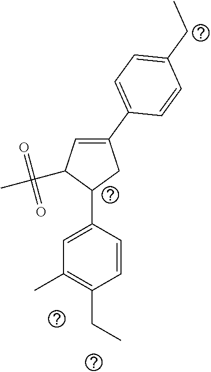 | PZ173 | 67.61 | 160.5 |
| 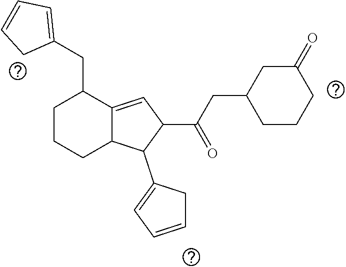 [SMILES: O=C1CCCC(CC(=O)C2C=C3C(CC4=CC=CC4)CCCC3C2C2=CC=CC2)C1] | PZ174 | 90.34 | 111 |
| 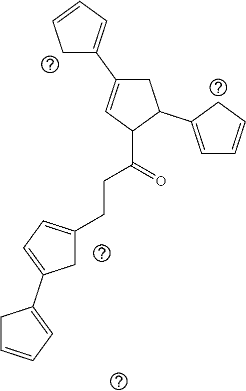 [SMILES: O=C(CCC1=CC=C(C2=CC=CC2)C1)C1C=C(C2=CC=CC2)CC1C1=CC=CC1] | PZ175 | 91.85 | 73.84 |
| 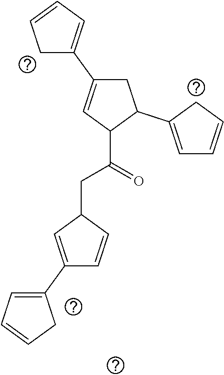 [SMILES: O=C(CC1C=CC(C2=CC=CC2)=C1)C1C=C(C2=CC=CC2)CC1C1=CC=CC1] | PZ176 | 94.88 | 116.4 |
| 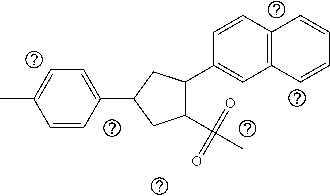 | PZ177 | 43.89 | 71.59 |
| 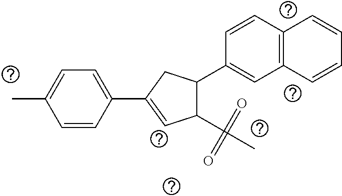 | PZ178 | 60.25 | 119.3 |
| 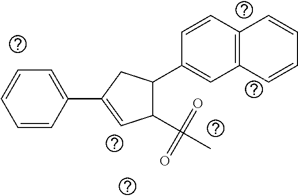 | PZ179 | 66.51 | 95.65 |
| 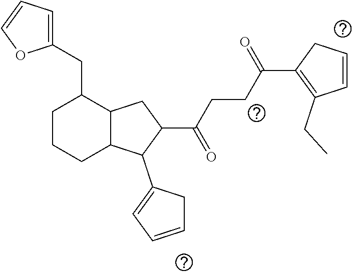 [SMILES: CCC1=C(C(=O)CCC(=O)C2CC3C(Cc4ccco4)CCCC3C2C2=CC=CC2)CC=C1] | PZ180 | 64.82 | 102 |
| 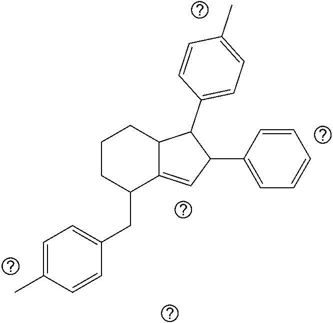 [SMILES: Cc1ccc(CC2CCCC3C2=CC(c2ccccc2)C3c2ccc(C)cc2)cc1] | PZ181 | 76.64 | 103.2 |
| 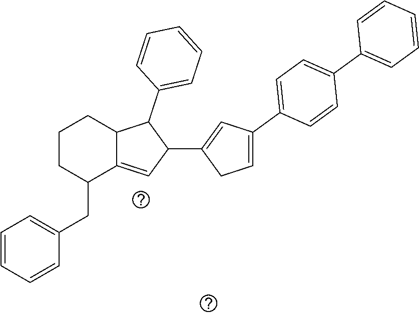 [SMILES: C1=C(c2ccc(-c3ccccc3)cc2)C=C(C2C=C3C(Cc4ccccc4)CCCC3C2c2ccccc2)C1] | PZ182 | 85.4 | [symbol] |
| 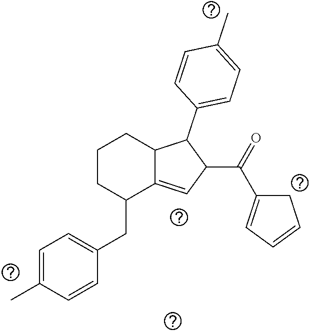 [SMILES: Cc1ccc(CC2CCCC3C2=CC(C(=O)C2=CC=CC2)C3c2ccc(C)cc2)cc1] | PZ183 | 99.34 | 124.3 |
| 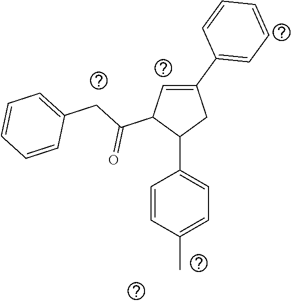 [SMILES: Cc1ccc(C2CC(c3ccccc3)=CC2C(=O)Cc2ccccc2)cc1] | PZ184 | 70.4 | 146.7 |
| 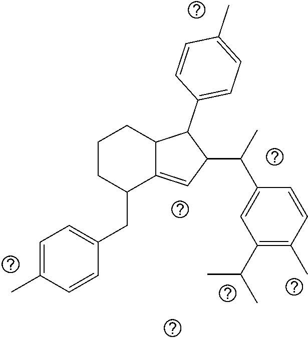 | PZ185 | 67.06 | 67.28 |
| 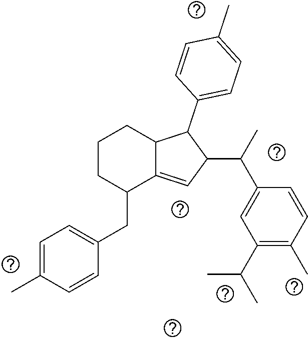 | PZ186 | 66.63 | 105.6 |
| 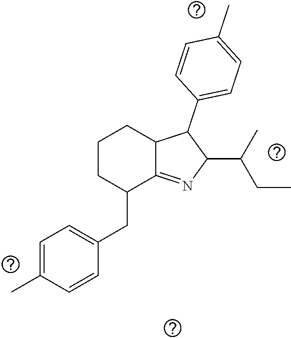 [SMILES: CCC(C)C1N=C2C(Cc3ccc(C)cc3)CCCC2C1c1ccc(C)cc1] | PZ187 | 49.69 | 207.5 |
| 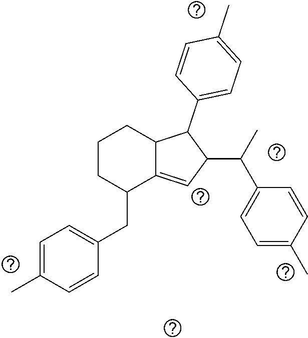 | PZ188 | 67.6 | 91.78 |
| 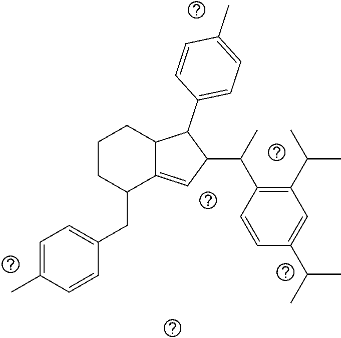 [SMILES: Cc1ccc(CC2CCCC3C2=CC(C(C)c2ccc(C(C)C)cc2C(C)C)C3c2ccc(C)cc2)cc1] | PZ189 | 80.95 | 77.24 |
| 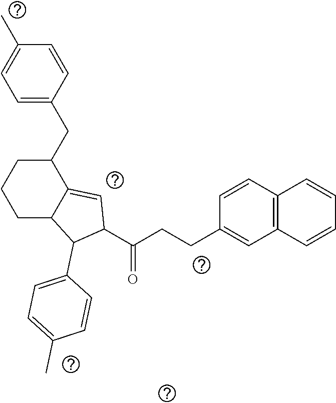 [SMILES: Cc1ccc(CC2CCCC3C2=CC(C(=O)CCc2ccc4ccccc4c2)C3c2ccc(C)cc2)cc1] | PZ190 | 94.65 | 96.65 |
| 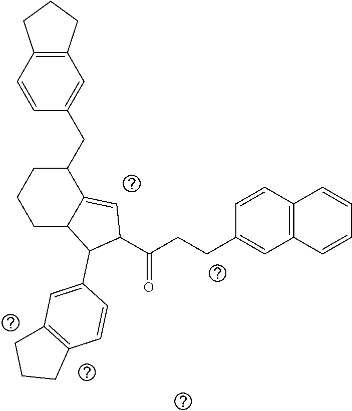 [SMILES: O=C(CCc1ccc2ccccc2c1)C1C=C2C(Cc3ccc4c(c3)CCC4)CCCC2C1c1ccc2c(c1)CCC2] | PZ191 | 93.34 | 115.8 |
| 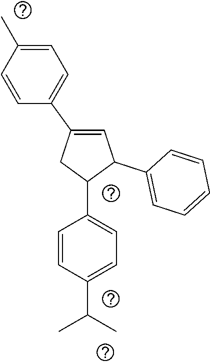 [SMILES: Cc1ccc(C2=CC(c3ccccc3)C(c3ccc(C(C)C)cc3)C2)cc1] | PZ192 | 70.39 | 95.42 |
| 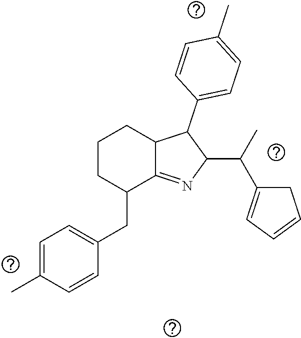 [SMILES: Cc1ccc(CC2CCCC3C2=NC(C(C)C2=CC=CC2)C3c2ccc(C)cc2)cc1] | PZ193 | 74.47 | 89.03 |
| 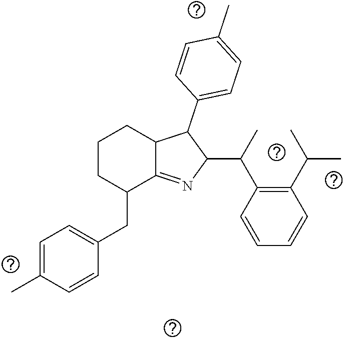 [SMILES: Cc1ccc(CC2CCCC3C2=NC(C(C)c2ccccc2C(C)C)C3c2ccc(C)cc2)cc1] | PZ194 | 69.93 | 99.31 |
| 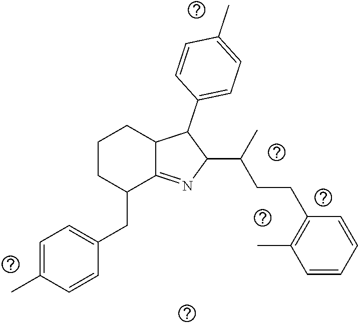 [SMILES: Cc1ccc(CC2CCCC3C2=NC(C(C)CCc2ccccc2C)C3c2ccc(C)cc2)cc1] | PZ195 | 70.1 | 101.3 |
| 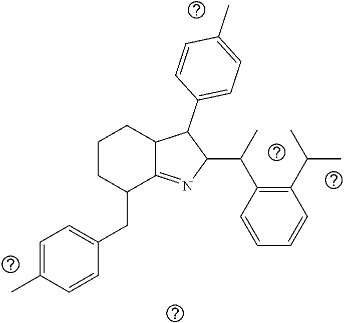 [SMILES: Cc1ccc(CC2CCCC3C2=NC(C(C)c2ccccc2C(C)C)C3c2ccc(C)cc2)cc1] | PZ196 | 77.37 | 101.5 |
| 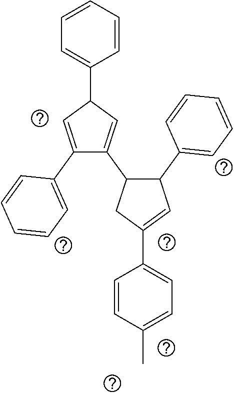 [SMILES: Cc1ccc(C2=CC(c3ccccc3)C(C3=CC(c4ccccc4)C=C3c3ccccc3)C2)cc1] | PZ197 | 88.85 | 111.7 |
| 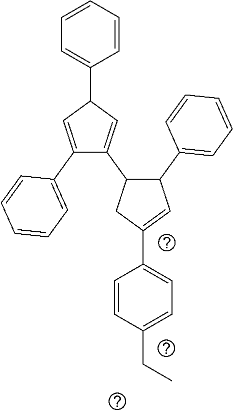 [SMILES: CCc1ccc(C2=CC(c3ccccc3)C(C3=CC(c4ccccc4)C=C3c3ccccc3)C2)cc1] | PZ198 | 60.12 | 113.6 |
| 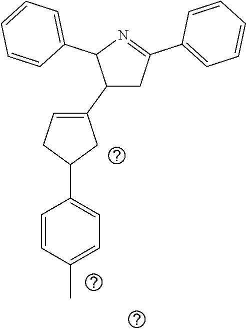 [SMILES: Cc1ccc(C2CC=C(C3CC(c4ccccc4)=NC3c3ccccc3)C2)cc1] | PZ199 | 91.75 | 171.7 |
| 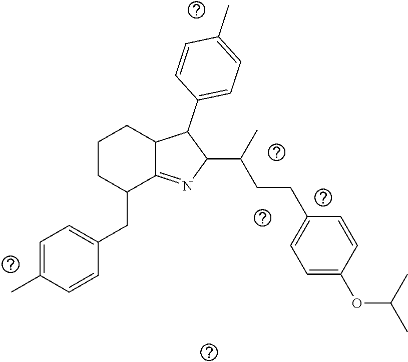 [SMILES: Cc1ccc(CC2CCCC3C2=NC(C(C)CCc2ccc(OC(C)C)cc2)C3c2ccc(C)cc2)cc1] | PZ200 | 79.98 | 98.91 |
| 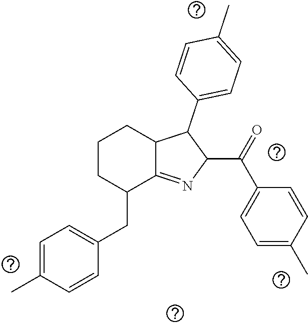 | PZ201 | 62.07 | 78.89 |
| 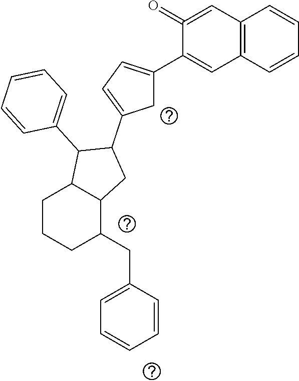 | PZ202 | 76.17 | 107.4 |
| 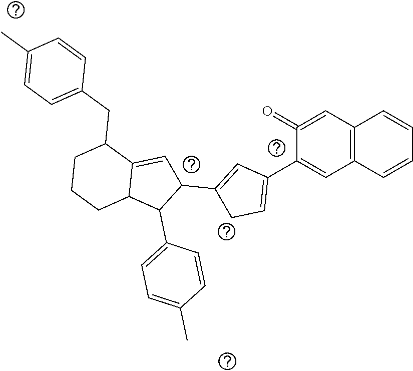 | PZ203 | 77.97 | 113.6 |
| 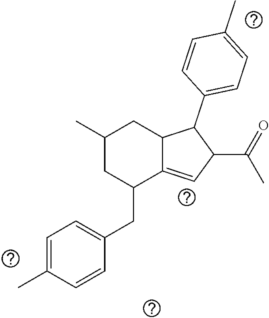 [SMILES: CC(=O)C1C=C2C(Cc3ccc(C)cc3)CC(C)CC2C1c1ccc(C)cc1] | PZ204 | 76.42 | 96.77 |
| 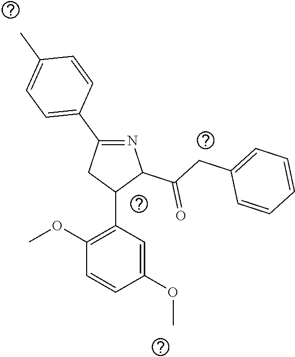 [SMILES: COc1ccc(OC)c(C2CC(c3ccc(C)cc3)=NC2C(=O)Cc2ccccc2)c1] | PZ205 | 79.6 | 111.5 |
| 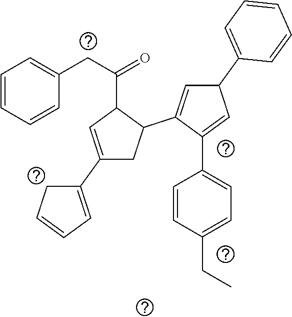 [SMILES: CCc1ccc(C2=CC(c3ccccc3)C=C2C2CC(C3=CC=CC3)=CC2C(=O)Cc2ccccc2)cc1] | PZ206 | 41.73 | 53.38 |
| 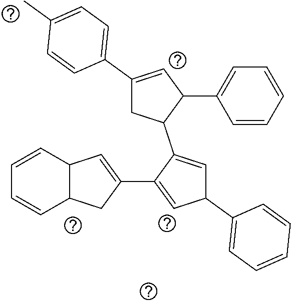 [SMILES: Cc1ccc(C2=CC(c3ccccc3)C(C3=CC(c4ccccc4)C=C3C3=CC4C=CC=CC4C3)C2)cc1] | PZ207 | 80.61 | 106.1 |
| 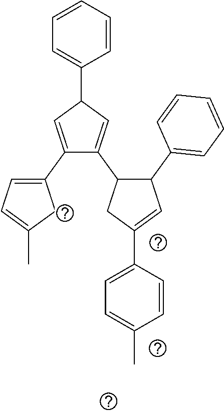 [SMILES: CC1=CC=C(C2=CC(c3ccccc3)C=C2C2CC(c3ccc(C)cc3)=CC2c2ccccc2)C1] | PZ208 | 77.98 | 140.8 |
| 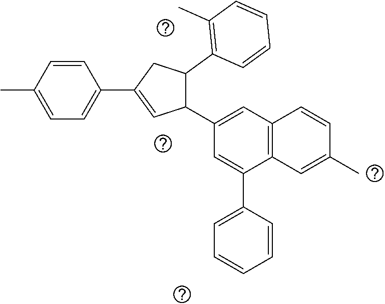 | PZ209 | 80.06 | 110.6 |
| 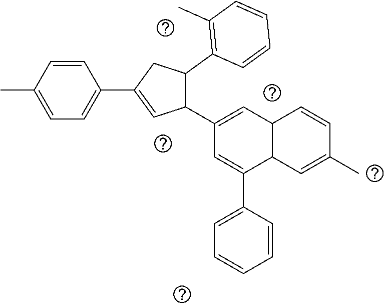 | PZ210 | 73.31 | 119.9 |
| 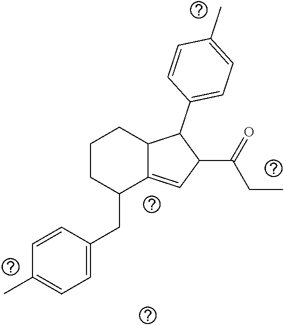 [SMILES: CCC(=O)C1C=C2C(Cc3ccc(C)cc3)CCCC2C1c1ccc(C)cc1] | PZ211 | 69.65 | 362.5 |
| 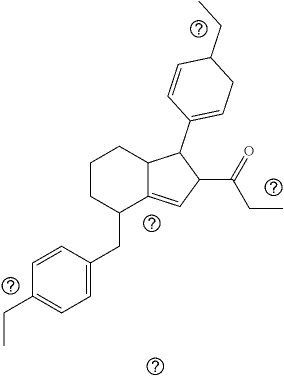 [SMILES: CCC(=O)C1C=C2C(Cc3ccc(CC)cc3)CCCC2C1C1=CCC(CC)C=C1] | PZ212 | 38.67 | 195.7 |
| 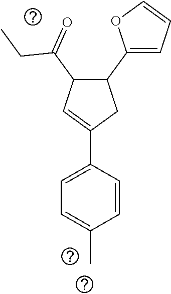 [SMILES: CCC(=O)C1C=C(c2ccc(C)cc2)CC1c1ccco1] | PZ213 | 94.53 | 1683 |
| 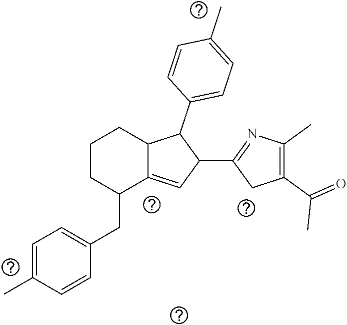 [SMILES: CC(=O)C1=C(C)N=C(C2C=C3C(Cc4ccc(C)cc4)CCCC3C2c2ccc(C)cc2)C1] | PZ214 | 67.65 | 80.27 |
| 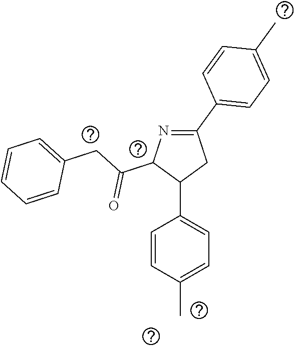 [SMILES: Cc1ccc(C2=NC(C(=O)Cc3ccccc3)C(c3ccc(C)cc3)C2)cc1] | PZ215 | 62.91 | 75.95 |
| 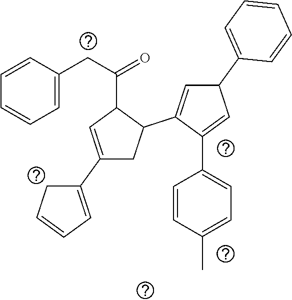 [SMILES: Cc1ccc(C2=CC(c3ccccc3)C=C2C2CC(C3=CC=CC3)=CC2C(=O)Cc2ccccc2)cc1] | PZ216 | 41.03 | 50.78 |
| 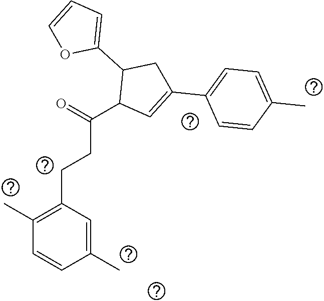 | PZ217 | 78.59 | 95.02 |
| 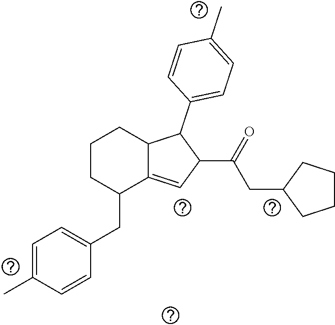 [SMILES: Cc1ccc(CC2CCCC3C2=CC(C(=O)CC2CCCC2)C3c2ccc(C)cc2)cc1] | PZ218 | 83.16 | 124.1 |
| 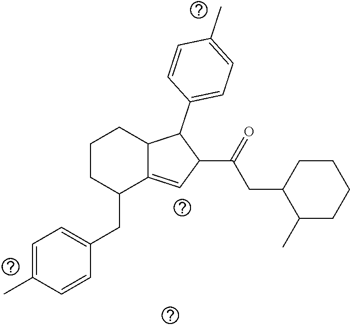 [SMILES: Cc1ccc(CC2CCCC3C2=CC(C(=O)CC2CCCCC2C)C3c2ccc(C)cc2)cc1] | PZ219 | 87.8 | 114.8 |
| 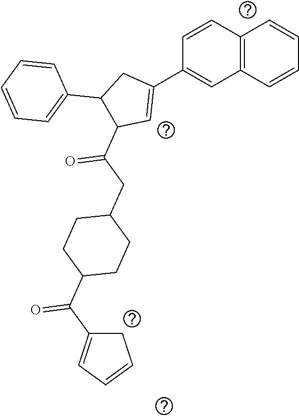 [SMILES: O=C(C1=CC=CC1)C1CCC(CC(=O)C2C=C(c3ccc4ccccc4c3)CC2c2ccccc2)CC1] | PZ220 | 73.18 | 96.21 |
| 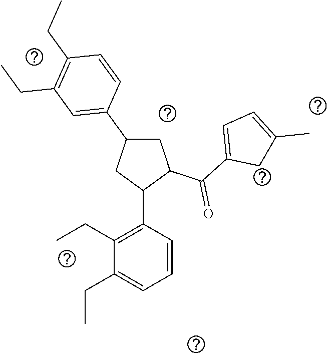 [SMILES: CCc1ccc(C2CC(C(=O)C3=CC=C(C)C3)C(c3cccc(CC)c3CC)C2)cc1CC] | PZ221 | 76.72 | 59.71 |
| 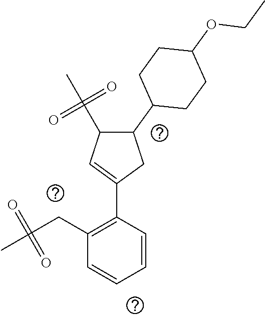 | PZ222 | 63.74 | 139.2 |
| 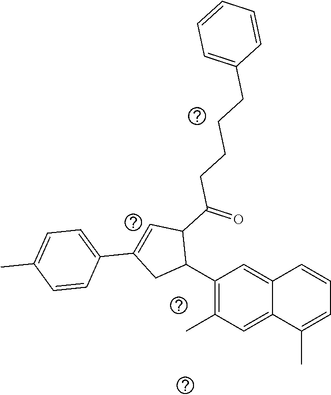 [SMILES: Cc1ccc(C2=CC(C(=O)CCCCc3ccccc3)C(c3cc4cccc(C)c4cc3C)C2)cc1] | PZ223 | 73.86 | 73.56 |
| 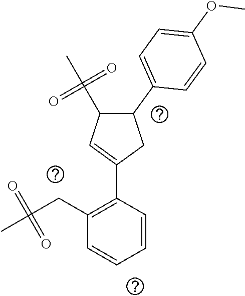 | PZ224 | 53.62 | 82.76 |
| 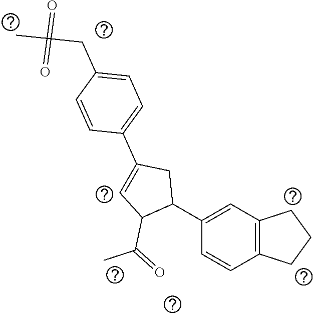 | PZ225 | 87.2 | 157.4 |
| 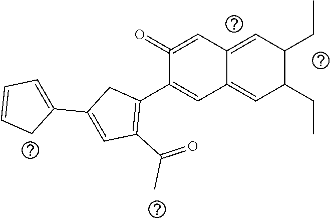 | PZ226 | 74.88 | 95.27 |
| 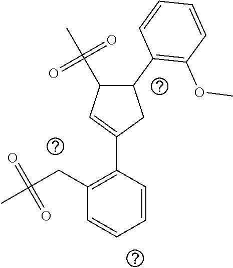 | PZ227 | 70.55 | 77.69 |
| 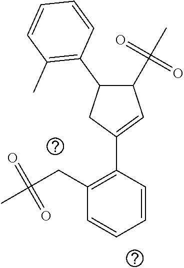 | PZ228 | 69.08 | 111.1 |
| 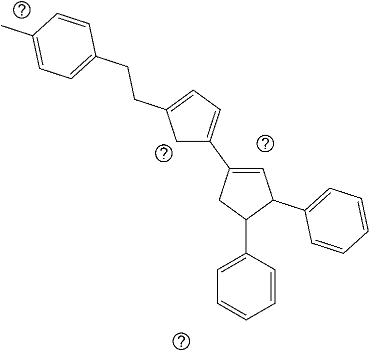 [SMILES: Cc1ccc(CCC2=CC=C(C3=CC(c4ccccc4)C(c4ccccc4)C3)C2)cc1] | PZ229 | 87.98 | 92.47 |
| 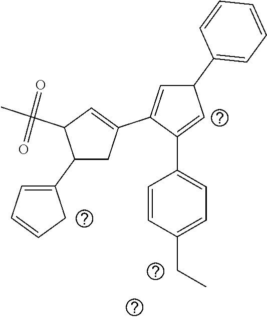 | PZ230 | 42.61 | 46.85 |
| 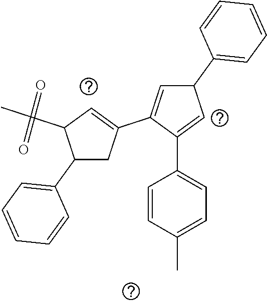 | PZ231 | 45.9 | 53.77 |
| 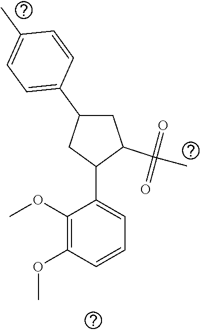 | PZ232 | 25.29 | 62.24 |
| 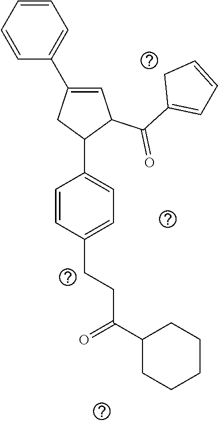 [SMILES: O=C(CCc1ccc(C2CC(c3ccccc3)=CC2C(=O)C2=CC=CC2)cc1)C1CCCCC1] | PZ233 | 70.56 | 91.66 |
| 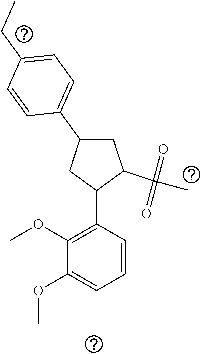 | PZ234 | 32.52 | 41.51 |
| 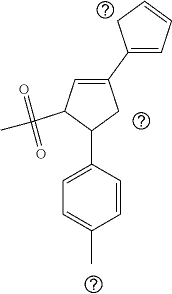 | PZ235 | 88.43 | 127.6 |
| 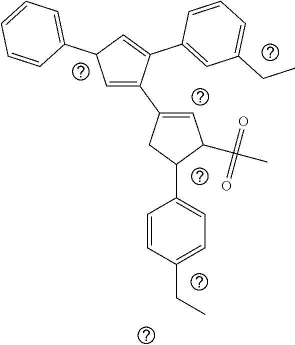 | PZ236 | 39.45 | 60.32 |
| 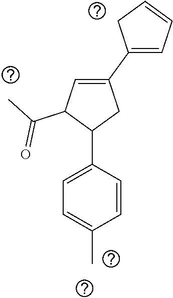 [SMILES: CC(=O)C1C=C(C2=CC=CC2)CC1c1ccc(C)cc1] | PZ237 | 94.31 | 124.4 |
| 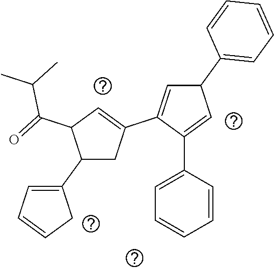 [SMILES: CC(C)C(=O)C1C=C(C2=CC(c3ccccc3)C=C2c2ccccc2)CC1C1=CC=CC1] | PZ238 | 62.63 | 61.33 |
| 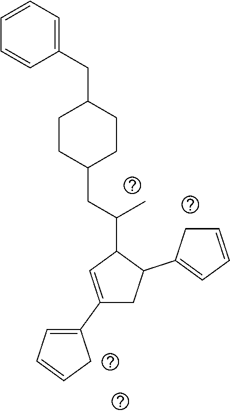 [SMILES: CC(CC1CCC(Cc2ccccc2)CC1)C1C=C(C2=CC=CC2)CC1C1=CC=CC1] | PZ239 | 92.67 | 93.52 |
| 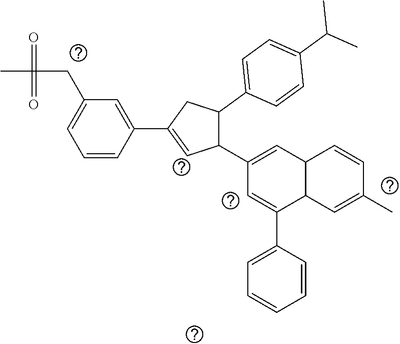 | PZ240 | 87.09 | 104.4 |
| 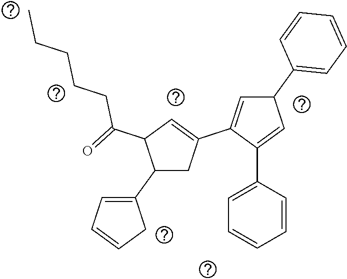 [SMILES: CCCCCC(=O)C1C=C(C2=CC(c3ccccc3)C=C2c2ccccc2)CC1C1=CC=CC1] | PZ241 | 92.13 | 108.3 |
| 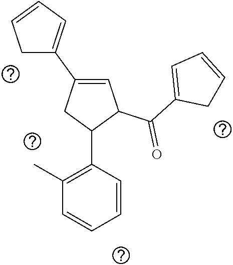 [SMILES: Cc1ccccc1C1CC(C2=CC=CC2)=CC1C(=O)C1=CC=CC1] | PZ242 | 96.43 | 95.19 |
| 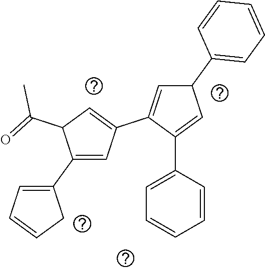 [SMILES: CC(=O)C1C=C(C2=CC(c3ccccc3)C=C2c2ccccc2)C=C1C1=CC=CC1] | PZ243 | 71.52 | 68.72 |
| 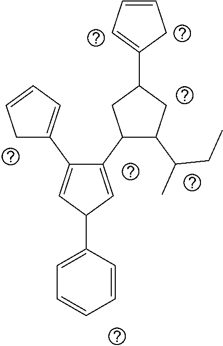 [SMILES: CCC(C)C1CC(C2=CC=CC2)CC1C1=CC(c2ccccc2)C=C1C1=CC=CC1] | PZ244 | 62.63 | 59.28 |
| 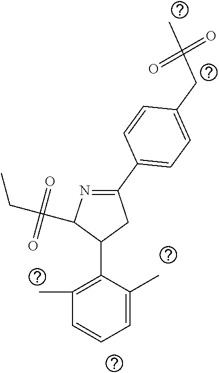 | PZ245 | 53.16 | 65.72 |
| 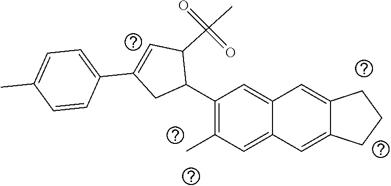 | PZ246 | 45.43 | 52.54 |
| 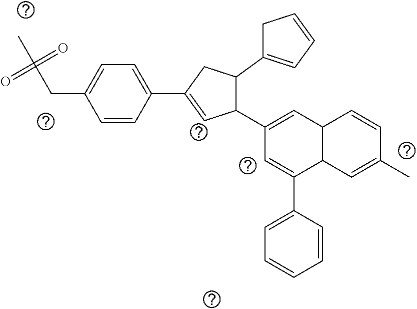 | PZ247 | 81.84 | 113.1 |
| 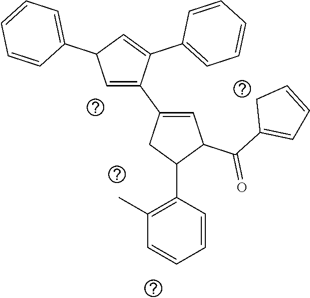 [SMILES: Cc1ccccc1C1CC(C2=CC(c3ccccc3)C=C2c2ccccc2)=CC1C(=O)C1=CC=CC1] | PZ248 | 72.92 | 92.41 |
| 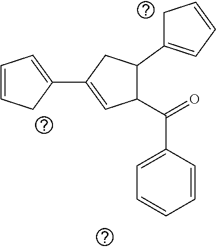 [SMILES: O=C(c1ccccc1)C1C=C(C2=CC=CC2)CC1C1=CC=CC1] | PZ249 | 63.27 | 61.4 |
| 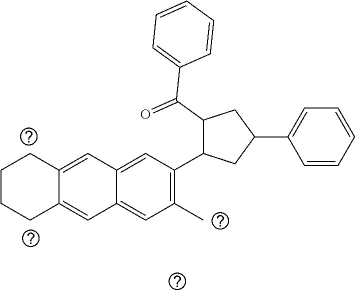 [SMILES: Cc1cc2cc3c(cc2cc1C1CC(c2ccccc2)CC1C(=O)c1ccccc1)CCCC3] | PZ250 | 61.56 | 77.96 |
| 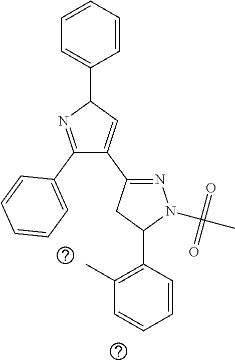 | PZ251 | 45.32 | 51.87 |
| 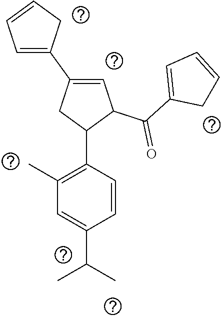 [SMILES: Cc1cc(C(C)C)ccc1C1CC(C2=CC=CC2)=CC1C(=O)C1=CC=CC1] | PZ252 | 81.53 | 108 |
| 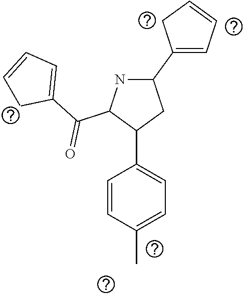 [SMILES: Cc1ccc(C2CC(C3=CC=CC3)NC2C(=O)C2=CC=CC2)cc1] | PZ253 | 86.58 | 94.65 |
| 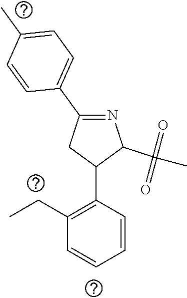 | PZ254 | 44.33 | 53.54 |
| 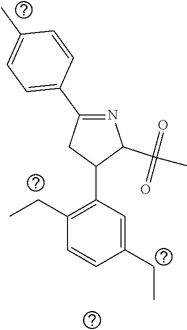 | PZ255 | 48.49 | 58.15 |
| 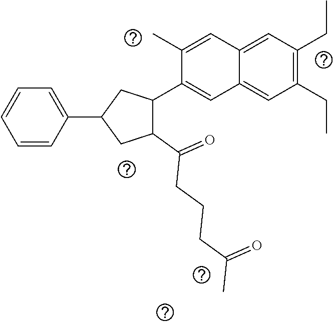 [SMILES: CCc1cc2cc(C)c(C3CC(c4ccccc4)CC3C(=O)CCCC(C)=O)cc2cc1CC] | PZ256 | 90.01 | 82.99 |
| 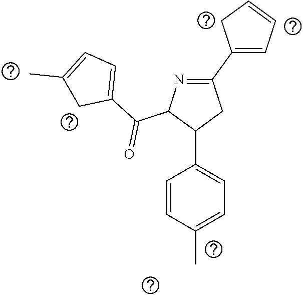 [SMILES: CC1=CC=C(C(=O)C2N=C(C3=CC=CC3)CC2c2ccc(C)cc2)C1] | PZ257 | 74.67 | 95.47 |
| 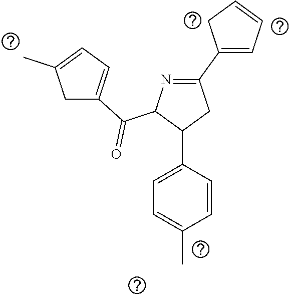 [SMILES: CC1=CC=C(C(=O)C2N=C(C3=CC=CC3)CC2c2ccc(C)cc2)C1] | PZ258 | 77.72 | 71.72 |
| 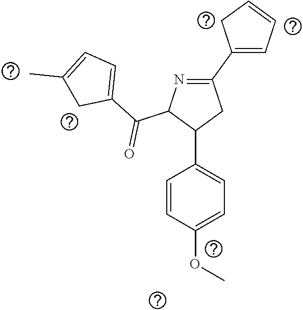 [SMILES: COc1ccc(C2CC(C3=CC=CC3)=NC2C(=O)C2=CC=C(C)C2)cc1] | PZ259 | 75.77 | 97.52 |
| 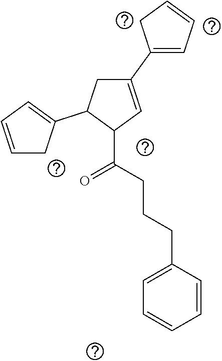 [SMILES: O=C(CCCc1ccccc1)C1C=C(C2=CC=CC2)CC1C1=CC=CC1] | PZ260 | 82.91 | 106 |
| 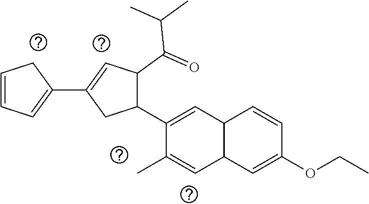 [SMILES: CCOC1=CC2C=C(C)C(C3CC(C4=CC=CC4)=CC3C(=O)C(C)C)=CC2C=C1] | PZ261 | 92.1 | 134.2 |
| 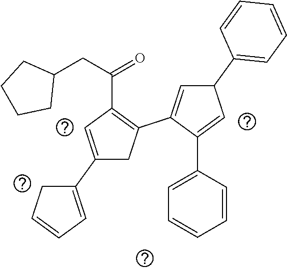 [SMILES: O=C(CC1CCCC1)C1=C(C2=CC(c3ccccc3)C=C2c2ccccc2)CC(C2=CC=CC2)=C1] | PZ262 | 41.46 | 46.26 |
| 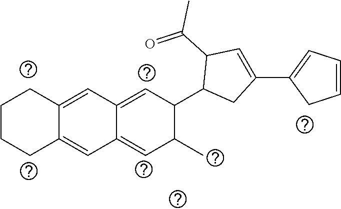 [SMILES: CC(=O)C1C=C(C2=CC=CC2)CC1C1C=c2cc3c(cc2=CC1C)CCCC3] | PZ263 | 86.73 | 91.88 |
| 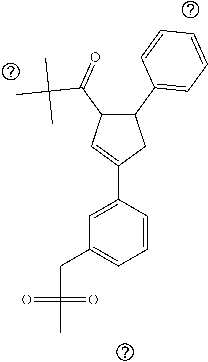 | PZ264 | 87.11 | 93.83 |
| 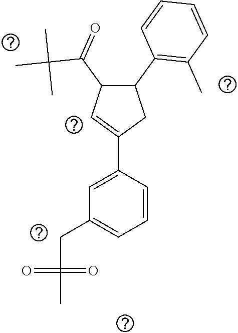 | PZ265 | 80.69 | 90.44 |
| 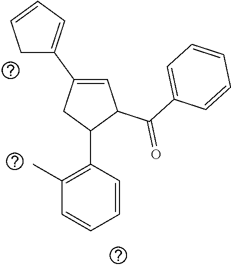 [SMILES: Cc1ccccc1C1CC(C2=CC=CC2)=CC1C(=O)c1ccccc1] | PZ266 | 70.06 | 67.53 |
| 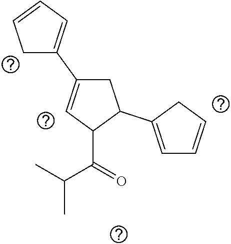 [SMILES: CC(C)C(=O)C1C=C(C2=CC=CC2)CC1C1=CC=CC1] | PZ267 | 79.52 | 92.77 |
| 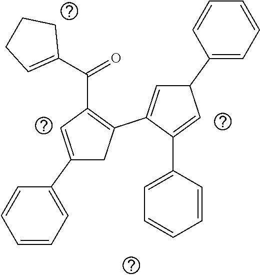 [SMILES: O=C(C1=CCCC1)C1=C(C2=CC(c3ccccc3)C=C2c2ccccc2)CC(c2ccccc2)=C1] | PZ268 | 48.96 | 70.35 |
| 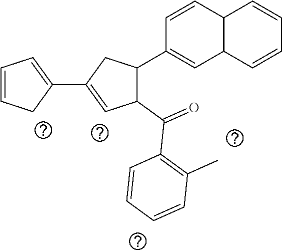 [SMILES: Cc1ccccc1C(=O)C1C=C(C2=CC=CC2)CC1C1=CC2C=CC=CC2C=C1] | PZ269 | 69.21 | 71.96 |
| 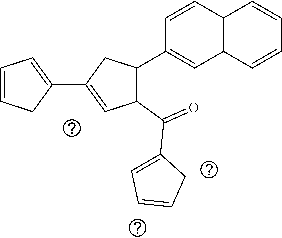 [SMILES: O=C(C1=CC=CC1)C1C=C(C2=CC=CC2)CC1C1=CC2C=CC=CC2C=C1] | PZ270 | 66.12 | 101.9 |
| 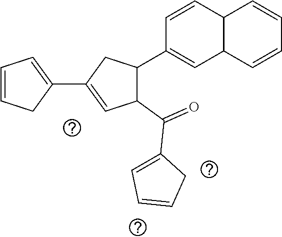 [SMILES: O=C(C1=CC=CC1)C1C=C(C2=CC=CC2)CC1C1=CC2C=CC=CC2C=C1] | PZ271 | 82.14 | 77.23 |
| 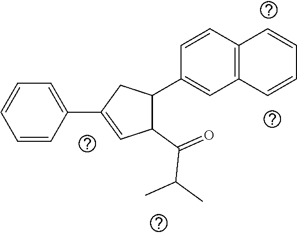 [SMILES: CC(C)C(=O)C1C=C(c2ccccc2)CC1c1ccc2ccccc2c1] | PZ272 | 97.81 | 119 |
| 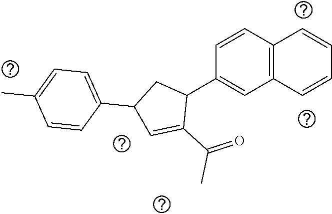 [SMILES: CC(=O)C1=CC(c2ccc(C)cc2)CC1c1ccc2ccccc2c1] | PZ273 | 35.6 | 103 |
| 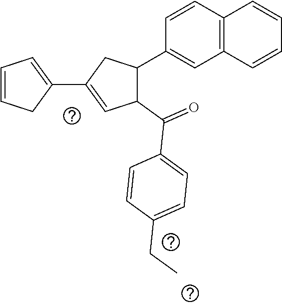 [SMILES: CCc1ccc(C(=O)C2C=C(C3=CC=CC3)CC2c2ccc3ccccc3c2)cc1] | PZ274 | 92.63 | 127.2 |
| 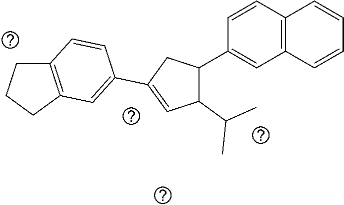 [SMILES: CC(C)C1C=C(c2ccc3c(c2)CCC3)CC1c1ccc2ccccc2c1] | PZ275 | 65.52 | 73.3 |
| 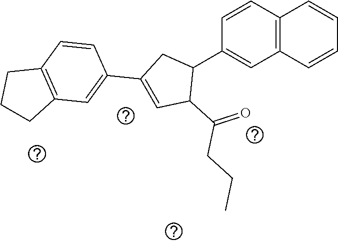 [SMILES: CCCC(=O)C1C=C(c2ccc3c(c2)CCC3)CC1c1ccc2ccccc2c1] | PZ276 | 66.48 | 76.46 |
| 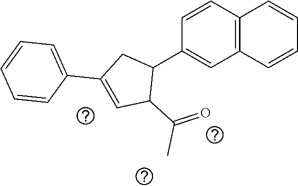 [SMILES: CC(=O)C1C=C(c2ccccc2)CC1c1ccc2ccccc2c1] | PZ277 | 83.42 | 88.84 |
| 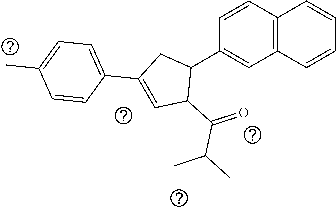 [SMILES: Cc1ccc(C2=CC(C(=O)C(C)C)C(c3ccc4ccccc4c3)C2)cc1] | PZ278 | 94.12 | 110.9 |
| 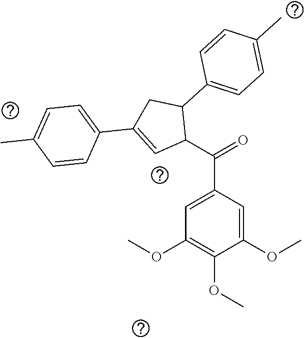 | PZ279 | 102.7 | 70.85 |
| 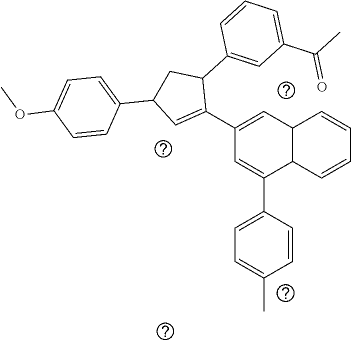 [SMILES: COc1ccc(C2C=C(C3=CC4C=CC=CC4C(c4ccc(C)cc4)=C3)C(c3cccc(C(C)=O)c3)C2)cc1] | PZ280 | 94.96 | 124.2 |
| 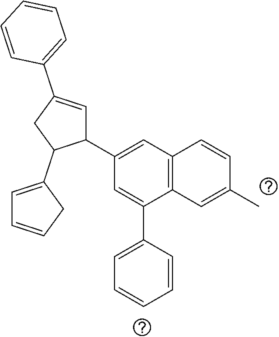 [SMILES: Cc1ccc2cc(C3C=C(c4ccccc4)CC3C3=CC=CC3)cc(-c3ccccc3)c2c1] | PZ281 | 89.52 | 103.9 |
| 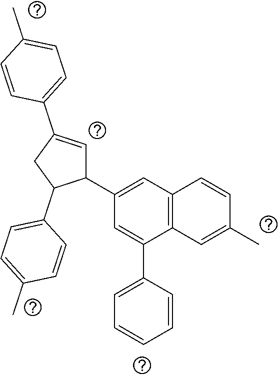 [SMILES: Cc1ccc(C2=CC(c3cc(-c4ccccc4)c4cc(C)ccc4c3)C(c3ccc(C)cc3)C2)cc1] | PZ282 | 97.55 | 117.3 |
| 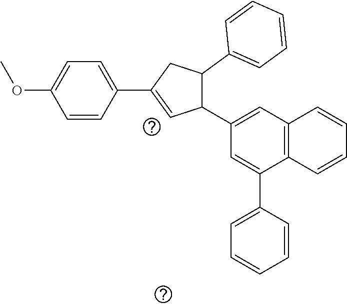 [SMILES: COc1ccc(C2=CC(c3cc(-c4ccccc4)c4ccccc4c3)C(c3ccccc3)C2)cc1] | PZ283 | 96.81 | 121.7 |
| 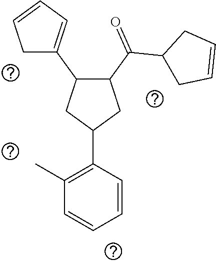 [SMILES: Cc1ccccc1C1CC(C(=O)C2CC=CC2)C(C2=CC=CC2)C1] | PZ284 | 94.15 | 119.2 |
| 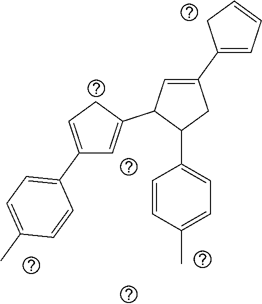 [SMILES: Cc1ccc(C2=CCC(C3C=C(C4=CC=CC4)CC3c3ccc(C)cc3)=C2)cc1] | PZ285 | 89.82 | [symbol] |
| 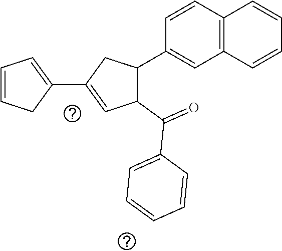 [SMILES: O=C(c1ccccc1)C1C=C(C2=CC=CC2)CC1c1ccc2ccccc2c1] | PZ286 | 90.92 | 76.61 |
| 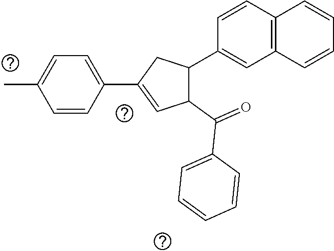 [SMILES: Cc1ccc(C2=CC(C(=O)c3ccccc3)C(c3ccc4ccccc4c3)C2)cc1] | PZ287 | 105.9 | 108.8 |
| 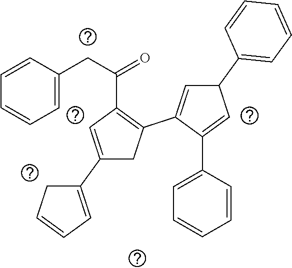 [SMILES: O=C(Cc1ccccc1)C1=C(C2=CC(c3ccccc3)C=C2c2ccccc2)CC(C2=CC=CC2)=C1] | PZ288 | 67.38 | 67.19 |
| 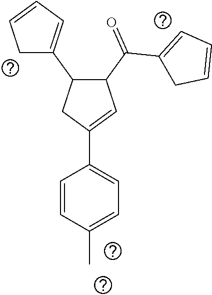 [SMILES: Cc1ccc(C2=CC(C(=O)C3=CC=CC3)C(C3=CC=CC3)C2)cc1] | PZ289 | 85.3 | 75.35 |
| 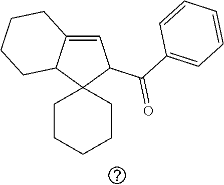 [SMILES: O=C(c1ccccc1)C1C=C2CCCCC2C12CCCCC2] | PZ290 | 90.26 | 80.27 |
| 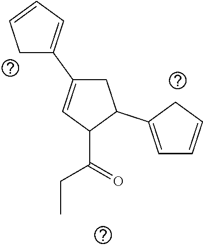 [SMILES: CCC(=O)C1C=C(C2=CC=CC2)CC1C1=CC=CC1] | PZ291 | 96.35 | 99.27 |
| 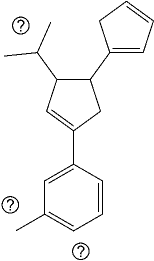 [SMILES: Cc1cccc(C2=CC(C(C)C)C(C3=CC=CC3)C2)c1] | PZ292 | 126.5 | 82.11 |
| 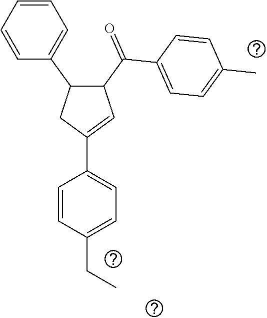 [SMILES: CCc1ccc(C2=CC(C(=O)c3ccc(C)cc3)C(c3ccccc3)C2)cc1] | PZ293 | 110.3 | 83.33 |
| 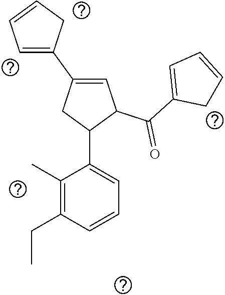 [SMILES: CCc1cccc(C2CC(C3=CC=CC3)=CC2C(=O)C2=CC=CC2)c1C] | PZ294 | 80.69 | 76.35 |
| 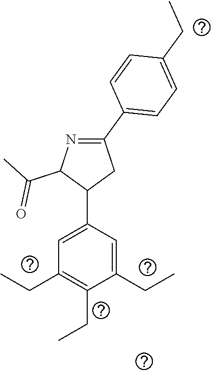 [SMILES: CCc1ccc(C2=NC(C(C)=O)C(c3cc(CC)c(CC)c(CC)c3)C2)cc1] | PZ295 | 85.97 | 69.96 |
| 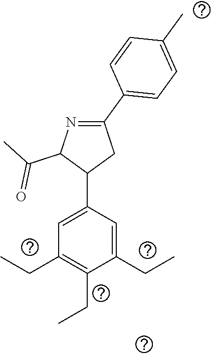 [SMILES: CCc1cc(C2CC(c3ccc(C)cc3)=NC2C(C)=O)cc(CC)c1CC] | PZ296 | 91.93 | 92.75 |
| 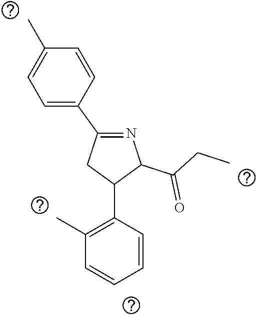 [SMILES: CCC(=O)C1N=C(c2ccc(C)cc2)CC1c1ccccc1C] | PZ297 | 98.3 | 99.96 |
| 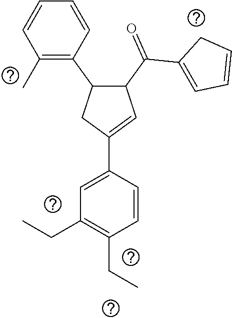 [SMILES: CCc1ccc(C2=CC(C(=O)C3=CC=CC3)C(c3ccccc3C)C2)cc1CC] | PZ298 | 140 | 73.73 |
| 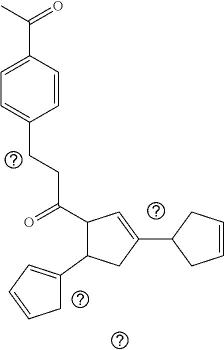 [SMILES: CC(=O)c1ccc(CCC(=O)C2C=C(C3CC=CC3)CC2C2=CC=CC2)cc1] | PZ299 | 81.74 | 72.07 |
| 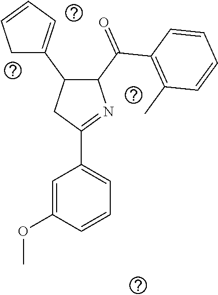 [SMILES: COc1cccc(C2=NC(C(=O)c3ccccc3C)C(C3=CC=CC3)C2)c1] | PZ300 | 83.02 | 63.9 |
| 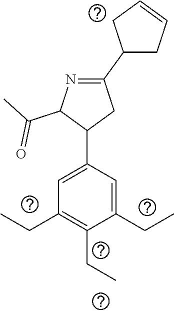 [SMILES: CCc1cc(C2CC(C3CC=CC3)=NC2C(C)=O)cc(CC)c1CC] | PZ301 | 104.9 | 74.68 |
| 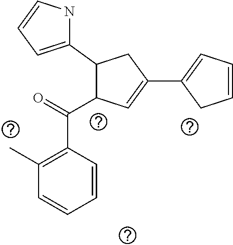 [SMILES: Cc1ccccc1C(=O)C1C=C(C2=CC=CC2)CC1c1ccc[nH]1] | PZ302 | 74.35 | 68.66 |
| 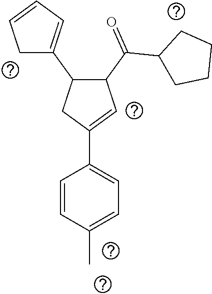 [SMILES: Cc1ccc(C2=CC(C(=O)C3CCCC3)C(C3=CC=CC3)C2)cc1] | PZ303 | 138.1 | 91.57 |
| 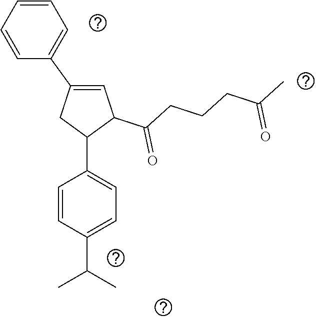 [SMILES: CC(=O)CCCC(=O)C1C=C(c2ccccc2)CC1c1ccc(C(C)C)cc1] | PZ304 | 92.98 | 85.34 |
| 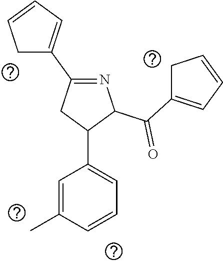 [SMILES: Cc1cccc(C2CC(C3=CC=CC3)=NC2C(=O)C2=CC=CC2)c1] | PZ305 | 93.27 | 88.01 |
| 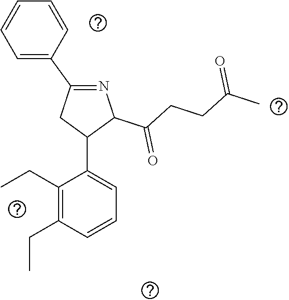 [SMILES: CCc1cccc(C2CC(c3ccccc3)=NC2C(=O)CCC(C)=O)c1CC] | PZ306 | 90.26 | 34.92 |
| 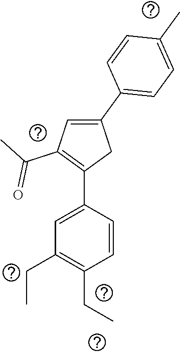 [SMILES: CCc1ccc(C2=C(C(C)=O)C=C(c3ccc(C)cc3)C2)cc1CC] | PZ307 | 111.1 | 93.63 |
| 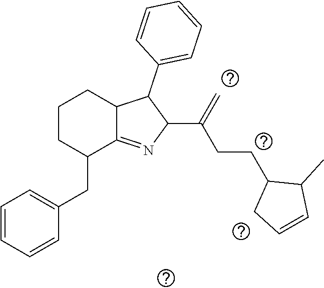 [SMILES: C=C(CCC1CC=CC1C)C1N=C2C(Cc3ccccc3)CCCC2C1c1ccccc1] | PZ308 | 115.5 | 87.62 |
| 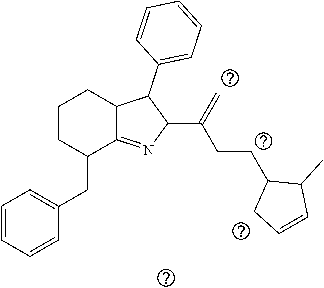 [SMILES: C=C(CCC1CC=CC1C)C1N=C2C(Cc3ccccc3)CCCC2C1c1ccccc1] | PZ309 | 66.59 | 69.38 |
| 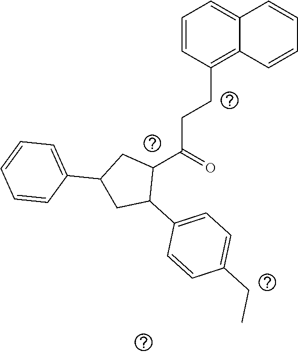 [SMILES: CCc1ccc(C2CC(c3ccccc3)CC2C(=O)CCc2cccc3ccccc23)cc1] | PZ310 | 70.63 | 26.92 |
| 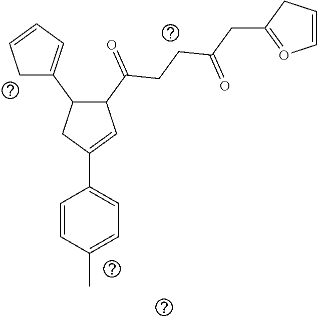 | PZ311 | 101.3 | 86.77 |
| 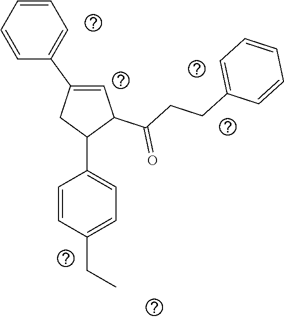 [SMILES: CCc1ccc(C2CC(c3ccccc3)=CC2C(=O)CCc2ccccc2)cc1] | PZ312 | 86.96 | 43.96 |
| 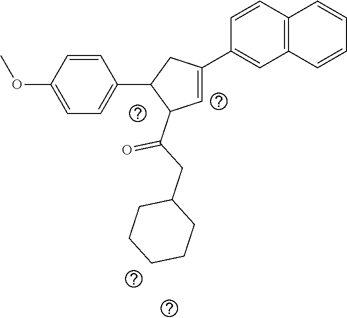 [SMILES: COc1ccc(C2CC(c3ccc4ccccc4c3)=CC2C(=O)CC2CCCCC2)cc1] | PZ313 | 85.96 | 111.6 |
| 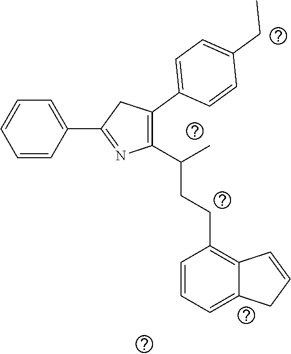 [SMILES: CCc1ccc(C2=C(C(C)CCc3cccc4c3C=CC4)N=C(c3ccccc3)C2)cc1] | PZ314 | 95.24 | 37.67 |
| 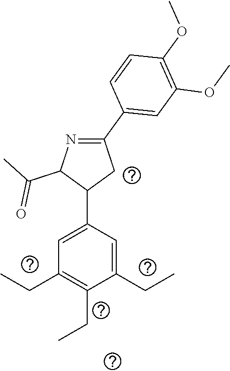 [SMILES: CCc1cc(C2CC(c3ccc(OC)c(OC)c3)=NC2C(C)=O)cc(CC)c1CC] | PZ315 | 130.3 | 75.28 |
| 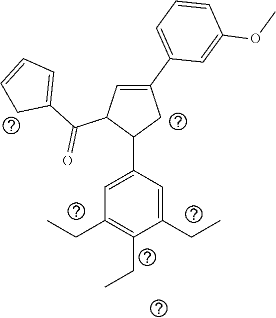 [SMILES: CCc1cc(C2CC(c3cccc(OC)c3)=CC2C(=O)C2=CC=CC2)cc(CC)c1CC] | PZ316 | 79.03 | 48.9 |
| 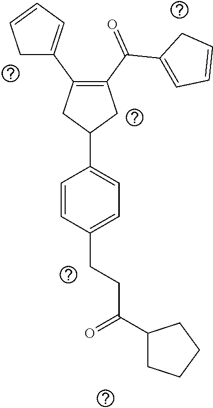 [SMILES: O=C(C1=CC=CC1)C1=C(C2=CC=CC2)CC(c2ccc(CCC(=O)C3CCCC3)cc2)C1] | PZ317 | 87.02 | 85.72 |
| 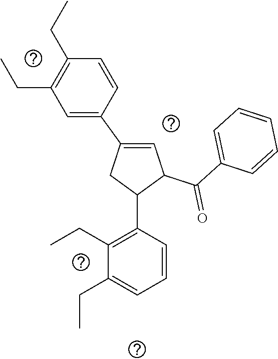 [SMILES: CCc1ccc(C2=CC(C(=O)c3ccccc3)C(c3cccc(CC)c3CC)C2)cc1CC] | PZ318 | 91.48 | 68.81 |
| 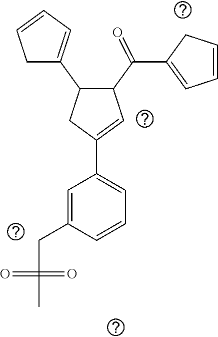 | PZ319 | 81.22 | 56.89 |
| 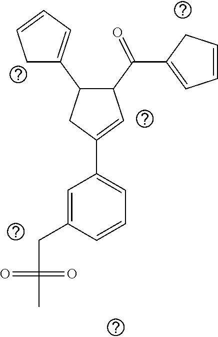 | PZ320 | 72.62 | 64.35 |
| 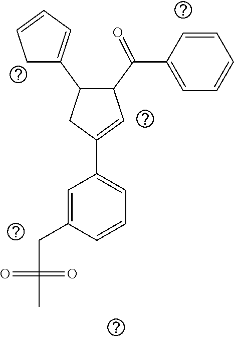 | PZ321 | 82.59 | 64.48 |
| 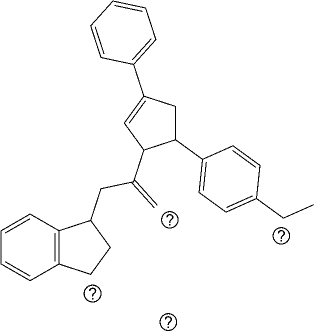 [SMILES: C=C(CC1CCc2ccccc21)C1C=C(c2ccccc2)CC1c1ccc(CC)cc1] | PZ322 | 84.65 | 64.86 |
| 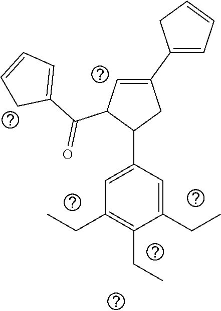 [SMILES: CCc1cc(C2CC(C3=CC=CC3)=CC2C(=O)C2=CC=CC2)cc(CC)c1CC] | PZ323 | 131.4 | 97.84 |
| 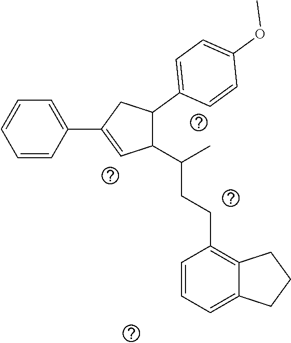 [SMILES: COc1ccc(C2CC(c3ccccc3)=CC2C(C)CCc2cccc3c2CCC3)cc1] | PZ324 | 96.69 | 38.72 |
| 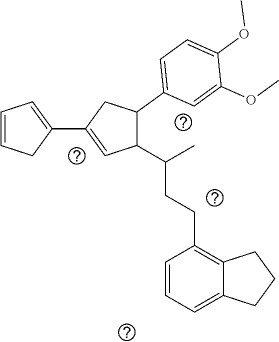 [SMILES: COc1ccc(C2CC(C3=CC=CC3)=CC2C(C)CCc2cccc3c2CCC3)cc1OC] | PZ325 | 90.31 | 61.64 |
| 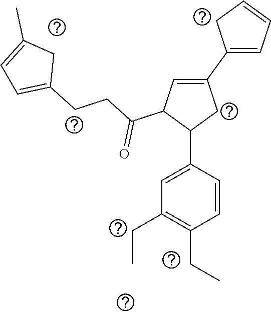 [SMILES: CCc1ccc(C2CC(C3=CC=CC3)=CC2C(=O)CCC2=CC=C(C)C2)cc1CC] | PZ326 | 68.06 | 78.84 |
| 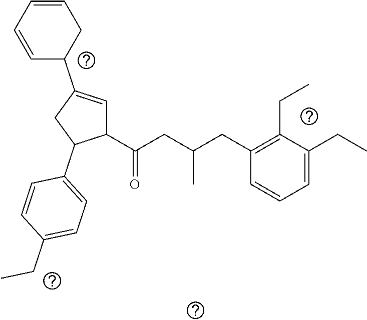 [SMILES: CCc1ccc(C2CC(C3C=CC=CC3)=CC2C(=O)CC(C)Cc2cccc(CC)c2CC)cc1] | PZ327 | 79.39 | 79.35 |
| 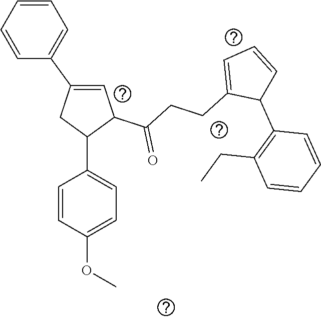 [SMILES: CCc1ccccc1C1C=CC=C1CCC(=O)C1C=C(c2ccccc2)CC1c1ccc(OC)cc1] | PZ328 | 53.18 | 41.7 |
| 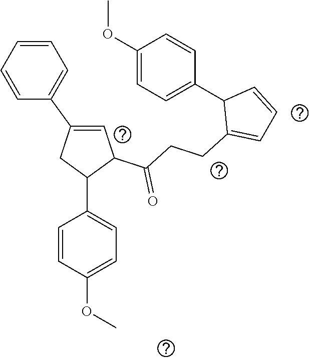 [SMILES: COc1ccc(C2C=CC=C2CCC(=O)C2C=C(c3ccccc3)CC2c2ccc(OC)cc2)cc1] | PZ329 | 75.2 | 31.1 |
| 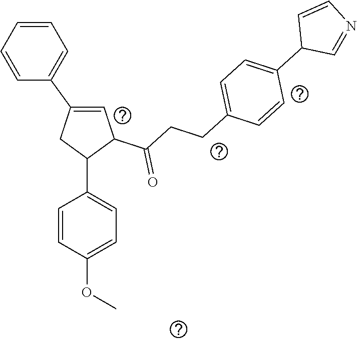 [SMILES: COc1ccc(C2CC(c3ccccc3)=CC2C(=O)CCc2ccc(C3C=CN=C3)cc2)cc1] | PZ330 | 126.3 | 48.49 |
| 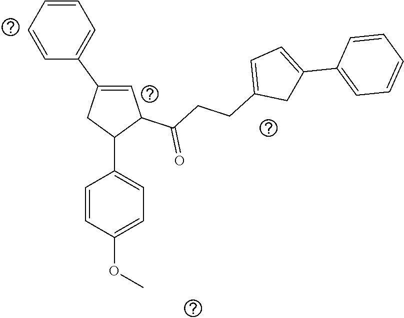 [SMILES: COc1ccc(C2CC(c3ccccc3)=CC2C(=O)CCC2=CC=C(c3ccccc3)C2)cc1] | PZ331 | 91.76 | 58.96 |
| 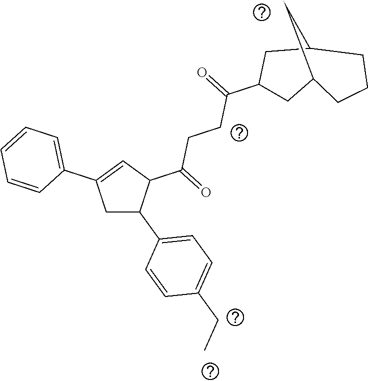 [SMILES: CCc1ccc(C2CC(c3ccccc3)=CC2C(=O)CCC(=O)C2CC3CCCC(C3)C2)cc1] | PZ332 | 97.1 | 72.85 |
| 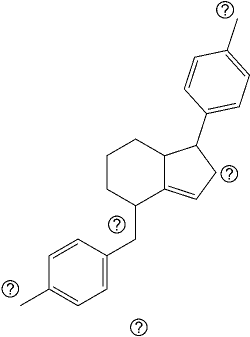 [SMILES: Cc1ccc(CC2CCCC3C2=CCC3c2ccc(C)cc2)cc1] | PZ333 | 90 | 76.95 |
| 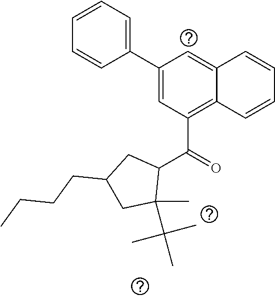 [SMILES: CCCCC1CC(C(=O)c2cc(-c3ccccc3)cc3ccccc23)C(C)(C(C)(C)C)C1] | PZ334 | 94.56 | 86.03 |
| 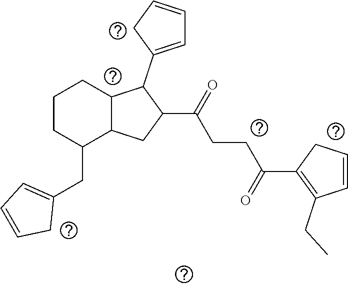 [SMILES: CCC1=C(C(=O)CCC(=O)C2CC3C(CC4=CC=CC4)CCCC3C2C2=CC=CC2)CC=C1] | PZ335 | 38.09 | 102.9 |
| 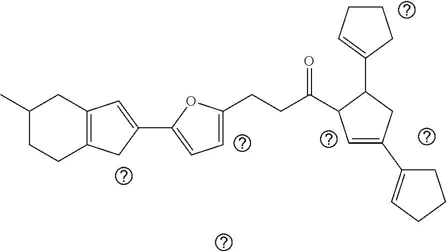 [SMILES: CC1CCC2=C(C=C(c3ccc(CCC(=O)C4C=C(C5=CCCC5)CC4C4=CCCC4)o3)C2)C1] | PZ336 | 97.36 | 85.86 |
| 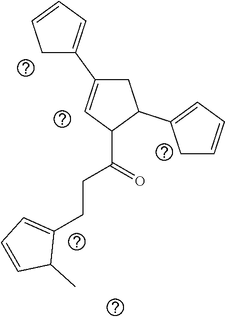 [SMILES: CC1C=CC=C1CCC(=O)C1C=C(C2=CC=CC2)CC1C1=CC=CC1] | PZ337 | 77.16 | 61.97 |
| 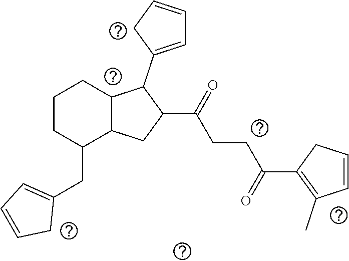 [SMILES: CC1=C(C(=O)CCC(=O)C2CC3C(CC4=CC=CC4)CCCC3C2C2=CC=CC2)CC=C1] | PZ338 | 91.78 | 83.38 |
| 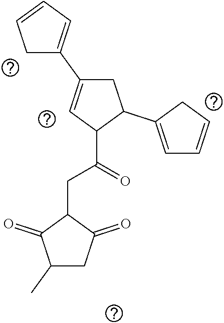 [SMILES: CC1CC(=O)C(CC(=O)C2C=C(C3=CC=CC3)CC2C2=CC=CC2)C1=O] | PZ339 | 97.79 | 147.7 |
| 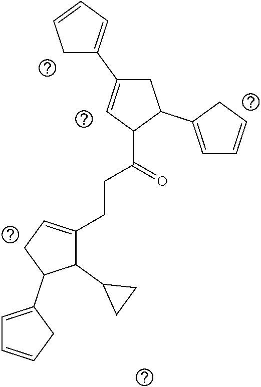 [SMILES: O=C(CCC1=CCC(C2=CC=CC2)C1C1CC1)C1C=C(C2=CC=CC2)CC1C1=CC=CC1] | PZ340 | 92.71 | 88.36 |
| 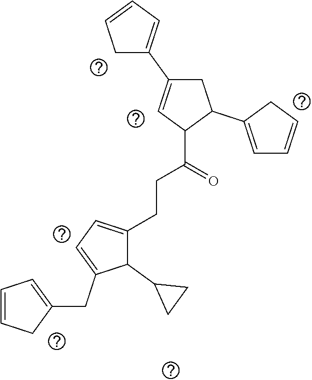 [SMILES: O=C(CCC1=CC=C(CC2=CC=CC2)C1C1CC1)C1C=C(C2=CC=CC2)CC1C1=CC=CC1] | PZ341 | 74.62 | 79.22 |
| 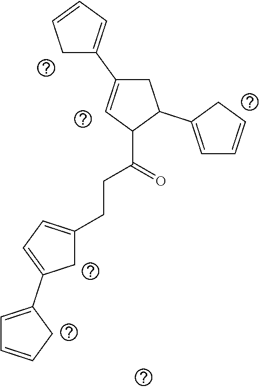 [SMILES: O=C(CCC1=CC=C(C2=CC=CC2)C1)C1C=C(C2=CC=CC2)CC1C1=CC=CC1] | PZ342 | 82.48 | 78.86 |
| 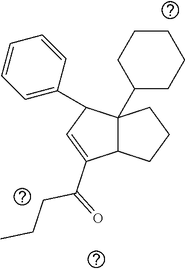 [SMILES: CCCC(=O)C1=CC(c2ccccc2)C2(C3CCCCC3)CCCC12] | PZ343 | 105.1 | 97.02 |
| 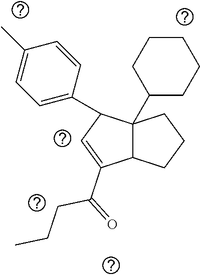 [SMILES: CCCC(=O)C1=CC(c2ccc(C)cc2)C2(C3CCCCC3)CCCC12] | PZ344 | 119.2 | 98.33 |
| 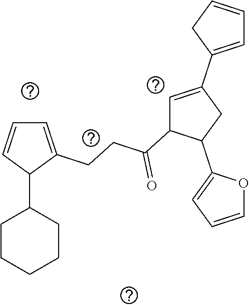 [SMILES: O=C(CCC1=CC=CC1C1CCCCC1)C1C=C(C2=CC=CC2)CC1c1ccco1] | PZ345 | 83.02 | 81.29 |
| 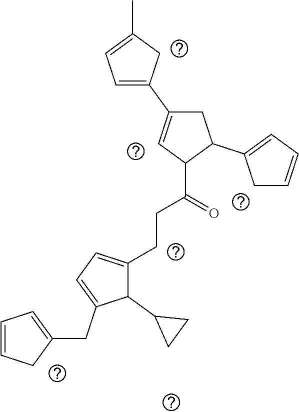 [SMILES: CC1=CC=C(C2=CC(C(=O)CCC3=CC=C(CC4=CC=CC4)C3C3CC3)C(C3=CC=CC3)C2)C1] | PZ346 | 79.91 | 93.88 |
| 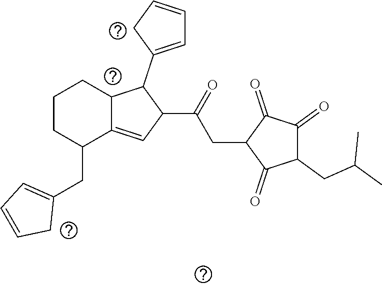 [SMILES: CC(C)CC1C(=O)C(=O)C(CC(=O)C2C=C3C(CC4=CC=CC4)CCCC3C2C2=CC=CC2)C1=O] | PZ347 | 87.15 | 126.9 |
| 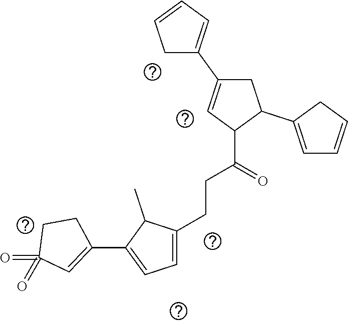 | PZ348 | 105.3 | 99.16 |
| 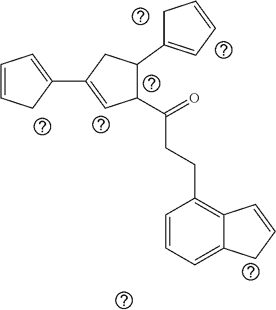 [SMILES: O=C(CCc1cccc2c1C=CC2)C1C=C(C2=CC=CC2)CC1C1=CC=CC1] | PZ349 | 91.29 | 67.91 |
| 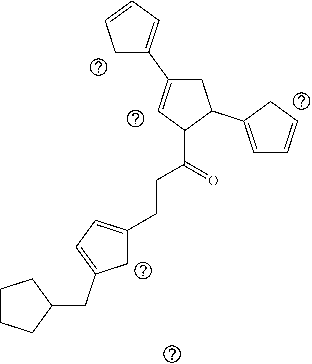 [SMILES: O=C(CCC1=CC=C(CC2CCCC2)C1)C1C=C(C2=CC=CC2)CC1C1=CC=CC1] | PZ350 | 72.37 | 70.86 |
| 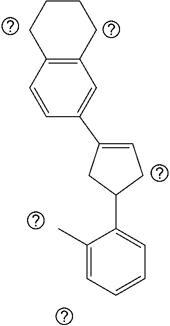 [SMILES: Cc1ccccc1C1CC=C(c2ccc3c(c2)CCCC3)C1] | PVZF0024 | 15.8 | 23.4 |
| 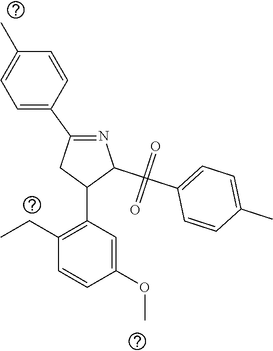 | PVZF0043 | 35.33 | 36.69 |
| 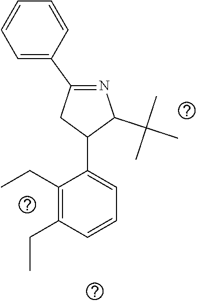 [SMILES: CCc1cccc(C2CC(c3ccccc3)=NC2C(C)(C)C)c1CC] | PVZF0044 | 42.08 | 55.91 |
| 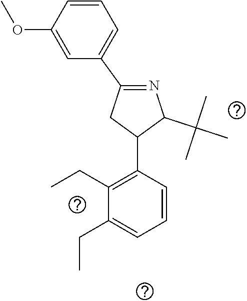 [SMILES: CCc1cccc(C2CC(c3cccc(OC)c3)=NC2C(C)(C)C)c1CC] | PVZF0045 | 38.8 | 60.68 |
| 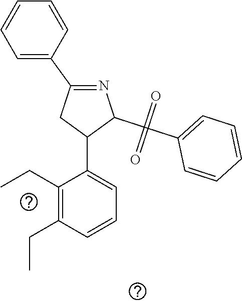 | PVZF0046 | 47.04 | 54.68 |
| 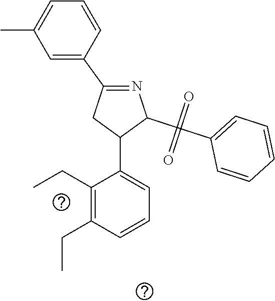 | PVZF0049 | 67.85 | 91.32 |
| 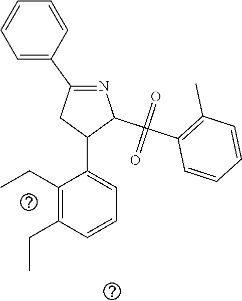 | PVZF0050 | 58.65 | 106.2 |
| 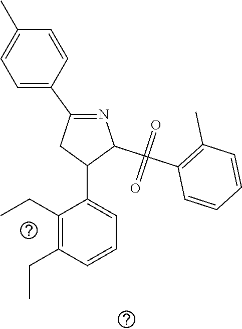 | PVZF0051 | 40.74 | 36.59 |
| 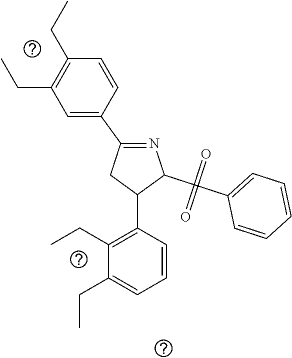 | PVZF0052 | 71.62 | 54.38 |
| 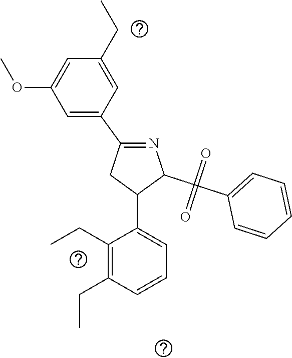 | PVZF0053 | 68.68 | 58.96 |
| 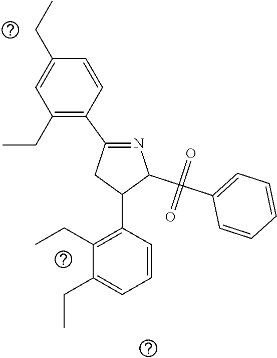 | PVZF0054 | 56.4 | 49.48 |
| 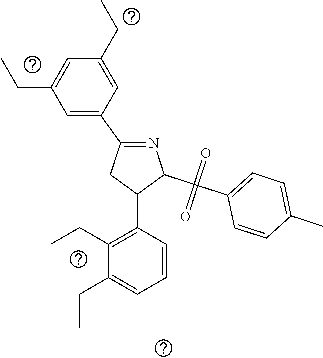 | PVZF0055 | 46.42 | 45.63 |
| 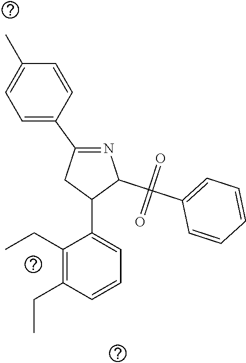 | PVZF0056 | 48.33 | 53.76 |
| 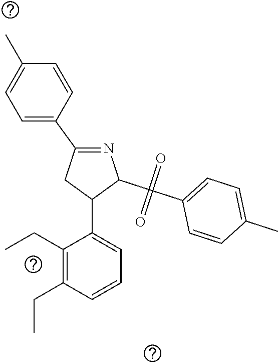 | PVZF0057 | 50.71 | 61.81 |
| 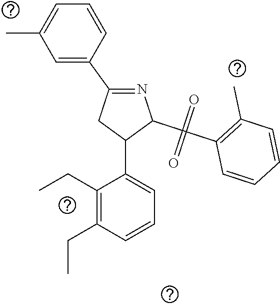 | PVZF0058 | 54.21 | 66.66 |
| 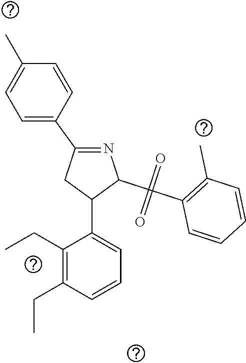 | PVZF0059 | 43.94 | 38.46 |
| 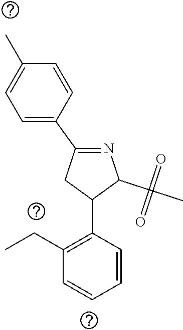 | PVZF0060 | 38.44 | 44.13 |
| 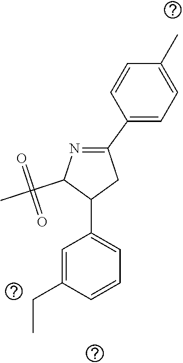 | PVZF0061 | 69.94 | 121.9 |
| 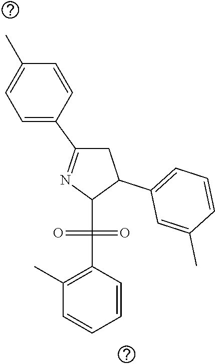 | PVZF0062 | 54.91 | 73.79 |
| 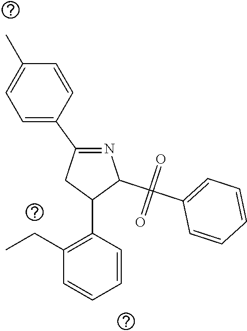 | PVZF0063 | 43.6 | 56.3 |
| 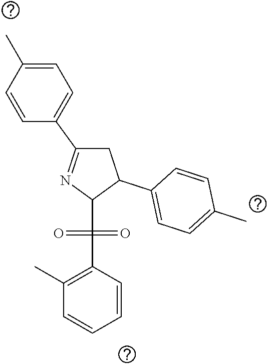 | PVZF0064 | 58.28 | 93.3 |
| 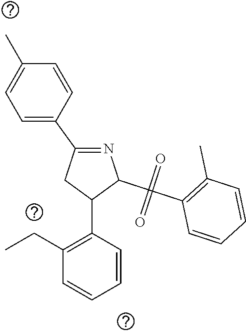 | PVZF0065 | 48.96 | 67.6 |
| 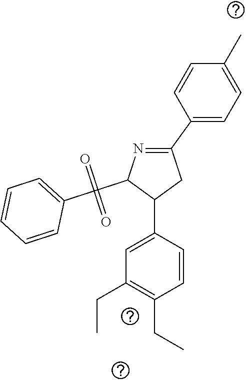 | PVZF0066 | 56.14 | 65.56 |
| 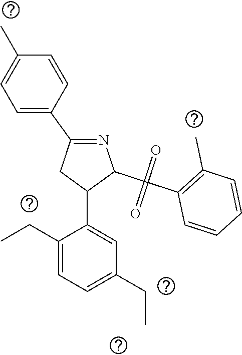 | PVZF0067 | 32.14 | 34.58 |
| 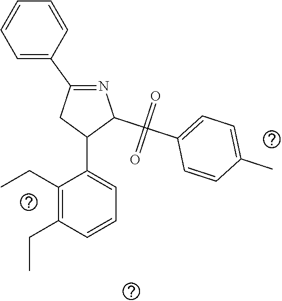 | PVZF0068 | 39.33 | 51.65 |
| 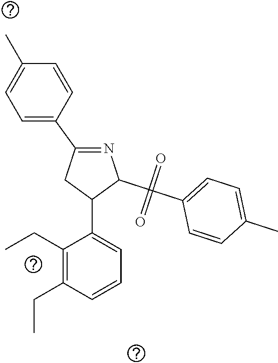 | PVZF0069 | 46.34 | 49.99 |
| 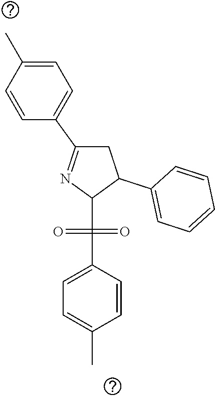 | PVZF0072 | 60.73 | 76.88 |
| 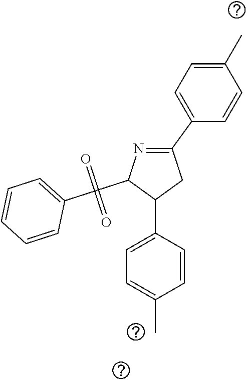 | PVZF0073 | 60.62 | 86.43 |
| 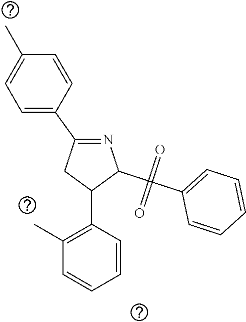 | PVZF0074 | 79.16 | 96.63 |
| 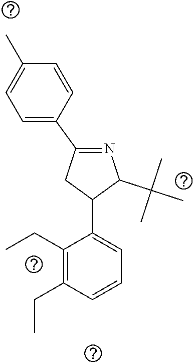 [SMILES: CCc1cccc(C2CC(c3ccc(C)cc3)=NC2C(C)(C)C)c1CC] | PVZF0075 | 60.92 | 51.94 |
| 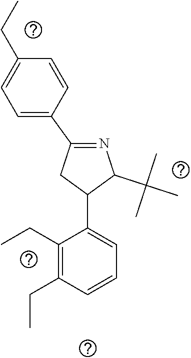 [SMILES: CCc1ccc(C2=NC(C(C)(C)C)C(c3cccc(CC)c3CC)C2)cc1] | PVZF0076 | 39.28 | 56.82 |
| 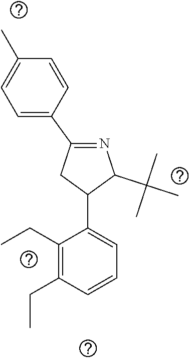 [SMILES: CCc1cccc(C2CC(c3ccc(C)cc3)=NC2C(C)(C)C)c1CC] | PVZF0077 | 37.39 | 49.7 |
| 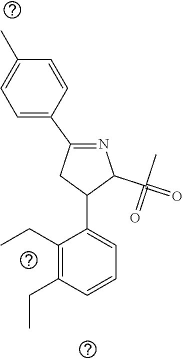 | PVZF0079 | 53.25 | 79.74 |
| 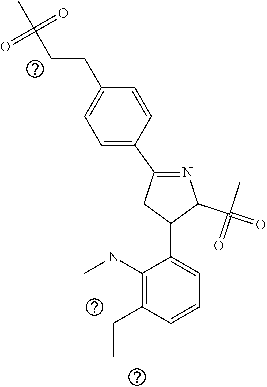 | PVZF0080 | 99.06 | 137.4 |
| 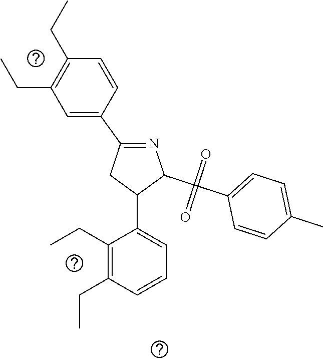 | PVZF0081 | 57.42 | 56.18 |
| 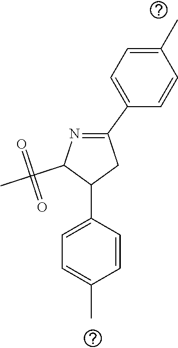 | PVZF0082 | 87.78 | 172.9 |
| 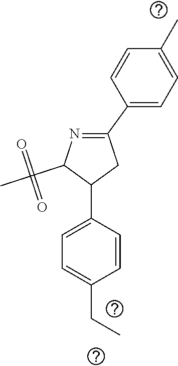 | PVZF0083 | 57.89 | 104.5 |
| 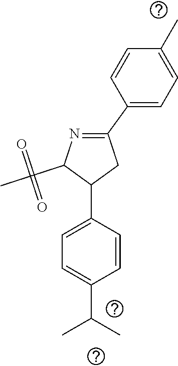 | PVZF0084 | 71.08 | 146.9 |
| 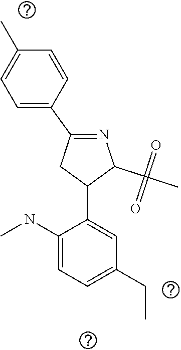 | PVZF0085 | 44.61 | 65.42 |
| 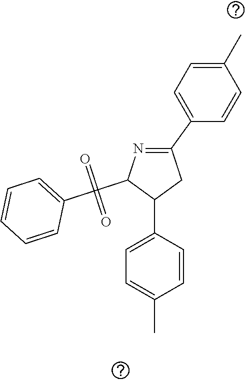 | PVZF0086 | 58.32 | 57.06 |
| 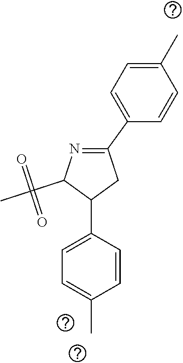 | PVZF0087 | 81.35 | 187.1 |
| 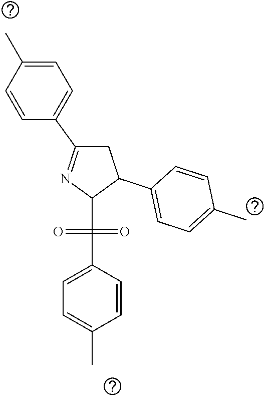 | PVZF0088 | 57.94 | 72.24 |
| 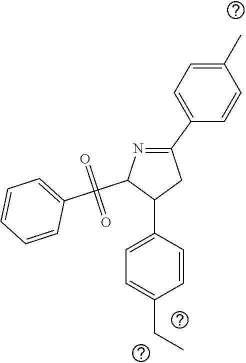 | PVZF0089 | 67.1 | 70.13 |
| 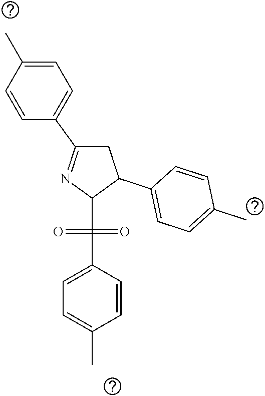 | PVZF0090 | 77.12 | 114.3 |
| 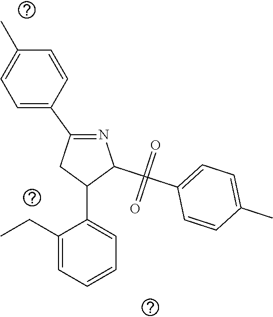 | PVZF0091 | 34.27 | 40.68 |
| 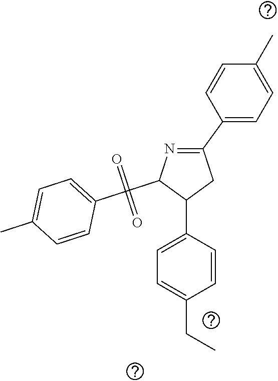 | PVZF0092 | 48.91 | 54.44 |
| 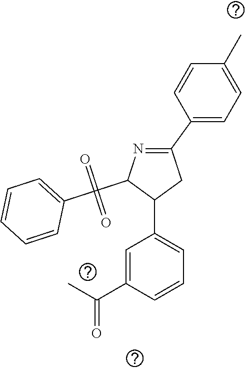 | PVZF0093 | 45.45 | 59.77 |
| 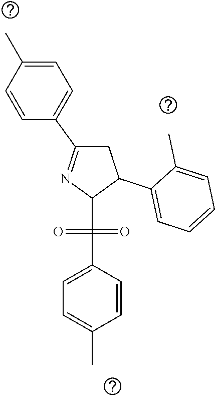 | PVZF0094 | 69.05 | 97.79 |
| 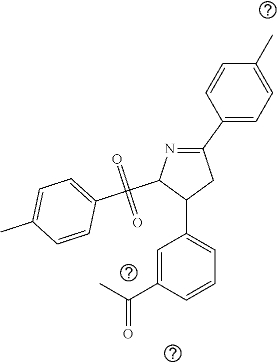 | PVZF0095 | 47.01 | 66.76 |
| 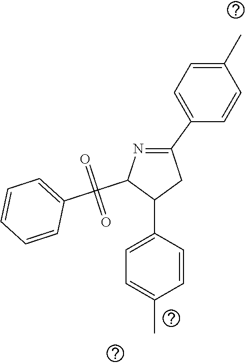 | PVZF0096 | 73.68 | 103.6 |
| 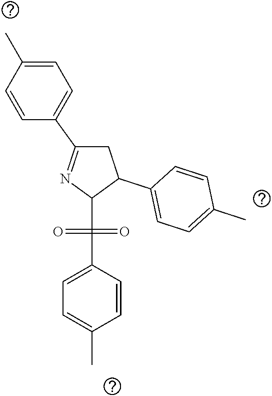 | PVZF0097 | 63.4 | 63.96 |
| 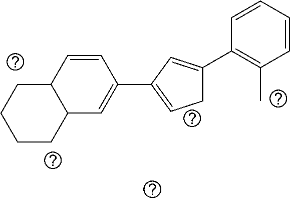 [SMILES: Cc1ccccc1C1=CC(C2=CC3CCCCC3C=C2)=CC1] | PVZF0101 | 13.0 (5 μM) 10.0 (10 μM) | 16.1 (5 μM) 12.5 (10 μM) |

[symbol]

indicates data missing or illegible when filed

### Screening Test

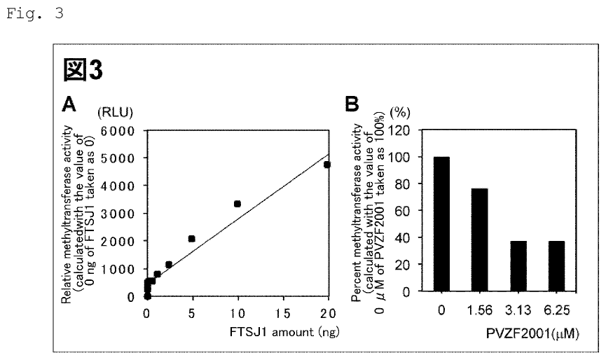

HEK293 human embryonic kidney cells that constantly express FLAG-tagged FTSJ1 were produced, and from its cell lysate, FLAG-FTSJ1 was separated by adsorption using an anti-FLAG M2 antibody affinity gel (Sigma-Aldrich Co. LLC, catalog number: A2220-10ML), and eluted with FLAG peptide (Sigma-Aldrich Co. LLC, catalog number: F3290-25MG). After measuring the protein concentration in the eluate, serial dilutions were performed within the range of 0 to 20 ng/reaction to obtain enzyme dilutions of 12 different concentrations. Further, the total RNA was extracted from HEK293-FTSJ1-KO cells, in which FTSJ1 was knocked out, using TRIzol Reagent (Thermo Fisher Scientific Inc., catalog number: 15596018). In order to measure the activity which FLAG-FTSJ1 caused transmethylation with RNA as a substrate, the enzyme dilutions, the total RNA (1,000 ng/reaction), and a methyltransferase activity assay kit (Promega Corporation, catalog number: V7601) were used. The conversion reaction from SAM (s-adenosyl methionine) to SAH (s-adenosyl homocysteine), which occurred when FLAG-FTSJ1 in each dilution methylated 1,000 ng/reaction of the total RNA, was measured as a luciferase luminescence value using MTase-Glo Reagent in the assay kit. Taking the luminescence value at the time when FLAG-FTSJ1 was 0 ng as a background value, a graph was drawn using the numerical values obtained by subtracting the background value from the luminescence value at each dilution step. As shown in FIG. 3A, a dose-dependent increase in the luminescence value of FLAG-FTSJ1 was observed, confirming that detecting the enzyme activity of FTSJ1 was possible.

Using the above system, the inhibitory effect of transmethylation by PVZF2001, which is an FTSJ1 inhibitor, was confirmed. The luminescence values were measured as described above by mixing different concentrations of PVZF2001 dilutions with the use of 10 ng/reaction of FLAG-FTSJ1, 1,000 ng/reaction of the total RNA, and the methyltransferase activity assay kit. As shown in FIG. 3B, a decrease in the luminescence value was observed in a concentration-dependent manner of PVZF2001, confirming that PVZF2001 certainly inhibited transmethylation caused by FTSJ1.

### Anti-Tumor Effect Evaluation Test

A cell suspension (1×10⁶ cells/100 μL), in which triple-negative breast cancer cell line MDA-MB-231 was suspended in phosphate buffer (PBS, produced by FUJIFILM Wako Pure Chemical Corporation), was inoculated subcutaneously in the dorsal lumbar region of immunodeficient mice (BALB/c-nu/nu, female, 6 weeks old, purchased from Shimizu Laboratory Supplies Co., Ltd.) using a syringe with a 23 G injection needle (Terumo Corporation). After confirming that the cancer cells engrafted under the skin of the mice to form tumor tissues, and that the tumor volume reached 100 mm³, the obtained tumor model mice were used for the following evaluation test. First, each compound for evaluation was dissolved in corn oil (produced by Sigma-Aldrich Co., LLC) and administered intraperitoneally. PVZF0024 was administered at 100 mg/kg and PVZF2001 was administered at 20 mg/kg to the tumor model mice every other day. At each administration, the tumor diameter was measured with a caliper. The tumor volume was calculated according to the following formula: V=(3.14×D×d²)/6 (wherein V is the tumor volume, D is the tumor major axis, and d is the tumor minor axis) (Wu. et al., Clin. Cancer Res., 2013 Oct. 15; 19(20): 5699-5710).

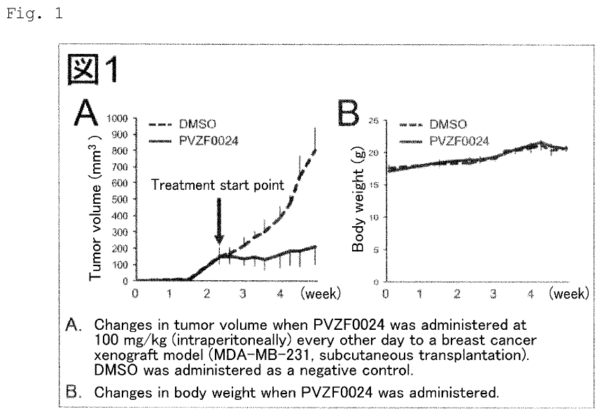

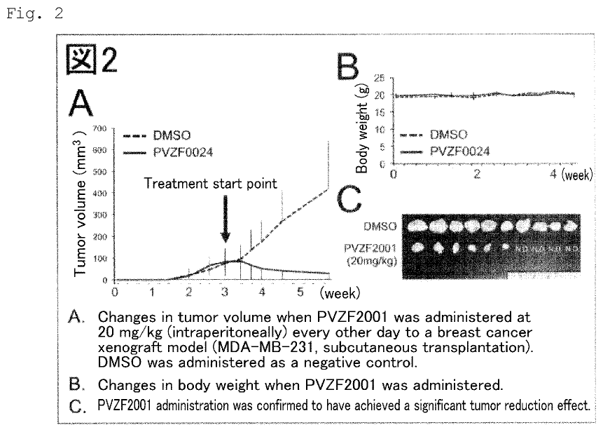

FIGS. 1 and 2 show the antitumor effects of PVZF0024 and PVZF2001. As shown in each figure, both of the compounds were confirmed to significantly inhibit tumor growth; additionally, no weight loss due to administration was observed.

### Test for Evaluating FTSJ1 Inhibitor Sensitive Genetic Marker

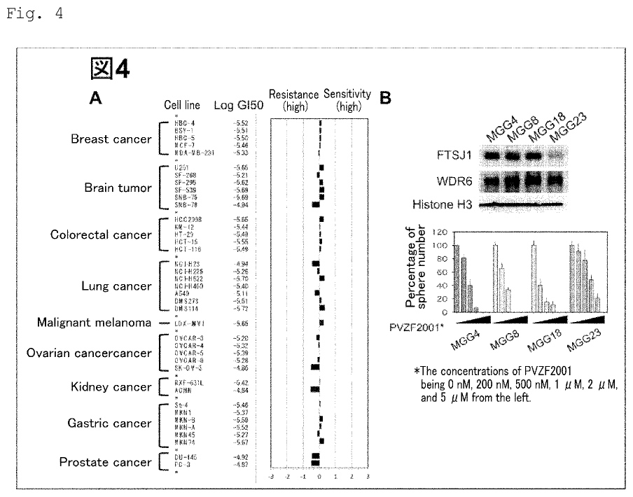

In order to extract genes that prescribe the sensitivity and resistance with respect to the FTSJ1 inhibitors, PVZF2001 was used to proceed with the analysis with a cell line panel (JFCR39). Each of 39 types of human cancer cell lines was treated with PVZF2001 at concentrations of 0.01 μM, 0.1 μM, 1 μM, 10 μM, and 100 μM for 48 hours, and cell proliferation was measured by colorimetric quantification with sulforhodamine B. FIG. 4A shows the obtained Log GI50 values. The GI50 value is defined, based on the number of cells before drug exposure, as a concentration at which the increase is inhibited by 50% by drug treatment, with the increase in a sample cultured for 48 hours without drug treatment (negative control) taken as 100%. In order to extract the target gene clusters, a COMPARE analysis was first performed using gene expression and the Log GI50 values in all of the cell lines. The gene clusters of SEQ ID NOs: 14 to 28 were then identified as FTSJ1 inhibitor resistance-related genetic markers, while the gene clusters of SEQ ID NOs: 34 to 47 were identified as FTSJ1 inhibitor sensitive-related genetic markers. Furthermore, focusing on the malignant brain tumors and the lung cancers in which clearly distinguishable sensitivity and resistance by PVZF2001 were observed, gene expression data of the cell lines with high sensitivity (e.g., U251, SF-539, SNB-75, NCI-H522, DMS114) and the cell lines with high resistance (e.g., SNB-78, NCI-H23) were each used to analyze gene clusters that underwent increased expression in each of the cell lines in common. The gene clusters of SEQ ID NOs: 29 to 33 were then identified as FTSJ1 inhibitor resistance-related genetic markers, while the gene clusters of SEQ ID NOs: 48 to 56 were identified as FTSJ1 inhibitor sensitive-related genetic markers. Using these genetic markers, a database of gene expression information (TCGA) obtained from patient samples was analyzed. Patients with a high FTSJ1 gene expression level showed a high expression level of the FTSJ1 inhibitor sensitive-related genetic markers; conversely, patients with a low FTSJ1 gene expression level showed a high expression level of FTSJ1 inhibitor resistance-related genetic markers. That is, the results reveal that the FTSJ1 gene expression level itself also represents sensitivity and resistance with respect to the FTSJ1 inhibitors (SEQ ID NO: 57).

To verify whether the expression analysis of these gene clusters contributes to predicting the anti-cancer effect by the FTSJ1 inhibitors, the FTSJ1 gene was actually used as an example to analyze the anti-cancer effect of the FTSJ1 inhibitor PVZF2001 on human malignant brain tumor cell lines. Human malignant brain tumor cell lines MGG4, MGG8, MGG18, MGG23 were treated with stem cell medium (Neurobasal, B-27, N-2, 20 ng/mL EGF, 20 ng/mL bFGF) containing different concentrations (0 nm, 200 nM, 500 nM, 1000 NM, 2000 NM, 5000 nM) of PVZF2001 for 1 week, and anchorage-independent cell proliferation was evaluated based on sphere-forming ability. As a result, as shown in FIG. 4B, MGG4, MGG8, and MGG18, which showed a high FTSJ1 expression level, showed proliferation inhibition at low concentrations of PVZF2001; whereas MGG23, which showed a low FTSJ1 expression level, showed cell proliferation even when treated at a concentration as high as 2000 NM.

### Production Example 1

### (E)-1-(2,3-dihydrobenzo[b] [1,4]dioxin-6-yl)-3-(2-hydroxyphenyl)prop-2-en-1-one (PVZF0024)

1-(2,3-Dihydrobenzo[b][1,4]dioxin-6-yl)ethan-1-one (178 mg) and 2-hydroxybenzaldehyde (122 mg) were dissolved in 20 ml of ethanol. A 40% sodium hydroxide solution (0.5 mL) was added thereto, and the mixture was stirred at 60° C. for 10 hours. After neutralization with acetic acid, the resulting product was extracted with chloroform and purified by silica gel chromatography to give (E)-1-(2,3-dihydrobenzo[b][1,4]dioxin-6-yl)-3-(2-hydroxyphenyl)prop-2-en-1-one. The above compound (141 mg) was dissolved in 10 mL of ethanol, 121 μL of hydrazine monohydrate was added thereto, and the mixture was stirred at 80° C. for 5 hours. Ethanol was distilled off under reduced pressure, and the obtained residue was purified by silica gel chromatography to give 31 mg of the target compound in a yield of 31%.

ESI (m/z): 297 (M+H)⁺

¹H-NMR (400 MHz, DMSO-d₆) δ 9.66 (d, J=25.4 Hz, 1H), 7.25-7.27 (m, 1H), 7.05-7.12 (m, 3H), 6.74-6.86 (m, 3H), 4.95 (td, J=10.5, 2.8 Hz, 1H), 4.25 (s, 4H), 3.32-3.38 (m, 1H), 2.66 (dd, J=16.3, 10.5 Hz, 1H)

### Production Example 2

### 2,4,6-Triisopropyl-N-(piperidin-4-yl)benzenesulfonamide (PVZF2005)

2,4,6-Triisopropylbenzenesulfonyl chloride (151 mg) was dissolved in 10 mL of dichloromethane, 50 mg of piperidin-4-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 158 mg of the target compound in a yield of 86%.

ESI (m/z): 367 (M+H)⁺

¹H-NMR (400 MHz, DMSO-d₆) δ 7.29-7.48 (s, 2H), 4.05 (td, J=13.4, 6.7 Hz, 3H), 3.43 (m, 8H), 1.91-1.95 (m, 1H), 1.44 (td, J=11.8, 3.1 Hz, 1H), 1.05-1.38 (m, 18H)

### Production Example 3

### 2,4,6-Triisopropyl-N-(piperidin-3-ylmethyl)benzenesulfonamide) (PVZF2008)

2,4,6-Triisopropylbenzenesulfonyl chloride (151 mg) was dissolved in 10 mL of dichloromethane, 57 mg of piperidin-3-ylmethanamine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 129 mg of the target compound in a yield of 68%.

ESI (m/z): 381 (M+H)⁺

¹H-NMR (400 MHz, DMSO-d6) δ 7.22-7.28 (m, 2H), 4.08 (td, J=13.3, 6.7 Hz, 2H), 3.57-3.64 (m, 1H), 2.90-2.97 (m, 1H), 2.62-2.74 (m, 3H), 1.70-1.91 (m, 3H), 1.09-1.29 (m, 22H)

### Production Example 4

### N-(2-(cyclohexy-1-en-1-yl)ethyl)-[1,1′-biphenyl]-4-sulfonamide (PVZF2035)

[1,1′-Biphenyl]-4-sulfonyl chloride (126 mg) was dissolved in 10 mL of dichloromethane, 62.5 mg of 2-(cyclohex-1-en-1-yl)ethan-1-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 123 mg of the target compound in a yield of 72%.

ESI (m/z): 342 (M+H)⁺

¹H-NMR (400 MHz, DMSO-d₆) δ 7.85-7.90 (m, 4H), 7.74-7.76 (m, 2H), 7.64 (d, J=23.2 Hz, 1H), 7.52 (t, J=7.7 Hz, 2H), 7.42-7.46 (m, 1H), 5.33 (s, 1H), 2.83 (t, J=7.4 Hz, 2H), 2.01 (t, J=7.2 Hz, 2H), 1.89 (s, 2H), 1.80 (s, 2H), 1.43-1.56 (m, 4H)

### Production Example 5

### N-(2,2,6,6-Tetramethylpiperidin-4-yl)-[1,1′-biphenyl]-4-sulfonamide (PVZF0036)

[1,1′-Biphenyl]-4-sulfonyl chloride (126 mg) was dissolved in 10 mL of dichloromethane, 78 mg of 2,2,6,6-tetramethylpiperidin-4-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 153 mg of the target compound in a yield of 82%.

ESI (m/z): 373 (M+H)⁺

¹H-NMR (400 MHz, DMSO-d₆) δ 7.88-7.92 (m, 4H), 7.75 (t, J=8.4 Hz, 2H), 7.49-7.55 (m, 2H), 7.42-7.47 (m, 1H), 3.46-3.57 (m, 1H), 1.54-1.61 (m, 2H), 1.39 (m, 2H), 1.13-1.37 (m, 12H)

### Production Example 6

### N-Cyclohexyl-4-(tert-pentyl)benzenesulfonamide (PVZF0039)

4-(tert-Pentyl)benzenesulfonyl chloride (123 mg) was dissolved in 10 mL of dichloromethane, 50 mg of cyclohexanamine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 105 mg of the target compound in a yield of 68%.

ESI (m/z): 310 (M+H)⁺

¹H-NMR (400 MHz, DMSO-d₆) δ 7.72-7.81 (m, 2H), 7.56 (s, 1H), 7.53 (d, J=8.0 Hz, 2H), 2.91 (s, 1H), 1.63 (q, J=7.4 Hz, 2H), 1.55 (d, J=4.6 Hz, 2H), 1.41-1.44 (m, 1H), 1.27 (s, 6H), 1.05-1.15 (m, 4H), 1.01 (d, J=11.0 Hz, 2H), 0.58 (t, J=7.3 Hz, 3H)

### Production Example 7

### 4-Cyclohexyl-N-pentylbenzenesulfonamide (PVZF2065)

4-Cyclohexylbenzenesulfonyl chloride (129 mg) was dissolved in 10 mL of dichloromethane, 44 mg of pentan-1-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 121 mg of the target compound in a yield of 78%.

ESI (m/z): 310 (M+H)⁺

¹H-NMR (400 MHz, DMSO-d₆) δ 7.67-7.70 (m, 2H), 7.47 (s, 1H), 7.43 (d, J=8.0 Hz, 2H), 2.57-2.72 (m, 3H), 1.79 (d, J=10.7 Hz, 4H), 1.71 (d, J=12.7 Hz, 1H), 1.25-1.56 (m, 7H), 1.12-1.23 (m, 4H), 0.79 (dd, J=7.0, 6.0 Hz, 3H)

### Production Example 8

### N,4-Dicyclohexylbenzenesulfonamide (PVZF2066)

4-Cyclohexylbenzenesulfonyl chloride (129 mg) was dissolved in 10 mL of dichloromethane, 50 mg of cyclohexanamine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 135 mg of the target compound in a yield of 84%.

ESI (m/z): 322 (M+H)⁺

¹H-NMR (400 MHz, DMSO-d₆) δ 7.71 (d, J=8.3 Hz, 2H), 7.56 (s, 1H), 7.42 (d, J=8.3 Hz, 2H), 2.91 (s, 1H), 2.56-2.67 (m, 1H), 1.78-1.85 (m, 4H), 1.71 (d, J=12.4 Hz, 1H), 1.56 (d, J=6.6 Hz, 4H), 1.32-1.43 (m, 5H), 1.25 (t, J=12.2 Hz, 1H), 1.00-1.16 (m, 5H)

### Production Example 9

### 4-Cyclohexyl-N-(cyclohexylmethyl)benzenesulfonamide (PVZF2069)

4-Cyclohexylbenzenesulfonyl chloride (129 mg) was dissolved in 10 mL of dichloromethane, 58 mg of cyclohexylmethanamine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 131 mg of the target compound in a yield of 78%.

ESI (m/z): 336 (M+H)⁺

¹H-NMR (400 MHz, DMSO-d₆) δ 7.68 (d, J=8.3 Hz, 2H), 7.49 (s, 1H), 7.43 (d, J=8.3 Hz, 2H), 2.53-2.67 (m, 6H), 1.77-1.84 (m, 3H), 1.71 (d, J=12.4 Hz, 2H), 1.61 (d, J=11.2 Hz, 3H), 1.35-1.46 (m, 2H), 1.22-1.32 (m, 1H), 1.07-1.15 (m, 3H), 0.74-0.82 (m, 2H)

### Production Example 10

### 4-Bromo-2-isopropyl-N-(2,2,6,6-tetramethylpiperidin-4-yl)benzenesulfonamide (PVZF2074)

4-Bromo-2-isopropylbenzenesulfonyl chloride (148 mg) was dissolved in 10 mL of dichloromethane, 78 mg of 2,2,6,6-tetramethylpiperidin-4-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 175 mg of the target compound in a yield of 85%.

ESI (m/z): 417 (M+H)⁺

¹H-NMR (400 MHz, DMSO-d₆) δ 8.14 (bs, 1H), 7.78 (d, J=8.5 Hz, 2H), 7.57-7.60 (m, 1H), 3.75-3.82 (m, 1H), 1.60 (s, 2H), 1.13-1.39 (m, 22H)

### Production Example 11

### 2-(tert-Butyl)-N-(2,2,6,6-tetramethylpiperidin-4-yl)benzenesulfonamide (PVZF2075)

2-(tert-Butyl)benzenesulfonyl chloride (116 mg) was dissolved in 10 mL of dichloromethane, 78 mg of 2,2,6,6-tetramethylpiperidin-4-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 141 mg of the target compound (PVZF2075) in a yield of 80%.

ESI (m/z): 353 (M+H)⁺

¹H-NMR (400 MHz, DMSO-d₆) δ 8.05 (d, J=8.0 Hz, 2H), 7.66 (d, J=8.0 Hz, 1H), 7.53-7.56 (m, 1H), 7.43 (t, J=7.7 Hz, 1H), 1.68-1.75 (m, 2H), 1.52 (s, 9H), 1.15-1.29 (n, 16H)

### Production Example 12

### 2-Isopropyl-4-methoxy-5-methyl-N-(2,2,6,6-tetramethylpiperidin-4-yl)benzenesulfonamide (PVZF2076)

2-Isopropyl-4-methoxy-5-methylbenzenesulfonyl chloride (131 mg) was dissolved in 10 mL of dichloromethane, 78 mg of 2,2,6,6-tetramethylpiperidin-4-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 145 mg of the target compound in a yield of 76%.

ESI (m/z): 383 (M+H)⁺

¹H-NMR (400 MHz, DMSO-d₆) δ 7.77 (bs, 1H), 7.64 (s, 1), 7.03 (s, 1H), 3.88 (s, 3H), 3.76-3.83 (m, 1H), 2.20-2.08 (3H), 1.59 (d, J=12.4 Hz, 2H), 1.13-1.33 (m, 22H)

### Production Example 13

### 2,5-Diisopropyl-N-(2,2,6,6-tetramethylpiperidin-4-yl)benzenesulfonamide (PVZF2077)

2,5-Diisopropylbenzenesulfonyl chloride (130 mg) was dissolved in 10 mL of dichloromethane, 78 mg of 2,2,6,6-tetramethylpiperidin-4-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 164 mg of the target compound in a yield of 86%.

ESI (m/z): 381 (M+H)⁺

¹H-NMR (400 MHz, DMSO-d₆) δ 7.93 (bs, 1H), 7.71-7.79 (m, 1H), 7.44-7.54 (m, 2H), 3.76-3.83 (m, 1H), 3.54 (d, J=25.6 Hz, 1H), 3.05-2.86 (1H), 1.54 (d, J=11.5 Hz, 2H), 1.03-1.39 (m, 26H)

### Production Example 14

### 2-Cyclopropyl-N-(2,2,6,6-tetramethylpiperidin-4-yl)benzenesulfonamide (PVZF2078)

2-Cyclopropylbenzenesulfonyl chloride (108 mg) was dissolved in 10 mL of dichloromethane, 78 mg of 2,2,6,6-tetramethylpiperidin-4-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 133 mg of the target compound in a yield of 79%.

ESI (m/z): 337 (M+H)⁺

¹H-NMR (400 MHz, DMSO-d₆) δ 8.02-7.91 (1H), 7.89 (d, J=7.8 Hz, 1H), 7.52 (t, J=7.4 Hz, 1H), 7.32 (t, J=7.7 Hz, 1H), 7.05 (d, J=7.8 Hz, 1H), 3.51 (s, 2H), 2.64-2.70 (m, 1H), 1.60 (d, J=10.0 Hz, 2H), 1.03-1.35 (m, 16H), 0.80-0.84 (m, 2H)

### Production Example 15

### 5-Chloro-2-cyclopropyl-N-(2,2,6,6-tetramethylpiperidin-4-yl)benzenesulfonamide (PVZF2079)

5-Chloro-2-cyclopropylbenzenesulfonyl chloride (125 mg) was dissolved in 10 mL of dichloromethane, 78 mg of 2,2,6,6-tetramethylpiperidin-4-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 152 mg of the target compound in a yield of 82%.

ESI (m/z): 371 (M+H)⁺

¹H-NMR (400 MHz, DMSO-d₆) δ 8.14 (d, J=28.8 Hz, 1H), 7.86 (d, J=1.2 Hz, 1H), 7.59 (d, J=8.5 Hz, 1H), 7.08 (d, J=8.5 Hz, 1H), 2.60-2.68 (m, 1H), 1.72-1.47 (2H), 1.44-1.04 (18H), 0.91-0.74 (bs, 2H)

### Production Example 16

### N-Benzhydryl-2,4,6-triisopropylbenzenesulfonamide (PVZF2082)

2,4,6-Triisopropylbenzenesulfonyl chloride (151 mg) was dissolved in 10 mL of dichloromethane, and 92 mg of diphenylmethanamine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 101 mg of the target compound in a yield of 45%.

ESI (m/z): 450 (M+H)⁺

¹H-NMR (400 MHz, DMSO-d₆) δ 8.73 (d, J=9.3 Hz, 1H), 7.15-7.35 (m, 10H), 7.05-7.11 (m, 2H), 5.46 (d, J=9.3 Hz, 1H), 4.07-4.13 (m, 2H), 2.84-2.92 (m, 1H), 1.14-1.24 (m, 6H), 1.05 (m, 12H)

### Production Example 17

### 2,4,6-Triisopropyl-N-(1,2,2,6,6-pentamethylpiperidin-4-yl)benzenesulfonamide (PVZF2085)

2,4,6-Triisopropylbenzenesulfonyl chloride (151 mg) was dissolved in 10 mL of dichloromethane, 85 mg of 1,2,2,6,6-pentamethylpiperidin-4-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 118 mg of the target compound in a yield of 54%.

ESI (m/z): 437 (M+H)⁺

¹H-NMR (400 MHz, DMSO-d₆) δ 7.23 (s, 2H), 3.94-4.08 (m, 3H), 2.89-2.96 (m, 1H), 2.60-2.62 (m, 3H), 1.29-1.41 (m, 5H), 1.20 (d, J=6.8 Hz, 18H), 0.96-1.06 (m, 12H)

### Production Example 18

### 2,4,6-Triisopropyl-N-methyl-N-(2,2,6,6-tetramethylpiperidin-4-yl)benzenesulfonamide (PVZF2086)

2,4,6-Triisopropylbenzenesulfonyl chloride (151 mg) was dissolved in 10 mL of dichloromethane, 85 mg of N,2,2,6,6-pentamethylpiperidin-4-amine and 120 μL of pyridine were added thereto in an ice bath, and the mixture was stirred at room temperature for 4 hours. Methanol was added to terminate the reaction, and the dichloromethane was distilled off under reduced pressure. The obtained residue was purified by silica chromatography to give 50 mg of the target compound in a yield of 23%.

ESI (m/z): 437 (M+H)⁺

¹H-NMR (400 MHz, DMSO-d6) δ 7.27 (s, 2H), 4.02 (td, J=13.4, 6.7 Hz, 3H), 2.93 (td, J=13.6, 6.7 Hz, 1H), 2.64 (s, 3H), 1.49-1.63 (m, 3H), 1.04-1.37 (m, 32H)

### Production Example 19

### N-(2,6-Dimethylpiperidin-4-yl)-2,4,6-triisopropylbenzenesulfonamide (PVZF2132)

2,4,6-Triisopropylbenzenesulfonyl chloride (151 mg) was dissolved in 10 mL of dichloromethane, 64 mg of 2,6-dimethylpiperidin-4-amine and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 158 mg of the target compound in a yield of 80%.

ESI (m/z): 395 (M+H)⁺

¹H-NMR (400 MHz, DMSO-d₆) δ 7.77 (s, 1H), 7.25 (s, 2H), 4.13 (td, J=13.3, 6.6 Hz, 2H), 3.30-3.21 (m, 2H) 3.09-2.86 (m, 2H), 1.76-1.63 (bs, 1H), 1.36-1.09 (m, 28H)

### Production Example 20

### 1-Ethyl-3-(trifluoromethyl)-N-(2-((2,4,6-triisopropylphenyl)sulfonamido)ethyl)-1H-pyrazole-5-carboxamide (PVZF2133)

2,4,6-Triisopropylbenzenesulfonyl chloride (151 mg) was dissolved in 10 mL of dichloromethane, 64 mg of N-(2-aminoethyl)-1-ethyl-3-trifluoromethyl)-H-pyrazole-5-carboxamide and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 139 mg of the target compound in a yield of 54%.

ESI (m/z): 517 (M+H)⁺

¹H-NMR (400 MHz, DMSO-d₆) δ 8.40 (t, J=5.9 Hz, 1H), 7.61 (s, 1H), 7.22 (s, 2H), 7.18 (s, 1H), 4.30 (q, J=7.2 Hz, 2H), 4.16-4.06 (m, 2H), 3.45-3.26 (m, 2H), 2.95-2.85 (m, 3H), 1.43-1.32 (m, 3H), 1.27-1.01 (m, 18H)

### Production Example 21

### 2,4,6-Triisopropyl-N-(1-(5-(trifluoromethyl)pyridin-2-yl)piperidin-4-yl)benzenesulfonamide (PVZF2134)

2,4,6-Triisopropylbenzenesulfonyl chloride (151 mg) was dissolved in 10 mL of dichloromethane, 123 mg of 1-(5-(trifluoromethyl)pyridin-2-yl)piperidin-4-amine and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 174 mg of the target compound in a yield of 68%.

ESI (m/z): 512 (M+H)⁺

¹H-NMR (400 MHz, DMSO-d₆) δ 8.37 (s, 1H), 7.75 (dd, J=9.1, 2.3 Hz, 1H), 7.65 (d, J=7.3 Hz, 1H), 7.25 (d, J=12.4 Hz, 2H), 6.93 (d, J=9.0 Hz, 1H), 4.33-4.12 (m, 4H), 3.01-2.87 (m, 3H), 1.69 (d, J=10.5 Hz, 2H), 1.39-1.30 (m, 3H), 1.22-1.03 (m, 18H)

### Production Example 22

### 2,4,6-Triisopropyl-N-(3-(3-(trifluoromethyl)-5,6-dihydrocyclopenta[c]pyrazol-1(4H)-yl)propyl)benzenesulfonamide (PVZF2135)

2,4,6-Triisopropylbenzenesulfonyl chloride (151 mg) was dissolved in 10 mL of dichloromethane, 117 mg of 3-(3-(trifluoromethyl)-5,6-dihydrocyclopentane[c]pyrazol-1(4H)-yl)propan-1-amine and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 155 mg of the target compound in a yield of 62%.

ESI (m/z): 500 (M+H)⁺

¹H-NMR (400 MHz, DMSO-d₆) δ 7.59 (s, 1H), 7.22 (s, 2H), 4.12-3.99 (m, 4H), 2.95-2.85 (m, 1H), 2.76 (t, J=6.2 Hz, 2H), 2.63-2.56 (m, 4H), 2.51-2.42 (m, 2H), 1.93-1.86 (m, 2H), 1.30-1.05 (m, 18H)

### Production Example 23

### 2,4,6-Triisopropyl-N′-(1-(3-(trifluoromethyl)benzyl)-1H-pyrazol-4-yl)benzenesulfonohydrazide (PVZF2136)

2,4,6-Triisopropylbenzenesulfonyl chloride (151 mg) was dissolved in 10 mL of dichloromethane, 128 mg of 4-hydrazinyl-1-(3-(trifluoromethyl)benzyl)-1H-pyrazole and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 91 mg of the target compound in a yield of 35%.

ESI (m/z): 523 (M+H)⁺

¹H-NMR (400 MHz, DMSO-d₆) δ 9.45 (s, 1H), 7.72-7.46 (m, 5H), 7.15 (s, 2H), 7.09 (s, 1H), 5.31 (s, 2H), 3.94 (t, J=6.5 Hz, 2H), 2.93-2.82 (m, 1H), 1.23-0.87 (m, 18H)

### Production Example 24

### N-(6-(2,3-difluorophenoxy)pyridin-3-yl)-2,4,6-triisopropylbenzenesulfonamide (PVZF2137)

2,4,6-Triisopropylbenzenesulfonyl chloride (151 mg) was dissolved in 10 mL of dichloromethane, 244 mg of 6-(2,3-difluorophenoxy)pyridin-3-amine and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 142 mg of the target compound in a yield of 58%.

ESI (m/z): 489 (M+H)⁺

¹H-NMR (400 MHz, DMSO-d₆) δ 10.13 (s, 1H), 7.68 (d, J=2.0 Hz, 1H), 7.54-7.52 (m, 1H), 7.36-7.08 (m, 6H), 3.95 (s, 2H), 2.88 (td, J=13.7, 6.8 Hz, 1H), 1.25-0.85 (m, 18H)

### Production Example 25

### 2,4,6-Triisopropyl-N-((1-((trifluoromethyl)sulfonyl)piperidin-4-yl)methyl)benzenesulfonamide (PVZF2138)

2,4,6-triisopropylbenzenesulfonyl chloride (151 mg) was dissolved in 10 mL of dichloromethane, 123 mg of (1-((trifluoromethyl)sulfonyl)piperidin-4-yl)methanamine and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 200 mg of the target compound in a yield of 78%.

ESI (m/z): 513 (M+H)⁺

¹H-NMR (400 MHz, DMSO-d₆) δ 7.65 (s, 1H), 7.24 (s, 1H), 7.23 (s, 1H), 4.17-4.07 (m, 2H), 3.74 (d, J=12.9 Hz, 2H), 3.10 (t, J=12.4 Hz, 2H), 2.96-2.86 (m, 1H), 2.73-2.67 (m, 2H), 1.77-1.74 (m, 2H), 1.70-1.62 (m, 1H), 1.37-1.18 (m, 18H), 1.15-1.02 (m, 2H)

### Production Example 26

### 2,4,6-Triisopropyl-N-(1-(3-(trifluoromethyl)phenyl)-1H-pyrazol-3-yl)benzenesulfonamide (PVZF2139)

2,4,6-Triisopropylbenzenesulfonyl chloride (151 mg) was dissolved in 10 mL of dichloromethane, 114 mg of 1-(3-(trifluoromethyl)phenyl)-1H-pyrazol-3-amine and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 69 mg of the target compound in a yield of 28%.

ESI (m/z): 494 (M+H)⁺

¹H-NMR (400 MHz, DMSO-d₆) δ 10.85 (s, 1H), 8.49 (s, 1H), 7.95-7.92 (m, 2H), 7.66 (t, J=7.9 Hz, 1H), 7.57 (d, J=7.6 Hz, 1H), 7.20 (s, 2H), 6.09 (d, J=2.2 Hz, 1H), 4.27 (s, 2H), 2.93-2.83 (m, 1H), 1.16 (dd, J=6.7, 2.3 Hz, 18H)

### Production Example 27

### (E)-N′-hydroxy-3-(3-methyl-5-(trifluoromethyl)-1H-pyrazol-1-yl)-N-(2,4,6-triisopropylphenyl)sulfonyl)propanimidamide (PVZF2140)

2,4,6-Triisopropylbenzenesulfonyl chloride (151 mg) was dissolved in 10 mL of dichloromethane, 118 mg of (E)-2-amino-4-(3-methyl-5-(trifluoromethyl)-1H-pyrazol-1-yl)but-1-en-1-ol and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 53 mg of the target compound in a yield of 21%.

ESI (m/z): 503 (M+H)⁺

¹H-NMR (400 MHz, DMSO-d₆) δ 8.55 (s, 1H), 7.23 (s, 2H), 6.82 (s, 2H), 6.55 (s, 1H), 4.15-4.06 (m, 4H), 3.57 (d, J=1.2 Hz, 1H), 2.97-2.87 (m, 1H), 2.11 (s, 3H), 1.21-1.15 (m, 18H)

### Production Example 28

### N-(3-(3-Cyclopropyl-4,5-dihydroxy-2-oxo-2,3-dihydro-1H-imidazol-1-yl)phenyl)-2,4,6-triisopropylbenzenesulfonamide (PVZF2141)

2,4,6-Triisopropylbenzenesulfonyl chloride (151 mg) was dissolved in 10 mL of dichloromethane, 137 mg of 1-(3-aminophenyl)-3-cyclopropyl-4,5-dihydroxy-1,3-dihydro-2H-imidazol-2-one and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 108 mg of the target compound in a yield of 42%.

ESI (m/z): 514 (M+H)⁺

¹H-NMR (400 MHz, DMSO-d₆) δ 10.29 (s, 1H), 7.49 (s, 1H), 7.28-7.07 (m, 5H), 6.79 (d, J=8.0 Hz, 1H), 5.44 (d, J=9.8 Hz, 1H), 4.20 (s, 2H), 3.57 (d, J=1.2 Hz, 1H), 2.89 (td, J=13.6, 6.6 Hz, 1H), 1.18-1.13 (m, 18H), 0.90-0.79 (m, 4H)

### Production Example 29

### 2,4,6-Triisopropyl-N-(((2,2,6,6-tetramethylpiperidin-4-yl)methyl)benzenesulfonamide (PVZF2142)

2,4,6-Triisopropylbenzenesulfonyl chloride (151 mg) was dissolved in 10 mL of dichloromethane, 135 mg of (2,2,6,6-tetramethylpiperidin-4-yl)methanamine and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 194 mg of the target compound in a yield of 89%.

ESI (m/z): 437 (M+H)⁺

¹H-NMR (400 MHz, DMSO-d₆) ¹H-NMR (400 MHz, DMSO) δ 7.80-7.67 (m, 1H), 7.37-7.18 (m, 2H), 6.95 (s, 1H), 4.17-4.07 (m, 2H), 2.96-2.86 (m, 1H), 2.75 (t, J=6.2 Hz, 2H), 1.91-1.86 (m, 1H), 1.61-1.43 (m, 2H), 1.26-1.15 (m, 32H)

### Production Example 29

### 2,4,6-Triisopropyl-N-(1,2,3,4-tetrahydroquinolin-4-yl)benzenesulfonamide (PVZF2143)

2,4,6-Triisopropylbenzenesulfonyl chloride (151 mg) was dissolved in 10 mL of dichloromethane, 124 mg of 1,2,3,4-tetrahydroquinolin-4-amine and 120 μL of pyridine were added thereto, and the mixture was stirred at room temperature for 4 hours. Methanol was added to terminate the reaction, and the dichloromethane was distilled off under reduced pressure. The obtained residue was purified by silica chromatography to give 155 mg of the target compound in a yield of 75%.

ESI (m/z): 415 (M+H)⁺

¹H-NMR (400 MHz, DMSO-d₆) δ 8.00 (d, J=8.8 Hz, 1H), 7.24 (s, 2H), 6.94-6.86 (m, 1H), 6.70 (d, J=7.6 Hz, 1H), 6.47-6.32 (m, 2H), 5.84 (s, 1H), 4.34-4.30 (m, 1H), 4.22-4.10 (m, 2H), 3.22-3.17 (m, 1H), 3.06 (d, J=12.0 Hz, 1H), 2.97-2.90 (m, 1H), 1.80-1.65 (m, 2H), 1.32-1.16 (n, 18H)

1. An RNA methyltransferase inhibitor comprising at least one compound selected from the group consisting of sulfonamide-based compounds represented by the following formula (1) and pyrazoline-based compounds represented by the following formula (2): 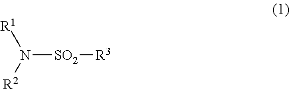
wherein R¹ represents any of the following groups (1-1) to (1-5):
(1-1) an optionally substituted nitrogen-containing heterocyclic group,
(1-2) optionally substituted cycloalkyl,
(1-3) optionally substituted alkyl,
(1-4) pyrazolylamino, and
(1-5) phenyl;
R² represents (2-1) hydrogen or (2-2) alkyl; and
R³ represents any of the following groups (3-1) to (3-9):
(3-1) phenyl,
(3-2) naphthyl,
(3-3) a nitrogen- or sulfur-containing heterocyclic group,
(3-4) dihydrocarbostyril,
(3-5) tetrahydronaphthyl,
(3-6) indanyl,
(3-7) benzoxolyl,
(3-8) benzothiadiazolyl, and
(3-9) dihydrobenzodioxepinyl;
wherein each group shown in (3-1) to (3-9) further optionally has one or more substituents, or R¹ and R², taken together with the nitrogen atom to which they are attached, optionally form a ring; and 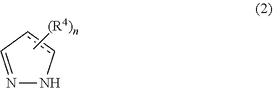
wherein n represents an integer of 2 to 4, and R⁴ is the same or different, and represents any of the following groups (4-1) to (4-35):
(4-1) phenyl,
(4-2) phenyl sulfonyl,
(4-3) alkyl carbonyl,
(4-4) aminothiocarbonyl,
(4-5) benzodioxolyl,
(4-6) alkyl sulfonyl,
(4-7) adamantylcarbonyl,
(4-8) benzopyrazyl,
(4-9) phenylcarbonyl,
(4-10) naphthyl,
(4-11) furylcarbonyl,
(4-12) thienylcarbonyl,
(4-13) quinazolyl,
(4-14) quinoxalyl,
(4-15) hydroxyl,
(4-16) alkenyl,
(4-17) thiazolyl,
(4-18) cycloalkylcarbonyl,
(4-19) aminocarbonyl,
(4-20) furyl,
(4-21) thienyl,
(4-22) pyridyl,
(4-23) cycloalkenyl,
(4-24) alkyl,
(4-25) pyrazolyl,
(4-26) quinolyl,
(4-27) alkenylcarbonyl,
(4-28) benzopyranyl,
(4-29) benzopyrimidyl,
(4-30) pyrrolidinoalkylcarbonyl,
(4-31) quinolylcarbonyl,
(4-32) alkoxy carbonyl,
(4-33) morpholino,
(4-34) pyrrolidinocarbonyl alkoxy, and
(4-35) benzodioxy-6-yl;
wherein each group shown in (4-1) to (4-35) further optionally has one or more substituents; the bond between the carbon atom at 4-position and the carbon atom at 5-position in the pyrazole skeleton is a single bond or a double bond, or two adjacent carbon atoms constituting the pyrazoline ring are optionally bonded to each other to form a ring, or the nitrogen atom constituting the pyrazoline ring and the carbon atom adjacent to the nitrogen atom are optionally bonded to each other to form a ring.

2. The RNA methyltransferase inhibitor according to claim 1, wherein the one or more substituents on the nitrogen-containing heterocyclic group shown in (1-1) above are at least one member selected from the group consisting of alkyl, hydroxyl, cyclopropyl, phenylthiopropylcarbonyl, phenyl sulfonyl, alkyl sulfonyl, thienyl sulfonyl, alkyl carbonyl, alkoxy carbonyl, phenyl sulfonylamino, aminocarbonylalkyl, pyrazolylcarbonyl, cyclopropylcarbonyl, piperidyl sulfonyl, and morpholinosulfonyl.

3. The RNA methyltransferase inhibitor according to claim 1, wherein the one or more substituents on the cycloalkyl shown in (1-2) above and the one or more substituents on the alkyl shown in (1-3) above are each at least one member selected from the group consisting of phenyl, biphenyl, cycloalkyl, cycloalkenyl, nitrogen-containing heterocyclic groups, and hydroxyl.

4. The RNA methyltransferase inhibitor according to claim 1, wherein the one or more substituents on each group shown in (3-1) to (3-9) above are at least one member selected from the group consisting of alkyl, alkoxy, halogen, carboxyl, amino, nitro, phenyl, and cycloalkyl.

5. The RNA methyltransferase inhibitor according to claim 1, wherein the one or more substituents on the phenyl shown in (4-1) above are at least one member selected from the group consisting of halogen, alkyl, haloalkyl, alkoxy, hydroxyl, alkylsulfonylamino, nitro, amino, carboxyl, and phenyl.

6. The RNA methyltransferase inhibitor according to claim 1, wherein the one or more substituents on the alkyl carbonyl shown in (4-3) above are at least one member selected from the group consisting of phenylalkylamino, triazolylthio, phenoxy, oxadiazolylthio, esters, piperazinyl, carboxyl, pyrimidinylthio, quinazolyloxy, morpholinocarbonyl, morpholino, benzotriazolyl, pyrazolyl carbonyl, pyrimidyl, pyrrolidino, piperidino, tetrahydroimidazolyl, halogen, naphthyloxy, alkoxy, imidazolyl, tetrazolylthio, alkylamino, pyridyl, tetrazolyl, benzodioxonyloxy, aminocarbonyl, piperazinyl, phenylalkylthio, alkylcarbonyloxy, benzotriazolylthio, pyridazinyl, pyrrolylcarbonyloxy, piperidino, dihydrothiazolylthio, benzopyrazyl, thienopyridinoxy, thienopyrimidinylthio, cyclopentathienopyrimidinyl, thiadiazolylthio, azepinylthio, dioxoloquinolinyl, diazaspirononanyl, imidazolidinyl, triazolylthio, dihydropyridazinyl, and 1,3-diazaspiroundecanyl.

7. The RNA methyltransferase inhibitor according to claim 1, for use in the treatment of cancer.

8. A sulfonamide-based compound represented by the following formula (1a): 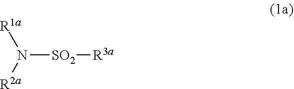
wherein R^{1a} represents optionally substituted piperidyl, optionally substituted pyridyl, optionally substituted pyrazolyl, cyclohexyl, optionally substituted C₁₋₅ linear alkyl, optionally substituted pyrazolylamino, or optionally substituted phenylamino; R^{2a} represents hydrogen or methyl; and R^{3a} represents optionally substituted phenyl.

9. A screening method for RNA methyltransferase inhibitors, comprising the step of measuring RNA methylation inhibitory effects of a test substance against cells or viruses.

10. The method according to claim 9, wherein the RNA methylation inhibitory effects are based on FTSJ inhibition.

11. The method according to claim 10, wherein the FTSJ is FTSJ1.

12. The method according to claim 9, wherein the RNA methylation inhibitory effects are measured by a reporter assay using a sequence in which a translation regulatory region is added to a reporter region,
wherein
the translation regulatory region comprises a sequence formed by bonding of at least one member selected from the group consisting of glutamine, phenylalanine, tryptophan, methionine, and leucine.

13. The method according to claim 12, wherein the translation regulatory region comprises a sequence in which 5 to 50 of at least one member selected from the group consisting of glutamine, phenylalanine, tryptophan, methionine, and leucine are continuously bonded.

14. The method according to claim 12, wherein the translation regulatory region comprises polyglutamine, polyphenylalanine, polytryptophan, polymethionine, or polyleucine respectively comprising continuously bonded 5 to 50 glutamines, phenylalanines, tryptophans, methionines, or leucines.

15. The method according to claim 12, wherein the translation regulatory region is any of SEQ ID No: 1 to 12.

16. The method according to claim 9, further comprising a reporter assay using a sequence comprising the transcription factor binding region and a reporter region represented by SEQ ID No: 13.

17. A screening method for FTSJ1 inhibitors, comprising, in this order, the step of adding a methyl group donor to a test substance to obtain a reaction product; and the step of measuring FTSJ1 activity of the test substance using the reaction product.

18. The method according to claim 17, wherein the methyl group donor is S-adenosylmethionine (SAM).

19. The method according to claim 18, wherein the FTSJ1 activity is measured by a luciferase assay.

20. A method for predicting the efficacy of an FTSJ1 inhibitor against a cancer, or a method for predicting prognosis after use of an FTSJ1 inhibitor against cancer, comprising step A of measuring the FTSJ1 expression level in a sample.

21. The method according to claim 20, wherein step A is performed by an immunological method or genetic method.

22. The method according to claim 20, wherein the sample is taken from a patient.

23. The method according to claim 20, further comprising step B for determining the efficacy of an FTSJ1 inhibitor against a cancer, or step B for determining prognosis of cancer pathology of the patient, based on the FTSJ1 expression level obtained in step A.

24. The method according to claim 20, wherein the cancer is at least one member selected from the group consisting of glioblastoma (malignant brain tumor), pancreatic cancer, acute myeloid leukaemia, lung cancer, liver cancer, kidney cancer, gastric cancer, and breast cancer.

25. A marker for determining efficacy of an anti-cancer agent, comprising an FTSJ1 inhibitor sensitivity-related gene marker or FTSJ1 inhibitor resistance-related gene marker.

26. The marker according to claim 25, wherein the FTSJ1 inhibitor sensitivity-related gene marker or FTSJ1 inhibitor resistance-related gene marker is an FTSJ1 modified nucleic acid RNA.

27. The marker according to claim 25, wherein the FTSJ1 inhibitor resistance-related gene marker is at least one member selected from the group consisting of AHNAK nucleoprotein 2 (AHNAK2, SEQ ID No: 14), extended synaptotagmin 1 (ESYT1, SEQ ID No: 15), SLIT-ROBO Rho GTPase activating protein 1 (SRGAP1, SEQ ID No: 16), ras homolog family member F, filopodia associated (RHOF, SEQ ID No: 17), microRNA 4746 (MIR4746, SEQ ID No: 18), UBX domain protein 6 (UBXN6, SEQ ID No: 19), cytochrome c oxidase assembly factor COX16 (COX16, SEQ ID No: 20), ferritin heavy chain 1 (FTH1, SEQ ID No: 21), lysophosphatidic acid receptor 1 (LPAR1, SEQ ID No: 22), ankyrin repeat domain 29 (ANKRD29, SEQ ID No: 23), twist family bHLH transcription factor 2 (TWIST2, SEQ ID No: 24), JNK1/MAPK8 associated membrane protein (JKAMP, SEQ ID No: 25), protein kinase AMP-activated catalytic subunit alpha 2 (PRKAA2, SEQ ID No: 26), cleavage stimulation factor subunit 2 tau variant (CSTF2T, SEQ ID No: 27), thrombospondin type 1 domain containing 4 (THSD4, SEQ ID No: 28), membrane associated guanylate kinase, WW and PDZ domain containing 1 (MAGI1, SEQ ID No: 29), ubiquitin conjugating enzyme E2 L3 (UBE2L3, SEQ ID No: 30), glycosylphosphatidylinositol specific phospholipase D1 (GPLD1, SEQ ID No: 31), FRY like transcription coactivator (FRYL, SEQ ID No: 32), and myosin IXA (MYO9A, SEQ ID No: 33).

28. The marker according to claim 25, wherein the FTSJ1 inhibitor sensitivity-related gene marker is at least one member selected from the group consisting of RNA binding motif protein 15 (RBM15, SEQ ID No: 34), nuclear autoantigenic sperm protein (NASP, SEQ ID No: 35), pre-mRNA processing factor 38A (PRPF38A, SEQ ID No: 36), chromosome 1 open reading frame 50 (C1orf50, SEQ ID No: 37), peroxisomal biogenesis factor 16 (PEX16, SEQ ID No: 38), zinc finger protein 213 (ZNF213, SEQ ID No: 39), fem-1 homolog B (FEM1B, SEQ ID No: 40), regulatory factor X associated protein (RFXAP, SEQ ID No: 41), Sin3A associated protein 18 (SAP18, SEQ ID No: 42), alanyl-tRNA synthetase 2, mitochondrial (AARS2, SEQ ID No: 43), regulator of chromosome condensation 2 (RCC2, SEQ ID No: 44), tyrosyl-tRNA synthetase 1 (YARS1, SEQ ID No: 45), RNA binding motif protein 10 (RBM10, SEQ ID No: 46), ribosomal protein L5 (RPL5, SEQ ID No: 47), zinc finger HIT-type containing 2 (ZNHIT2, SEQ ID No: 48), oxidative stress induced growth inhibitor family member 2 (OSGIN2, SEQ ID No: 49), egl-9 family hypoxia inducible factor 3 (EGLN3, SEQ ID No: 50), tRNA phosphotransferase 1 (TRPTI, SEQ ID No: 51), CRACD like (CRACDL, SEQ ID No: 52), capping actin protein, gelsolin like (CAPG, SEQ ID No: 53), RAB11 family interacting protein 3 (RAB11FIP3, SEQ ID No: 54), calcium homeostasis modulator family member 5 (CALHM5, SEQ ID No: 55), BICD cargo adaptor 1 (BICD1, SEQ ID No: 56), and FTSJ 1 (FTSJ1, SEQ ID No: 57).

29. A kit for predicting efficacy of an FTSJ1 inhibitor, comprising the marker according to claim 25.


SEQUENCE LISTING (57 sequences)

SEQ ID NO: 1 - DNA, 30 bp, Homo sapiens
caacaacaacaacaacaacaacaacaacaa

SEQ ID NO: 2 - DNA, 30 bp, Homo sapiens
cagcagcagcagcagcagcagcagcagcag

SEQ ID NO: 3 - DNA, 30 bp, Homo sapiens
ttcttcttcttcttcttcttcttcttcttc

SEQ ID NO: 4 - DNA, 30 bp, Homo sapiens
tttttttttttttttttttttttttttttt

SEQ ID NO: 5 - DNA, 30 bp, Homo sapiens
tggtggtggtggtggtggtggtggtggtgg

SEQ ID NO: 6 - DNA, 30 bp, Homo sapiens
atgatgatgatgatgatgatgatgatgatg

SEQ ID NO: 7 - DNA, 30 bp, Homo sapiens
ttattattattattattattattattatta

SEQ ID NO: 8 - DNA, 30 bp, Homo sapiens
ttgttgttgttgttgttgttgttgttgttg

SEQ ID NO: 9 - DNA, 30 bp, Homo sapiens
cttcttcttcttcttcttcttcttcttctt

SEQ ID NO: 10 - DNA, 30 bp, Homo sapiens
ctcctcctcctcctcctcctcctcctcctc

SEQ ID NO: 11 - DNA, 30 bp, Homo sapiens
ctactactactactactactactactacta

SEQ ID NO: 12 - DNA, 30 bp, Homo sapiens
ctgctgctgctgctgctgctgctgctgctg

SEQ ID NO: 13 - DNA, 8 bp, Homo sapiens
acattcca

SEQ ID NO: 14 - RNA, 18335 nt, Homo sapiens
  ref <308>: DATABASE ACCESSION NUMBER: NM_138420.4
  ref <309>: DATABASE ENTRY DATE: 2022-03-15
  ref <313>: RELEVANT RESIDUES IN SEQ ID NO: (1)..(18335)
aguccgaggucggccgcccgcggcagcgcagggcagcggggcagagcgcggcucccgcccgcccgccccgugcgccggagccgggaccagcgacuggacgcgacgggccgccaggcccgcaccgccuggccgccgcgaugugcgacugcuuccacauggugcugcccaccuggcccggaacccccggcagcguguccggccgucagcugcagcccggggagccaggugcagaaacggaagaugaccacucugugacugaagggccugcggaugagggcauucgaccacggccgcaggggucuucaccugucuacgaauacacgacugaggcugccgacuuuggacuccaggaagacgcccccggcaggcaagguucugcugggagacggagauccugguggaagcgagauucaggggacucacggacauuuuucaggaugagucguccagaggcuguccaggaggcaacagaggugacgcugaagacagagguggaggcaggagccaguggcuacagugucacaggugguggggaccaggggaucuucgucaagcaagugcugaaggacuccucagccgccaagcuuuuuaacuugagagaaggggaucagcugcucaguacaaccguguucuuugaaaacauaaaauaugaagaugcucucaaaauccuucaauauucagagccguacaagguucaguucaaaaucagacggcagcucccugccccacaggaugaagagugggcuuccagcgaugcccagcacggcccacagggcaaggagaaggaggacacggauguugcugaugggugcagagagacccccacgaaaacucuggaaggagauggggaccaagagagacucaucuccaaaccaaggguggggagaggcaggcagagccagagggagaggcucucuuggccaaaauuucaauccauaaagagcaagcgggggccgggaccccagaggucacacagcucgucagaggccuacgaaccuagggacgcacaugacguguccccuacaagcacagacacagaggcccagcucacgguggagcgccaagagcagaaggcagggccgggcagccagaggaggcggaaguuccucaaccucagauucaggacaggcucgggacagggcccuucaucgacaggacagccaggcaggggguuccagaguggggugggccgugcugggguccuggaagaguuggggcccuggggugauagccucgaggagacuggggcugccacaggcagcaggagagaggagagggcagaacaggaucgagaagugaugccugcucagagcaugccauugcccacagagcucggugacccuagacuuugcgagggaaccccucaggaagggggacucagggcagccaggcuccauggaaagacccuggaggggcaggcacaggagacagcaguggcccagaggaagcccagggcccagccaacuccuggaaugagccgggagggugaaggcgagggacugcagagccuggaaaucgggaucgccagacuguccuugagagacacaaccgaaggaggcacacagauuggcccaccagaaauuagggugcgaguacacgauuuaaagacaccaaaauuugcauuuuccacagaaaaagagccagaaagagaaaggcgccuuaguaccccacagcgagggaagagacaggaugcguccucaaaagcggguacuggccugaagggugaggagguggaaggagccggguggaugccgggcagggaaccaaccacacaugcagaagcacagggggaugaaggagauggagaggaaggacuacagaggacaaggaucacugaggaacaggacaagggcagggaagacacagaaggacagauaagaaugcccaaguucaagauacccuccuuaggauggucgccaagcaagcacacaaagacaggcagagaaaaagccacagaagacacagagcagggaagggaaggagaggccacagcaacagcugauagaagagaacagagacgcacagaggaaggauuaaaagacaaagaagacagugacucaaugacaaacacaacaaaaauacaacuaauacacgaugaaaaacgcuuaaaaaaggaacaaauucugacagaaaaggaaguggccaccaaagacagcaaguucaaaaugcccaaguucaagaugccauuguucggggcgucagccccaggcaaguccauggaggccucgguggaugugucugcgccgaagguggaggccgacgugagccuccucuccaugcagggggaccucaagaccacugaccucagcguccagaccccuuccgcugaccuggagguccaggauggccaaguggaugugaaacuuccggagggcccccugcccgagggagccagccucaaagggcaccugcccaaggugcagaggcccaguuugaagaugcccaaaguggaccucaagggccccaagcuggaccugaaaggccccaaggcggaagugacagcccccgaugugaagaugucucuguccagcauggagguggacguccaggccccgagagcaaagcuggauggugcgcggcuggagggggaccugucccuggccgacaaggaggugacugccaaagacagcaaguucaaaaugcccaaguucaagaugccaucauucggggugucggccccaggcaaguccauggaggacucgguggaugugucugcgccgaagguggaggccgacgugagccucuccuccaugcagggggaccucaaggccacugaccucagcauucagcccccuuccgcugaccuggagguccaggcuggccaaguggaugugaaacuuccggagggcccugugcccgagggagccggccccaaagugcaccugcccaaaguggagaugcccaguuucaagaugcccaaaguggaccucaagggcccccagauagauguuaagggccccaagcuggaccugaaaggccccaaggcggaagugacagcccccgauggcgaggugucucugcccagcauggagguggauguccaggcccagaaggccaagcuggauggugcguggcuggagggggaccugucccuggccgacaaggacgugacugccaaagacagcaaguucaaaaugcccaaguucaagaugccgucguucgggguaucggccccagggaaguccaucaaggccuugguggaugugucugcacccaagguggaggccgaccugagucuccccuccaugcagggggaccugaagaccacugaccucagcauucagccugcuucuacugaccugaagguccaggcugaccagguggaugugaagcucccggagggccaccugcccgagggagcuggccuuaaagggcacuugcccaagguggagaugcccaguuucaagaugcccaaaguggcccucaagggcccccagguggacgucaagggccccaagcuggaccugaaaagccccaaggcggaagucacagccccugauguggaggugucucugcccagcguggagguggacgucgaggccccgggagccaagcuggacagugcgcggcuggagggggaacugucccuggccgacaaggaugugacugccaaagacagcagguucaaaaugcccaaguucaagaugccaucguucggggcgucagccccaggcaaguccaucgaggccucgguggaugugucugcacccaaaguggaggccgacgugagucuccccuccaugcagggggaccucaagaccacugaccucagcauucagcccccuuccgcugaccuggagguccacgcuggccagguggacgugaagcuccuggagggccacgugccugagggagccggcuucaaagggcaccugcccaaggugcagaugccuaguuugaagaugcccaaaguggaccucaagggcccccagguggaagucaggggccccaagcuggaccugaaaggucauaaggcagaggugacggcccacgaaguggcugugucucugcccaguguggagguggacaugcaggccccgggagccaaguuggauggcgcacagcuggacggggaccugucccuggcugacaaggacgugacugccaaagacagcaaguucaaaaugcccaaguucaagaugccgucguucggggugucugccccaggcaaguccauugaggccuccguggaccugucugcacccaagguggaggccgacaugagccuccccuccaugcagggggaccucaagaccacugaccucagcauucagcccccuuccacugaccuggagcuccaggcuggccaauuggacgugaaacucccagagggccccgugcccgagggagccggccucaaagggcaccugcccaagcugcagaugcccaguuucaaggugcccaaaguggaccucaagggcccugaaauagacaucaagggccccaagcuggaccuaaaagaccccaagguggaagugacagccccugauguggagguuucucugcccagcguggagguggaugucgaggccccaggagccaagcuggaugguggacggcuggaggaggacaugucccuggccgacaaggacuugacuaccaaagacagcaaguucaaaaugcccaaguucaagaugccgucguucggggugucugccccaggcaaguccaucgaggccucaguggaugugucugcgccgaagguggaggccgacgugagccuccccuccaugcagggggaccucaaggccacugaccugagcauacagcccccuucugcugaccuggagguccaggcuggccaaguggacgugaaacucccagagggcccuguguccgagggagccggccucaaagggcaccugcccaaagugcagaugcccaguuucaagaugcccaaaguggaccucaaggggccccagauagauguuaagggccccaagcuggaccugaaaggccccaagguggaagugacagcccccgaugugaagaugucucuguccagcauggagguggacguccaggccccgagagcaaagcuggauggugcgcagcuggagggggaccugucccuggccgacaaggcggugacugccaaagacagcaaguucaaaaugcccaaguucaagaugccaucauuuggggugucggccccaggcaaguccaucgaggccucgguggaugugucugagccgaagguggaagcugaugugagccuccccuccaugcagggggaccugaagaccacugaccucagcauucaguccccuuccgccgaccuggagguccaggcuggccaagugaacgugaaacucccggagggcccccuucccgagggagccggcuucaaagggcaccuccccaaggugcagaugcccaguuugaagaugcccaaaguggcccucaagggcccccagauggacgucaagggccccaagcuggaccugaaaggccccaaggcggaggugauggcccccgacguggaggugucucugcccagcguggagguggacgucgaggcuccaggagccaagcuggacagugugcggcuggagggugaccugucccuggccgacaaggaugugacugccaaagacagcaaguucaaaaugcccaaguucaagaugccgucguucggggugucugccccaggcaaguccaucgaggccucgguggaugugucugcgccgaagguggaggccgaagugagccuccccuccaugcagggggaccucaagaccacggaccucugcauuccgcucccuucugcagaccuggugguccaggcuggccaaguggacaugaagcucccggagggccaggugcccgagggagccggccucaaagggcacuugcccaagguggauaugcccaguuucaagaugcccaaaguggaccucaagggcccccagacagauguuaagggcgccaagcuggaccugaaaggccccaaggcggaagugacagcccccgaugucgaggugucucugcccagcauggagguggauguccaggcccagaaggcuaagcuggauggugcgcggcuggagggagaccugucccuggccgacaaggacaugacugccaaagacagcaaguucaaaaugcccaaauucaagaugccgucguucgggguaucggccccagggagguccaucgaggccucgguggaugugccugcacccaagguggaggccgacgugagucuccccuccaugcagggggaccugaagaccacugaccucagcauucagcccccuucugccgaccugaagguccagacuggccagguggaugugaagcucccggagggccacgugcccgagggagcuggccucaaagggcaccugcccaagguggagaugcccaguuugaagaugcccaaaguggaccucaagggcccccagguggacaucaagggccccaaacuggaccuaaaagaccccaagguggaaaugagaguccccgaugucgaggugucucugcccagcauggagguggacguccaggccccaagagccaagcuggauagugcgcaucugcagggggaccugacccuggccaacaaggaccugacuaccaaagacagcaaguucaaaaugcccaaguucaagaugccgucguuuggggugucugccccaggcaaguccaucgaggccucgguggaugugucuccacccaagguggaggccgacaugagucuccccuccaugcagggggaccucaagaccacugaccucagcauucagccccuuuccgccgacgugaagguccaggcuggccagguggacgugaaacuccuggagggcccugugcccgaggaagucggccucaaagggcaccugcccaagcugcagaugcccaguuucaaggugcccaaaguggaccucaagggccccgaaauagacaucaagggccccaagcuggaccuaaaagaccccaagguggaagugacagccccugauguggaggugucucugcccagcguggagguggacgucaaggccccaggagccaagcuggauggugcgcggcuggagggggacaugucccuggccgacaaggacgugacugccaaagacagcaaguucaaaaugcccaaguucaagaugcugucguuuggggugucugcccuuggcaaguccaucgaggccucagcggaugugucugcguugaagguggaggccgacgugagccuccccuccaugcagggggaccucaagaccacugaccucagcguucagcccccuuccgcugaccuggagguccaggcuggccaaguggaugugaaacucccagagggccccgugccggagggagccggccucaaagggcaccugcccaagcugcagaugcccaguuucaagaugcccaaaguagaucucaagggcccccagauagaugucaagggccccaagcuggaccugaaaggccccaagacggacgugauggcccccgacguggaggugucucagcccagcguggagguggaugucgaggccccgggagccaagcuggauggugcguggcuggagggggaccugucuguggcggacaaggaugugacuaccaaagacagcagguucaaaauucccaaguucaagaugccgucauucggggugucugccccaggcaaguccaucgaggccucgguggaugugucugcgccgaagguggaggccgacgggagccucuccuccaugcagggggaccucaaggccacugaccucagcauucagcccccuuccgcugaccuggagguccaggcuggccaaguggacgugaaacucccagagggcccugugccggagggagccggccucaaagggcaccugcccaaggugcagaugcccaguuucaagaugccugaaauggaccucaagggcccccagcuagaugucaagggccccaagcuggaccugaaaggccccaaggcggaagugacagcccccgauguggagaugucucuguccagcauggagguggacguccaggccccgagagcaaagcuggauggugcgcggcuggagggggaccugucccuggccgacaagggugugacagccaaagauagcaaguucaaaaugcccaaguucaagaugccaucauucagggugucggccccaggcgaguccaucgaggcguugguggaugugucugagcugaagguggaagccgacaugagccuccccuccaugcaaggggaccuuaagaccacugacaucagcauucagccccccucugcccaacuggagguccaggcuggccagguggaugugaaacucccagagggccacguucccgagggagccggccucaaagggcaccugcccaagcugcagaugcccaguuucaagaugccugaaguggaccucaagggcccccagauagauguuaagggccccaacguggaccugaaaggccccaaggcggaagugacagcccccgaugugaagaugucucuguccagcauggagguggacguccaggccccgagagcaaagcuggauggugcgcggcuggagggggaccugucccuggccgacaagggcaugacagccaaagacagcaaguucaaaaugcccaaguucaagaugccgucauucggggugucggccccaggcaaguccaucgaggccucgguggaugugucugagcugaagguggaagcugacgggagcuuccccuccaugcaaggggaucuuaagaccacugacauccgcauucagccccccuccgcccaacuggagguccaggcuggccagguggacgugaaacucccagagggccacguucccgagggagccggccucaaagggcaccugcccaaggugcagaugcccaguuucaagaugcccaaaguggaucucaagggcccccagauagacgucaagggccccaagcuggaccugaaaggccccaaggcggaggugacggcccccgacguggaggugucucugcccagcguggagguggacgucgaggccccgagagcaaagcuggauggugcacggcuggagggugaccugucccuggccgacaaggaugugacugccaaagacagcaaguucaaaaugcccaaguucaagaugccgucguucggggugucugccccaggcaaguccauugaggucucgguggaugugucugcgccgaagguggaggccgaagugagccuccccuccaugcagggggaccugaagaccacugacaucagcauugagccccccucugcccaacuggagguccaggcuggccagguggaccugaagcucccagagggccacguucccgagggagcuggccucaaagggcaccugcccaaguugcagaugcccaguuucaagaugcccaaaguagaucgcaagggaccccagauagaugucaagggccccaagcuggaccugaaaggcccgaagacggacgugacggcccccgacguggaggugucucagcccggcauggagguggaugucgaggccccaggagccaaguuggauggugcacggcuggagggggaccugucccuggccgacaaggaugugacugccaaagacagcaaguucaaaaugcccaaguucaagaugccgucguucggggugucugccccaggcaaguccauugaggucuugguggaugugucugcgccaaagguggaggccgaccugagccuccccuccaugcagggggaccugaagaacacugacaucagcauugagccccccucugcccaacuggagguccaggcuggccagguggacgugaagcucccagagggccacguucucgagggagcuggccucaaagggcaccugcccaaguugcagaugcccaguuucaagaugcccaaaguagaucgcaagggcccccagauagacaucaagggccccaagcuggaccugaaaggcccgaagauggaugugacggcccccgacguggaggugucucagcccagcauggagguggacgucgaggccccaggagccaaguuggauggugcacggcuggagggggaccugucccuggccgacaaggaugugacugccaaagacagcaaguucaaaaugcccaaauucaagaugccgucguacagggcgucugccccaggcaaguccauccaggccucgguggaugugucugcgccgaaggcggaggccgacgugagccuccccuccaugcagggggaccucaagaccacugaccucagcauucagcucccuucuguggaccuggagguccaggcuggccagguggacgugaagcucccggagggccacgugcccgagggagcuggccucaaagggcaccugcccaagguggagaugcccaguuucaagaugcccaaaguggaccucaagagcccccagguggacaucaagggccccaagcuggaccuaaaaguccccaaggcggaagugacagucccugauguggaggugucucugcccagcguggagguggacguccaggccccgagagccaagcuggauggugcgcggcuggagggggaccugucccuggcugaaaaggaugugacugccaaagacagcaaguucaaaaugcccaaguucaagaugcccuccuucggggugucggccccaggcagguccaucgaggccucgcuggaugugucugcgccgaagguggaggccgacgugagccucuccuccaugcagggggaccucaaggccacugaccucagcauucagcccccuuccgcugaccuggagguccaggcuguccaaguggauguggaacuccuggagggccccgugcccgagggagccggccucaaagggcaccugcccaaaguggagaugcccaguuuaaagacgcccaaaguggaccucaagggcccccagauagauguuaagggccccaagcuggaccugaaaggccccaaggcagaagugagaguccccgaugucgaggugucucugcccagcguggagguggauguccaggccccgaaggccaagcuggaugcugggcggcuggagggagaccugucccuggcugacaaggacgugacugccaaagacagcaaguucaaaaugcccaaauucaagaugccgucauucaggguaucggccccagggaaguccauggaggccucgguggaugugucugcacccaagguggaagccgaugugagucuccccuccaugcagggggaccugaagaccacugaccucagcauucagcccccuucugccgaccugaagguccaggcuggccagauggaugugaagcucccggagggccaggugcccgagggagccggccucaaagagcaccugcccaagguggagaugcccaguuugaagaugcccaaaguggaccucaagggcccccagguggacaucaagggccccaagcuggaccuaaaagucuccaaggcggaagucacagccccugauguggaggugucucugcccagcguggagguggacguccaggccccaagagccaaacuggauagugcacagcuggagggggaccugucccuggccgacaaggaugugacugccaaagacagcaaauucaaaaugcccaaguucaagaugccgucauuuggggugucugccccaggcaaguccauugaggccucggugcacgugucugcacccaagguggaggccgaugugagucuccccuccaugcagggggaccucaagaccacugaccucagcauucagccccauucugccgaccugacgguccaagcucgccagguggacaugaaacuccuggagggccacgugcccgaggaagccggccucaaaggacaccugcccaaggugcagaugcccaguuucaagaugcccaaagucgaccucaagggcccugaaauagacaucaagggccccaagcuggaccuaaaagaccccaagguggaagugacagccccugauguggagguuucucugcccagcguggagguggacgucgaggccccaggagccaagcuggauggugcgcggcuggagggggaccugucccuggccgacaaggacaugacggccaaagacagcaaguucaaaaugcccaaguucaagaugccgucguucggggugucugccccaggcaaguccauggaggcaucaguggaugugaccgcgccaaagguggaggccgacgugagccucccuuccaugcagggggaccucaaggccacugaccucagcguucagcccccuuccgcugaccuggagguccaggcuggccaaguggacgugaaacucccagagggccccgugcccgagggagccagccucaaagggcaccugcccaaggugcagaugcccaguuucaagaugcccaaaguggaccucaagggcccccagauagauguuaagggccccaagcuggaccugaaaggccccaaggcggaagugacagccccugaugugaagaugucucuguccagcauggagguggacguccaggccccgagagcaaagcuggauggugugcagcuggagggggaccugucccuggccgacaaggaugugacugccaaagacagcaaguucaaaaugcccaaguucaagaugccaucauucggggugucggccccaggcaaguccauggaggcguccguggaugugucugagcugaaggcgaaagccgacgugagccuccccuccaugcagggggaccucaagaccacugaccucagcauucaguccccuuccgccgaccuggagguccaggcuggccaaguggacgugaaacucccggagggcccccugcccaagggagccggccucaaagggcaccuccccaaggugcagaugcccuguuugaagaugcccaaaguggcccucaagggcccccagguggaugucaagggccccaagcuggaccugaaaggccccaaggcggaugugaugacccccgucguggaggugucucugcccagcauggagguggacgucgaggccccgggagccaagcuggacagugugcggcuggagggugaccugucccuagccgacaaggacaugacugccaaagacagcaaguucaaaaugcccaaguucaagaugccgucguucggggugucugccccaggcaaguccaucgaggccucguuggaugugucugcgcugaagguggaggcugacgugagccuccccuccaugcagggggaccugaagaccacucaccucagcauucagcccccuuccgcugaucuggagguccaggcuggccaagaggaugugaaacucccagagggcccugugcaugagggagccggccucaaagggcaccugccgaagcugcagaugcccaguuucaagguacccaaaguggaccucaaggguccccagauagacguuaauguccccaagcuggaccugaaaggccccaagguggaggugacgucccccaaccuggacgugucucugcccagcauggagguggacauccaagccccaggagccaagcuggacaguacgcggcuggagggggaccugucccuggcugacaaggacgugacugccaaagacagcaaguucaaaaugcccaaguucaagaugccauccuuugggauguuguccccaggcaaguccaucgaggucucgguggaugugucugcgccaaagauggaggccgacaugagcauucccuccaugcagggggaccucaagaccacugaccuccgcauucaggccccuuccgccgaccuggagguccaggcuggccagguggacuugaaacuuccagaaggccacaugcccgagguagccggccucaaagggcaccugcccaagguggagaugcccaguuucaagaugcccaaaguggaccucaagggcccccagguggacgucaagggccccaagcuggaccugaaaggcccaaaggcagaggugauggcccccgauguggaggugucucugcccagcguggagacggauguccaggccccaggauccaugcuggauggugcgcggcuugagggggaccugucccuggcccacgaggauguagcugggaaagacaguaaguuucaaggaccaaaacugagcacgucugguuuugaauggucgucaaagaaaguuuccaugucuuccucugaaaucgaaggaaauguuacauuccaugagaagacuuccacauuucccauuguggaaucuguuguucaugaaggugaucuucaugauccaucucgcgaugguaacuuggggcuugcuguuggagaaguuggaauggauucgaaguuuaagaaacugcauuuuaaagugcccaaaguuucauuuucuucuaccaaaacuccuaaagauaguuuagucccaggugcaaagucuagcauaggucuuuccacgauuccuuuaucaucuucagaaugcucaaguuuugaauuacaacagguuucggcuuguucagagccauccaugcagaugccuaagguggguuuugcuggguuuccaucaucccggcuugaucucacugguccucacuuugaaucuucuauucucucucccugugaggauguuacacuuacaaaauaccaggugacuguucccagagcugccuuggccccugagcuugcucuggaaauuccuucugggucucaggcugauauuccucuucccaagacagagugcuccacugaccugcagccuccagagggaguuccaacaucucaagcugagagucacucuggcccacugaauuccaugauuccuguuucucuuggucagguaucuuuuccuaaauucuauaaaccaaaguuuguguuuucagucccccaaauggcaguuccugagggagaccuacaugcagcagugggugccccagucaugucuccucuuagcccuggagaaagagugcagugccccuugccaagcacccagcugccauccccaggcaccugugugucucagggcccagaagagcuuguggccuccuugcagacaucaguaguggccccuggagaagccccuucugaagaugcugaccacgaagggaaagggagucccuugaaaaugccuaagauuaagcuuccaucauuuaggugguccccgaagaaggaaacagggccaaagguggacccagaaugcagcguggaggacucaaaacucagccugguuuuagacaaggaugaaguggccccgcagucugccauccacauggaucugccuccugagagggauggagagaaggggaggagcacaaagccuggcuuugccaugccaaaacuugcacuucccaaaaugaaggcuucuaagaguggggucagccugccacagagagacguggauccuucccuuucuagugccacagcaggggguagcuuucaagacacagaaaaggccagcagugacggugguaggggaggacuuggugcaacagcaagugccacaggaagugagggugugaaccuccaccggccacagguccacauucccaguuugggcuuugccaaaccugaucucagauccuccaaggccaagguggaggugagccagccugaagcugaccugccucuucccaaacaugaucugucuaccgaaggugacagcagaggaugugggcucggggaugucccagugagccagccuuguggggaggggauagcccccacaccugaagauccccuccagccauccuguagaaaaccagaugcugaaguccucacaguggaaagcccagaggaggaagccaugaccaaguacucgcaggaaagcugguuuaaaaugcccaaguuccgcaugcccagccuuaggcgcucuuucagggacagaggcggggcuggaaagcuggaaguggcucagacacaggcaccggcagcaacagggggugaagcagcagcuaaagucaaagaguuccuuguuucugggucaaacguggaggcagcuaugucccuacagcucccagaggcagaugcagaagugacagcuucugagagcaaaucauccacagauauucuaaggugugaucuugacagcacaggcuugaagcugcaccucuccacugcugggaugacuggggaugagcuuuccacuucugaggucaggauccauccauccaaaggaccucucccuuuucagaugccuggcaugaggcuuccagaaacccagguucuuccaggagaaauagaugagacuccucuuuccaagccaggacaugaccuugccagcauggaggauaaaacagagaaauggucuucccagccugaagguccacuuaaauugaaagcuucaaguacugauaugccaucccagauuucugugguuaauguggaucaacugugggaagauucuguccuaacugucaaauuccccaaauuaaugguaccaagguucuccuucccugcccccagcucagaggaugauguguucauccccacugugagggaagugcaguguccagaggccaauauugauacagcccuuuguaaggaaaguccggggcucuggggagccagcauccugaaggcaggugcuggggucccuggggagcagccuguggaccuuaaccugccuuuggaagcucccccaauuucaaaggucagagugcauauucagggugcucagguugaaagucaagaggucacuauacacagcauagugacaccagaguuuguagaucucucaguacccaggacuuuuuccacucagauugugcgggaaucagagauccccacgucagagauucaaacaccuucguacggauuuuccuuauuaaaagugaaaaucccagagccccacacgcaggcuagaguguacacaacaaugacucaacacucuaggacucaggagggcacagaagaggcucccauacaagccaccccaggaguagacuccauuucuggagaucuccagccugacacuggagaaccauuugagaugaucucuuccagcgucaauguacugggacagcaaacacucacauuugaaguuccuucuggccaccagcuugcagacagcuguucagaugaggagccagcagaaauucuugaguuucccccugaugauagccaagaggcaaccacaccacuggcagaugaaggcagggcuccaaaagacaaaccagaaaguaaaaaaucuggucugcucugguuuuggcuuccaaacauuggguuuuccucuucuguugaugagacagguguugauuccaaaaaugacguccagagaucugcucccauucaaacacagccugaggcacgaccagaggcagaacugccuaaaaaacaggagaaggcaggcugguuccgauuucccaaauuaggguucuccucaucuccuaccaagaaaagcaaaagcaccgaagauggggcagagcuggaagaacaaaaacuucaagaagaaacaaucacguuuuuugaugcccgagaaaguuucuccccugaagagaaggaagagggugaacugaucgggccugugggcacugggcuggacuccagagugauggugacauccgcggcaagaacagaguuaauccugcccgagcaggacagaaaagcugacgaugaaagcaaagggucaggccugggaccaaaugaaggcugagagguauggcucaucaguacaagagagaugcaaaaaacuaaguuggaaaguaaaggcuacacacacauauggagcaccccaucccacagcacauuacauccaccucacuucacagaacggagaacagagcagaaaugaccagaacaccuuugucaccaucacacagcccuccuaaaauggaaccaaagcuucccagcucccucaaagcuuuggaugcaaagaaggcacccugacuuccacaagacaccagaauucacacgguacucagaggcacugcuggggaaguuuguuggucuuuauuagauaaauuuccagagaccuguccauaauacccaacagaacaugacuguuucuuugaggaaaggguuauaaugucugugguguacaagucguuuuugguauaacuucuuuccugcugcugcugcuucccggcaaacauaguuuuccuauuucaggcagagugcgguauauuccaggaaacacuguuuccuacucacuuagcuuacuucuuuguugaaugccucacuaauggcaaguuucaagauguuuugggugacaaugcacacaugcugggcaaaagggugauggccaguggcuggcagcugggccagcagaagcuaggacaucugugaguugucauucucaucuauccauguccacuggccugccagcauccgccagugccuugccagugugcacggucccacacuguggccccugaguccccuaauguacacgcugcagccagaaugcagauggagcuggcuuggcuguucccuggaugggcaauaaagaaagugcugcauccca

SEQ ID NO: 15 - RNA, 4285 nt, Homo sapiens
  ref <308>: DATABASE ACCESSION NUMBER: NM_001184796.1
  ref <309>: DATABASE ENTRY DATE: 2020-06-20
  ref <313>: RELEVANT RESIDUES IN SEQ ID NO: (1)..(4285)
uggcuugaaguccccuccuccaucccggggaucacguuuugccugggggagcugaucgcaagacuaggcaaccuccagccagucccugggucgggcggauccucccagagguggcacaauggagcgaucuccaggagagggccccagccccagccccauggaccagcccucugcucccuccgaccccacugaccagccccccgcugcucacgcaaagccagacccagguucugggggccaaccugcuggcccuggcgcggcgggugaggcccuggcggugcugacuucauucgggaggcgguugcuggugcugauaccuguguauuuggccggggcagugggacucagcguggguuucgugcucuucggccucgcccucuaccugggcuggcgccggguccgcgacgagaaagaacggagccuucgagcagcgaggcagcuacuggacgacgaggagcagcucacugcgaaaacucucuauaugagucaucgagagcuaccugccugggucagcuucccagacguggaaaaggcugaauggcucaauaagauuguggcccaggucuggcccuuccugggccaguauauggagaagcuucuggcugaaacuguggcuccggcuguuaggggaucuaacccccaucugcaaacauuuacauuuacacgaguggaacugggugaaaagccauugcgcaucauuggagucaagguucacccaggucagagaaaagagcagauccugcuggacuugaacaucagcuauguaggugaugugcagauugauguggaagugaagaaauauuuuugcaaagcaggagucaagggcaugcagcuacauggcguuuugcgggugauacuggagccacucauuggggaccuucccuucgugggggcugugucaauguucuucauccgacgcccgacccuagacaucaacuggacagggaugaccaaccugcuggauaucccaggacuuagcucacucucugacaccaugaucauggacuccauugcugccuuccucguguugcccaaccgauuacuggugccccuugugccugaccuucaagauguggcucaguugcguuccccucugcccaggggcauuauucgaauucaccugcuggcugcucgagggcugaguuccaaggacaaauaugugaagggccugauugagggcaagucagacccauaugcacuugugcguuuggguacccagacauucugcagucgugucauugaugaagaacucaacccacaguggggagagacuuaugaggugaugguacacgaggucccagggcaggagauugaaguggagguguucgacaaggauccagauaaagaugacuuucugggcagaaugaagcuggauguagggaagguguuacaggcuagcguucuggaugauugguucccucuacaaggugggcaaggccaaguucacuugaggcuagaauggcugucacuuuugucagaugcagagaaacuggagcagguucuacaguggaauuggggagucuccucucgaccagaucccccgucagcugccaucuuaguugucuaccuggaucgggcccaggaucuuccuauggugaccucugaauuguacccaccacagcugaagaaggggaacaaggaacccaacccuaugguacaacugucaauucaggaugugacucaggagagcaaggcugucuacaguaccaacugcccagugugggaggaagcguuccgguucuuccuacaagacccucaaagccaggagcucgaugugcaagugaaggaugauuccagggcccugacuuuaggagcacugacgcugccucuggcccgccugcugacugccccagaacucauccuggaccagugguuccagcucagcagcucugguccaaacuccagacucuauaugaaacuagucaugaggauccuguacuuggauucaucagaaauaugcuuccccacggugccugguuguccuggugcuugggacguggacagugagaauccccagagaggcagcaguguggaugccccaccucgacccugucacacgacuccugauagccaguuugggacugagcaugugcuucggauccauguauuagaggcccaggaccugauugccaaagaccguuucuuggggggacuggugaagggcaagucagaccccuaugucaaacuaaaguuggcaggacgaagcuuccggagccauguuguucgggaagaucucaauccccgcuggaaugagguuuuugaggugaucgucacaucaguuccaggccaagagcuagagguugaagucuuugacaaggacuuggacaaggaugauuuucugggcagguguaaagugcgucucaccacagucuuaaacaguggcuuccuugaugaguggcugacccuggaggaugucccaucuggccgccugcacuugcgccuggagcgucucaccccccgucccacugcugcugaguuagaggaggugcugcaggugaauaguuugauccagacucagaagagugcggagcuggcugcggcccugcuauccaucuauauggagcgggcagaggaccucccgcugcgaaaaggcaccaagcaccucagcccuuaugcuacucucacugugggagauaguucucauaaaaccaagacuauuucgcaaacuucagccccugucugggaugagagugccuccuuucucaucaggaaaccacacacugagagccuagaguugcagguucggggugagggcacuggcgugcugggcucauuaucccugccccucucagagcuccucguggcugaccagcucugcuuggaccgcugguuuacacucagcaguggucaggggcaggugcuacugagagcacagcuagggauccuggugucccagcacucgggaguggaagcucauagccacagcuacagccacagcuccucaucgcugagugaagaaccagagcucucggggggacccccucacaucaccuccucagccccagagcuccggcagcgccuaacacauguugacaguccccuugaggcuccagccgggccucugggccaggugaaacugacucugugguacuacagugaagaacgaaagcuggucagcauuguucaugguugccggucccuucgacagaauggacgugauccuccugaucccuaugugucacuguugcuacugccagacaagaaccgaggcaccaagaggaggaccucacagaagaagaggacccugaguccugaauuuaaugaacgguuugagugggaacucccccuggaugaggcccagagacgaaagcuggaugucucugucaagucuaauuccuccuucaugucaagagagcgugagcugcuggggaaggugcagcuggaccuagcugagacagaccuuucccaggguguagcccggugguaugaccugauggacaacaaggacaagggcagcuccuaggagcuggcgagucccagccugacugcucugucuuccugccuucgucucgcuccaucaccgccucaaugugaugagccuaaagcuaggguccaagggcagagccugugcccuucagcccuuucaccuaacaggcccauauucgggccuuugccugaccaaagagaagaaccguauguucccuuuacugcacggccuuuauccuucugggccccuggggcggggaccugagcuggcuguuuccugcuuugccugcacauuguucucccuuccucccaacuccucagggccuucuguaucugugccuggccaguggcagcacuagcagugguauuagcuuaugccaaauacagcuuuggaaggaucuuuuuuucuuuaacuagauggucaccuucuucccuaccacacaugggugggaagguggacaggcuaaccucuccagcugugagccucuuagacuacugcauguagcaaauguucagcagcucaggcccccauguccaguucuguccccacuguccucaacccuguccugaaaauucuacugcuuugauggcuggggccagucucuugucacuuuggaaacugaggacgcguggauucuacucaagccuccaaguaguggcauaucagucuuggagcuccuagcuggugauacggagagggcuuuggaggacuugggacagcagggccaauuuuuuugcccaagugccuaggcugcuaacucacugacuagaacuuaaucugguacuuuacaguuuugcaccaacucugccaagccacuggaucuuacauuaaacaucauacucaaaccagcaaaaaaaa

SEQ ID NO: 16 - RNA, 22920 nt, Homo sapiens
  ref <308>: DATABASE ACCESSION NUMBER: NM_020762.4
  ref <309>: DATABASE ENTRY DATE: 2022-06-03
  ref <313>: RELEVANT RESIDUES IN SEQ ID NO: (1)..(22920)
cggcgcugccucuggauugccugcgugugggaguacaacucugccucuccaaggagaacggguugugaccacugaacaaaacuugcccauugaaagcaaacccggaacagcuggauaauguccaccccgagccgauucaagaaggacaaagagaucauagccgaguaugaaagucaagucaaagaaauucgagcucaacugguagaacaacaaaaaugccuggagcagcaaacggagaugcgaguucagcuucuccaggaucugcaagauuucuuccgaaaaaaagcugaaauugagacggaauauucccggaaucuagagaaguuagcagaaagguucauggcaaaaacaagaagcacuaaggaucaucaacaauacaagaaagaccagaaccuguugucuccagugaacugcugguauuugcuccugaaccaaguaaggagagaaagcaaagaccaugcaaccuugagugacaucuaucugaacaaugugauuaugcgguucaugcagauaagugaggauucuaccaggauguuuaaaaagagcaaagagauugcauuccaacuucaugaggauuuaaugaagguucuuaaugagcuuuauacggugaugaaaacauaccauauguaucaugcagagagcaucagugcagagagcaagcugaaagaggccgaaaaacaagaggaaaagcaaauugggagaucuggugauccagucuuccauauucgacuagaggagagacaucaacggcgaagcucuguaaagaaaauugaaaaaaugaaagaaaaaagacaagcaaaauauucagaaaauaagcuaaaaucaauuaaggcacggaacgaauaucuccuaacacuugaagccaccaaugccucaguuuucaaguacuauauucaugaucuuucugauuuaauugauugcugugaucuuggcuaccaugcaagucugaacagagcccuaagaacauaucugucugcggaguacaaccuugaaaccuccagacaugagggcuuagacauuauugagaaugcaguugauaauuuagagcccaggagcgauaagcagagauucauggagauguacccugcugcguucuguccaccaaugaaguuugaguuucagucucacaugggugaugaggugugccaggucagugcccagcagccaguccaggcagagcucaugcucagguaccaacaguugcagucccgccuugccacgcucaaaaucgagaaugaagagguuaagaaaacgacugaagccaccuugcagacgauacaagauauggucaccaucgaggacuaugauguuucugaaugcuuccagcacagucguuccacagaaucagugaaguccacugucucugaaaccuaccugaguaaacccagcaucgccaagagaagagccaaccagcaggaaacugaacaguucuacuucaugaaacucagagaauauuuggaaggcaguaaucucaucacaaaacuucaagccaaacaugacuugcugcagaggacccugggagaaggucauagagcugaauauaugacuacaaggccuccaaauguucccccuaagccccagaaacacaggaaguccaggccccgcucacaguauaauacuaaguuguuuaauggggauuuggaaacauucgucaaggacucaggacagguuauuccccucauuguggaaagcuguauucgguucaucaaucucuauggucuucagcaucaggggauuuucagagugucugguucccagguggaagucaaugauauuaaaaauucauuugagagaggugaaaauccuuuggcugaugaccagaguaaccaugauauuaacucaguugcuggcguucugaagcucuauuuccgugggcuggaaaacccccucuuuccuaaggaaagauuuaacgaucugauuucuuguaucagaauagauaaucucuaugagagggcgcuucacauccgcaaacuccuccugacuuugcccaggucgguccuuauagugaugagguaccucuuugccuuccucaaucaucuaucacaguacagcgaugagaauaugauggacccuuauaaccuggccauuugcuuuggcccaacauugaugccugucccagaaauacaggaucaagugucuugccaggcacaugugaaugaaauuaucaaaaccaucaucauccaccaugagacuauuuucccagaugcuaaagagcuggauggcccuguuuaugagaaauguauggcuggagaugacuauugcgacagcccauacagugagcacgguacauuggaggaaguggaccaagaugcugguacagagccccacacaagugaagaugaaugugagccaauagaagcaauagccaaguuugacuauguugggcggucugccagagaacuauccuucaagaagggugccucccugcugcuguaucaccgugcaucugaggacuggugggaaggcaggcacaacgggauugacgggcuggugccucaccaguauauaguggugcaggauauggaugauacguuuucagacacucugagccaaaaagccgacagugaggccagcagugggccagucacggaagacaaguccucauccaaggacaugaacuccccgacagaccgucauccugacggcuauuuagccaggcaacgaaaaagaggagagccacccccuccaguaaggcguccuggcaggaccagugauggccauugcccgcuccacccuccacaugcccuuucuaacuccucaguugaccuaggguccccaagccuugccagucacccccggggccugcugcagaaccguggccucaacaaugacaguccugagcggaggcgcaggccuggccauggcagccugaccaacaucagccggcacgacucccucaagaagaucgacagcccucccauuagaagguccacgucaucagggcaauacacgggcuucaaugaccacaagccacuggacccagagacaauugcucaggauauugaagaaacgaugaacacagcuuugaaugaacuccgagaacuggagagacagagcacagcaaagcaugccccugauguggugcuggauacccuggagcaagugaaaaacucucccaccccugccacuuccacggaaucucucagcccuuugcacaacguugcccucaggagcuccgagccucagauucgacguagcacgagcuccuccagugacacaaugaguacuuucaagccuaugguggcacccagaaugggcgugcagcugaagccuccagcccuuaggccaaaaccugcuguucuuccaaaaacaaauccuaccauaggaccugccccaccuccccaggguccaacagacaagucaugcacaauguaaaaaccagccaagcaaggccauaaagggaggugacuuaaaaaagaaaauggauuagugacaaaagucacugauccauaacuuuccuuaguuuugugcuuauaacuggagaucuuuuggcuuuucuauguugucgaauguaaugucugagacuagcuaaauuaacacgggcauuuguauuuuguaauuuuuuuaaauaacuggacauaugucauuuuaaggacaauagaaacacuuagacuuacuugaaaauccaaugcugcaccacuuguaaugaaggcaacaccgcucuccacauuguacagagcuucagguuuaauguagcccagcugagucagaaagguugugaccugaaggcagaagaacccgaaugccacaccucauuggaguauagccaguguuggucuguggcacuugggcugaaaggugauaauggcauugcgugguagcugacaaugagcaccuucgguuccauguggagcgggguuuagcucaugcaaaagacuugcaauugucuccaugggacgaucccagugggacugucagcccacagcucgaguggguuggaugcuugccucuuuccuaacaguuauuuccccggguccagcuuaaagacucgauggaaggagguagaaccucugcuguuacugcuugaacuuaaccugggaaaggagaggaagacaccaucuccaaagcuauuaaugucacuccuuuugcgagcaugauuaggccccggagauuuccaaguccccccaucuacacuuacaaacgauuagaaggguuuaauuuuaaagacuuucugguuacacuacuccacgaacuccuccaaagauccguuauucaauaacugccuagaaaauguuuccaucuccucuaaaucccuguguucuccucuguggaaaugaaggcagcaagaagcaccugaggccuugguucaugcaguguucucuuuugacuaaaucaccuagguuccuuuaaacaugcuacaaagcccaggcaugguggugcacaccuguacucccagcuacucgggugguuuacacaggaggauggcuuugggccuaguaguucgaguccagccugggcagcauagugugagacccugucucuuaaaaaaaaaaaaaaugcuacaaaguccugaugauccuaaauuugaaaucccaacuguccucuccauuugucuuugagccaacccugaacuggcccugguaccugaguaagagccaugcagggccaucagcagaacauggccuugugguucucacccaguuguguaccucacaaccacaacuucuuuuucuuugucccuuuuccccuuuuugguuuugguuuuguaugugggcucuauguaacucccauagucagcugaacucucaauuggagacccucuuucucuggcauccuagcacacaggcaauuuuuuaagccuauauuuaugcuacauuuucccccaucuuaagaaaauuugcuuugauuuccauucuccuacuagcagcagcugauaucaccuguagaccuaugagcauuuuaucuauucuaucauugaaguuacugauaaagauuuuuguuguuguuguuguuguugggagaaauggauuucccacaacuaaucccucuggcccaccauuaauuuccuucuugguuucauuucaaacuggcagccaauugcacgcccaucuaagagcuacacaguguagauuuuacuucaucagcccauuggugaaugcaucgugcagaaaagaacugggaucuguacuaaaaucaagaucauuacuuugaucucuaccucaguuugccaguguagucauucuauugcaggaagagauuaaauugaugagauauuguuacuuucagagucacacugcugauugcuugccuggggccuaauuucacaaaugaauagugacuuuuuucccauguauuucuagguaugguaaacuuuuaaaccuccaaauuuucagagccauacuuuuugcccauuuugaaaaagaagcccaggacagcaccuugucuagccccuccccauuucuccugagcuuuugggguccuucagcacccuuggacaccguggagauccagccaagacugagcuuugaauagcacuagcccuuuuaaacaauccucacaaauuccuuaggaauuuaaacuuuccaaaauaaucccccacuuuuuuuuuuuuuuuuuuuuuuuuuugaguuggagucuugcucugucgcccaggccagaguacaguggugcaaucucggcucccugcaaccuccaucucccggguucaagcaauucuccugaguagcugggauuacaggcgccaccaccacgcccagcuaauuuuuguauuuuuaguagagauggggugucaccauguuggccaggcuggucucaaacuccugaccucaggugacccaccagccucagccucccaaagugcugggauuacagaugugagccaccaugcccggccauuccaccacuucuuuaaaauaguaagaauucuuaaaaaccauauuccuuguuugauggaagaguaagauguuaacagagggcaucaaagggccagaggugucuuugaauuuauuuuuuuucaaugucuuuucugagcuucagccuuagcuuuucagaucuuccuccuaccuguucauuuauucagccuucuaucuggucauccuccccagcggcuccucucucugauguguccaccuacgguggaacgaggauugccuuucugugcuccccaucaggacuuugcugucuuggccggcaggagucgugcugcugcagagccucacaggcugugccuuguuucaggaaacagcuccuaauacucucccaucacucacuuuaccugucuccaucuuauuucccuauacuccuauugucagcuugagacuuuuuuuuuuuuucaauuccaaaugaaggguaugaccucagagugagugaggacucaguaaagacucaccucuccuuacuugguuuauuaaaugucuucagauuccagagauaagacaagguggccacuucacaaagaauccagaaucaugcucaguaaagcucauuaaaagccacugcagcugagaagguucacagcccuucuuuauagccacagaggcagcacacaggggaggugggaagacacagggaaacgagagaagaaggauaaugaggccuugagguguucugcccccaauuucaaggagcuuaucaggcuucaugugcaauuuggggaggggagcuuuuugaugguggguaaauaauggcucuacaaagauuaccugacuccacugacacaagaauauguauucucagcugauauaacuguguguucacaucccauccauagaacucacugaaggagauucaucaagucagccuuuuggacuggguugggguuuggggaguggauuuggggauagagagaaagguguacuuaguacacauuuucuaaugggcguuacgugaacuguuugaaccugcugaguggcgcuuuguagagcuccauguucacguucagugucaacaccauucaacagcuuugcacaucuucguacaacauaaucuggcuuuggugccccuugagccaccuguagaauaauuuauagcagggucccacaaucuacugcaaugaucgugaacaguuaucaguggauauagugcuccaagagucaaauuguauccguuaaggguuuucuaacuuuugcuaucccccccgcgaccaaaaaaaggggggguuuauaauuuaaagaaaaggaaaagaggcguggguugguaguuguguccuuaacauuuaggaaugacauagccucuggagucuacaaacugaagaaacuuucaucugaucagccuuggaaauccaaaguuaaagagccuaugugucucuugaggcggggguuagaggauucaaauuugggaugaugcucauugauuccugaccauagcagugagaggccauuuuuugugcaggaaaugugcuuaggacucagucuuguuuucgauuauccaccacagaaacccugagacacagagcagcuuagaagucucuacccaggcguaaauagagcucccuacuccagaccaccugccacccaccucccaaguugagaacacaagcuccagcugggcuggagagucaggcuuggugcagggugacuuuggcgaaguuuugucagauccauaaagcaaacuggaauuugagcuuucacuuacccuaguauacguucuuaaaaaaaaaaaaaagucuaugugguauaaucgagauggauaccugugucuuuaaauuacguagggaauuuuguauguuuaaauaauuguacuggguuccauaaugcuuaucuuagaaacuuuaguaaaagaaaauauaugaaccgugcauuugugaaugccccccuuagagcauucauccaaguucaguguguagugugaugguuauuauagauauuuaaaguauaguaaguggcuguguaacaggagagacugcuguuuaccuacguacugaaggguaacugcugcccgcguaauuaaaucaguauuguccuuuccugucgguuugaucaauugcgguaccgguagcuuccugcuugugacuguuaccuaauugugucaauguacaucuguaguauguacaugugaaagugccuuucuauuuuagaggaguuuuagccuugcucuuguaccguugccccgcucugugccccacccccaccuccuccucccaauuccugcugcccacucgaguguuucgaagcuuccucccugucccccaccuuccccauccccgccuuccugugccucggugcucaguaaugucccaucucaccucccugucgguccucagacugucucguuuuccuuguagacgugauugugccuuucucaccccuguguccucccaucccaccgcaucagucccccgcucaugugcugcugacagcaucgcauucgcccgugcucuguacugccucucaccauuucauucccuccucucugaggugaaaauaaagggggaugcuuuuacagucuggaaagaaaaaaaauguuuuguuaaucuguugugacaaugcacuuuuauguauaguacuguacauuuuuggcauguaccauuacaggcuucaguauacagucagguuuguucuucacgguguaucuuuauaauaaauaagauaguucuggcugccuuugucugcuuccaguuugaugugcaucucuguggugucuuuguuccuuuccuuucauucaucacuacuuucuccuugacagcuagcuugcauuaaauaaugugaaacuuuuaccuuuaaaauauuuuuuuaucucacuuucauuucuuguucauuuggaaauaggguguccugcacagggcccugcccaugaagcugugguuuuucagucaugguuuggaggcucaauguguagucagagaaaagagucuuggcagacacuuaacugagaagcugagaguguacaagccuccuaaacauacaccagauugaaaucuuucuuuugagaaagacucuaguuaaccacggcaagcccaucuuugguuucauuugaauaaauugaaaaauaauccuuagguuauuuuucugugcuuagaacuccccguguauugguauuuuucuuuacuuuuuuuuuuuuuuuuuugagacgaaucucucugugucacccaggcuguagugcaguggugcaaucucagcucacugcaaccucccccuccuggauucaagcgauucuccugccucagccuccuaaguagcuguaauuacaggcacccgccaccacacccagcuacuuuucguauuuuuaguggaaacgggguuucaccauguuggccaggcuggucucaaacuccugaccuaaagugaucuaucuaccugccucagccucccaagugcugggauuacaggugugagccauugcaucuggccagguauuuuucuuuaaauauaaaaaaaagauaacuugaggucuagcaauauguaguagauagcuuuguaaaacaauguaaguugugcaagauugaauauugauaagauauaaaguuuaaaaaaugacaggcaaaucaaggaaaaacacuuuuuauuaaagugcagaauacuaacaaaagugugcaaaucauuaguguaacaguucucacgagcugacacacugauguauccaccacccaguuuaagcaauagcacauuaucagcaucccagaagcuacugugcucaugccucugugaauguaucaguauccugacuaaucacaccaugguguaguuuugccugucaucuuagcuuucuauaaauggguucauacaguauguagauuuugugucuggcuccuuuuguugaaauguauguuuguaagagucgucauuguuaguaaaauagugguuuuuccauuuuuacugcuauauagaauuccauuacaugaauaugcucaauauuauauuuauuuuaguauuuccugauuaaacuuuggguaguauaugcagauaucuuuaaacauggagaaaugaauggggggugagggacccaccugucaaaggaauuguuuucccaaacuaugacggaauaauguugaacuaaccuguugaccggaaggccuacucaaaggcaaggauacuuuucuuucuuuccaaaaaagcacugucugauauggagacuggaaauauucauagaugacacugaaacuaguugaagcuuuacuugaugcuuaucaagucauguucucuuacccgaugcuggaugcuuuuucuucaaugccuacuuuaggaccacagaguuggauuuagaggcuagcccaccaggaacaaaugcccaaaucagucuguagaguuguuccaggaaagacuccaccauccugcuacccucaaaucuuaagcuccucagccugccacgaccaucuccuguugagaaaauugcgccugccuccccaucucucacucccgaaccagagucccaacgggguguaacuggucgucaguucccacaguucauaugccugcacccuguaagaaucuuagaaaggcagagcaaggguguuugcuggcuucugccuugaguaguugggacuccuaaaaaggaaaauuccccaaguuugaccuagaaugucaaaaagguccuaaaagcacagaaugaugguaaauucuauccacaggccauauagcccuggaaaauauugggaaacugaggcauggguugguaauuuauugaaguuuaguaaacgaauuugaauuaauuaggaccagauaaucaauuuuuggcuuacuuauacuauaguauaagcaaauaaguauuuaaacuuuaguauaaauauuuuugcucacuuaauaaucacggugagguucaucaccucuucaguagccuuuuaucuguuucuacauaguaaauagaaaacuugccuucuauaauaaaaacaaaauaguuuuucuaucaaggucagucucagcaagagaaaugggacugggcuggauaucaacacaugugcuugugugcaugugcacgugugugggugggagggugcggagggguugggaagagagauugcuccucccgcagugcugaaacaacaugccuuguucaguauuaacaucuugaaggcaguuauggcagcaugauccaggagaucgucagcucuaggauauuugcgauccaauucuggcgacuuauggauggauccuucacacccaacagcugucuaucaggggugcuuccgagggucacuggguuucuuuuagcugcuuggcugugcagcugaaguuaggacuuuugaugaauuacccucucaaugaugguguauuguucuaaucuucugucucugacauacccaaggaaccccuuguguaaaaaucaugucccucuaaaugcaaguggaugauugugccaauaagccaggagagaaggaaacaucuucagugguccaguggcacauucaaaauuguacaaugacaugacauuaacucaaaaaugaaguuugagguucugggccuuugauggacugcaucuauuaaaaacaaaaaguggugucucaugccuguaaucccagcacuuugagaggccaaggcaggaggaucaaaaaaugcgaauuuuuauauuuuuuugugagaucccaucucuaccaaaaaaaaaaaaaauacaaaaauuagccagacauaguaguacaugccuauaguacuagguacuugggaggcugaggcaggaagauugcuugagcccagauguuugaggcugcaauaagcuaugauugcaccacugcacuccaaccuggguggaagagugagaagcuaucucaaaaaaaaaaaaaaaaaaaaaaaaaacauggucaagauuuguaauagaaauugccauuugaauuuuggucuguaagcccaaagauuguuacuguguuaguaacuaaauauuacgagaugcauuaggaaaagaaggaaaagaaauuuuaaguaagccaaaaaucuauuauauagucauaagucaaacucaguuacuagacuucaacaaauaccaacccaugccuccauuucccaaacagaaauaauaauaugaacaucuuaguauauugucuucuaauccuuacauuauguuauuuuuaagcagagucaaacucauauucuguauauaauuguauauacauuggguuuuucucccuuauagcaauucacacuaggagucucagucaacaugauuuuuuaauggcuguauaauauucuagcguaugaacacaucauuauuuaaccauuuuccugucacugagugagccuguuugaagguuuuguuuuuuuuucaauugaauguuauguuguggugagcagcucuguauauaauaauuaauugucuguguuuggaucauaaaguuagcauugacuccaaaaagugaaaucacugggucagaggaugcaauuaucuguaaggcucaugacaaauauugccgaccuguuuuccagcaaaguuguacaaauuuacaucccugccaguaaugcauaacaguacacgaguauaaaacauggucauguuucuuuaccaguguacuaggugagugauaggcucaaugcaaauucuuagcaguuuguuaaugugcacacccacauuuccagaguacaaaucucuauuucacucaccucuguguccuaaacuucagaugacucuuuauuguuucagcuaaacaucagcaaacagagauuuuccucaggcaccuuaagcucaacagacccaaaaauuaaacccacuuuguuucauuccaaaaaauauucugcagccccucuacauccuccaaucuguuccgcugccuaucucugcucuaagcuaauucugugcaccauuccccuugccacaacgauugccguagcuuccacggugguguggcugugcccacugucuccuccuugaaaugauuaucuacacugcugccaauaaaucaucuuucucaaaaacaaaaugagaaaccuaacaaacaaucauugaaaauguaccauguacugacaucguuuuccucaacacguugcaugucaauagccaacauguauugaguaccagacuccaugcuacaaauguuaacccucauaagaaagccaagagagccacaguuauuaucaucaucuuuauccguaagaaaauuaagccgcaggaguuaaacacuugcccuggucacacagcuagauagugacagagcaggauuugauuccacgccaucuggugcuggaggcuaugcucauaaccacugcugcauucugaccucuguaacaugcucaugucauuacccugcucaaaagccacacguggguccccacugcugcaggacggcauacuacugugucuuauucaucuuuguaucuucaagccuaaugcaucacuaacuaaauagcaaaucggcggaacugaaaugaauucaaugucugccgcuuugaccucagccugccauuucagccuuccugcucaccaccuccucuucggaaccuuuaguuucagccauagcaagcuaccuuucuuugaaaaugugaugcugugugcucccuccgcauauacuucaugucauuccucagccuaaaauguccccuacuucauccugacaacugccuguugaaauuauauucaucauuccaaacccaacaaaaugguuacauccuucaugaaggguuccccaaaccucccagcuggcauuaauuguucuguuacauuuuguuuucauuaaaucaggcucccuuccggggaucuagcugugaaugagacacacagggcuccuggucucauggaguguacuuccugguaugauuuaaaaaaaaaaaaaaaaaaaaaaaaaccacuacauaagccaacagcuaaaccggcaaaauaauuucagguguugauaacugcuacaaaaagcaaaagugaaauaaacagggugauagaauuuagagugucuaggagaagcauuuuagaugggacagcuugaaugccaccuuggaugugguagcauuugagcuaagaccugaauaaugagaaggaaccagccaugugaaaaggaagagaaagacccaagcagaaggaacugcacaugggaaaacaggaccaugcucacgugguuaaagagucaaacacauucacugugacuguaccaccauaagcaagaagcccuggagaugaaaauaaaggccagaauguggacagagaucagauuuuggaggccaaaguaagguacuuaaaauuagccgggcuugguggugcacaacuguaaucccaccuacucgggaggcugaagcaggggaauugcuugaacccaugagacggagguugcagugacccgagauugugccacugcacuacagccugggcggcacagugagacuccaucucaaaaaaaaaaaaaaaaguaagguacuuagaugucauucuaaguucaaggaaagccauuggaaguuuccaaaaaggaacaugacagcaucuauauuuuuagaagaugacccucaaaaccacaaugagauaucaccucauacccauuaggauggccacucucaaaugaucaaacuccagaaaauaacaaguauuggccaggguguagagaaacugacacccuugugcacuguuggugggaauguaaaaugguagagccacuaaggaaaacaguguggaaguuccucaaaaaauuaaaaauaaaacccucauaugauccaccaagcccacuucuggguacauauccaaaagaauugaaaacagaagagauauuugcacacucauguucacgcagcuuaauucacaauagccacaagagagaagcaacccaugaauggaugaauggguaaagaaaaugugccauacacauaaaauagaauauuaugcagccuuaaaaaagaaggaauuccugucauaugccacaauauggaugaaacuuaaggauacuaagcuaagugaaaugagccaaucaauaaaggacaaacauugcaugauuuuacuucuaugagauaucuaaaguagucaaacucuuauaaacagaaagcagaauggugauugccagggaucgggaugaggaagaaaauggaaauuguuguucaguguuuacaggguuucaguuuugcaagauaaaaaguucuaaagaucuguuguccaacaauaugcaugcagauaacaucauauuaaacaugguuaagauaguaaauuuuauacuauguaauuuuuuaaccacaauuuuaaacaucacuguagauguuguaguuguguuugggguccacggguggggaggagcuugggaaggaauggcaugagggaagugagagaccacaauuaaggagcaacacaggagaugaggguggcuuggacuagauagccagguagaaggagaggaagggaugaaccggggguucguuucagagucaaaaucaagauuugcuaauaaauuaaauaugggaauaaagaaagaaaagaaucaagaguggcuccuaguauuuguuugagcaaauggguggaauauauugauauauacuuaaaaagaaugacuggggaaaggaagaaguuggccaggauguacaaccaaguauucacguuugaauuuauuuaauuuaagauagguauuagacuuccaaguggagaagucuaggaggcagauggacuugaagcucaggggaggauuucuggccagaagguguaaauuugauauuuaaagcuaugggacucccggaaagagaaagagaaaggaagguacagagcacauaaccaagcccugggaagcccagcuugggagagcuuggguggagugaggagcuggcagaggagacagaaggagcagccagggcggaggaggaaaaccaggaggagcauguaaugugaucaguacccagggaagaaaugauugccauuucuagugugcauuuguuaacauaaauaugauaauaaaaauaguuaacauuuguuaaauauauauauuuuuuucuuuuucuuuuucuuuuuuuugagacagagucucacucugucacccaggcuggagugcaguggcaugaucuuggcucaccgcaaccucugccucccaggcucaagugauccucccaccucagccucccaaguacgagggauuacaggugcaugcaaccaugcccagcuaguuuuuguauuuuuuuguagagacgggguuucaccauguugcuuguuaaggccgggcacgguggcucacgccuguaaugccagcacuuugggaggccgagacaggcggaucaccugaggucaggaguuugagaccagccuggccaacauggcgaaaccccaucucuacuaaaaauacaaaaaaauuagccgggugugguggcgcccgccuauagucccagcuguucgggaggcugaggcaggagaauugcuugaaccagggagguggagguugcagugagccaaggucacgccacggcacuccagcuugggugauagagugggacuccgucucaaaauaaauaaauaaauaaauauauuaaaaaauaaacaaguucaggcuuguuaaauauuuuuuauguuuguuuguuugcuuuguuuuuuuugagacguagucccgcuccgucucccaggcuggagugcaguggcgcgaccucggcucaccacgaccucggcucaccgcaaccucugccucccgguuucaagcgauucuccugccucagccuccagagcagcugggacuacaagugcgcacaaccacacccagcuaauuuuuuuguauuuuuaguacagccgggguuucgccauguuggccaggcuggucuugaacuccugaccucaggugauccacccgccucggucucccaaacuguuggcauuacaggcaugagccaccacgccuggcugaguuaaauauuuucuaugugccaagcacugugcuaacugcuuucauauuaucggauuuaguccuguaagaccggaaucauuauuauccccauuuuucagauuaaaacaaacaaacaaaagcugagguuuaaagaaauuaucucuuguaaaaucacacagcuaacaauugagccaguaucuggaccuagaucuauugccuacagagccgcacguuuaacuacuguuaucuugccuccuuauugcuccacacacuugaaauauaaauuuuuaaagacauuuuauucuuuuuugucuucaucagaauuuauugaauccugauuauuuuuuccuuaauguugauuuuguagacucauuugcaggaagaacaaugucagguggagagaaggaauugaaugaauauuuuuaauaaauuuguagauguuaauaugauuuacuuauuugcaauuuucauagcuauaugauauucuaucccauuuccaguguuuuugcucuuuuaguagcaaauacucugaaucucugcauauguuacucauuuucuaucauccuauuccuugugauggauacccaaggguacucaucuacaggaugaaaggguaugagcacguuucuaggugcccugaccccaagauuagggguuuuguuagaggcaaagcuaaaaggaaaugauaaaauaaaugaauauuuauagaaguuaaaucuauuaaucuagccuuauuacaccgaguacaacagggaauaauaagccaucuaucugaacgugcacgguuagggccucaaggaaagugggcucagcaaugaguguugguacacauggaaaaucacauuugccauguuuucuauggcuuugggcugcugcugcaucacugcaguuuaauaggaggaggcuggugaaaauguuaccaacugccaggggucaauggcgaaagaacacauuucucuaggaaagauuaccuucucugaagaagugauagggggaauaaggcauaaaauuaucugaccugagagaccuuaacagcuaggccugcacugcucaugcuuugggaccagaaagcggaugguuugauaugguagcugcacuucuaaaaaaaaaagggggcauucuuggcaggagaaaggguguuacauggaaagaucagcgauaguacacacgccuucccauagagugagguggcuuucccaccccucaacacuccuucagacccccacuguaacaaagggaugugucuaccucuaaauucaucauuuacugaacaaauaacucaugaggguuaccauaugucugacacuguaggaagagcugaagcagggccuaaaacacuauagugcaaguaucuguuuacacaucuguaugagaguaauugaugcuagcugcuguaacaaacaaccccccaaacucaguaacacaaucaagcuguauuucuccuucacaucauagccuggugugggucagguaggugacucaggguucuaggcuccuuccauuuuuuugucgcugccacacuggaaaugggauagaauaucguauagcuaugagaauugcaaauaaguaaauuauauuaacauguacaaauuuauuaaaaauacucuuucaauuccuucucucugccugacauuguucuuccuggaaaugagucuacaaaaucaacauuaaggaaaaaaugaucaggauucaauaaauucuggauagaaaaaagaauaaaaugucuuuaaaaauuuauauuucaagugugugggggcaauaaagaggccagauaauaguaguuaaauguggggcucuguaggcaauagaucuaggucagauacuggcucaauuguuagcugugugauuucacaagagauaacugaugcuagcugcuguaacaaacaaccccccaaacucaguaacacaaucaagcugaauuucuccuucacaucacaacuugguguggaucgggcaggugacucaggguucugggcuccuuccauuuuuuuuguugcugccaccuuaguacguggccaccaugaagaggaagagaaagcacuugcaaugaugugagguuuucugucuugguauggaggagacaugaaucacuacuguacauguccaagucacauggcaagggaggccaggaaaugucaucuuccuauaaugcaagaaaaagaacccagggccgggcacaguggcucacgccuauaauccccacaauuugggaggcugaggcaagaggauugcuugagcccagagcaauuaaacacugccuggugcauaguuagugcucaagaauugugcuauaaucaauguuacuaaauuaucuaggauuagauguuucuaaauuaaaacuauuuccugcuuuguauuuguccuuuauuccaaaaacuuaaaaauuucucauuucugguuugucagggaugaaauacuaaagcaacuucauggauugugauuucaaguauaugaaaaguuuaagacccaauugcuuuugaaggagauucaucuagaaaaaccagggauucaaacccaacacaucgggugcuaaagucuacccucuuaguugacauagaauuaaauuaaaaucaguauugauuggauuaugcuuugauaaaaauguaacuucauuuaagucuaguaaauaauuaggauuaugaggucaggaguuugagaccagccuggccaauauggugaaaccccgucucuacuaaugauacgaaaauuagccgggcaugguggcgugcaccuguagucccagcuacucgggaggcugaggcagaaguaucacuugaacccgggagguggacguugcagugagccaagaucgcaccacugcacuccagccugggugacagggugagacuccaucuuaagaagaaaaaaaaagcaauuacuuuugcaccaaccuaauaaaaaugaaaaaucccaaugaaaaugcaugaauaucaugugccuugacaacuuacggagcauuuggaacaugucaaacaguaaucagaggucagguugaggaaaaaaugauaagaacccucucugccccaccauucacaucauucaguggaucuauguacuaaaucuagccaguggguugugagcaaaaaugaucugugucacuuguggguuaaaauauaaaagagugcuaggcacacuggcucgcaccuguaaucccagcacuuugggaggcuaagugggaggaucgcuugagccuaggaguucaagaccagccugggcaacauagcaagaccccaucccuacaaaacaguuuuuuaaaaaacuagcuguagggcaggcacggugguucacaccuguaaucccagaacuuuagaaggccgagguggguggaucacuugaggucaggaguucaggaccaaccugaccaacauggugaaaccccaucucuacuaaaaauacaaaauuagccaggcgugguggcgcaugccuguaaucccagcuacucaggaggcugaggcaggagacuugcuuuaacccaggaagcggagguugcagugagccaagauugcaccauugcacuccaaccugggcaacaaaagugaaacuccaucucaacaaacaaacaaacaaacaaacaguagcugguugugguggugugugacugugaucccagcuacuugggaggcugaagugaaaggaucgcuugagccugggagguagacgcugcagugagcugagaucauaccucugcacuucagccugggugacagagugagauccugucucaaaaaauaauaacaauaacguaugagagaggauucaagcuuuccaagcagucucuuccccugccauggcacucauagaagccucccguuaagauggcagagcuguaaugcuagaaaagcacagacuguuaagucacugcauggaggauggucauccuggagaauuguuuggacucgaguggacucuacaacagcaggaaauaaacuacuguuauaucaagcuacugacauugagaauuagguugcauaguuaugccacagcauaauguagccuuuccugaugcauacaauuacuuuuccuuucuuuuuucuuuucccuggaguucauaauuaccuuuguuucauuugcuuagcuuucuauuuacauaaauagguaaaaauuccuucucaaaaaucuuuuacagaaguguucucaaacucaaugcauugaaacaugcuauaguuuauaaaucuuggauccaucacuuaaagaggcauuugaccuguuucaguauggauaaguuguucucuaagucuacugcauagcugagagccuaagaucucuggggacucacuccacuuuucucuugaguugguuccacuauuuccuggaaccucauacagccaucucagcuagucaugacugaccuagauuuaacacaguauauccuuaaucagcuuccugauugagaguguauuuugagacauuccaugucuagaaagugucuuuaucucacauguaauugugauagucuggcugaguauaaaaucuuacuuaggauguuaacuuaaaaucaauguggucaaggaaaggaggaaaaauauguccuccuaguaauucccaguguaaguggaaaugacccagcccuagauuuacuuggcuuuaguuuccaucaccauagauuucucaaucaaaaggcaaauauaucucauaugucggaagauuaauguaugaauaaucaguaccacuggcauauggcuacucuccuauuuuccuuaccccuuugucuucacaaggaagcccagacucuguuugccaguggcuggguccuggggccgauaacuucaagcuaaacauucaauaucaguaugguaggugaggaagauuuucucagaguggcucauuaucuggaaaguguaagaaugagguggcagaaagagguagaaacagcaugaaaaggcagagccaaggagagaucgacaaacaagaaggauugugcccauauuucucuauucucucuagcauuaugaaucauuuccccucaggauuuuggagauguugcaucaguguugcuaccgaacauuccaaagccauucuaauucuguuacuuuguauguucuuuuuuuucccuauacauaauuuacuauugcauguuuuucucaggguuucugaaauuucagaauauccugauguggaucuauuuugauucauuguacugaagaccauuucagcccaucuuacacuuugauucuggaaagucuucuuuucccuggagcuuauaauuacuuuuauuucauuugcuuagcuuucuauguacauaaguagauaaaaauuaauucauucucaaaaucuuccacagaaaugcuuucaaacucaaugcauuaaaaacaugcuauaguuuauaaaccuucuuggaucuaucaaggaagauuuauuugauuauuuuaucuccuccauuccaucuguuuucuuauaauuccuaauauuuggagguuggaccucaugaacugauccuauaauuuuacuaauuuuccuccaguuuuucaucuauuugacuuuuugcucuauuuucagggaaauauccucaacuuuaucugcuaacucuucuguuaaguacuuuauuucugcucucaugcuuuuaauuuccaaaagcucuuuuugugagcuggcuguucauuuuuuuauagaauccuguccuuauuugacagguccuauauauuuuuuuauuuugagauuauuagugauaguucuucuaaguuuucuucucucugcacacuguauuuccucuaaguugcuuuuuuaaauuuuugaccuguguuugcuuugcuuucguuuauguuacaggcuuuccuaaaauuccuggugauccuuggcuaucugcucgaauuuaaaaacaagcacuaaagaagggcacuaaaaaacagauugucauagcaguauuauccauaauagccaaaugauagaagcaaugcaaauguccaucaacagagggacaaaugaauaagcaaaugugguauauugauacaauggaauacuauucuguaaugaagaugggaaagaacggaguacugauacacgcuacaacauggguaaacugaaaacauuauuacgugaaauaagccagucauaaaccacauauuguguauauuuuauccauaagaaauguucagaauaggcaaaucuauaaaaauacaaaguagauuuguaguugcugagggcugggaaaaggguuuggggagagaaaugacugcuaaugggaguaugggauguucuuuuggggcugauaaaauguuccaaaauucauugugugauaacuacacaucuguaaauauacuaaaaaccacugaauuguauccaauaaaguacuaaauuguaugaua

SEQ ID NO: 17 - RNA, 2435 nt, Homo sapiens
  ref <308>: DATABASE ACCESSION NUMBER: NM_019034.3
  ref <309>: DATABASE ENTRY DATE: 2022-05-31
  ref <313>: RELEVANT RESIDUES IN SEQ ID NO: (1)..(2435)
cucuuggcuccgcuagugcccggcgcgccgccgccagugcugcgggcuccgggcaauggaugcccccggggcccuggcccagaccgccgcccccgguccgggcaggaaggagcugaagaucgugaucgugggcgacggcggcugcggcaagaccucgcugcucaugguguacagccagggcuccuuccccgagcacuacgccccaucgguguucgagaaguacacggccagcgugaccguuggcagcaaggaggugacccugaaccucuacgacacggccgggcaagaagacuaugaccggcugcggccccuguccuaccagaacacccaccucgugcucaucugcuaugacgucaugaaucccaccagcuacgacaacguccucaucaagugguucccugaggucacgcauuucugccgcgggauccccauggugcucaucggcugcaagacagaccugaggaaggacaaggagcagcugcggaagcuccgggccgcccagcuggagcccaucaccuacaugcagggccugagcgccugcgaacagauccgagcugcucucuaccuggaauguuccgccaaguuucgggagaauguggaggacgucuuccgggaggccgccaagguggcucucagcgcucugaagaaggcgcaacggcagaagaagcgccggcucugccugcugcucugacccagggcagacagaccucacgacagcacugacagggcccgggggcccaggugccgauugcaccagggaggcugccccaucccgacccuccagcucauggugucuggggccugcggcuagacucuuggaacauucuggaacucucuccuuuccuggcuggggcucugaccacaaacuccccuccaggcugccccugggacaugguggugaugugggugcaggagccagugucuguugucgggacucgcaagugcccucaucacagccacccccaccacgagugucuccccagugcagacucaaguuaugcuugaaaugaaaaagucuaucugguaguggguaaacguagaccuggcacuguuccacgcgggcgccccaagccugccacuccugugucccugccucccugggcucccgagauaggcaccacuguauccuccagcuccuuccuuccuucccccaggaacacggaggccaccgagggggcugggccuaucaggaggacacaggcugcagccuggcacccaccccucccaucuccaccccacauggaagacuugucuucaacuuggccacugucuuugcagagcugggcacaugugcccagggggcagaaaaaauggcuucucaguuguuaaaaguauacccuggaccacccaucugaccaucuccuuaagguuucacccaaagcccccagugucagagacuugggaugggaagaacagucggcucccaguguggucaugagcaaguuuucuguggacuguuccccccacugugguaggacaggacuggcccacuuaguccucaagcucccaaccugcugucccucaagccccgcuucuaccagccuguggaguucaggaggcgagacauccuggccuccuuugagaacugaugggaucuacccccuguccacgcgggacaguuucucagaacugguucauagaccaccugugucaccaacagccagauaccuaaucccugagccuccuuugggaaggucuggggccgagggucugggaauuuuuuuuuuuuuuuuuuuuugggacagagucucauucugucacucaggcuagagugcaguggcaugaucuuggcucacugcaagcuccaccuccuggguucaagcgauucucuugccucagccuccugaguagcugggacuacaugcgcacaccaccacguccggcuaauuuuuguauuuuuaguagagacaggguuucaccauguuggccaggcuggucuugaacuccugaccucgugauccacccgccuuagccucccaaagugcugggauuacaggcgugagccaccgugcccggcccaggacuuuuaaaacaagcaucccaggggauuccggugccuaccagaagucgcaagcugcuuggcucuggagggcgccuaggagucggugucgccuaagaugggauaauggcuucaggcaacuccuagccuuccccauaucuccugccucagccccccuggcccagugcuggggcgucagcuaaaccugagcuuccaagaagucagguugccaaaggaaggagccuucugagacccuccccaacccacccagguucccaaggcccugccguacccuggggaaucaaccaaggaguucuuguucucgaagaaccccaggccucaugauggucuacgguagagguccaacaacuacagccugugggccacaucuggccacagccugauuuguaaauaaaguuuuauuggaacacagcaa

SEQ ID NO: 18 - RNA, 71 nt, Homo sapiens
  ref <308>: DATABASE ACCESSION NUMBER: NR_039901.1
  ref <309>: DATABASE ENTRY DATE: 2022-04-19
  ref <313>: RELEVANT RESIDUES IN SEQ ID NO: (1)..(71)
gugucugugccggucccaggagaaccugcagaggcaucgggucagcggugcuccugcgggccgacacucac

SEQ ID NO: 19 - RNA, 1915 nt, Homo sapiens
  ref <308>: DATABASE ACCESSION NUMBER: NM_025241.3
  ref <309>: DATABASE ENTRY DATE: 2022-08-14
  ref <313>: RELEVANT RESIDUES IN SEQ ID NO: (1)..(1915)
auuuucuuccgguggcuggucuccggcggccccguccccgacugggccccgugcccccccgcccccgcggccccccgccgccgggccagccgccaccaugaagaaauucuuucaggaguucaaggccgacaucaaguucaagagcgcgggacccggucagaagcucaaagaguccgugggggaaaaggcccacaaagagaagcccaaccagccagcccccaggccgccccgccagggacccaccaaugaggcacagauggcagccgcugccgcccuagcccggcuggagcagaagcagucccgggccuggggccccacaucgcaggacaccauccgaaaccaggugagaaaggaacuucaagccgaagccaccgucagcgggagccccgaggccccagggaccaacgugguaucugagcccagagaggaaggcucugcccaccuggcugugccuggcguguacuucaccuguccgcucacuggggccacccugaggaaggaccagcgggacgccugcaucaaggaggccauucucuugcacuucuccaccgacccaguggccgccuccaucaugaagaucuacacguucaacaaagaccaggaccgggugaagcuggguguggacaccauugccaaguaccuggacaacauccaccugcaccccgaggaggagaaguaccggaagaucaagcugcagaacaagguguuucaggagcgcauuaacugccuggaagggacccacgaguuuuuugaggccauuggguuccagaagguguugcuucccgcccaggaucaggaggaccccgaggaguucuacgugcugagcgagaccaccuuggcccagccccagagccuggagaggcacaaggaacagcugcuggcugcggagcccgugcgcgccaagcuggacaggcagcgccgcgucuuccagcccucgccccuggccucgcaguucgaacugccuggggacuucuucaaccucacagcagaggagaucaagcgggagcagaggcucagguccgaggcgguggagcggcugagcgugcugcggaccaaggccaugcgggagaaggaggagcagcgggggcugcgcaaguacaacuacacgcugcugcgcgugcgccuccccgauggcugccuccugcagggcacuuucuacgcucgggagcggcugggggcgguguacggguucguccgggaggcccugcagagcgacuggcugccuuuugagcugcuggccucgggagggcagaagcuguccgaggacgagaaccuggccuugaacgagugcgggcuggugcccucugcccuccugaccuucucgugggacauggcugugcuggaggacaucaaggccgcgggggccgagccggacuccauccugaaacccgagcuccugucagccaucgagaagcucuugugaaauaaaagcaggguuggccucagcccugugggucugucucaugcucucccuguuccucuccccgccaccccagggccuccaagccaccucuggaaauacuuggcucugccccaugggcacgggaggggcgccagccguggagcuguggaauugggccccguggcagagcccccaucccuugggggcuguggggaugcgcccaagcccccgagggagaggccuggggacaccaacaaaucuaagcccucccuagcugcuugguaacugugucaugaagcugccggacagacacacguggcaucucccugggcaggagagcaggccugcagcauggguccuguucccgugugccguggguggcaguggcugcaccuggcacuagggcugcucuguggaugugggugacaacggcaggaggggaugcuggccuuccugcacauagaccugcaguuaguaaaucauaagcccaaauaaacagguuguuugaauaua

SEQ ID NO: 20 - RNA, 1669 nt, Homo sapiens
  ref <308>: DATABASE ACCESSION NUMBER: NM_016468.7
  ref <309>: DATABASE ENTRY DATE: 2022-06-01
  ref <313>: RELEVANT RESIDUES IN SEQ ID NO: (1)..(1669)
aaggacguacucgucuuggugagagcgugagcugcugagauuugggagucugcgcuaggcccgcuuggaguucugagccgauggaagaguucacucauguuugcacccgcggugaugcgugcuuuucgcaagaacaagacucucggcuauggaguccccauguuguugcugauuguuggagguucuuuuggucuucgugaguuuucucaaauccgauaugaugcugugaagaguaaaauggauccugagcuugaaaaaaaacugaaagagaauaaaauaucuuuagagucggaauaugagaaaaucaaagacuccaaguuugaugacuggaagaauauucgaggacccaggccuugggaagauccugaccuccuccaaggaagaaauccagaaagccuuaagacuaagacaacuugacucugcugauucuuuuuuccuuuuuuuuuuuuuuaaauaaaaauauuauuaacuggacuuccuaauauauacuucuaucaaguggaaaggaaauuccaggcccauggaaacuuggauauggguaauuugaugacaaauaaucuucacuaaaggucauguacagguuuuuauacuucccagcuauuccaucuguggaugaaaguaacaauguuggccacguauauuuuacaccucgaaauaaaaaaugugaauacugcuccaaaaacagagucacguauuccacucuccaacuacccacauauuccuuuugcaauagccauuagggcaucauuuugauauuucauucugauuucugauucucugauuucugauuccuaaugaggacaguaggucuggauccaaauucucacaguaaaaucaagcaguaauuuucucucauaucuauuagggaaagaaaaaugaucacagucugcuaagagucuugauuuucuuuguaaugccucacauaguaugauaaucagucuccaaagcaucacaugauaauuacaaugauaccauuaacaugucaaggaaauuauauuauuuaugguugucaaaaauuaugaaguaguguaugauuauaagcagauauggcaaauuuguucaguaaauccauagaugacuacauuuugagaaauacuaagauaauacuaaaaauuaugccuuagcauaauuugcaugcaaaauugcccucuaguguuuuuguuuuguuuugagacauagucucgcucuguucgcccaggcuggagugcaggggcacgaucucugcucacugcaagcucugcuucccggguucacaccauucuccugccucagcauccugaguagcugggacuacaggcacaugcugucacacccggcuaauuuuuuguauuuaguagagaugggguuucaccacguuagccaggauggucuccaucuccugaccuugugauccgcccaccucggccucacaaauugcugggauuacaggugugagccaccacgccuggccgaaaguuaguuguuuugaacucauuuucuccuuuuuaccccugccuuuaucagcuucuaccuucugguuucaugcugaguucuaguugccuaaagucuccauugaaacacuccuguuauaauugccauaaaaucauaaauccacucccccuuucuccuccuagucaaaauuuguauucuuuucagcagguugguuuuaauaaaaaguaaauuaaguuga

SEQ ID NO: 21 - RNA, 1203 nt, Homo sapiens
  ref <308>: DATABASE ACCESSION NUMBER: NM_002032.3
  ref <309>: DATABASE ENTRY DATE: 2022-05-22
  ref <313>: RELEVANT RESIDUES IN SEQ ID NO: (1)..(1203)
gccagacguucuucgccgagagucgucgggguuuccugcuucaacagugcuuggacggaacccggcgcucguuccccaccccggccggccgcccauagccagcccuccgucaccucuucaccgcacccucggacugccccaaggcccccgccgccgcuccagcgccgcgcagccaccgccgccgccgccgccucuccuuagucgccgccaugacgaccgcguccaccucgcaggugcgccagaacuaccaccaggacucagaggccgccaucaaccgccagaucaaccuggagcucuacgccuccuacguuuaccuguccaugucuuacuacuuugaccgcgaugauguggcuuugaagaacuuugccaaauacuuucuucaccaaucucaugaggagagggaacaugcugagaaacugaugaagcugcagaaccaacgagguggccgaaucuuccuucaggauaucaagaaaccagacugugaugacugggagagcgggcugaaugcaauggagugugcauuacauuuggaaaaaaaugugaaucagucacuacuggaacugcacaaacuggccacugacaaaaaugacccccauuugugugacuucauugagacacauuaccugaaugagcaggugaaagccaucaaagaauugggugaccacgugaccaacuugcgcaagaugggagcgcccgaaucuggcuuggcggaauaucucuuugacaagcacacccugggagacagugauaaugaaagcuaagccucgggcuaauuuccccauagccguggggugacuucccuggucaccaaggcagugcaugcauguugggguuuccuuuaccuuuucuauaaguuguaccaaaacauccacuuaaguucuuugauuuguaccauuccuucaaauaaagaaauuugguacccagguguugucuuugaggucuugggaugaaucagaaaucuauccaggcuaucuuccagauuccuuaagugccguuguucaguucuaaucacacuaaucaaaaagaaacgaguauuuguauuuauuaaacucauuaguuugggcaguauacuaagguguggcugucuuggauucagauagaacuaaggguucccgacucugaauccagagucugaguuaaauguuuccaaugguucagucuagcuuucacaguuuuuaugaauaaaaggcauuaaaggcugaa

SEQ ID NO: 22 - RNA, 3681 nt, Homo sapiens
  ref <308>: DATABASE ACCESSION NUMBER: NM_057159.4
  ref <309>: DATABASE ENTRY DATE: 2022-04-17
  ref <313>: RELEVANT RESIDUES IN SEQ ID NO: (1)..(3681)
acacggcgcgcugggcucacacugucccgccgcggacgggcuuugugguugggggcgcgcgugcgagugccagugagagugugggugcgcgcugugggccgcggcgcggguggguggccgugcguucuugcgagccggccugcaggaggcgaggcuccccuggccucccgcacccagcggcggaccgagccccuggagggaaguugccgcagccgcccgggccgccggcccuccugucccgcgccagguggacgucugauuuaugaagcuccccauccaccuaucugaguaccugacuucucaggacugacaccuacagcaucagguacacagcuucuccuagcaugacuucgaucugaucagcaaacaagaaaauuugucucccguaguucuggggcguguucaccaccuacaaccacagagcugucauggcugccaucucuacuuccaucccuguaauuucacagccccaguucacagccaugaaugaaccacagugcuucuacaacgaguccauugccuucuuuuauaaccgaaguggaaagcaucuugccacagaauggaacacagucagcaagcuggugaugggacuuggaaucacuguuuguaucuucaucauguuggccaaccuauuggucaugguggcaaucuaugucaaccgccgcuuccauuuuccuauuuauuaccuaauggcuaaucuggcugcugcagacuucuuugcuggguuggccuacuucuaucucauguucaacacaggacccaauacucggagacugacuguuagcacauggcuccuucgucagggccucauugacaccagccugacggcaucuguggccaacuuacuggcuauugcaaucgagaggcacauuacgguuuuccgcaugcagcuccacacacggaugagcaaccggcggguagugguggucauuguggucaucuggacuauggccaucguuaugggugcuauacccagugugggcuggaacuguaucugugauauugaaaauuguuccaacauggcaccccucuacagugacucuuacuuagucuucugggccauuuucaacuuggugaccuuugugguaauggugguucucuaugcucacaucuuuggcuauguucgccagaggacuaugagaaugucucggcauaguucuggaccccggcggaaucgggauaccaugaugagucuucugaagacuguggucauugugcuuggggccuuuaucaucugcuggacuccuggauugguuuuguuacuucuagacgugugcuguccacagugcgacgugcuggccuaugagaaauucuuccuucuccuugcugaauucaacucugccaugaaccccaucauuuacuccuaccgcgacaaagaaaugagcgccaccuuuaggcagauccucugcugccagcgcagugagaaccccaccggccccacagaaggcucagaccgcucggcuuccucccucaaccacaccaucuuggcuggaguucacagcaaugaccacucugugguuuagaacggaaacugagaugaggaaccagccguccucucuuggaggauaaacagccucccccuacccaauugccagggcaagguggggugugagagaggagaaaagucaacucauguacuuaaacacuaaccaaugacaguauuuguuccuggaccccacaagacuugauauauauugaaaauuagcuuaugugacaacccucaucuugauccccaucccuucugaaaguaggaaguuggagcucuugcaauggaauucaagaacagacucuggaguguccauuuagacuacacuaacuagacuuuuaaaagauuuugugugguuuggugcaagucagaauaaauucuggcuaguugaauccacaacuucauuuauauacaggcuucccuuuuuuauuuuuaaaggauacguuucacuuaauaaacacguuuaugccuaucagcauguuugugauggaugagacuauggacugcuuuuaaacuaccauaauuccauuuuuucccuuacauaggaaaacuguaaguuggaauuaucuuuuguuuagaaagcaugcauguaauguauguaugcaguaugccuuacuuaaaaagauuaaaaggauacuaauguuaaaucuucuaggaaauagaaccuagacuucaaagccaguauuuguuuaggucaugaagcaaacaaugcucuaaucacaauauuaacuguuuaauuaaaauguuguaacaaguauaaaacagggaauguaaguuuauuaccaaagugauauguauuccaaaaaagucauagaagaugaagcacuauaauauuguucccauauauuuaaaauacccaaguacauucuaauuaccaguauaucagaggaaaauuuucguagucuuuguaaaauaauauacucaucauagaaaacuugaaaaaugcagaaauguauaaaaaagcaaaaaugauuacugauaauaucacaacccagaaguaaccaccuuuaaaaagcaacccccauguaugccuauauguguauuguauacuuuuuuuacauaauuggagucauacuguaaacaguuuuauaaguagaucuuuuucauugcaaaauugccacauuuucuuauggcauuaaaaauuuuacaaaaacauaauuuuaauggcuauauuauauuccauuuaauggaugcaacucaguuuauuuaaccauucccauguuguuaacuauuuagguuguuucuaauuuucauuauuauaaaguugcagaaauuugguguacauaaaacugucuccauauaauugauuauuaggauauauucccaugaaggauucuuuuuuuaaaaaaaugugaaaugucaucuuguacuuacaccuuucaugaaaagggauuuccugcuuuuguacugcauggguggcaguugugaggaaaagccagucaaaugaccuuuuuacaaaagaaaugcaguggucacuucaguugagagugacuuuuuaauacaacaagaucaacuagaagaauucaacugucucaagaaucaagguaccccaauauaucucgcaauuccaaacuuuguuugagggacucguuauccagcucuugguagccacaccugcaauguaaaauggaagaaaaugcaaagaaaccaaaugugccgagugaauaaaggauugucauaucaauuaguguggguccaucuguagcaaauggguugagugugucaguaugugguugguuacuguguauucgccaggaaucaccccgauaggcugccacccuauuaggugauaccuguuuaauauguuguccagguagacuaguaguugcaucaguuugcuguaacaaguaaccagugagguaacacaguggugaagcaggucaggggaggucaggaggaugucugagagaaagaaguccgggagaugaauggcugucuaggaaggaggaugucagugcacgguuaguguuugagcagagggcagacuuguaaaguaccuguagugaaaagaauguggggacccgauuagcagaaagguguuugcacguacuuuauacaaaauacagaauacuuuauauuggaagugaaagaaaugaacguggacuuuuacacaugugcauauuuuucuggaggcuauauggauuauuugaaaugaaaagcauacuaauuggacuuuaauaaaucauuuucaauca

SEQ ID NO: 23 - RNA, 3387 nt, Homo sapiens
  ref <308>: DATABASE ACCESSION NUMBER: NM_173505.4
  ref <309>: DATABASE ENTRY DATE: 2022-06-02
  ref <313>: RELEVANT RESIDUES IN SEQ ID NO: (1)..(3387)
gcgccgccccuucggcccggcccugugugcggcggcugcugccgggccgggcggcgggagcgcgcggcgucggaggccgccccucugcggaacgccgagagccccgggagagugaagggccggggaggacaaggcgccccgggcccaaagcgcgccggcucccgcucgggcggccgcggacaugugcaggauguccuucaagaaggaaacuccacuugccaaugcugcauucugggcagccaggagaggaaaccuggcgcugcugaagcugcuguugaacagcggccggguggacguggacugcagagacagccauggcaccacacuccugaugguugcugccuacgcuggccacauagacugugugagggaacugguucugcaaggagcagacaucaaucuccagagagagucagguacaacugcccuauucuuugccgcccagcaaggccauaaugaugucgugagauuucucuuuggauuuggagcauccacugaauuuaggaccaaagacgggggcaccgcccuguuggcugccagucaguacgggcacaugcaggugguggagaccuugcugaagcacggagcaaacauccaugaccaacuuuaugauggagccacugcccucuuccuagcugcccaaggugguuacuuggauguuauucgauuacugcuggcuucaggagcaaaagucaaccagccaaggcaggacgggacagcgccccuguggaucgcgucccagaugggccacagcgagguggugcgggugaugcugcugcgcggagccgaccgcgacgcugcgcggaacgauggcacaacagcauuauugaaagcagccaacaaaggguauaaugaugucauaaaagaguugcuuaaauucucacccacucuugguauuuugaagaaugggacaucagcgcuccaugcagcagugcucaguggaaacauuaaaacaguugcgcugcuccuagaagcaggggcagacccaucccugagaaacaaggccaaugaacuuccggcagaacuaaccaaaaaugaacguauauugcgucuccugagaaguaaagaaggucccagaaagagcuaacuuagcuccauauuugacagaaagauagaaagcuuaaccacauuguccaaaaagaaauugcauuucaagcaguguuggaaauucuuuuaugaaaaaaaaagaugcccagaaugcccauccugugggucccugacaaagaagagcuacgcucugugcacgaagucaagaaccaaacagcucagggacccucuugccccuucaccauggacuucucaugguguccuguaacucaucucccggggggccuggcauguucacagauuccacagaaacucauuuucaacaaugcuaacuuggaccugucaguuaaacucuaagguggacaggguucucaguacuaagcaaggagacagaaugcuuuguuccuuuaaaagacugaaaagcugaccuucaauggauugaggcacuuuugcuuuuguguuaaauguagaugugcuaaaauauauagaucuaucauauuuuaccuacauauguaugucauuccaguauaaaacauucuccucuacccaagaaccauagccaugauuguuauaaaucaaugaaguguaaacauacauuauuaaaaaaccacuucugacauuccauuaugugcuauucaaagauggacuauugaacuauagaaaagacagacugugcauuuguucguugauccucaucuuauuccugacauguaaaaaucaauuuuacguagagucaacauuguagguagguuaaaauaccaguggcaaauuuggaaauucagaaacuuauaaaccacgagaaauauauaggcuugucucuuuggucuuuuauuuuggcucuauuguugggaaucuauuuccuauucuauaaguaaguauaccuaacaugcuguggaaucuugaguuuccaacaccgugcugcuugauagaaugacuuugagguccuuggauaaaaugugauauaugcaaguacaguauguugcuauuacuauugcaggaauauaaauaauaaaagacuguuauuagcacuuaguaagucuucaucuaugcauguuuuugaguugacugauuccaagaaugaaauaugagguuuauugaauuauuccuuugaaagggaucaaaacuuauauucaaugcacuuuauaauuaauggugucuaaaugccucagucagugccuaacugcacauacaaaaauaaaaccuucuuucuguaaucuaccaaaauaaacgcaaugguauuucugcuauuuaaacacaucuuuagcuuauuucuucauguucugcucugguuuucagucagugucuauguaaagacacuuuuagaggaaaauguuguaaucccaaaagaauuuuuguuuaaccaacuuucagaguuacuaaaaggauuaucuuuaauucaguaaauaauagaauauaaucuucaaaauuagaagauauucucuuaaaauuuauaacaauuaaugcaacaaaaaucagcauauuuugauaaaugucucaagcaaaugagcaacucacaggccccacaggugccuacagccugaccagcuauacaaaaagccugcaaccugauucuaggauaaaauauuagaaagcucucuguugcuaaaacauccagauauaaacauuuugguggcuaaaaucaaugccauuuccucauugacauucuuuuauucagcaaauuuguacaaauauacauuuucuccugugcgaauuucaaaaagcuauauucauuuugaagaggaaaauaauauggcauuaaaugcuuuguauuuuccccuuaguuagcccuuauggcaaaaauaguugcuaaucacugauacaaagcaaaacguuauggguacugauaacaaaguuccaaaagccaaaaugggaugaaucacaaacuuacuguuuuucacuaaaacauguaauuggccuauuacaucaauaguuuuaaguggguaauguucuugaacaauauuagagccauaaaauugaagccaguagcuugaacaauauggaggccuucuuacuguagggaagaggugucuagcuuagaaggaucacauuugaucuagaugagauuacccauagcagggaaaaugaaucaucaccuaccaaagacuguacggaaagguguguuauuuucaaaauugcauuuuaguuugcacacauguaaaugauuauucuauguuaaucuaauugacaauucaguuagcuccgcauccuaagacuuaaaacuagcaauuccuaugcaaaucaucugcuuaacugaacuacaaacguuuuucauuaauucauucuuugaugacauauuccauguuauauaauaauucaaaauaaaguuaauauacuacccca

SEQ ID NO: 24 - RNA, 1342 nt, Homo sapiens
  ref <308>: DATABASE ACCESSION NUMBER: NM_001271893.4
  ref <309>: DATABASE ENTRY DATE: 2022-05-22
  ref <313>: RELEVANT RESIDUES IN SEQ ID NO: (1)..(1342)
gcacaagccggccuugaaaucagagccuuuccagcaacuccgagagcgugugcucggcgaccgcgggcuuggccagcggcgcgcgcucggcgccccggcgcccccagccccacgcgcgccgggcgggcgccauggaggagggcuccagcucgcccgugucccccguggacagccugggcaccagcgaggaggagcucgagaggcagcccaagcgcuucggccggaagcggcgcuacagcaagaagucgagcgaagauggcagcccgaccccgggcaagcgcggcaagaagggcagccccagcgcgcaguccuucgaggagcugcagagccagcgcauccuggccaacgugcgcgagcgccagcgcacccagucgcucaacgaggccuucgcggcgcugcgcaagaucauccccacgcugcccucugacaagcugagcaagauccagacgcucaagcuggccgccagguacauagacuuccucuaccagguccugcagagcgacgagauggacaauaagaugaccagcugcagcuacguggcccacgagcgccucagcuacgccuucuccguguggcgcauggagggcgcgugguccauguccgccucccacuagcgccgcgccacccaccuccggaccggcgcgccagggcuguccgucgcgucggcggcgcaaguggaauugggaugcauucgagucuguaacuucugaaaccugaacaaccucaggaggcccccaccucugcccuccaccagcgucgagagaagggacagcagugacaucggacagaagacccgggcucccguccucccccaggacgguccccacauaggaagggcacucccagcccucuugcuggugacauugucauggucaucuuguuucuguuuggauuuuucuucugggucuuauguuuggggggagguuuauucuuucugaaaaugucuagauucaggaacacauuuaugaggauuuggauuuugaauuuguauuucccucuaagugccuuuuuuaaugucuauuuuuuuaauaaaacagaaaugcauucuuguacaauucuguugaaacuggaccaaggcucucagaagaggacccccgaguuccuuccccucccccgagccucugcaugauuguuucaagucagccuggaauucuuacuuucacgccgcuauucuuuuccuuucuccgugauugcuuggcuagccauuuaaaaaaaaauauucucuguucaguguauauguugcuuguuuguuuuauuuauugagauauuuuuacaagcuaagugacugcaguguggcuguguauccugcuccccacccaggaaaaauaaagacguccgcgca

SEQ ID NO: 25 - RNA, 2347 nt, Homo sapiens
  ref <308>: DATABASE ACCESSION NUMBER: NM_016475.5
  ref <309>: DATABASE ENTRY DATE: 2021-02-16
  ref <313>: RELEVANT RESIDUES IN SEQ ID NO: (1)..(2347)
guggcccggauguucggugcagcugccagauccgcugaucuagugcuucucgaaaaaaaccuucaggcggcccauggcugucgauauucaaccagcaugccuuggacuuuauugugggaagacccuauuauuuaaaaauggcucaacugaaauauauggagaaugugggguaugcccaagaggacagagaacgaaugcacagaaauauugucagccuugcacagaaucuccugaacuuuaugauuggcucuaucuuggauuuauggcaaugcuuccucugguuuuacauugguucuucauugaaugguacucggggaaaaagaguuccagcgcacuuuuccaacacaucacugcauuauuugaaugcagcauggcagcuauuaucaccuuacuugugagugauccaguugguguucuuuauauucguucaugucgaguauugaugcuuucugacugguacacgaugcuuuacaacccaaguccagauuacguuaccacaguacacuguacucaugaagccgucuacccacuauauaccauuguauuuaucuauuacgcauucugcuugguauuaaugaugcugcuccgaccucuucuggugaagaagauugcauguggguuagggaaaucugaucgauuuaaaaguauuuaugcugcacuuuacuucuucccaauuuuaaccgugcuucaggcaguugguggaggccuuuuauauuacgccuucccauacauuauauuaguguuaucuuugguuacucuggcuguguacaugucugcuucugaaauagagaacugcuaugaucuucuggucagaaagaaaagacuuauuguucucuucagccacugguuacuucaugccuauggaauaaucuccauuuccagaguggauaaacuugagcaagauuugccccuuuuggcuuugguaccuacaccagcccuuuuuuacuuguucacugcaaaauuuaccgaaccuucaaggauacucucagaaggagccaauggacacugaguguagacaugugaaaugccaaaaaccugagaagugcuccuaauaaaaaaguaaaucaaucuuaacaguguaugagaacuauucuaucauauaugggaacaagauugucaguauaucuuaauguuuggguuugucuuuguuuuguuuaugguuagacuuacagacuuggaaaaugcaaaacucuguaauacucuguuacacaggguaauauuaucugcuacacuggaaggccgcuaggaagcccuugcuucucucaacaguucagcuguucuuuagggcaaaaucauguuucuguguaccuagcaauguguucccauuuuauuaagaaaagcuuuaacacguguaaucugcaguccuuaacaguggcguaauuguacuuaccuguuguguuucaguuuguuuuucaccuauaaugaauuguaaaaacaaacauacuuguggggucugauagcaaacauagaaaugauguauauuguuuuuuguuaucuauuuauuuucaucaauacaguauuuugauguauugcaaaaauagauaauaauuuauauaacagguuuucuguuuauagauugguucaagauuuguuuggauuauuguuccuguaaagaaaacaauaauaaaaagcuuaccuacauaaaauuucaauguuuugacacuuaauuguuguuuggcacaauaguauggaaguaauucaaacugguaaauaguuuccucucauaucucggguauauauacauaccauauuuuauugauccagagauacuuauuucacuuugugacaucucugaauuaggaugcaucuuacaacugauggcuuauuagauuuaaugaaauacagaagauacacagaauaaaaaggguuuuccugugguugguuugugguuugugauagguguucugugauguuuaugcuuugaaggccuuaagacucaugguugcaaccauggaagcaaaaugaaauuuuuagcucuuaaccuaacaaccugaccauguuuauccauuuuuauuguuuagaaguuuauuuacugauacuugguggagguugugugaauuaguuaaauuuuaaauguuuaagacuucuauuaacagcugcaaaauaugaaaguaagugcacucacuuuuccuguaguagucugucuuuugaauucacagcaguuguauccuugaguuacuuuguuaauguauuuuucucaguacauuuaaccacugggaaaugaacccuuguacgaauguguuucuucuucucuguaggaauaaaaaauaaauauaaaaauuuuauuuguauugcacacauuugggggguuauuaguguucauuaaaauucugaguugcccaaagaa

SEQ ID NO: 26 - RNA, 9355 nt, Homo sapiens
  ref <308>: DATABASE ACCESSION NUMBER: NM_006252.4
  ref <309>: DATABASE ENTRY DATE: 2022-07-24
  ref <313>: RELEVANT RESIDUES IN SEQ ID NO: (1)..(9355)
gcacuguggguaggcggcggcggcggcggcuacgcggagcggcaggcgguggagcgaggccgcgcgcgccgaagauggcugagaagcagaagcacgacgggcgggugaagaucggacacuacgugcugggcgacacgcugggcgucggcaccuucggcaaagugaagauuggagaacaucaauuaacaggccauaaaguggcaguuaaaaucuuaaauagacagaagauucgcaguuuagauguuguuggaaaaauaaaacgagaaauucaaaaucuaaaacucuuucgucauccucauauuaucaaacuauaccaggugaucagcacuccaacagauuuuuuuaugguaauggaauaugugucuggaggugaauuauuugacuacaucuguaagcauggacggguugaagagauggaagccaggcggcucuuucagcagauucugucugcuguggauuacugucauaggcauaugguuguucaucgagaccugaaaccagagaauguccuguuggaugcacacaugaaugccaagauagccgauuucggauuaucuaauaugaugucagauggugaauuucugagaacuaguugcggaucuccaaauuaugcagcaccugaagucaucucaggcagauuguaugcagguccugaaguugauaucuggagcugugguguuaucuuguaugcucuucuuuguggcacccucccauuugaugaugagcauguaccuacguuauuuaagaagauccgagggggugucuuuuauaucccagaauaucucaaucguucugucgccacucuccugaugcauaugcugcagguugacccacugaaacgagcaacuaucaaagacauaagagagcaugaaugguuuaaacaagauuugcccaguuacuuauuuccugaagacccuuccuaugaugcuaacgucauugaugaugaggcugugaaagaagugugugaaaaauuugaauguacagaaucagaaguaaugaacaguuuauauaguggugacccucaagaccagcuugcaguggcuuaucaucuuaucauugacaaucggagaauaaugaaccaagccagugaguucuaccucgccucuaguccuccaucugguucuuuuauggaugauagugccaugcauauucccccaggccugaaaccucauccagaaaggaugccaccucuuauagcagacagccccaaagcaagauguccauuggaugcacugaauacgacuaagcccaaaucuuuagcugugaaaaaagccaaguggcaucuuggaauccgaagucagagcaaaccguaugacauuauggcugaaguuuaccgagcuaugaagcagcuggauuuugaauggaagguagugaaugcauaccaucuucguguaagaagaaaaaauccagugacuggcaauuacgugaaaaugagcuuacaacuuuaccugguugauaacaggagcuaucuuuuggacuuuaaaagcauugaugaugaaguaguggagcagagaucugguuccucaacaccucagcguuccuguucugcugcuggcuuacacagaccaagaucaaguuuugauuccacaacugcagagagccauucacuuucuggcucucucacuggcucuuugaccggaagcacauugucuucaguuucaccucgccugggcagucacaccauggauuuuuuugaaaugugugccagucugauuacuacuuuagcccguugaucugucucuaguuucuuucuguuauugcacuaugaaaaucaguuauauucuuuaaauuuuuaucuuacuuuuggauaauauccacugcaauacuaauugagaaacaugaauuauuuccaggggcacacaaugcuauugaaauuacugaaaacaaaauaucugacaucuuauuuacuuguagaaaucuguaauucuauugugccuaugauaaauucacauaggcaauaucuuuaauagguuaauaucaaugaagauuuuuaauuacaauaaugaguucacuacagacgauuaacacaccacacuggcgaaccaucucaauguaagggugguuuggcaacaccuccuugcuuugcuguuugguguagguaaaucuaguuuacuuccuaaauuucaguaggcuuuaugcuguguuuaugcccccaauuuauuuuaacaaaagaagauuaaaaaguaaaagaaccacgaguaagauauuauuuaaauguugaaaucuuaaaaaccugccuccaagauuucagaagccaaguuuuucuaacaguauuuguacaaauacugccuaguguauucaacagaaggacuguggucauguaacagguaaccacaauuuucagguuucuuaaaaacagcuguaacuaacucaggauuuuuaucuugagauuucccugaauaauauauuuaucuuaagagccuucaaguuucaaauuaauauuggaacaucuggaauugcaacaacuuuugucuuuuacauaaacuuacgucauuuaaaaaaugucuucaaaaucuaccuuucucaaauucuuuuugccucuauuuauuuuugcauuucaccaacagugauaaaauaguuaaaugaaacaaagcaaaguaucaacagucccuuaaaugagaauccuuaucuuugaucuuuauuuucuguguuagguguuaggguccuggugcagcucauaaugcuaauucuucauuggaagccacucccuucaccucaccucaccuagucacuauugucuuuguucauuguuugauccugagugguugauugauauagcuuugaaucuuuucuaguccaaguuugaaaacacuguucuggcccuaagggcuggcuaugaccuuuacuguugaaccugauagggcagggaagcuuugaacaucaagaaaaaauuuuuaucuuaaauaaauaaauauauauauucacacaccagugcuuuuaagcaaaaaccaguuuuuuuguuuguuuguuuugcuuugugcagguuuucuuuaagauuaaaaaaacacaaacuauagcaggguagauaauaguuuauugauuauauuuuguuuuaguaaaaguauuuaauuuuaaauucuguguaauuuaucuaauuuguauuuauucauuuguuauuuuuauguuguaauuaacacuuauuuaccuggauuacccuaaaaaaauuaagcucuugaaaacaaaaaaaugacauuaccuugacaggcuucucuuugccaaaaguaauguauguucugccaagaagcaaaugagaauguuagacuaaguuucuccuguguuaguggaaaacaagugugucuaucuuuaaauauucuuugaaucaggaguuugcguugugucacaucauauggcuuugcagaucuuuuaaaaacugauguuaaauaacuuauaaguccaacuucuuaauaucaagauaaaugaauaguucaccaauuugggaaggagggcggguugcaauuugagauaguuuaagguuuaauuauugaaagacugccaucuuguggcaccaaaagguaauuuuuacaugauuauuuguauagugauaagcaguccugcuguguuagccugguccauuauagauauggcauaguuuuggguucucuaaauggguccauaccucuuuugggaaaaaacagggccaccucauaauauuugccugauuaugaauggaauuaccuuagaaauaaggaacuaaucagauuacuuaaccccaaugacaaaauccacaaaaauuuugaaggcagagaaacagaaggaauccagugauguuuuagcuccauuagucuaauaggucagauauuaaaaaauuguucauaucaaaauuaccuuauauggauuauugccauguuuuuugagaguuaauuauuuacuguuuucuaauucuugccaguauuuaugaacagcuguagcuugauauuuaccuacugaauuuuaggagaacuaauggucacaguuuggguucuuuuauguguauguuuuuaaaacagcuauuuugugaaucuaggugguugguuuuuagaagauuucaggagaugcaguccagcacaauuagagcuggaacauuguuacagcaggcuuuuuguugcucaugggcagauagagggaaagaaucaguuguuagccccaaauuuccacauuucaguguuguaaacucugaaugugauagguagaugugggcuaagaauaauuuccuccagugaagacacgggagaagagcuucuauuaugagguuauagauaaggccuuaguuauaucauggaaauggacuccauguaguucuagaacugcauuucccaaaguauauccugugagaugcuccacagaaaaaaugauugcauggacuaaguuugaaaaauguugcaugcuacucuuccccucgaagaugacaguggaugucagcauuaaagacuuugacaggucuuucugugaaaaaaagaaagccacauaacuuuguugaacuuugcccaaauuaauuuuucauuuuaaaaaacuuaauagcccaugaaaagcaaauuugggaaauacauuuuuuaggaaaauacugucccagaaugccauaauuaggguaguuaauaauuaacacuuccauuucucuuacugguuguugggagaaucugauugaaaauuagaauuacuuuguuuuaacuugcagacauguucaucgugagugaaugcuggauuaaauagaauuuucugugucugcaacuuguuucaugacaccuugguuuucuuaaucaucuuguauuauuauuugagugcaacauuauucuuaugcaugggugacugaaaaaccugaaacaagauuacuuguuaaaaaaaagucuaagagacuaaugacucaugcuuauggcucaguggguaaugaaucagugucacugauucuuacuuuuaaaauguguagucagugaacauuuucuaaauacuuuucuuugggauaccauaugaagaucuaauuccuuauguuucaguguaggaacagcuaccuucucaaauagaauaugcagggagggaaguaggcaaaccauuuacaaguuggguuuuuguuuugauuaaccuguuaagagacacuuuuguaaucucuaguguuuacauuccuuuauacuagcuaucuauaaagagaauccagaaggcuaaauuaaucagaaauaauuauauuucuaugucaggguauuggcuauauguagcaugaacaauucuccauuuucugcaaauggguuuuucaggacaguuuuauaauucugaguuugcuuuuucuuauucccuagacaguuuguacucagggaauacaaauugauuuuuacuguuaaaugagacauaguuuagaaaaaaucauaagcaacuauuguagauuuuugaucuguuuggauuuaucuguagcauugaauaaugugcaguguacugaguuaauguaggcaccucacuuuggauuuauaaaauguaaaaaccuggaacaugccugccacagacaccacuuugucuauucaccggguacuaauuuacauccuauuaaugguuguaaaagcugagugucuaaaugcuagacauguucacaauuuugucuguuuuuguuuauuuguuuuuaagacccaaaccuuuucaaucuugcauuuauuuacuugguucuccugugucacccuguggcuuuuuaaauagggaauguaauuuauuuuaauaguaguucucaaagacucuauuaaaaacucuggacgagggauaugugcaaucuguugguaagucacauccuauguuuggccucauuuuuauaacuccuaaaaugagaaagguagauuuuaacacuucuaagguaccuuugagcuuuaaaguucuuuucugcuuuuccauaugauuuguguuaguguucacuuuuuauauauuuagaguuuuuauaugagugugaauguuucuguuuugauuuggugaggcaugucagagguauauacuugaauauuuuucuaaauacagcugcuggaggaaugcaaauggcaaaaaucauccagaaaaccaaacacggugaaacaucacauccauuuggcaugaagcaaagcauuacaaauuuggagugaugaaaguguuccauauauuuuuuauagugauuuuucgaucuagagacccuucauauuguuuacuuacuauuacgauaacuuugagagguaaccagagaaagcauuuuugccauuuccagaugaggaagcaggaccagaaagcucuucauaaagcuuuaacaccauacaaguauccagcagugaucaauugcucuuuauaggaaaccaccuuuccucuggggaagaacagucugaauuaauaaaacaucagagugguugaguaguaaaaauaaacacagauguauugcuuucaagucuuagagugacauaaaguagucuuaaauaaauaguucuuaguuuauuuuaagcaguugauuguacuaacgagauguaauuaucugaaguauuuaugauuagcauauugguuuuuauaaguauuucugaguuauugagggugcuuaaaaaugcugaagaaacuuuaaaacaugauucuucuuauaauuuuaugugauucuuauagcacaaucauuuuugaaaaaggaaaacuaggcuggguguaguggcucaugccuauaaucccagcacuuugggaggccaaaguggguggaucacccgaguucaggaguucgagaccagucuggccaacauggugaaaccucgucucuacuaaaaauaccaaaauuauccgguguggugacaggcugcuuguaaucccagcuacucgggaggcugaggcacgggaaucucuugaaccugggaggcagagauugcaauaagccaagaucaugccacugcaugccagccugggugacagcacaagacugucucaaaaaaaaacaaaaaaaggaaaaagaaaacuagccccagucccugucuaaauuguuauaguuuuugacuauuaucugaauuagucuuauccaaauuucauaugcagauugauuuuucauuuuguauguauauuacaugauauaacuauuuucauagaauauauuucuaauuuacaaacacugaauuauuagcuugauagaguuucucccaaaccuuauaaaacuuugaaauguacuuauguuugagcuuuaaaaauuauaaaaacuucauuuaaacaaguugcuggcaucauuacuagccuuuccaccuauaauuaugaugcauuugaugccacugaacaguaauagcacaaagaacaggucacacagucuagacuggauugaguuuuaauuaaacuucuucauccauagccugauaagaaucaggcugggucuagaagaguuaucagacuguaggccuguauuaauuuugcaagcuaauguuaauguuuacugccagccuugcauagcaaugccaguuugcaguugugacuaugaugugacuuuugguaaaaaugguuuauaucuaaaguaguuuagauuuuauggaaguggcucuugauuguuuggacaagcaggguagguaaauuucaauuacguuugaaguguaauuaaauccuagaauagagcacucuccagaaaaaaaaaugaaaaaagaauuuggagaauauccuggcaugaaaacuuuuuugaauaauacguagaauuuugacaagguucauacagugcauacagugugaccuuuucugaggugggcagggaggguugcuacguuguguugauuuaaaauuucaaggaggaacaaaaacauucuagccaaggagaauugcauuguuauuuuuuuuucuacuaauuuaaacgugcauuuuucacaguuacacauuuaaguuuugcuaccaaaaauggccauuuuucuagacagauaacauuuuccuaaaguggaagagagauccacuguucuaaaaaaaaaaaaaaaaaaaaacacuaaauugugccuucucccuuaaauuauaagaagaguaauaguaaaaacuuguuuaaacuauauuuucuuuuuuucuaaucccucucaguucuccuuagaucugccuccuacaaaugaucauguuguuugcaucugccugauugaguucccuuggucaaaccucucccuaucacuaucagaaguuguucacuaaauguauuuuccuauuucuuucauguugauugacuuuauaagucaccuugguaaacauaacauuuggcuaccuucucaugcuuuauaaaugaauuugaugaauggcaguuuuuauauuuuaacccuuuaaaaaguuugucaaggaaacaacucagagcuaagaacaaaaucuaacuuggaaucaguuucuuccuuuggcugcugaagcagcagcaagucacguaaccucauuuaguuuuuugauuuacaaaauguggauaauaaccuaccucacagaugugugugagcaacuguuugaaagcccuuguuuaaauacaggguuuauauccccacauucaauguaaauuccuuuuuuuaaaaaaaauaaguaucuuugauacugauaggaugaguaucuugauuuuuagguaagaucacaguuucacaaauugugguguuguuaggugacauuuaauauuucuugagaccucaaguauucaaauaucaaacuuguccuguguuguaacaaauauucagaaucucuuaaaauguuggcugaagcugccuuaauguaccaaaauacagcucucaagacuguucacaaauaucugugguuguacaauguuuugaacauuuaauguaaaguuguuguagaacugugaacucuaaggauaguaucuuuauuguuuuaggaagaaugguuguguucuuugucauguaugcuuaaauauacaguggauuuugaaugcaacaaauaaaacugaaaacagccauuugguugugucaugacaaucauaauauaggauauaauauuuacuuuuuuucaaaaacauccugcauucauucauucuuagcauaccuuugaaaaagaaaaaaaaauccauagguugauacauugacccauugaaguacuucauuacguauuuauugaaaauuuuuuuuaauuguugaaauugggaccacuuuuuauaauugguuucaagaaaaaaaaaugaacagacuuccagugcuuuucuguucaaauaaacuccgauucaaauuucaguuuuguuuguuauauacuuuuugugaguguuaaaugauaugggaaggauuugucuuucauuaaccauauggaaaagaugucuuccugaauuacauagcauuucauaguuacagauucuuccuaauuaauguuugcaccauaugaaacucauagguaacaaacacaaaggagaaaagcagccugucugauugcuuauaagcuuuuuucacaaaaucaaauuaaaagucucugauucauuugaaagcua

SEQ ID NO: 27 - RNA, 4110 nt, Homo sapiens
  ref <308>: DATABASE ACCESSION NUMBER: NM_015235.3
  ref <309>: DATABASE ENTRY DATE: 2022-03-16
  ref <313>: RELEVANT RESIDUES IN SEQ ID NO: (1)..(4110)
agacgccucgaagaauccgcuaucggcugucugcacaaccggaaucaugucgaguuuggcggugagagacccggcaauggaucgaucacugcguuccguguucguggggaacauuccauaugaggcaacugaggagcaguuaaaggacauuuucucggagguugguucuguugucaguuuccggcugguauacgauagagagacgggaaaacccaagggcuauggcuucugcgaauaccaagaccaggagaccgcgcuuagugccaugcggaaccucaaugggcgggaguucagugggagagcgcuucggguggacaaugcugccagugaaaagaauaaggaggaguuaaagagccuugggccugcagcgcccauuauugacucacccuauggggaucccaucgauccagaagaugccccugaaucgauuaccagagcaguagccagucuccccccggagcagauguuugagcugaugaagcagaugaagcucuguguccaaaacagccaccaggaagcucgaaacauguuacuucaaaauccacaacuggcuuaugcacuguugcaggcacaaguagugaugagaaucauggauccagagauugcucugaaaauucugcaucggaagauacaugucacaccacugaucccaggcaaaucucagucugugucugucucuggcccuggcccuggcccuggcccugggcucugcccaggaccuaauguucugcugaaccagcagaauccuccagcuccucagccucagcauuuggcuagaagaccugugaaggacauuccuccucugaugcagacuccuauccaggguggaauuccagcuccagggccaauaccagcugcaguucccggagcugguccugguuccuuaacuccuggaggagcaaugcagccccaacuuggaaugccagggguuggcccagugccuuuagagcggggacaagugcagaugucagauccuagagcuccuauaccucgcggacccgugacuccugguggucugccuccucgaggacuguuaggagaugcuccaaaugacccacguggagggacuuugcuuucagucacuggagaaguggagcccagagguuaucuggguccaccccaucaggguccccccaugcaucaugccucuggucaugacacucguggcccuuccucacaugagaugaggggagggccauuaggagaucccagacugcuaauuggagagcccagaggccccaugauagaucaaaggggucuaccuauggaugguagaggugguagagauucucgagcgauggagacucgugccauggaaacugaggucuuagagacacguguaauggagaggagaggaauggagaccugugcgauggaaaccagagggauggaagcaaggggcauggaugcaagaggauuggagaugaggggcccuguccccaguucaagaggcccuaugacugguggaauucaggguccuggucccauuaauauaggggcagguggcccuccucagggacccagacaggucccaggcauuucagggguggggaauccuggagcugguaugcaggguacaggcauacaaggaacaggcaugcagggagcaggcauacaaggaggagggaugcagggggcaggcauacaaggagucaguauacaaggaggagguauacaaggaggagguauacagggggcaagcaagcaagguggaagccagccuagcaguuuuaguccugggcagagccaggucacuccacaggaucaggagaaggcagcuuugaucaugcagguucuucaacugacugcagaucagauugccaugcugcccccugagcaaaggcagaguauccugauuuuaaaggaacaaauccagaaauccacuggagcgucuugaaagguuuuagaaaauauuuggcuguagucucaaauuuuauucuguagcauggagaaugggugcaaaaagcugacuucuguauccccacacuuggauuaggguuucccuccuccuagaaccuaaucuuauuuuuuguucuuuuucuuucuuucuguuuuccuuuuuuuuuuaauugaggguggggggaggagggagugcgucuguucacuuuaaguuacuuuaaaauaacucugaacaugauuauauuaugccaaauaagauuacaaagaauaagcagcaauauugaagcaucuacaguauguuaacuacauuuuuuaaaugucgaguaaaacuucgugaaaacugcucauaaagacuaaaaguugaccuguuaaaacguuaauguacuaagauaguuuuaagauuuuugguuguauaacaaaauaaaaguuuacccaaaaguauaagauacauuuuuugguaaaccauuuaaaauacaaaauccuauuuuggaggcaauaggauaagcauugucuuaguguuuuagaauuauuuuugaaacagaguuaacaggauaagguuacacuuguauuagaauuguuuuuuucuaaaguuauguuaccugaaaaaucaaggagugcaccugauuuacuguugcuucauuuaucccauccuucagagaccagcaagguucugccaggccccaagcaucugagcaugaugauuuugcugugacaauucuaagaucaaccagguuacuuaaguccgcucugaugaaacccagucucucaauaccuuccuauuucucaauagucuuuaagcccuaaaucugagucaguaauucucuaaaaauccuucccaaaaaucagugucuagggacugguugaucuagaugaguuauaaaugguauuugacuuuucauaaguaguggaagguuucacuaaguaaagaucugaguuucuugguaucugacguuuguauacagaugguguccauuugcucaaccagacaggaguuaacuuguauuagaauuguuuuuucuaaaguuauguuaccugagaaauuaaggacugcaccugguuuaauguugcuucacuuaucccacccuacagagaccagcaagguucugccaggccucgagcauccaagcaugauuuugcugugacaaaucuaaaaucaaccaggcuacuuaagucugcucugaugaaacccaauaaucucucaauaccuuccuauuucucaauagucuuuaaacccuaaaucugagucaguaauucucuuacauccuucccaaaaaucagugucuagggacuaguugaucuggaugaguuauacaugauauuugacuuuucauaaguaguggaagguuucacuaaguaaaacucucaguuucuugguaucuggcuuucuuauacagaugguguccauuugcuccaccagacaggaguuaaaaaccuuuaaagagcauuuaacaugccuuuuaguuuucuuauuucauaaucuacuuuucuaucauuaaucaucuuaccaggaauaaucaaggcccagguagguagaaguuuuuauauagccugaauacucacuugacuuacugcucauuuuacuuuuugcauggaauacagccauuuaguccuaauauacauaccaaugagacaauuaaaaauugguuggaagauggagcuuaucacauugugcuacuacgacuuuauuuuuucuugaaaauaaagccuugaggaugugaacauaaauguugguaucauaaaacaaguaguaauaaaagcauaaauuaucaaauaucaggugaucuuacuauggagaagaaaaaccaauaucuaguuacagaugaaaaaauguuuaauacaaagagcugaaaauguagaaauugaguuaucuucccuuugcaaaauuuuuacacuuuaaauuaguaauucuuuguuugcaagguuuucaucccuaugcuguuguuuucuacuaccuguuuuacucuuguucauacugcuacuuucccauguuuccuauaugccuccccuauauuggagaggauaacucgcguuuacauaugugauaguuaugcuuaauacuaauucacagaguuggcucgaucauuuugagauucuaguugcuuuaucucaaagcuuucacugaagggcuguaacugaaaauuauuaaauguuagucuuuagucaaaucucugcauuguuucaugcagaaaauaaagauacaauuaaggcuga

SEQ ID NO: 28 - RNA, 9145 nt, Homo sapiens
  ref <308>: DATABASE ACCESSION NUMBER: NM_024817.2
  ref <309>: DATABASE ENTRY DATE: 2021-02-13
  ref <313>: RELEVANT RESIDUES IN SEQ ID NO: (1)..(9145)
gacuugaacgcaacucccaauugcagaaaauuggcaacgucucugaagagcccuugcuuuugccuggacccccagcaucaugguuucccauuucauggggucucucaguguccuguguuuccuucugcugcuuggauuccaguucgucugcccacagcccuccacucaacacaggaaggucccgcagcggauggcggcggagggcgcccccgaggacgacggcggcggcggcgccccgggaguguggggcgccuggggccccuggucggccugcucgcguagcugcagcggcggcgugauggagcagacgcggcccugccugccccgcuccuaccgccugcgcggcggccagcggccuggcgccccugcgcgcgccuucgcggaccacguggugucggcggugcgcacgucggugccacugcaccggagccgcgacgagacgccagcgcuggccgguacggacgccagccgccagggccccacggugcugcgaggcagccggcacccacagccccagggccucgaagucacuggggacagaaggagcaggacccgugguaccauuggcccuggcaaguauggcuaugguaaggccccauauaucuuaccacugcagacagacacugcacacacgccacagaggcuccggagacagaagcucucaucccgccauuccaggucccagggagcaucuucugcuaggcauggcuacaguucaccagcccaccaggucccccaacaugggccuuuguaccaaagugacaguggcccucgcucuggacugcaggcugcggaggcccccaucuaccagcuaccuuugacccaugaucaaggcuacccugcagcuucaagucucuuucacagcccagaaacaagcaacaaccacgguguggggacccauggggcaacucagagcuucucucagccugcccgaucuacagcaaucucaugcaucggggccuaucggcaguacaagcugugcaacaccaacguauguccagaaagcaguagaaguauccgggagguacagugugcauccuacaacaacaagccauucaugggccgguuuuaugagugggaaccauuugcagaaguaaaaggcaaucgcaaaugugaguugaacugccaggcaaugggcuaccgcuucuauguacggcaagcugagaaagucaucgauggcacccccugugaccagaacggcacggccaucugugugucugggcagugcaagagcauuggcugugaugacuacuuaggcuccgacaaagucguggacaaaugugggguguguggaggagacaacacgggcugucagguugugucgggcguguuuaagcaugcccucaccagccugggcuaccaccgcgucguggagauucccgagggagccacgaaaaucaacaucacggagauguacaagagcaacaacuauuuggcccugagaagucguucuggacgcuccaucaucaaugggaacugggcaauugaucgaccaggaaaauacgagggcggagggaccauguucaccuacaagcguccaaaugagauuucgagcacugccggagaguccuuuuuggcggaaggucccaccaacgagaucuuggaugucuacaugauacaccagcagccaaacccaggcgugcacuacgaguacgugaucauggggaccaacgccaucagcccccaggugccaccccacaggagaccaggggaacccuucaauggccagauggugacagaaggcaggagccaggaggagggagaacagaaagggaggaacgaggagaaggaagacuugcguggggaggccccugagauguucaccucagaaucggcacagaccuucccagucaggcauccagacagauuuucuccccaucgaccggacaacuuggugccaccagcaccgcagcccccacggcgcagccgggaucacaacuggaagcagcuugggacaacagaauguuccacgaccugugggaaaggaucgcaguacccuauuuuccgcugugugcacagaagcacucaugaagaggcuccugagaguuacugugacuccagcaugaagccgacccccgaggaggagcccugcaacaucuucccuugcccagccuucugggacaucggggaguggucugagugcagcaagaccuguggccugggcaugcagcaccgccagguucugugccgccagguguacgccaaccgcagccugacggugcagcccuaccgcugccagcaccuggagaaaccugagaccaccagcaccugccaacucaagaucugcagcgaguggcagauccggaccgacuggaccucgugcucggugcccugcggcgugggacagaggacccgugaugugaagugugugagcaacauuggggaugugguugacgaugaggaaugcaacaugaagcuccggccgaaugacauugagaacugcgacaugggacccugugccaagagcugguuccucaccgaguggagcgaaaggugcucagcggaguguggggccggagugcggacacgcucgguggugugcaugaccaaccaugucagcagccugccccuggagggcugugggaacaaccggccggcagaggccaccccaugugacaacggacccugcacgggcaagguggagugguuugccgggagcuggagucaguguuccaucgagugugggagcgggacgcaacagagggaggugauuuguguuagaaagaaugcagacaccuuugaaguguuggaccccucugaauguucuuuccuggagaaaccccccagccagcaauccugccaccucaagccuugcggagccaaaugguuuagcaccgaauggagcauguguuccaagagcugccaggguggcuuucggguccgggaagugcggugucugucugaugacaugacucuaaguaaccucugugacccucaguugaaaccagaagagagagaaucuuguaacccucaggacugugucccugaaguugaugaaaacugcaaggacaaguacuacaacugcaacguggugguccaggcaagacucugugucuacaacuacuacaagaccgccugcugugccuccugcacccguguggccaacaggcagacgggcuuccuggggagcagauaacacuccugcacccccaucaguagggcagcaucacugccuucccgggggcuucagcagugcgccuggcuggcugcugcuccaccacgggcccccuggcccaggcgcugccaaccaacuuagucaccaccccugccuccggugaaugcaccccgugguacccaggggcuuuuuacacaagauguuugaaagccacagucaguccuuuaagcaucaccauguacugaugauccccuccuuggaccuggcaucugcuaauggugcccuuugaaagucaagcagugggaaguacauggagcucucagcccugcucccaucuggcaccuucaagucagcagaugggccacugacugagcacugccccgucccuggugcuacuggucuuucuaaacuuagcacccuggagaguccaaggaggcagcgcccccaacccagcgccccacuaagccuugcugacacgcgugcaucccucugugaccucagcccagaugugccuguuuucauucucaaagacauuagacuguuuuccugcccuaugacacagauagcucacaugaauauugugcuuuauuuagcagguguacucacagauacuagcuccuuagcagcucacaacaucccagaaugggaggcagggggugacucauuauccccauuuuacugacagggaaacugaggcucaacuuaaguaauugaccugccagguauauucacccauccaguggaagagcugaguccccgccccagucaucuaccaguauccagccuggggccuguacuuagaugugaaaggugcugcuucauuucugaccaagagacugagaaguuucccagaaugcaaacaaagcccaggccccugaaaucuuuccggucaagccuuuaucccagcacucaguuguuuuggaugucuguuccuacuugcccuuacccccaaaguuacagauccuaguuacaggacucugccagcuuuguuaaacuguccgugagacaagaaagccauuggggaaaccaggugauugccugaaauucuuacuccguuccaagugcuguuccucccaggaaaucaaaggccaggguccuuauggccguggagccuucccgaccacagagccaacuugugaagcacacagcucugcagccugggcucugcccugccucagccgccucccccacgcucuucaccacguuccuggagaguccggccaaccugucccagccgaaacacugcuguauuagaaaaagucucuuucuggucuuucugguuuuguuuaugaauuucccucuguggccacaaauuccuccccucccccaugacucacaguccauauggcccacccccagacuugagcaccaagcucugcauuaaugcaguuggccugcgacaaggagcuguggacccuuccccaucucuuccaauucacuuuccccaacuauccaguuccagaggccgcaggccuggaaggaugcagugcauauugaaagguggacccucugaaaacaguuaagaggaauauauguauguuuuacccauuaagaaaaaauggcaagcuaaacaaauguuaaacuuacagaaaauuugucuuaugguccugagcauauuucccuuuuagagcaagccuggauucuuagcaaaguguuucccccauuugcucuuuuagcugacaaaucugccacugugaugaugguuugcagcuuuuggaagcaguauggcaaccuggccugacaugcucuuuaggcuuccacuaaccuggggcuuucagaaauucuauuuggccuuucuguggguagcuuuccagcuucucuucuagggagccccaggcaucauuucccaaaagcauccccaucuccugauucucuuggaacuccuacagauaagcauccuggcagaggcccaggcucccaaaccgacaaagugaaaagagaccagagaggccaagcauauugacuggugcuguucagggccugcucuuuuccacucaccacuuguuuugcugcuugucacgaggagaguuguuccuguauguggcugcucucagaucuuuccaagcaagccagucauuugaagagguuuucuuuucaugcuggagggcaggcuaagaucaaugaguggaagagagaaaggcuguuuuagcucaaguuaaaggaacaccuucuagccaucaaagccgcccaacagaggcaagggccaccacacaugagagagcgcucuguccuuaaagggaauucucuguugagugggaggugaacacccugguucuuccaacucaggaauucucguggcugggcugggucagugauggcuuugucucuuuaugucuaaagugcccuauggcugcugaagguuaccuaaccauucuuuaaaaggagaaugacccuccaugggaauggccagccugccaacugugcaauugaagaagacccgauggaucaaccccaugucucccuuggggagaaagugcauaaaccaggggucucuuuuuuuuuuucaacaaaccauugagcuguucuuggaguucaucucuggagagguuauacauuauuagaaguuugauuauuauuauaguuugaucaauuuauuugucuuagagauccaauuuuuacuaauucccuaguuuuuuauuucagcaucugaaugucuuucucccuagcacagugcauacaaucagggccuuggguauuuccagugauaacuuuccuuggagaggaucuaagaaaagcccagauuucgguagccaucucccuccaaauaugucucuuucugcuuucuuagugcccauuauuucccccucuccuuucuucugucacugccaucuccuucuuggucuucccauuguucuuuaacuggccguaauguggaauugauauuuacauuuugauacgguuuuuuucuuggccuguguacgggauugccucauuuccugcucugaauuuuaaaauuagauauuaaagcugucauaugguuuccucacaaaagucaacaaaguccaaacaaaaauaguuugccguuuuacuuucauccauugaaaaaggaaauugugccucuugcagccuaggcaaaggacauuuaguacuaucgauucuuuccacccucacgaugacuugcgguucucucuguagaaaagggauggccuaagaaauacaacuaaaaaaacaaacaaaaacaccaaaagaaaaaaaaaagccauuuaaagccagccacuagagggagucaguucaguuccguaaagguaugcucagugcccgcugccugcaagcuguuggggaccccagggagggcaaggcagccuguccccgcccccagggaacuagaacaugacaagaauucuccgcacugugccuaccugucccuuuaccuuaccucucuggcccagaguucuuggaggguuuuuucuuuauuuucuuauguacucaucuacuuauucucaaaguauuuagcauucaacacucuuuuugcuuuaaaaagaauggccuuacaaagggacagaaaagagaagacacgagcuugguguauuuucaucaaguuauguggcagagaaauccagauauuaccaggaccugucuaaacaaauguuguggguuuucuuuucauucggauagccacuuuauaguuggaauaucaauucuaaugaggaggaagacauaaauauaagugguaaaaagaaacaugacuucccuuaaaacaggcuggauaaucuauaucagccuuguggguggagacuaguauuugauccuugccauauaaaacauuuuaauaugguuuacaugggaaaauaucgauggcuuccucacaaaauguaugggugacgugaaguugaagagccaauggcuugggugacacgugcuggauccaaaaagaucagggagacuagaauaaaacuuggauguuaaaaauucaccaggaauccacauaaaguacuauauuugggcuaaaaugaaaaacuaaauacaaggugggagagaggcaagaauuucaguugacuaagcucagugugagucaaagugggauggaaccaugcaaaaacaaaacccacagacaugcaggcuacgugaggagaaaacaguggugaggaucacaucacauuguguuugcauuugccggaaccauacuuuaagaagaaaaccgaucaucuauaauaacaucaguuuaucaaugccccguccugaugaagugugcagacucucagaaacagcaggaaggacuucaugagaacccucaggcuggagaaggcacuagggcacaaggagagcucuccuaggaccaggaccaagaagcuacaggcaggcacaguuuagcuccugcagagacccagcuuuucacaaguuggagccuuccagagauagagggacugugguagguggugacccacccaucacuggagguggaagcagaggccguuugccagggaugcuggagaggggauucaagcaucuggcugggcaacgugaugcucagggccgucuccacucagggcuuaggggagucugugaguagaagagcuuuaggugauuuguuuggugggggaaggcaaguacacagcuaugcacuuuccguuucugacuuuugccacccugucagccauggggagcccacugugggacugaaacccugagcugaaugcggccucaugucucagagaaacacuggcaaguuggucagagccgccgucugcaucgaggcguagcugagcggcaggauggggggcugccugcccagggucucucaccgugguguaagcagagccauggcuugccuaggacccuauagauaccaucacucuuucucagcucgacuggaguuucugcaccuuugcaggggcaaaguaacucccugcacccugaaccaccccccauuccuguucauuucagcagauaaugauggaggggggggguguccaucgugcugagggugugaccgcaagagggugaaaacuuccagccaacuuucucaguccuuucucuugcgagagggaagccaccugcuauacaaacuaauacccccugccuugaccccuuccccacgacucaguugacagaaggauauacuuuguuauaacuuauuauuuuguucucuguaaauacaagauguuuauaggaaauauguauucugaacucuaucugcagaaugagucacuacaccaaaauaguucuauuauuuagaauguguuaauuuuaaagggaccugauagguauuuauuuacauaugcgauccacauuugugugaaagcaugugaucauacuaacccagccuccuggaaugucgcuguacgaugauugaugucuuuuucucaguccauaguuacaauuguuuaguaugcuaaucaguccaguucccugagguuuaagaucaaauauaaauuacucugcuuuucgacucauucagguagcauuguaccugaaccugauugcuacuuuuucaucuuaaauauuauauuuccucaucuaaucugccuuccccucauccacagacauuuggagaaggaaaugggagggugucuguuaucccuuucucuuugcuuuguccccguuguuagacuggcagcgucaguugcucggugggcuugguuagagccgugggugaggcagguggcuggcggggacagggagaggcugagagggaagugguggcauuuacugcucugacacuuccacugucccugcuggggaugcuggggccaaggccuguggggccugugaacugcacagccaggagcaaggaacccacuaaauacuccgucaccuccauguccccucuacaguguuaaauuauuacauaagcaggugaaagguagaaggcgaauuaugugaguaaauauggucuguuuucucuucagcaaaaaugacuauuuuugugugugacuaauuuauuuuuauuauuguaaagauacaauaaaccgguugaaauaucugcuuuguugacaagcgugugcuuucucuggccuuauucgcguucuguucuccugcaaauagcgcccucuaaaaagaagagucagacaauaaacugguugaaauauc

SEQ ID NO: 29 - RNA, 8024 nt, Homo sapiens
  ref <308>: DATABASE ACCESSION NUMBER: NM_015520.2
  ref <309>: DATABASE ENTRY DATE: 2022-04-17
  ref <313>: RELEVANT RESIDUES IN SEQ ID NO: (1)..(8024)
augcgcgcgcgucuccugucgccggccgcugcgcuccgcggcgcggaauagaaugaacuguaacaaaacaagccgagccuuuguaucugcuuaaaggggccgcgagcacuuaccucccgcccugcuccccgccgccccgccaccgcgucuucagggcucccggcaacuggcuggaagggcuucccucacccucaguaacagccugccagcgaggagaggcgagccgggcgggaggcaaagaggaggcaccgcauuuguaaaaggcaagagagaaaggaaggaaggaaaaaaaaauaacccgagcggcgcagaguggacucugguccgggagagcacgggcgggcgccggaacguggacccagaggcaccggaaugcaaacaaagcucgcgggcgccugugcggggcucgcggggaagcccagaaaguuuguuuuaugauggcuugagugcgcgagcgugugcaggggagcgaggcugccaaguuucucucuccuguuuugugauuuggggagagauguuucucccaugaacaagcggaggcuugggggcucgugcuguggggggcaccugucucucguuuuauuuuuuuggaggagggguguaacucaucauguccaaagugauccagaagaagaaccacuggacuagcaggguucacgaaugcaccgugaagcggggaccccagggcgagcugggggugacggugcugggaggcgcggagcacggggaguuuccguacgucggagcgguggcggcggucgaggcagcggggcuucccggcggcggcgagggcccgaggcugggcgaaggggagcugcuucuggaggugcaggggguccggguguccggcuugccccgcuaugacgugcugggggucaucgacagcugcaaggaggccgucaccuucaaggccgucagacaaggaggaaggcugaacaaggaccugcgacauuuucucaaucagcgauuccagaaggggucuccugaucaugagcuccagcagaccauaagggauaaccuuuaccgccaugcugugccuugcacaacccgaucucccagagaaggagaagugccuggcguggacuauaacuuucugacugugaaggaguucuuggaccucgagcagagugggacucuucuggaagucggcaccuaugaaggaaacuauuaugggacacccaagccuccuagccagccagucagugggaaagugaucacgacggaugccuugcacagccuucagucuggcucuaagcagucgaccccgaagcgaaccaaguccuacaaugauaugcaaaaugcuggcauaguccacgcggagaaugaggaggaggaugacguuccugaaaugaacagcagcuuuacagccgauucuggugaacaagaggagcacacucuccaagaaacagcauuaccaccugugaauaguagcaucaucgcugcucccaucacggacccuucucagaaguucccucaauaccuaccucuuucugcagaggauaauuuagguccucuaccugaaaacugggagauggccuauacugaaaauggagaagucuauuuuauagaccauaacacgaaaacaacaucuugguuagacccucggugccuaaacaagcagcagaagccacuggaagagugugaagaugaugaagggguacacaccgaggagcuggacagugaacuagaacugccugcugguugggaaaagauugaagacccugucuaugguaucuacuauguagaccacaucaacaggaagacacaauaugagaacccgguucuagaagccaaacggaagaagcagcuugagcagcagcagcagcagcaacagcagcagcaacagcagcagcagcagcagcagcagcagcagacagaagaauggacagaagaucacucagcccuugugccuccuguuauuccaaaccacccuccaagcaauccagagccagccagagaaguuccacuucagggcaaacccuuuuuuacacgaaacccuucugaguugaaaggcaaguucauucacacaaagcugcggaaaagcagucguggcuuuggcuucacggugguuggaggggaugaaccugaugaguuucuccagaucaagagcuugguccuagaugguccugcugcauuggauggcaagauggaaacaggggaugugauuguaagugugaaugacaccuguguuuugggacacacacaugcucaaguugugaagaucuuccaguccauccccauuggugccagcguggaccuugaacucugccgagguuauccauugccuuuugauccagaugaccccaauacaaguuuagugaccucgguagccauuuuggauaaagaaccaauuauugugaaugggcaagagaccuaugauucaccagcuagccacaguaguaaaacaggcaaagucaauggcaugaaggaugccaggccaagcagcccagcggacguggcuucaaauaguucucaugguuauccuaaugacacuguuucuuuggcuuccuccauagccacucagccagaacucauaacuguucauauugucaaagggccaaugggcuuugguuuuacuaucgcagacaguccugguggggguggccaaagagugaaacagauuguugacagcccaaggugccgaggccugaaagaaggggaucucauaguggaaguuaauaagaagaacgugcaggcccuaacucacaaccaagugguggauaugcugguugaguguccuaagggaagugaggucacauuguuggugcaacgaggagggcugccaguucccaagaagagcccaaagucgcaaccacuggaaaggaaagacagccagaauaguucucagcacaguguuuccagccaccgaagccugcacacagcauccccaagccacagcacacaggugcuccccgaguucccaccugcagaggcccaagcuccagaucaaacugacagcucuggccagaaaaaaccagauccuuuuaaaaucugggcccaguccaggagcauguaugaaaaccgaccuaugucaccuucaccugcgucgggauugagcaagggugagagagagagagaaaucaauuccacgaauuuuggagaauguccgauuccagauuaccaggaacaggacaucuuccucuggagaaaagagacuggauuuggauuuaggauucuggguggaaaugaaccaggggaaccuauuuauauuggucacaucguaccacugggugcugcugauacugacggccgccugaggucuggagaugaauuaaucuguguggaugggacgccaguaauuggaaaaucacaccagcuugugguccagcuuaugcaacaagcugccaagcaaggccacgucaaucucacggugcggcguaaagugguuuuugcggugcccaaaaccgagaacgaggugcccucgccagccuccucucaucacaguagcaaccagccggccucgcugacagaagagaagcgcacuccgcagggcagccagaacucgcugaacacggugagcucgggcagcggcagcaccagcggcaucggcaguggcggcggcgggggcagcggcguggucagcaccguggugcagcccuacgacguggagauccggcgcggggagaacgagggcuucggcuucgucaucguguccucggugagcaggcccgaagcaggcacgaccuuuggcaaugcauguguggcuaugccucacaaaauaggucggauuauugaggggagcccugcugaccgcuguggcaagcugaaaguaggagaccggaucuuggcaguaaauggauguuccaucaccaacaaaucccauucagacauugugaaccuaaucaaggaagcgggaaacacaguuacccuccgcaucauuccuggggaugaguccucgaaugccaccuugcugaccaaugcagagaagauugccaccaucaccaccacacacaccccuucucagcaagggacccaggagacaaggaauaccaccaaaccaaagcaggaaucucaauuugaguucaaagcaccccaagcaacacaggagcaagauuuuuacacuguggaacuggaaagaggagccaagggauuuggcuuuagccuucgaggaggccgagaguauaacauggaccucuauguucugcgcuuagcagaggacgguccugcggagagguguggaaagaugaggauuggugaugaaauuuuagagaucaauggugagaccaccaaaaacaugaagcauucucgagcuauagaacugauuaagaaugguggccgcagaguucgucuguuucugaagcggggagacggcucaguaccagaauaugcgaugauaccuccuaauaucgcugcauguaugagaaaugaaaagcucggggaggcuugcuucuaccuuaugggccauaaucaaacuacgaccccagcagcgaccgccacggccccgccaccgguccacaagguguuccggaagugagggccgggcccgaccgccggcagcauccgucauuggaguccaguuacccacccgaucuucacaaaucaucaccacauggggaaaagcgggcacacgcaagggauccgaaaggcagcagagaguauagcagacaacccaaugaacaccacaccuggaaugggacuucgaggaaacccgacagcggggcaugccgacccaaggaccgggcgccggagggacggagggacgcgcaggccgagcgcgcggcagccgccaacggccccaagaggcgguccccagagaagcggagggagggcacccgcagcgccgacaacacuuuggagaggagggagaagcacgagaagagacgagacgucucuccggagcggaggcgagagcggucacccacccgcaggagagacggcucccccagccggcggagacggucucuggagagacuccuggagcagaggaggucccccgagcgcaggagagggggcucgcccgagcgcagggccaaguccaccgaccggaggcgcgcacgcucccccgagcgcaggagagagcggucccuggacaaaaggaacagagaggacagagccagccaccgagaaagggaagaggcgaaucugaaacaggaugccggcagaaguuccagacaucccccggagcagagaaggcgaccuuacaaagaauguagcaccgaccucaguaucugagacgcugagucacauuccaaccuuuaccgugucaaagguucuaagaggaaagucacaaaccugaaauuauuuaguuucuuaccuaaugaagcaucugacaccugaugauccuaugaauaacaacaaacauuuuaugcauuugaaaucuuauaagaaaaaauauauaugaaaaguauugugccugauguaucauauuaaagaaaguauuuuuaaaugcauacuuuuuuggaaauuauuugccaaaugcuggcccaaagggauauaaauuuuguuucuacguaaccuguagaaucgucaagaauuguucccguuuuggggcaaucuuuuucucuccugguuaaauggggcuguugaucauuuucucuacguaagggaauuuauugauuaaguuuaauuaauuugauauagacugucauguagugauguagugcuauacuguagucggaaguuuuucuuaaaagcaaagagcaaaaaugcaaaguuuuauuuguaaaagcugaggacccuuggggauggauuagguuugccgaugauccuaaaaguaagcaaacuguaugacaagacuacuauugcaaaugaaggauauuuauagcuaaacugcgcagcacaaagaaguuuuauauugugaguuauaguugguucagaaaacaguuuguacucucccuggcccaggaaggcacacagacaaaaaugucggccacuuuuacucaaauacagcccaagcacagacagucagguucgugaucaucaccaacaucugauaaaaucucauugaaggcaaugccaucacagcuuugcucaauuacuacgaggaagagacaacaagcaucuuucguguugucucgucugauuggaggcugaauaguagauggaauggggggacagugugccuggugugaggaagacguaagauccccaauuuggaaagcaugccccccucccuuuuaguagaagcccaugguugcccuuugccaaacuggggaggaggcaaugagccuugguggaaggaaccucucuguugauauuuaaagaagugagggcuguggguauucauuguuagaaaugccaauuucacuuugaaaccauaguccaagucucuagguugguagaagggaaaggaaggaaguggucccagugauucuaggucugguugggaaacuucugccucaugacucuguccuuugaguccuuuggacagcagcacaaaacauaacaauuuuuauuuuuaaacagacccauucuuuuugaucccacaggagcugugguugggucggccguagccccgggaugugguuuaaguggauuacugccuagcugagccaaaauguuugcuuguaccuguuggaccacuacagcaaaccgcugcuuacagugcauuguguauuucuguaguacuguuuugcauuuuccauagagacagaaaacuuugcaagucaaucacuguuguucccaugguacuguaagaaaaaaaaaaaggaaaaagaaaaaaaaaaagaaaaccagccaaucauugcguguacagagcuaaaaauuguaauuaauagagccuguugggaaaaaaaagaaaacaacuguugccuuuuuuucuuguauaaaagagaauuuaugacaaaauuuagcugugaggaaugugauacguguuuauauuucugaauauggaacaaauugauucauggggauauauuuuaauguaaacuaaaucagguauguaaaguuguuuuaaaaugggagacuauauaaguaauucucuaaagcuuuaguugguuugaauaucaucauuuccuccauggugagccugcuugugaauuauuaagcacuuguuugcauucucuguucuucacucauuuauuucuugcagugugcuauggacuuaaugcucuuucuguauugaugaaaagcaguaugugggccaaucuuuuuauaaaacacuaugcauauauaaauauuacauuguucauagcuuuauuugacuuauggguuuauacauaacauuagaaugaguagcuguaguuguguggacauuuauaaaacaaguuccuuuccuggaacagacacaauauacauucuguagcuaaaaaggagggaaaagucugugaaugcuauuuuuauuauuaaaguuuuccauacaaagcauacauugagcuguaguaaggcugaggaagaguacaacugaauaaugauguucuuuuucaucaaguuccuaagagcugacuuggagauuuuauucccagggcuccacugcuaggauucuucucacuaguucuaacaaaaucugugauguuaaaugacugaugcucucaauugugauccagaguuuuaaauaaaugaaaucaagguggauuuugggaauauauccugaaguuaacaucuugauguccuucuuguuguaagaugauauuuggcaauuuaaaaucuaugugcuacccaccugggcuugucucaauccguggacuuuuugguguggcaguuucaccugaauuccacacaucagauuuguuuggaaauauuguacuguacagggacuaugcauuaugcagaaagauauaguuuucuuugaacugcuguaaccuuaaauuggaaacugauucuugugagccuacuuuccuuaaucccgccgccccccucccccacuuaauguaaaugucuugaugagagauaauggaugaguauuuuguaugaugaaaucaaacgauuuaggaauuugucucccuccguuacacaguugaugccugaaagaauguugggggggaggacucaacacccuaauuguauuuuuuaaguuucuuuugugaaagauuuuuuaaugcauuuuuacuguaaaaauacauggaaauguaugcauuccacaacca

SEQ ID NO: 30 - RNA, 2861 nt, Homo sapiens
  ref <308>: DATABASE ACCESSION NUMBER: NM_003347.4
  ref <309>: DATABASE ENTRY DATE: 2022-05-04
  ref <313>: RELEVANT RESIDUES IN SEQ ID NO: (1)..(2861)
auucuggggaaggagcagcaccaaauccaagauggcggccagcaggaggcugaugaaggagcuugaagaaauccgcaaaugugggaugaaaaacuuccguaacauccagguugaugaagcuaauuuauugacuuggcaagggcuuauuguuccugacaacccuccauaugauaagggagccuucagaaucgaaaucaacuuuccagcagaguacccauucaaaccaccgaagaucacauuuaaaacaaagaucuaucacccaaacaucgacgaaaaggggcaggucugucugccaguaauuagugccgaaaacuggaagccagcaaccaaaaccgaccaaguaauccagucccucauagcacuggugaaugacccccagccugagcacccgcuucgggcugaccuagcugaagaauacucuaaggaccguaaaaaauucuguaagaaugcugaagaguuuacaaagaaauauggggaaaagcgaccuguggacuaaaaucugccacgauugguuccagcaagugugagcagagaccccgugcagugcauucagacaccccgcaaagcaggacucuguggaaauugacacgugccaccgccuggcguucgcuuguggcaguuacuaacuuucuacaguuuucuuaaucaaaaguggucuagguaaccuguaaagaaaggauuaaaaauuuaagauguucuaguucugcucucuuuguuuuaaaaaucacugcuucaaucuacuucaaaagaaugguguuucuuuucuuguccaauuuuauccaaaaucuucaaguuacauuuaacccauaagguuuaaaaaaaaggaaaaaaaacgguugugguucccuuucuucccuacccuugccacucccacuuucuggcaccgaguuuauuuuucacuuacuuacuuccccagaccccgggcucgccuccacaaaggagaagagacugcccuggcgguccugguggcuuuucuuagcauguguggcacuguugcccagugugggaguugguuuaaauucuccugacuccaguuuauaacauccuuuuaaaaaauuuaaaaacaaacagccacaccccuccuccaguccuucuccucaguucuugugugaaacuccagcugauguuaccacaguaacaucaguuaauugggcaagcccugaugucaguguguguaacugaccucuggccuggccugcacagagaagcccuauaaucacaggucugugguggccccgaaauggggggccugcuagucaggaggaugcugugcacacugugugugaugaaucucgccagaaaggcuccugaggucccagguuggcacuucucccugcagccauuguagaagaucugcugguccuugcaggcaaagcuacagccagaauguccguuugaaacuccuagcucaucugucaccgagcuucauccgaaugugccacggagcuugcucuccacuuccuccgugcaguggcccugccacagcccucccucggcacacuuugacccuuuguaggauuggaauuagcaggacucggcuauuuaaagcaccagucuggggucgccugggccccugcugacccccuccuccagagcagccagcccagcccgggaacaagacggacuuccucucccuucggacucacagccuuugcagagucaagcuccacuugaagcucacucaguaauauccuuucaauguguuuuauauuguuuugacugccuuuuuuuguagaaauaaaaauugaccuuagaauuuaucgucagauaaacuuguaaagauuugaauauuaaugucuuuucaaggcaaaugggauuguccccgcacuaguagagaauccaugucgcucugacaccccaaggaagccgacgauccaaaugccgugugucaccaaccccgcuucugccacuggcggcuucccuucuuggcucuugggggggacuagauccuguggagaagaugacuuaaacuuugcuuuuuguuuuaauuuuaauucuauaacuugagaucuuuccggggccuacaggcguguaagacagcuuggucuggucugugcagaaguggggagugaugggcagguucggcagccuaacauuguucaggcgcauggccccugcgguguguacacgaacucggcuucuuuuguccuagguacgccaagggcagguuucuggagacucccuugugcccgggauggcaagggcaccgggcuggcguuuccacaucugucuucauuagcagaaaagugaugauggauuuuauuucacucacacuccaguuuguaauaaaaugccaaauucugucagcuauccaaacaagccaccauuuguucuuguugcuucucuggauccagaaauguugccauucuuggaaacugucccauugcuucguauuucugccaacguagcucugccugccugucaaccccucacugcacucugcucaucacgggaggauaccugugugccggcagccccucagggacucucagcccuggcacuggcaccccaggguuggccccgucagcagaggcuuggcuuucgagccagugggugucucuccuuugggccugggcggcuugcuccugccagccaugccuucaggguaggcucugagcaagcuggcgaacagcccuggcugcuccaaaaccaaaaagcuggguccucuggaggaggggcgagcuguggagcagccacccacugcugccccaagcucacucaggaauucacacccgccugguuucuugaagugugcuggguccuucccucugcucccuacuccccaccacggcagagaauaggcuuucuaagaugcugcgaucccguucugcugcccguaauaaaaaugcucucagacacug

SEQ ID NO: 31 - RNA, 5790 nt, Homo sapiens
  ref <308>: DATABASE ACCESSION NUMBER: NM_001503.4
  ref <309>: DATABASE ENTRY DATE: 2022-06-02
  ref <313>: RELEVANT RESIDUES IN SEQ ID NO: (1)..(5790)
gagucccagugcugcugcagcucugagcauucccacgucaccagagaagccggugggcaaugagaucaugucugcuuucagguuguggccuggccugcugaucauguuggguucucucugccauagagguucaccguguggccuuucaacacacguagaaauaggacacagagcucuggaguuucuucagcuucacaaugggcguguuaacuacagagagcuguuacuagaacaccaggaugcguaucaggcuggaaucguguuuccugauuguuuuuacccuagcaucugcaaaggaggaaaauuccaugaugugucugagagcacucacuggacuccguuucuuaaugcaagcguucauuauauccgagagaacuauccccuucccugggagaaggacacagagaaacugguagcuuucuuguuuggaauuacuucucacauggcggcagaugucagcuggcauagucugggccuugaacaaggauuccuuaggaccaugggagcuauugauuuucacggcuccuauucagaggcucauucggcuggugauuuuggaggagauguguugagccaguuugaauuuaauuuuaauuaccuugcacgacgcugguaugugccagucaaagaucuacugggaauuuaugagaaacuguauggucgaaaagucaucaccgaaaauguaaucguugauuguucacauauccaguucuuagaaauguauggugagaugcuagcuguuuccaaguuauaucccacuuacucuacaaaguccccguuuuugguggaacaauuccaagaguauuuucuuggaggacuggaugauauggcauuuugguccacuaauauuuaccaucuaacaagcuucauguuggagaaugggaccagugacugcaaccugccugagaacccucuguucauugcauguggcggccagcaaaaccacacccagggcucaaaaaugcagaaaaaugauuuucacagaaauuugacuacaucccuaacugaaaguguugacaggaauauaaacuauacugaaagaggaguguucuuuaguguaaauuccuggaccccggauuccauguccuuuaucuacaaggcuuuggaaaggaacauaaggacaauguucauagguggcucucaguugucacaaaagcacgucuccagccccuuagcaucuuacuucuugucauuuccuuaugcgaggcuuggcugggcaaugaccucagcugaccucaaccaggaugggcacggugaccucguggugggcgcaccaggcuacagccgccccggccacauccacaucgggcgcguguaccucaucuacggcaaugaccugggccugccaccuguugaccuggaccuggacaaggaggcccacaggauccuugaaggcuuccagcccucaggucgguuuggcucggccuuggcuguguuggacuuuaacguggacggcgugccugaccuggccgugggagcucccucggugggcuccgagcagcucaccuacaaaggugccguguaugucuacuuugguuccaaacaaggaggaaugucuucuuccccuaacaucaccauuucuugccaggacaucuacuguaacuugggcuggacucucuuggcugcagaugugaauggagacagugaacccgaucuggucaucggcuccccuuuugcaccagguggagggaagcagaagggaauuguggcugcguuuuauucuggccccagccugagcgacaaagaaaaacugaacguggaggcagccaacuggacggugagaggcgaggaagacuucuccugguuuggauauucccuucacggugucacuguggacaacagaaccuugcuguugguugggagcccgaccuggaagaaugccagcaggcugggccauuuguuacacauccgagaugagaaaaagagccuugggaggguguauggcuacuucccaccaaacggccaaagcugguuuaccauuucuggagacaaggcaauggggaaacuggguacuucccuuuccaguggccacguacugaugaaugggacucugaaacaagugcugcugguuggagccccuacguacgaugacgugucuaagguggcauuccugaccgugacccuacaccaaggcggagccacucgcauguacgcacucacaucugacgcgcagccucugcugcucagcaccuucagcggagaccgccgcuucucccgauuugguggcguucugcacuugagugaccuggaugaugauggcuuagaugaaaucaucauggcagccccccugaggauagcagauguaaccucuggacugauugggggagaagacggccgaguauauguauauaauggcaaagagaccacccuuggugacaugacuggcaaaugcaaaucauggauaacuccauguccagaagaaaaggcccaauauguauugauuucuccugaagccagcucaagguuugggagcucccucaucaccgugagguccaaggcaaagaaccaagucgucauugcugcuggaaggaguucuuugggagcccgacucuccggggcacuucacgucuauagccuuggcucagauugaagauuucacugcauuuccccacucugcccaccucucucaugcugaaucacauccauggugagcauuuugauggacaaaguggcacauccaguggagcggugguagauccugauagacauggggcuccugggaguagagagacacacuaacagccacacccucuggaaaucugauacaguaaauauaugacugcaccagaaauaugugaaauagcagacauucugcuuacucaugucuccuuccacaguuuacuuccucgcucccuuugcaucuaaaccuuucuucuuucccaacuuauugccuguagucagaccugcuguacaaccuauuuccucuuccucuugaaugucuuuccaauggcuggaaaggucccucugugguuaucuguuagaacagucucuguacacaauuccuccuaaaaacauccuuuuuuaaaaaaagaauuguucagccauaaagaaagaacaagaucaugcccuuugcagggacauggauggagcuggaggccauuauccuucauaaacuauugcaggaacagaaaaccaaacacuccauauucucacuuguaagugggagcuaaaugagaacacguggacacauagagggaaacaacacacacuggggccuaugagagggcggaaggugggaggagggagagaucaggaaaaauaacuaauggauacuuagggugaugaaauaaucuguguaacaaacccccaugacacaccuuuauguauguaacaaaccagcacuuccugcgcauguaccccugaacuuaaaaguuaaaaaaaaguugaacuuaaaaauaacagauuggcccaugccaaucaaaguauaauagaaagcauaguauacagaaaaaaaaaaaaaaaagaaacugagggaggaaaaauaguacagaguaaagcaugggggugagggauaaccucacauuccaaaucacuugugacuuaguccuuuuuaacuuuuauuuuagguucagugguacaugugaagguuuuaucuuuucuguuccucccaccuuccccacucaaguagaccccaguguguguuguuuuguguucauaaguucuuuuaacuuagcacccacuugugagaacaugcuauacuugguauuugguuuucuguuccuggucguguguuaguuugcuaaggaugauaguccuuuuuuuuuuuuuuuuuuuuugagacggagucuugcucuguugcccaggcuggagugcagugaugugaucucagcucacugcaaccuccgccucccagguucaagugauucuccugucucagccucccaggcagcugggacuacaggugcacaauacuacgcccggcuaauuuuuguauuuuuaguagagacaggguuucaccauguuggccaggcuggucucgaacuccugaucucaggugauccacccaccuugaccucccaaagugcugggauuacaugugugagccacaggaccuggccgauaauccuuucuuaaugaggauuaauaccuuuucaauguuuauauagugaaggacaaauauaaaaaccaagacuaucacccuucugugcccuccaauacuuuuguagaguuggcuguucuaacaccuaaagagccuagcgacacugcuaguuuuaagccaucucaugagggaccuuugucgcauuagguuacaacuugugccaaaagaacuagagggaaaagauguggccugagccacacgcuacuguaagggccggagaggaaagcaggccuuggccucgcuuguuccugggcuguuuuucguaggauggauuucauuugucaguccugcagcguggcacacuuaagggaauggaauguaucauaggguuucaguuucaguaaacauuagcgggcauuaucuguaaacaguaacuaguuuauuuuguguuaaauugggaagaaccucuuucuucugccacgggugagaugauacuaaaagcaggagaaaauaacaaaugagaagagcccuucucuucucagcuucucucaagaguagagacuggugaaaaggggacaggaagaggaauccagcugaagaguggaaaguuaacaagaacaucaagagguacaucccuagagagcaucucugggucacucccaggaugaccucauuuuucuggaagagguacaugugcuagcugcauaguaaccuauggcacagcaaaagcacaaggaagcgccauuugguuuuguuuagggacaauuauugguagcccaaccuaucugaagaauuacuuuuaauuaaaaggaugguuauuuuuaggggaccagaaauuacuuaguuguauucacuuaugaguugagcaguuagagcacucugacaucugacacuugaguaaagggauguuuuucucaguacugggucuuuucauuagacggaugcauuuauauagugucaguuuuuaugcuucacuaaaugauaaggccucuguuuaaacaugaccauuauuuaccucacgauuuauauuuuucucaaugccugaauacacccaauagcaagcaccagguggagguuaagacccacaaaaauggaagcaagcuucaaguuuauaggacauuuaaccauacuaaccccaaugucaaaaacaauguaaauuauacuccuacuguaaaaggucaacugauauggcacguauguaaaaaaccuggcauacaggaaaaaauuaaugcaugacagucuaauauauuauccugauacauggauuaaauuuucuuuccacuaaguauuaacuauaguuuuaagcauugauucugcuuaauaacuaaaauguaaagaccuuaccuuguaggguuguaauuucucauuaacagaaaucugaacuuauacaaguuuaacugaaauuuaucuuuauuucaagauaucagaagacauuuacauauuugcauuuucugucuugcuacuaucugcguauauguaacagcuuuucucaaggcuucaagauaggaauuguuuuaugccuuuguaucacagaccuaagagaccaaaauuauuugaagauggaguggggacaaaacuagggucucugcagucccuacuccauguaccauccauuagcucaccucuguagguauuacuaaagucaugagaguuaaauccaacgugcaacuuuaauauugcaauuccaaaaaauaaagcuguauacaacucuuggaauaacucaccaaaauaaauagcuuuuuaauauaaaa

SEQ ID NO: 32 - RNA, 11692 nt, Homo sapiens
  ref <308>: DATABASE ACCESSION NUMBER: NM_015030.2
  ref <309>: DATABASE ENTRY DATE: 2022-04-19
  ref <313>: RELEVANT RESIDUES IN SEQ ID NO: (1)..(11692)
gggcgcccgucgggcucugggagagagcgagagagaaauccgguuaaaaucagagucggagggagguuuaaccacagaucgguuccgaucggauuauuccuuaaaggggacgcgccauugucaagagaacggagccaggggcccgagggcggggaccggcggcgcuggaggagcgaggcagcuguacuaggacugggaacggccuagaacauuccauauucugcuucacacacggugacucacucacuacugcuuuguaccagugugcccacucgagugccuguaaaguacaagcuccagaucaaaaaagcuucuagauggauccuuuccaaacuuggugucuuuucuacaaacucugugccucaacaaccauuucuaagugagcugaagccauuuauugguaucuuggaugguuaacaucuaaaugcuuaauuccugaaaccacucaagagucauaggauuagaugauccaagucaccaagaaauuacugucucaagugauacgcuucagcgccagucagcaaaguaaauaaauacugugcuucuuccaacuuucauuccacuuggggaaaaaaaaauaucaucaugucaaacauuacgauugacccagaugucaaaccuggugaauaugucaucaagagccucuuugcagaauuugcuguucaagcugaaaagaaaauugaaguuguaauggccgaacccuuggagaagcuauuguccagaucucuucagaggggugaagaucuucaguuugaucaguugauaagcucuaugagcucaguagcagagcacugucucccuuccuuacuucgcaccuuguuugacugguacagacgccaaaauggaacggaagaugaaucuuaugaauauaggccucggucuagcacaaagucuaagggggaugaacagcaacgugaaagagauuaucuucuugaaaggagggacuuagcaguagacuucauuuuuuguuuaguuuuaguugaaguucuaaagcagauuccuguucauccuguacccgaucccuuaguucaugaaguucuaaacuuagcuuuuaagcacuuuaaacauaaggaaggauauucaggaaccaacacugggaaugugcauauuauugcugauuuauaugcagaggugauagggguucuugcccaaucaaaguuucaggcuguaaggaagaaguuugugacagaauuaaaagaacugcgacaaaaggaacaaagcccacaugugguacaaagugucaucagcuuaauaaugggaaugaaauuuuuucgaguaaaaauguauccuguagaagauuuugaagcaucauuucaauuuaugcaggaaugugcucaguauuucuuagaagugaaagauaaagauauaaaacaugcacuugcugguuuauuuguggagauucuuaucccuguagcugcugcuguuaaaaaugaagugaauguucccuguuugaaaaauuuuguggagaugcuuuaucagacuacuuuugaacugagcucgagaaagaagcauucauuggcuuuauauccacuaauuaccugccuuuuaugugucagucagaaacaauuuuuuuuaaauaacuggcauauuuuccuacagaacuguuugucacauuuaaagaauaaagauccgaaaaugucucgaguugcacuggaaucuuuguauagauuauuguggguuuauguaauuagaauaaaaugugaaagcaacacuguaacucaaagucgucuuaugagcauagugucagcacuuuuuccaaaaggcucacgaagugugguuccucgugacacaccucucaauauauuugugaagauuauucaguucauugcucaggaacgcuuggauuuugcaaugaaagaaauaauauuugaucuucucaguguuggaaaaucuacuaaaacuuucaccauuaauccagagagaaugaacauaggucucagagucuuccuuguaauagcagacaguuugcagcagaaagauggugaaccacccaugccaacaacaggaguuauucuucccucaggaaauacucuucguguaaagaaaauauuucucaauaaaaccuugacagaugaagaagcaaaagucauaggaaugucaguuuacuauccucaaguaagaaaagcauuagauagcauccucagacauuuggacaaagaaguugggagaccaauguguaugaccagugugcagaugucuaauaaggagccugaagacaugauuacgggggaaagaaaacccaagauugauuuguuuagaacuuguauugcugcgauuccaagguugauuccugacgguaugagcagaacugaccugauugaauuguuagcaaggcucacaauucauauggaugaagaacugcgugcucuggcuuucaauacucugcaggcacuaaugcuugauuuuccagauuggcgggaggauguucuuucaggauuuguuuauuuuauuguucgugaagugacugauguccaucccacacuucuugauaaugccguaaagauguugguacaauuaauaaaucaguggaaacaagcagcccaaaugcauaauaaaaaccaggacacucagcaugguguagcuaauggagcuucucaucccccuccucuggaaaggagcccauauuccaauguauuccaugugguugaaggcuuugcgcuugucauucucuguagcagucgaccugccacuaggagacuagccgucaguguccuuagagaaauacgggcuuuauuugcacuucuggaaauaccuaagggugaugaugaauuagccauagaugugauggacaggcuaagcccauccauucuugagagcuucauacaucucacuggggcugaucagacuacuuugcucuauugcccuagcucgauagauuuacaaacuuuagcagaauggaacucuucuccuauuagccaccaguuugaugugauuaguccaucacauauauggauauuugcacaugugacccaaggccaagacccauggauuauaagucucuccaguuuuuuaaagcaagaaaaucuuccuaaacacugcucuacagcugugagcuaugcuuggauguuugcauacacaagacuucaguuguuguccccucaggucgauauaaauagccccaucaaugcuaagaaaguaaauaccaccacaagcagugacucauacauuggccuguggagaaacuaucugauccuuugcugcagugcagcaacaucgucaucuuccacaucugcagguucugugagauguucuccuccugagacgcuggcgucuaccccagauagcggcuauagcauugauucuaaaauuauuggcaucccauccccuucauccuuguuuaagcacauaguuccaaugaugcguucugagagcauggaaaucacagaaucccuuguucuaggucuuggcaggaccaacccaggagcuuuuagggaacuaauagaggaauuacaucccauaauuaaagaagcacucgaaagaaggccagagaauaugaaacggcgcaggcgucgagacauuuuacgaguacaacugguacgaauauuugaacugcuggcagaugcuggugucauuagucacagugcaagugguggccuugauaaugaaacacauuuucucaacaacacuuuauuggaauauguagauuuaacuagacaacuccuggaagcagaaaaugaaaaagacucugacacacugaaggauauacgaugccauuuuagugccuuaguggcgaauauuauucagaauguuccagugcaccagagaagaaguauuuuuccucaacagagccuucgucacagucuauuuaugcuguucagucacugggcagguccuuuuagcaucauguuuacgcccuuggacagauacagugauagaaauaugcaaauuaauagacaucaauacugugcguuaaaggcuaugucugcuguacuguguuguggcccuguugcagauaauguaggacuuucaucagauggcuauuuguacaaaugguuggauaacauuuuggauucucuggacaaaaagguucaccagcugggcugugaagcaguuacguuguuacuggagcugaacccugaucagagcaaccugauguacugggcuguggaccgcugcuacacgggcuccgggaggguggcggccggcugcuuuaaagccauugcuaauguuuuccagaacagggauuaucaaugugacacagugaugcuucuaaaucugauacuguuuaaagcagcugauucuucuagaaguaucuaugaaguugcuaugcaacuuuuacagauucuggaaccgaagauguuucgcuaugcucacaaauuggagguucagagaacagauggaguacucagccagcugucuccucuaccacaucucuauucuguuucauauuaucaguuguccgaggaacuagcaagggcguauccugagcuaacucucgccauauucucagagauaagccagagaauccagacagcucacccugcugggcggcaggugaugcugcacuaccugcuaccauggaugaacaacaucgagcugguggacuuaaaaccucuccccacagcaaggcgacaugaugaagaugaagaugauuccuuaaaagaccgagaacuuauggugacuaguaggcgcugguuacggggagaaggauggggaucuccacaagccacugcaaugguuuugaacaaucugauguauaugacagcaaaguauggcgaugaacuggccuggucggagguggagaauguguggaccacacuugcagauggcuggcccaaaaaccugaaaauaauuuugcacuuuuugaucagcauuuguggggugaauagcgaaccaagccucuugccuuacgugaagaaggucauuguauauuuagguagagauaaaacaaugcaguugcuagaagagcuggugagugagcuucagcugaccgauccugucaguucaggggucacucacauggauaaucccccguauuaucgcaucacuuccagcuauaaaaucccuucugucaccucaggaacuacuuccaguagcaauacaaugguagcucccacagauggcaauccugauaauaagcccauuaaagagaauauugaagagagcuaugugcaccuggacauuuacaguggacuaaacagucauuugaaucggcaacaucacagacuagaaucccgauacaguagcagcucuggaggaucuuaugaagaagaaaaaagugauucaaugccacuuuauucuaauuggcgacugaaagugauggagcauaaccaaggagagccacugcccuucccaccagcuggaggcugcuggucaccacugguggauuacgugccugaaacgucaucaccuggauuaccucuucacagguguaacauagcagugauccuuuugacugaucucaucauugaucauagugugaagguggaauggggaagcuaccuccaucuucuucuucaugcaauuuuuauaggguuugaccacugccacccugagguguaugaacauuguaaacgccugcuucugcacuuauuaauaguaaugggacccaauaguaacauccgaacuguugcuucuguccuucucaggaacaaggaguuuaaugagcccagggugcuuacagucaaacaaguugcacacuuagauuauaauuucacagcaggcauuaacgauuuuauaccugauuaccagcccuccccuaugacugacucagggcuuagcucaaguucuaccucuucuaguaucagcuuaggaaauaacagugcugccauuucacaucugcacaccacuauccucaaugagguugacaucucaguggagcaggauggaaaagucaaaacccucauggaauucauuaccucaagaaaaagagggccccuuuggaaccaugaggauguuucugccaagaauccuagcauaaagagugcugaacaguuaacuacauuuuugaaacaugugguuucuguuuuuaagcagucaagcucagaaggaauucaucuggaacaucaucuuagugaaguugcucugcaaacagcacuuuccuguucuucucgacacuaugcugggagauccuuucagauuuucagggcccuaaagcagccucucacugcaacuacacuuucugauguucucuccagacuuguagaaacuguaggggauccaggagaagaugcacagggauuugugauugagcuucuucucacauuggaaucugcaauugauacuuuggcugaaaccaugaagcauuaugaucuucuuucugcccuuucucaaaccucauaucaugauccuauaaugggaaacaaguaugcagcuaacaggaaaagcacuggacaacucaaucuaagcacaagucccauuaauaguagcaguuauuugggauauaacaguaaugcaagaaguaacucuuugagauuaaguuugauuggugaccgacgaggugaccggcggcggaguaacacacuggauauaauggauggacggauaaaccauagcaguaguuuagcaaggacuagaagccuuuccucucuaagagagaaaggaauguaugacgugcaguccacuacugagccuaccaacuugauggccaccauuuuuuggauagcagcaucuuuauuagaaucagauuaugaauaugaauaccuccuggcucucaggcuucucaacaaacugcuuauccauuugccuuuggauaaaucagagagucgagagaagauugaaaauguacaaagcaaauugaaauggacuaauuuuccaggacuucagcagcucuuccuuaaggguuuuaccucagcaucuacacaagaaaugaccgugcaccuccucaguaaacucauuucugucuccaaacauacauugguggauccuucccaauugucaggcuuuccucuuaacauccuuugcuuauugccucacuuaauccagcauuuugacagcccaacucaguuuugcaaagaaacagcuagucgaauagcaaagguuugugcagaagaaaaaugcccaacacuugucaaucuggcacacaugaugaguuuguacaguacacacacguauuccagagacuguucuaacuggaucaaugucgugugcagauaccugcaugacuccuucucagauacaacauuuaaucuugugacuuaucuugcagagcuguuagagaaaggauuguccaguaugcagcaaucauuacuacagauuauuuauagucuauugagucauauugaccugucugcagccccagccaagcaguuuaaucuggagaucauaaagauuauuggcaaauauguacagaguccuuacuggaaggaagcccuuaacauauuaaagcugguggugucacgcucugcgagucuugucguacccagugauauccccaagaccuauggaggagauacagguucuccugaaauauccuucacuaaaauuuuuaauaauguuucuaaggaguugccugggaagaccuuagauuuucauuuugauauaucugagacaccaauuauuggaaacaaauauggugaucagcacagugcggcuggaagaaaugggaaaccaaaaguuauugcugucacuagaaguacuuccucaacuucuucugguucuaauucuaaugccuugguuccuguuaguuggaaaaggccacaguuaucacagcgaagaacaagagaaaagcuaaugaaugugcuuucucucugugguccagaaucuggccucccaaagaacccaucaguuguauuuucuucuaaugaggauuuggaagucggugaccaacagacuagccuaauuucuacaacagaagacauaaaucaagaggaagaaguagcuguggaagauaauagcagugaacaacaguuugguguuuuuaaggauuuugacuuuuuagauguugaauuggaagaugcagagggugaaaguauggacaauuucaacuggggaguucgcaggcgcucacuggacaguauugacaaaggggacacuccaucccuccaggaguaccagugcucuaguagcacccccagccugaaccucaccaaucaggaggauacagaugaguccucggaagaagaagcggcacuuacagcaagccagauacucucacgcacacagauguuaaacagugauucugccacugaugaaacaauaccagaccauccugacuuacuucuccagucugaagauuccacuggcagcaucacaacagaggaagugcuucaaaucagggaugagaccccaacuuuggaggcuucucuagauaaugcuaacagccggcugccugaggauacaacuucaguauuaaaggaggaacauguuacaaccuuugaagaugaaggauccuauauaauucaagaacagcaggaaucucuugugugucaaggaauucuugauuuagaagaaacugaaaugccagagccucuagcuccugaaaguuaccccgagucagucugugaagaggauguuaccuuagcucugaaagagcuagaugaaagaugugaagaagaagaagcggauuucuccggacugucuagucaagaugaagaagagcaagaugguuuuccagaaguacagacgucgccucugccgucaccauuucuuucugccaucauagccgccuuucagcccguggcauaugaugaugaagaggaagccuggcgcugccacgucaaucagaugcugucugacaccgacggguccucugcaguguuuacuuuucauguguuuucuaggcuguuucagacaauucaaagaaaguuuggagaaauaacuaaugaggcagucagcuuucuuggugauagucugcaacgcauugguaccaaauuuaaaaguuccuuggaagugaugaugcuguguucagaaugcccaacagucuuuguggaugcugaaacacugaugucaugugguuugcuggaaacacucaaguuugguguuuuggaguugcaagaacaccuggauacauacaaugugaaaagagaagccgcugagcaguggcuagaugauuguaagaggacauuuggugccaaagaagacauguauaggauaaacacagaugcacaacaaauggaaauacuagcagaauuggagcucugccgaagauuauacaaauugcauuuucaauugcugcuucuguuccaggccuacuguaaacuuaucaaccaaguaaauacgauaaaaaaugaagcagaggucaucaacaugucagaggaacuugcccaacuggaaaguauccucaaagaagcugaguccgcuuccgaaaacgaagaaauugacauuuccaaagcugcacaaacuacuauagaaacugccauucauucuuuaauugaaacuuugaaaaauaaagaauuuauaucagcuguagcacaagucaaagcuuucagaucucucuggcccagugauaucuuuggcaguugugaagaugacccuguacagacacuguuacauauauauuuccaucaucagacgcugggccagacaggaagcuuugcaguuauaggcucuaaccuggacaugucagaagccaacuacaaacugauggaacuuaaucuggaaauaagagagucucuacgcauggugcaaucauaccaacuucuagcacaggccaaaccaaugggaaauauggugagcacuggauucugagacacuucaggccuuuaggaaagaaacuaaacugaagaugaugaagaauauuaaccaagcaccuuuuauggacccuugcauucacugauaacuuucuggcagcaucuacuuuuuaguguaacuaaugucaaacuguaucaucaaaaacaaagaucugaaagaaaaaaacaucugauauuuuaacagcugccaauaucucccacaauaacugcgugaagaaggaauuuuuuaauuacuuaaccuacgugaaaagaaaagggcuaaaagugaugccuacaaauacauuacuuucuggggaaagaaaagaauuccaagaauguuugcaauaauggccuccaauacugaaacaaccaaaagcaggugaaaugaggcugaaaucaaggcuguuucauuuuagcugaagaccugcaaagcugcuuggauuucuagcaguugaaaaccuaaccugcaauugaugcuuaaucauccauccacaugaacaugcagucagcuauacgcaucaagacucguuugcaaaaaccauugauuuuucaguaugugggauaaggguuuggguuuuuuuguuuuuuggucuguauaaggaauuaugugugugugaguugggauguauggauaugugauagucauauuuucaagguauggauguuuggugauaacgucucagagcaugccuaaaagagcacugcaagauuauuuuugaagaaauguuauuuuauuagaucuaacucuucauaguauguaaauguuagaacauuuuaauaauauuuuaaaacugggcuuucccaguauuucaaaaagaaauaauauaucuguuaacguuauuggaaugcugcucaguucucugaucagugcuuauguuaugauuguugauaacuaaccaaaguagaugccugcagagacuuuaaaauguaaaauaaagauguaugcugccugucagcuauucucauuagaaaaguuuuaacuuauuuuacuccauauagaaacuguagagacuaaaaccaguuauuuucuuguacauuugugccaugcacuguuguuauacaagaauagguguacaaagcuaaagaaaaauuguggucauugaugauggaauauaugacuugcaggcuuugaagucugcagaauucaagaaaagagcugcaaaugcauuuuuguaucuuuauucaggacucacaugcuuuacccuaaagaaaccuggggcuaggggaaaugaaaggaagccugaagacugacuaccaaaacaugcaauauacuuauucacugucuaagucuguaguauaacaugaacuggagucucuauccuuuucuaaaaucgcauuuuguaagaaaagaaagaaaaaaaaaaaaaaagaauguagucccacaacuccugauuugaaagaaacaccuuguauuuuuuauauacaucucuuauccacugugacuuuucuuuuaaacugggcucugugauuccagaguuuagaauguagaaaugaaaugcuuagcuuuaccuuuucuugggggguguggacccugccagaaauguaauuacguaugcucaagugcauuaauugaaggcacugugcacaccauuggacugcugaccacaguuaaacauuuccaguaaaauuucguaacuggucacagcucauucauaaucacuguauuuuuugacccugauaaagacaauuauuuugaugguguucugguacuguaauugguauccuuuaaauuauuuaaaacuuuuggguguggggcaggcagagagggaugguguccagagauacaucacacuuuacauccuaucuuacuguccucuguuuuucuuacccuccuuacauccuuuuuguuaaaaauaaaaagcauuguagcuguugguuuguguuguauuuuaacaaagaaugaaugauuacagugugcaucauuuagaaaccuuucaaauguuaccuucuugaauugcccuagaaaagaacaaaauuaguugacuacccaaccaaaccuuucaguugauauuugcuuuuuguguuuauuuauauguaacuaagucuauuaauauaauaaaaaugaauauaccugcagcaagcaa

SEQ ID NO: 33 - RNA, 12478 nt, Homo sapiens
  ref <308>: DATABASE ACCESSION NUMBER: NM_006901.4
  ref <309>: DATABASE ENTRY DATE: 2022-08-12
  ref <313>: RELEVANT RESIDUES IN SEQ ID NO: (1)..(12478)
gcgcgcgcgccugcucgccugcagccggggagucgcgcggggcggggccgcgugcgcggggucgggggcgcgcgaccgaagcggauaagggacgcggcggcgguuggcugugucuguugagagaguacaggacagcccggacugcgagaggcggcggcggaggcaguaguugcaccggcagaggcggccgcuggcagcgguugaggcggcgguccaacgcgugcucucugggcggggugcggcgggggaugugccugcucaggccgggcggagucucucugacagggcgggggaugcgccgcggccgcucgcccggacccugaggcugcugggcccacccucccggaaccguccgacccucgguggccucggccucuucugccaucuccgguccuacccuggggcggaggguggaaggcagcuuccgucgaagaggagggggcugcgguggccaccgcggcggagcccgaguuauuuuaccaagaaaaugguuugcacgacuuugaacauauacuauccaugcugaugggacaggauccaauaugaauauaaaugauggaggaagacgacgcuuugaagauaaugaacauacauuacggauauauccuggggcuauuucagaagggacaaucuacuguccgauuccugccagaaaaaacuccacagcugcugaggugauugagucucuuauaaacaaacuucaucuugacaaaacaaaauguuauguucuagcagagguaaaggaauuugguggagaagaauggauucucaauccaacagauuguccaguucagcgaaugaugcuguggccccgaauggcucuggaaaaucgcuuaaguggagaggacuaccgcuuccuucugagagagaaaaaccuugauggaucaauccauuaugguagccugcagucauggcuacggguaacagaagaacgucgcaggaugauggaacgggguuuucuuccacagccucaacagaaagacuuugaugauuuauguaguuuaccugauuugaaugagaaaacucucuuagaaaaccuacgaaaucgcuuuaagcaugaaaaaauuuauaccuauguuggcaguauucuaauaguuauuaacccauucaaguuucuuccuauuuauaaccccaaauaugucaaaauguaugauaaccaccaacugggaaaacuugagccccacauuuaugcuguggcugauguagcuuaucaugccaugcuucagcgcaaaaagaaucagugcaucgugauuucaggagagagugguucugggaagacucaaagcacaaacuuucuuauucaccaccuuacugcucucagucagaaaggauuugccaguggaguagaacagauuauucuuggagcuggaccaguacuugaggccuuuggaaaugcaaagacagcucauaauaacaauucaagucguuuugggaaguuuauucaaguaaauuaccaggaaacaggcacuguacuuggugccuauguugaaaaauaucuacuggagaaguccagacucguuuaucaggagcauaaugaacggaacuaucauguauucuauuaccuccuggcaggagcaagugaagaugagagaucagcauuccaucuuaagcaaccagaggaauaucauuaucucaaucagauaacaaagaaaccccucagacagagcugggaugauuauugcuaugacucugagccggauugcuucacgguggaaggagaagauuugagacaugacuuugagcgccuacaacuugccauggaaaugguaggauuucuucccaagacacgaagacagauuuucucucuucucucagccauacuacauuuggguaauaucuguuacaaaaagaagacauaccgggaugacuccauugauaucuguaauccugaaguucugccuauugucucagaauuauuagagguuaaagaagagaugcuauuugaagcauuaguuacaaggaagacggugacagugggagaaaagcuuauuuugccauacaaguuggcagaggcugugacagugaggaacuccauggcuaagucucuguauagugcccuguuugacuggauaguuuuucgaauuaaucaugcacuucugaauaguaaagauuuagagcauaauaccaagacauugucuauugguguucuugauauuuuuggguuugaagauuaugaaaauaacagcuuugaacaguucuguauuaauuuugcuaaugaacguuuacagcacuacuuuaaucagcauaucuuuaaauuggaacaagaggaauauagaacugaagguaucagcuggcacaacauagauuacauugauaauaccugcugcauaaaucuuauuagcaaaaaaccaacaggacugcuucaucuuuuggaugaagaaagcaacuuuccacaggcuacaaaucaaacauugcuagacaaguuuaagcaucaacaugaagauaauucuuacaucgaauuuccagccgugauggagccugcuuucauuauaaaacauuaugcuggaaaaguaaaauaugggguaaaggauuuccgggaaaaaaauacagaucauaugcgcccagacauuguagcucuucugagaagcagcaagaaugcauuuaucucugggaugauuggaauugauccuguagcuguuuuccgaugggcaauucuccgagcuuuuuucagagccaugguugcuuucagggaagcugggaaaagaaacauucacagaaaaacuggacaugaugauacagcgccaugugcaauuuugaaaaguauggauaguuuuagcuuucuccaacacccaguccaccagaggagcuuagagauucugcagagaugcaaggaagagaaguacaguauaacccggaaaaaucccagaacaccucuuucugaucuccagggcaugaaugcucuaaaugaaaaaaaccaacaugauacauuugauauugccuggaauggcagaacugggauucgccagagcagacuaucaaguggcaccuccuugcuugauaaagauggaauauuugcuaauucaacuagcagcaaacuccuggagagagcccauggaauucucacgagaaacaaaaauuucaaauccaagccugcccuuccaaagcacuugcuagaaguaaauucuuuaaagcaccugacaagacugacacuacaagaucgcauuaccaagucucuucuucauuuacacaagaagaaaaaaccucccagcaucagugcccaguuucaggcaucauuaagcaagcuaauggaaacacuuggucaagcagaaccauauuuuguaaaaugcauucgcucuaaugcugaaaagcugcccuuaagguucagugaugucuugguacuuagacagcuucgauacaccgggaugcuggaaacaguucgaauucgccaaucaggauacagcuccaaauauucuuuccaggauuuugugagccacuuccauguacuucuuccccgaaauauuauuccauccaaauuuaacauucaggauuucuucaggaaaauaaaucuuaauccagauaauuaucaaguuggaaaaaccauggucuuucuaaaggagcaggaacgacagcacuuacaagaucugcuucaccaagaggugcuccgcagaaucauauuguugcagcgaugguucagggucuugcuguguaggcagcauuuccuccaucugagacaagcaucuguuauuauccagagauucuggaggaauuaccuaaaucagaagcaagucagagaugcagcugugcagaaggaugcuuuuguuauggcuagugcagcugcucuucuccaagcuuccuggcgugcucacuuagagaggcagcgguacuuggaguuacgggcugcagccaucguuauccagcagaaauggagagauuacuauaggcgcaggcacauggcugcuauuugcauacaagcaagauggaaagccuacagggaaaguaaaagguaccaagaacaaaggaaaaaaauuauccuuuugcaaucaacauguagaggauucagagcaagacaaagauuuaaagcuuuaaaagaacaaaggcuaagagaaacaaagccagaaguuggauuggugaauauuaagggauauggaucucuggaaauucaggguucagacccuucaggaugggaggauuguucuuuugacaacagaauaaaagccauagaggaauguaaaucuguaauagagaguaaucgaauuagccgugaaaguucaguggacugcuugaaggagucaccaaacaagcagcaggagagagcccaaagccagagugguguggacuugcaggaagaugugcuuguaagagagagacccagauccuuggaggaucuccaucagaaaaaaguaggccgggcuaagagagaaaguaggagaaugagagaacuagagcaagcuauauuuagcuuagaauugcugaaaguucguucucuuggugguauuucuccuucagaggaucgcagauggucuacagaauuggugccugaaggccuucagucuccacgggguacaccugauagugagagcucucaaggaagcuuggaacuucugagcuaugaggaaagccaaaagagcaaacuagagucugucauuucagaugaaggagacuugcaguuuccaucaccuaagauauccagcaguccaaaauuugauucacgggacaaugcccucagugccucaaaugagacuagcagugcagagcauuugaaggauggaacuaugaaggaaaugguggucugcaguucugagucuauuaccuguaaaccacagcugaaagacuccuucauuucaaauagucuaccuacuuuuuuuuauaucccccaacaagacccacugaaaacaaauucccaacuagacacaaguauccaaagaaacaaacuauuggaaaaugaagacacagcgggggaagcucuuacuuuggauaucaacagggaaacuagaagguaucacugcucaggaaaagaucagauuguuccuucuuugaauacagagucuucuaauccugugcuuaagaaguuagaaaagcuaaacacugagaaggaagaaaggcaaaaacaguugcagcaacagaaugaaaaagagaugauggaacagauucgccagcaaacagauauuuuagagaaggagcgcaaagccuucaagacaauugaaaagccaagaauuggagaguguuugguggcaccaucuuccuaucagucaaagcaaagaguagagaggccauccucucuccucagcuuaaauaccucaaauaagggagaacuuaauguacuggggucccuaucauuaaaagaugcagcucuugcccaaaaagacaguuccucugcucacuuacccccaaaggaccgaccugucaccguguucuuugaaagaaaaggaaguccaugccaaucuaguacugucaaggaauuauccaagacagacagaaugggcacccagcugaauguagccuguaaacucucaaauaaucgcauuucaaaaagagaacacuuuaggccaacucagucuuacagccacaauucugaugaccuuuccagagagggaaaugcuaggcccauuuucuucacuccaaaggacaauaugaguauuccccuugucagcaaagaagccuuaaacaguaaaaauccucaacuccauaaagaagaugaaccagcauggaaaccugugaaguuagcugggccaggccaaagagagacaucacagcgauuuucgucaguugaugaacaagcaaaacuucauaagacuaugucucaaggagagauuaccaaguuggcagugagacagaaggcuucagauucagauauaagaccucagagagcuaagaugagauucugggccaaagggaaacaaggggagaagaagacuaccagagugaaaccuacuacccagucagagguuucgccacucuuugcaggcacagaugugauuccagcucaucaguuuccagaugaauuagcugcauaucacccaacaccuccuuugagcccagaacugcccggcaguugccggaaggaauucaaagagaacaaagaaccuucuccaaaggcuaagcgcaagcgaagugugaagauuagcaacguggcuuuggauucuaugcauuggcaaaaugacucuguccagaucauagcaagugucagugauuuaaaaagcauggaugaauuucuucugaaaaaggugaaugaccuagauaaugaagacagcaagaaggauacacuaguggauguuguauuuaaaaaagcccugaaggaauuucggcagaauaucuucagcuuuuauucaucugcauuggcgauggaugaugggaaaagcauacgguauaaagaccucuaugcacuauuugaacagauucuggaaaagacgaugaggcuugagcagcgugauucacugggugaaucuccagugagaguuugggucaacacuuuuaaaguguuuuuagaugaauauaugaaugaauucaagacuucagauugcacagccacaaaggugccaaaaacagaaagaaagaaaagaaggaaaaaggaaacugauuugguggaagaacacaauggucacaucuuuaaagccacccaauauagcaucccuacauacugugaauacuguucuucuuugauauggauaauggaccgagccucuguuugcaaauuaugcaaguaugcuugccauaagaagugcugucugaaaaccacagccaagugcucuaaaaaguaugauccagagcugucaucucgacaauuugggguugaacugucccguuugaccagugaagaccgaacuguuccuuuaguaguggaaaagcucauaaacuacauugaaaugcauggacuguauacagaagguauuuaucgaaagucugguucgacuaauaaaaucaaggagcuucggcagggucuagauacagaugcugagaguguaaaucuagaugacuauaacauacacgucauugcaaguguauucaaacaauggcuucgagauuugcccaauccucucaugaccuuugaacucuaugaggaauuucuucgagcuaugggccuucaggagaggaaggagacaauccgugguguauacucugugauugaucaacucucccgaacucaucucaauacacuggaacgccucaucuuucaucuagucaggauugcucugcaggaagacacuaaucgaaugucugcuaaugcuuuggccauuguguuugcgcccugcauucuccgcugcccugacaccacugacccacuacaaaguguacaggacaucaguaagacuaccacuuguguggaacugauuguuguggaacaaaugaauaaauacaaggcucgucucaaagauaucaguagcuuggaauuugcugagaauaaggcaaagaccagguugucacugauucguagaucaaugggaaaggggcguauucgucgaggaaacuauccagguccaucgucuccuguuguaguucgguugccuucugugucugaugucucagaggagaccuugacuagugaggcagccauggagacugacaucacagaacagcagcaagcagcuaugcagcaggaggagagaguacugacugagcagauugagaaccuacagaaggagaaggaggagcuaacauuugagaugcuuguacuggaaccccgugccucugaugaugaaacccuugagucugaggccuccauugggacugcugauagcucagagaauuugaauauggagucugaauaugcuaucucugagaaaucagaaagaagcuuagcccuuagcucccugaagacagcuggcaagucugaaccuuccagcaaguugcgaaagcaacuuaaaaagcagcaagacucuuuagaugucguggacucuucggucuccucuuuaugucugucuaacacggcaucaucucaugggaccagaaaacuauuucagauuuauuccaaaucuccauucuaccgagcugccucagguaaugaggcccugggaauggaaggaccauugggccagaccaaauuccuggaagacaagccucaguucaucagcagaggaaccuucaacccggaaaagggcaaacaaaaauuaaagaaugugaaaaacucaccucagaaaaccaaagagaccccagaggggacagucaugucuggccgcagaaaaacuguggacccagacugcaccuccaaccaacagcuagcacucuuuggaaauaaugaauuuauggucugaaccggcagaugugugucccuccguggcuacagagugguaaacaaaucucaccuuuggggcugcguuucaucaccucguccacaauagucaauccuaauugugguccugccucuuuucuaagcauauggcuaagacuguaugugcugaauuccugggccuccugcagaagcagaaagccugcuggggauggugccagcugugccuuggcuguuguauuugaauugagauuuuuacuauacaaagccaccuagggccuggggauuugggucaguuguaguugccucucccccacccucuuuucccuucccaaagguggguguugaacuaggggggauauugcuguccugagggacccucucauuucugacauuugaagaaaacguauaaaucuuucuuaaccgugaaagcaaaagccuuuggguuuauuuugggauaguuaggagcuaggguagaauauaauuuuuuuccaaaaacuuacuuacaaacaaaaagccuaaucccucuauuuuaagauuucugaaaaaacacuccauguuauauucuggggaaagcaaaaacaaaaaaaaaaaacaaaaaaaaaaaaacaaaaaaaaaccgugauuguuuccauuaagccaucagagcgugucucagauaucuuguuauacagugaagaagagaauaccuauuuuuuuugaugauguuugaugucauaguaaucagugcugguuuaacuccuguuuaguuccugccaaauacauugcaugggcugaagugucaguagccauacuguucucauguuucuugguccuaggaucagauucuucuacauuauuuuuuaguuucccagagacauggugaucccuaaacuuaugaagguuucauccuaagccauucaggcuuuucucuuuguauagauuaaaccgaaaaccgcauaugagccuguucaucugcugguggauucccaccacagacucagacucaaacaaacaaacaaauaaugugaccguuuaguuaguuuccaaaaacauucacauacaauauugcaugaauaaguaaguaaguguuacagaaagcugccaaaaaauaaaaagguuuuuaauaugaccuaucuaacuaacuaacuuuuaucuuccuuuucucagacuaucaguauuaaauagauggaaaaaaaucaaggacuacuugaagcuauuuuuguuaauauucugguuacugaaguuuauuguaaauauguauguauaguaggcauauuucuuuuuaaccuugugcuucccuccuucccauggaauuucuucccacagaugaggggccacuggacagggggaugagagccauucgucccuggguugagggaauuaguaagacaucauuuucaccacuggguggucuucauacacaugaguuauaauagcauuuuaaguaucuuagcauuugugggauuagaaaucuguuuuugcuauuuauaauauauaaaaaaugaaacaugauagccuuucuugagauaaguaugugaaauguuuauuuuuaaaguuacuaaaugcaucuugucuugcuacuacaugcacugcucugaaaaagagccuuuaccaucuauaaccuguauuuuggguauucuuccuuuucauacuucuuugaaguuguggaaauauuaagucuuuuguuaaggcagagucucucucuccuguuacacaacacugaaccucacgccacugccucagugguuauuggccagccccagagguuagacuuggagcauaauucugaguggcagcuaaagguagcaggagucuuaccucucccuacuuuggguucagaauugagccagagaaaggaagccauuaaaaucuccagaauaaugauaguuaaugagcaaauguuaugcuuuauaucccacagacaucugccaccuuguucaucuuguccugaggaguaauaaauaauguuaagcuuuuaaaaaucugaauuucagagaaucuccuagaggauagaggaaaaccccucagcaugguguguuagagugggauugcugguuccucagccccgugccagguugcacagucuguguuucaguguucuugggagaguaguguacugcugggcauuaauuguuacucugcacuugcuccagaauguugguuaaauccuguaugaaaaaaagagauugcccuuauggaauaauuuccuguggcccagaggaauggccagaagaacauauguuggcacauguucuuccuucuaaaucuuuguggggcuuuuuuggcaaaauaaguuuuuuggcaguuaaaaaacgacugauguaggucacuuuaaaggaacuaggugauggauguuaagagacuuuauaauuuuucucagggaccugaaacuccaguuuggauaaaauucaauuaaaaaaacuuguuuuuucaguugcuagauuuguggauuucuaguaauuuaaucugccaggauuggggugggugggugaggaggagagagccacaaaaugauaagaguggagagcaguguggggcccucccuuugagggaaagccguuguuuacauaggguaguaggccuuguuggccugaucaagagaugaagccauaacucuuaaaaaccuucagaaccaaacgcugugucugcauccaucuguucucacuggcccucugcaugguaaaguuaugucaccacaaagugaugccgcauuacaaucacuuaugaaacuguuagcccuuaauaugcaucacauuugugguuccauagugcuucuccuuugcuuguuugaucucugcauggcagcaguggggcagcuccacauuuggagcccauacuccaaaaugcagagcacucuucucacagcacacauucccagccccaaacaaaaccugaauuuucaacucugcccauuaaagugaauguaucccuguaauuuaguauguacuuauuauggaaaauguaacaaaagcagagaagugugauuucauucuguaucacaaauugaagcaaaucaacagcaaauacagcaggauucagggaggcuuaguuuacaacguacuuuguaccuccugccauuuaaauuauuuugccacucugggcuucugaaaacacaaagggagauacagcccuccaacuaugagccuugcagagagagacaaaagggccagagguccugggauggaauacuagagcccccaccuucucaugcuuuggaaaccugccagagcacuuacccugcuggaagauuaaugaagacuccauucuguguuucauaacaguagcguuguuuucuccaucaaaaacagagggucacagaaaugugcugaaaagaugugguccagcaagguucuguagucccaccucagugccugugggcuguguuuuucacuguaacaagcacuuugguaaacuagaaagaagaaaggauucucaggauguguucaaagcuaagagaguuuucauuuuaaaaagaaacauacauacucacuccauggcuuuccucauccuccucucuuuuuucuucuuggugggaagcuggaugcggggcaugcaucauucuguauggacaaauguguuauguccacccauacucuugugacaacuccccagcacuguccugcaauaccaugcuggccuugguaguaaaacuccuugguaucugugagaguccucuuuuucuaugcccuagacuacuuuuaaccacuuuucucuggaaugacucccacauuugauuggguauugaaaggguaacagcaaaaugaugauuguggagauggaucuauuaggucucauguugguccccugguugaaacuauuuucaaaaaagccccaacaaugggccugucaccuugcuagucauccauucuuaaugccuauuuaucagcccucugaggucauauaaaaguugcaccuuuugaaccaaguauuuguauuuacuaugggaaagguguggacuacagauucguauuauauuauuuuaauggauuuuaaguaauuuauaaguaugcugaguguaaauuuuuuuuaaaaacuguauuauaaaaggacauauccugugaaauauaacauuuuacuguuuaauagaauaauucuauaagaagaaugauuuaucucacccacggaacugauuacauuccggaugcaaucaauaggcacuuuguaauuuccuuuuuguuuuuuuuucuuugcugggggguugagggguaacaagauaccaucuguauaaagugcaguuugugucacucaaaauaugcacuguauggcuacacaaaagcagcuugaugaacaaaucaccccauggcucauuaaagaacaaagcuuccagaagca

SEQ ID NO: 34 - RNA, 3373 nt, Homo sapiens
  ref <308>: DATABASE ACCESSION NUMBER: NM_022768.5
  ref <309>: DATABASE ENTRY DATE: 2022-07-18
  ref <313>: RELEVANT RESIDUES IN SEQ ID NO: (1)..(3373)
gagacuguagaagagagagcaauuggccaaugaggacugcggggcgggacccugugccgcggcggaguccaagauggcggcgugcgguuccgcugugugaaacgagcgcggggcggcggguuacucagcuccgcggagacgaccuccgacgacccgcaacaaugaagggaaaagagcgcucgccagugaaggccaaacgcucccgugguggugaggacucgacuucccgcggugagcggagcaagaaguuagggggcucugguggcagcaaugggagcagcagcggaaagaccgauagcggcggugggucgcggcggagucuccaccuggacaaguccagcagucgagguggcagccgcgaguaugauaccggugggggcagcuccaguagccgcuugcauaguuauagcuccccgagcaccaaaaauucuucgggcgggggcgaaucgcgcagcagcucccgggguggaggcggggagucacguuccucuggggccgccuccucagcucccggcggcggggacggcgcggaauacaagacucugaagauaagcgaguuggggucccagcuuagugacgaagcgguggaggacggccuguuucaugaguucaaacgcuucggugauguaagugugaaaaucagucaucugucggguucuggcagcggggaugagcggguagccuuugugaacuuccggcggccagaggacgcgcgggcggccaagcaugccagaggccgccuggugcucuaugaccggccucugaagauagaagcuguguaugugagccggcgccgcagccgcuccccuuuagacaaagauacuuauccuccaucagccaguguggucggggccucuguagguggucaccggcaccccccuggaggugguggaggccagagaucacuuuccccugguggcgcugcuuugggauacagagacuaccggcugcagcaguuggcucuuggccgccugcccccuccaccuccgccaccauugccucgagaccuggagagagaaagagacuacccguucuaugagagagugcgcccugcauacagucuugagccaagggugggagcuggagcaggugcugcuccuuucagagaaguggaugagauuucacccgaggaugaucagcgagcuaaccggacgcucuucuugggcaaccuagacaucacuguaacggagagugauuuaagaagggcguuugaucgcuuuggagucaucacagaaguagauaucaagaggccuucucgcggccagacuaguacuuacggcuuucucaaauuugagaacuuagauaugucucaccgggccaaauuagcaaugucuggcaaaauuauaauucggaauccuaucaaaauugguuaugguaaagcuacacccaccacccgccucugggugggaggccugggaccuuggguuccucuugcugcccuggcacgagaauuugaucgauuuggcaccauacgcaccauagacuaccgaaaaggugauaguugggcauauauccaguaugaaagccuggaugcagcgcaugcugccuggacccauaugcggggcuucccacuugguggcccagaucgacgccuuagaguagacuuugccgacaccgaacaucguuaccagcagcaguaucugcagccucugcccuugacucauuaugagcuggugacagaugcuuuuggacaucgggcaccagacccuuugaggggugcucgggauaggacaccacccuuacuauacagagaucgugauagggaccuuuauccugacucugauugggugccacccccacccccaguccgagaacgcagcacucggacugcagcuacuucugugccugcuuacgagccacuggauagccuagaucgcaggcgggaugguugguccuuggaccgggacagaggugaucgagaucugcccagcagcagagaccagccuaggaagcgaaggcugccugaggagaguggaggacgucaucuggauaggucuccugagagugaccgcccacgaaaacgucacugcgcuccuucuccugaccgcaguccagaauugagcaguagccgggaucguuacaacagcgacaaugaucgaucuucccgucuucucuuggaaaggcccucuccaaucagagacagacgagguaguuuggagaagagccagggugacaagcgagaccguaaaaacucugcaucagcugaacgagauaggaagcaccggacaacugcucccacugagggaaaaagcccucugaaaaaagaagaccgcucugaugggagugcaccuagcaccagcacugcuuccuccaagcugaaguccccgucccagaaacaggauggggggacagccccuguggcaucagccucucccaaacucuguuuggccuggcagggcaugcuucuacugaagaacagcaacuuuccuuccaacaugcaucuguugcagggugaccuccaaguggcuaguagucuucuuguggaggguucaacuggaggcaaaguggcccagcucaagaucacucagcgucuccguuuggaccagcccaaguuggaugaaguaacucgacgcaucaaaguagcagggcccaaugguuaugccauucuuuuggcugugccuggaaguucugacagccgguccuccucuuccucagcugcaucagacacugccacuucuacucagaggccacuuaggaaccuuguguccuauuuaaagcaaaagcaggcagccggggugaucagccucccuguggggggcaacaaagacaaggaaaacaccgggguccuucaugccuucccaccuugugaguucucccagcaguuccuggauuccccugccaaggcacuggccaaaucugaagaagauuaccuggucaugaucauuguccguggguuugguuuucagauaggaguuagguaugagaacaagaagagagaaaacuuggcgcugacccuguuauagugguuauaguggugucccuaaagggaggaaaugauuucagcaaaacugguugaacagcggaugaagauauggaauucaaagcucuaauggaccuuuuugaagagaaguuguggcuuauguggaguuuacaugggccucugauggaagaaagcuaaucuguuuaguauuugugcauuuuacuaaaauggcagcuuaaaguuguguaucugcuauugugaugccaaugccgguguuuuaaguggaaaaaaaaugaccucuuugauuugugcuguguacacaagauuucuggaaaaguaaagaaaaacccuuuuuauggcucacacagcuuaagaguagcugucucucaaacgugcgcucacaguugagcugcuuuuguuuuauucuaaauaaauuguuucuuuugaggaaaa

SEQ ID NO: 35 - RNA, 3225 nt, Homo sapiens
  ref <308>: DATABASE ACCESSION NUMBER: NM_002482.4
  ref <309>: DATABASE ENTRY DATE: 2022-01-23
  ref <313>: RELEVANT RESIDUES IN SEQ ID NO: (1)..(3225)
cucuaaucugccauuuucugucccugagugagucucuggcgucccaaauugccuguuuuucucgcaggcucuauuccguucgcugguucgccaccucaggggaacgauggccauggaguccacagccacugccgccgucgccgcggagcugguuucugccgacaaaauugaagauguuccugcuccuucuacaucugcagauaaaguggagagucuggauguggauagugaagcuaagaaacuauuggguuuaggacagaaacaucuggugaugggggauauuccagcagcugucaaugcauuccaggaagcagcuagucuuuuagguaagaaguauggagagacagcuaaugaguguggagaagccuucuuuuucuaugggaaaucacuucuggaguuggcaagaauggagaaugguguguugggaaacgccuuggaaggugugcauguggaagaggaagaaggagaaaaaacagaagaugaaucucugguagaaaauaaugauaacauagaugaggaagcaagggaagaguugagagaacagguuuaugacgccaugggagaaaaagaagaagccaaaaaaacagaagacaagucuuuggcaaagccugaaacugauaaagaacaggacagugaaauggagaaggguggaagagaagauauggauauaaguaaaucugcagaggagccacaggaaaaaguugacuugacucuagauugguuaacugaaaccucugaagaggcaaaaggaggagcagcaccagaaggaccgaaugaagcugaggucacuucugggaagccagaacaggaaguaccagaugcugaggaagaaaaaucaguuucuggaacugauguccaagaagagugcagagaaaaaggaggucaggagaagcagggagagguaauugugagcauagaggagaagccaaaagaaguuucagaagagcagccuguggugacucuagaaaagcagggcacugcaguggagguagaagcagagucuuuagacccgacagucaagccaguggauguggguggggacgagccagaggagaagguaguuaccucugaaaacgaggcaggaaaggcgguucuugaacaacugguaggucaagaaguaccaccugcugaagagucaccagaggugacaacagaggcugcagaggccucagcuguagaggcuggaucagaagucucugaaaagccugggcaggaggcuccaguucucccuaaggauggugcagucaauggaccgucaguuguaggagaucagacuccuauugaaccacagacuucuauagaaagacugacagaaacaaaagauggcucaggacuagaggagaaggucagggcaaagcugguuccuagucaggaggagacuaagcugucuguagaagagucugaggcagcuggagaugggguugauaccaagguagcccagggagcuacugagaaaucaccugaagacaaaguucagauagcugcuaaugaagagacacaagagagagaagaacagaugaaagagggugaagaaacugaaggcucagaagaggaugauaaagaaaaugauaagaccgaagaaaugccaaaugauucaguccuugaaaacaagucucuucaagaaaaugaggaggaggagauugggaaccuagagcuugccugggauaugcuggauuuagcaaagaucauuuuuaaaaggcaagaaacaaaagaagcacagcuuuaugcugcccaggcacaucuuaaacucggagaaguuaguguugaaucugaaaacuaugugcaagcuguggaggaguuccaguccugccuuaaccugcaggaacaguaccuggaagcccacgaccgucuccuugcagagacccacuaccagcugggcuuggcuuauggguacaacucucaguaugaugaggcaguggcacaguucagcaaaucuauugaagucauugagaacagaauggcuguacuaaacgagcaggugaaggaggcugaaggaucgucugcugaauacaagaaagaaauugaggaacuaaaggaacugcuacccgaaauuagagagaagauagaagaugcaaaggagucucagcguagugggaauguagcugaacuggcucugaaagcuacucugguggagaguucuacuucagguuucacuccugguggaggaggcucuucagucuccaugauugccaguagaaagccaacagacggugcuuccucaucaaauugugugacugauauuucccaccuugucagaaagaagaggaaaccagaggaagagaguccccggaaagaugaugcaaagaaagccaaacaagagccggaggugaacggaggcaguggggaugcuguccccaguggaaaugaaguuucggaaaacauggaggaggaggcugagaaucaggcugaaagccgggcagcaguggaggggacaguggaggcuggagcuacaguugaaagcacugcauguuaagagggggcacagcccuccucccaagggaaaguguuuuuguauauaauguauuuuuucacuuuuggaggauucuuuuuguauaacuucaauaaagauuguaagcaaagguugaggcuuugaugguuuuuuucuuaauuauuggcugaaucugccuuggagcacugcugguuuuauauauuagccaaagguuuuguucuggccuucuguacugaucuguguuccugauccuaauuccuaucugucuaacguggaggugaucaaguguggcuguaggccuuuguuuuccaauggugcuauauucuguuuucaaacacuucacugaacccagcugucuugcaaacuuucaguggugcugucccuggaugggggcuacaaaaacaagaauuggugaagaucuugcucuucagugcugaaaauggaugauggacuuuggcugugagccaggccuaggaugguucuuguccuauauccaccuagucuucaccuggggcuauaauucuguccuggaaaaagaacucugaaaaccugggucaggggaaugauuccuaaggaaaacggucugcauuugagcucugguuugaaaguagccaaggggacugaugguggacacuccagaugugguuggaagcauauguggggaggcuggcugguugaguuuuguuauuuucuguauagaaagguugagauauaucaacacuuggaauuguuacccaucugcagaauugacuucucaaauaaagaugcuaaaaauc

SEQ ID NO: 36 - RNA, 5233 nt, Homo sapiens
  ref <308>: DATABASE ACCESSION NUMBER: NM_032864.4
  ref <309>: DATABASE ENTRY DATE: 2022-05-22
  ref <313>: RELEVANT RESIDUES IN SEQ ID NO: (1)..(5233)
acacuacgguguuuccggcuucaagauggucgccuaagcuguuuagugaaacuucuuccaccuuucuccauuccucuaggugcuuuuucugaaccuggaugugaggcauuaaaggauccgacggaaauagaauugaaggcauucuaaaauggcuaaccguacagugaaggaugcgcacagcauccauggcaccaacccucaauaucugguggagaagaucauucgaacgcgaaucuaugaguccaaguacuggaaagaggagugcuuuggacuuacagcugaacuuguagucgauaaagccauggaguuaagguuuguggguggcgucuaugguggcaacauaaaaccaacacccuuucuguguuuaaccuugaagaugcuucaaauucaacccgagaaggauaucauuguagaguuuaucaaaaaugaagauuucaaguauguccgcaugcugggggcacuuuacaugaggcugacaggcacugcaauugauugcuacaaguacuuggaaccuuuguacaaugacuaucgaaaaaucaagagccagaaccgaaauggggaguuugaauugaugcauguugaugaguuuauugaugaacuauugcacagugagagagucugugauaucauucugccccgacuacagaaacgcuauguauuagaggaagcugagcaacuggagccucgaguuagugcucuggaagaggacauggaugauguggaguccagugaagaggaagaagaggaggaugagaaguuggaaagagugccaucaccugaucaccgccggagaagcuaccgagacuuggacaagccccgucgcucucccacacugcgcuacaggaggaguaggagccggucucccagaaggcggagucgaucucccaaaaggagaagccccuccccucgccgagaaaggcaucggagcaagaguccaagacgucaccgcagcaggucccgagaucggcggcacagaucccguuccaaguccccaggucaucaccguagucacagacacaggagccacucaaagucucccgaaaggucuaagaagagccacaagaagagccggagagggaaugaguaauggacucaguuugguuuuaguccacauggccuccuguggauauaaggauaucuguauguggaaggauuaagaucucccccaggcagcuauaagaauauuuuaguuuuuuucuuaucaaguuucucaaccuuuauuuuuaaugaaggaggugcugaguuuuguaucuuuuuaaucauaaucaacaucaguuuuugacccaacuaaccuugacuguauucaaacuuaugagaguauaaaggaucuggagguuggggauaugacugacaaggaaaggcuguggccaccugaugacccuuucccuuuuuauuaaaccggacacaccuguuucccauuucgcuguaguuuaguuuuugguuuguugugguuggaacugcuuugagaauccugggauuugugcugcugcuguuauucaaagaucaaaggaguaaaacauaguugcuccuaacuuuuuuccagcagcagcaagugguaauaaacaugaaaacugguuuguagcaguuuugaaagaauagaaugcauucaaauguaaggcugcuucuggaucauuaaagccaguuucaucaaacaguucaacagagagcagcacuuaauacccuuuauacagcccauuuuuucauaguuucauuuguucuugcccacaagcuugaaauccagguuaagguauccagccuuuaucauauaagcauugacauuauccaggccuagucaguagcaguaggguaacgggauugaaaaagauuugauggagaggaaaguaucuaauauuagucaugguuuugaccuaaauugcuagacagucgugccauucacaaagucagaaaauacagcaggaagagacagcuuuuagaggggcagagaauuagaggauggugguaguaaugaaaaugaugcauucaguuuaacaaguuuaauuugagacagcuaugguauagcuaaaaacaaaagcccauaaaguuggagauagggaccagaguuuaacauagcgaucuaggccagaauugacaauguuuaaguaaugguggaaucugucaauaagacuucccagaguguuaauauauaucagaaaugcacacaacagaaccauaggcaacaccaacacaggaagaaauagaagucaccuauugaaaacuggaauggcccauucagaaagacaggagaauuaggagagagugagugaaagaagccaagggucaagaaugcagaagcaaaggaaggacuaaaaagccuccacggacaucgauaaucaagagauuauuaagaucuuugcuagaagaguuccuuuaccuguacuuaacucccuuaaaaagagaaaagugauggaaugacuucugcaacuguagucccagcaggaacugugaagaccagcugcccugcaggauccaguuuuucuugggagguuguaucuggucauaagguaaacauucuauauauucuaugccugcucuagaauugaaagacuucagcaguauuaaagcauuuuuuaaucuuauuugagcuuucauauucuucaaugguuccuuucguaauaccugaguuaauaaaaguggucugaaagagcucuuacgguuucugauagaacuauauaggacuaccuuuuaagauuaaauauaagcaucuguacguaaauguauuacagugucauagcaguuauuucuuuaugaaagugaucuagguagaaagaaaugucuucuggcauggacagaaaugacccauggaccaaccaagcugucauccaguacacucuuaaaauaaaacuuugucuuaaaauauguuuuaaaguaaaaauaaacccacgcacaaagcccaauaaauuuuaguaacagaaauaaaccucccuaaacaaacauuaaaacauacuuguuuagacauaugguaauauguggaccaaaauugagguaccacagccaaucaauagcaggaaagagaaugucccuauucuuuaauuagaagaaggagguaagaguaagacauguugcaugucuguagauaucguuuugauaaaauuacacaggagauggagcuugaggacaaaccuugcuguaaggcucuuucugucaucuaugugcuugccuauggaugaagcauuuuaaacuuuuugacuacagcauacauuuuaaaaacguauuuuacaucuuaccucuguacacaucuguauucuguacugagacaaauuucaggaaagaauacuuacuaugcaaagugaauucugguguuuccuacuuguuaacaguuuuuaauauuggaugaaaccacuuaaucgauuucacuuccaauaauuugaaauacacugacuuaaaagcuuugccauagggcacaaucgccuuuuaauacuaagaaaguaagggagauguguuccaaggggcaaaagaaauaaugcauuuuuggccaggugcgguggcucacgccuguaaucccagcacuuugggaggccgaggcgggcagaucaccugaggucgggaguuugagaccagccuggccaacauggagaaacccugucucuacuaaaaaauacaaaauuagccgggcaugguggcgcaugccuguaaucccaguuacuugggaggcugaggcaggagaaucgcuugaaccugggaggcagaggaugcggugagcugagaucgugccacugcacuucagccugggcaaaaagagcgaaacuccaucucaaaaaaaaaaaaaaaagaagaagaagaauggguuuggugagaggguuccaauauuaacaacccaacuucaagcucauuccucuaaccucuuccuguuuguuauucuucaguagaccgaaguuuaaauaagcauuauuuagaaaagaacgaucacagauugcccacucccucaaaaagguuaaugugacagaaauucuccaccugcucaaaaaagacagcagacaugaucucuuuaaaaaaaaaaaguucaaaagagauuaagagaugaaaaauaagauugguggaguuuaaaggccuuauacuagaaaccacaaaacauaaaacagcacagagaaaauaggcuggaggaaauaaaaaauggaaaagagaaauauguagaauaaagaaauauucugaugguuuaaaaagaauuggccgggugcgguggcucacgccuguaaucccagcacuuugggaggccgaggcagacggaucaugaggucaggagaucgagaccauccaggcuaacacagugaaacccgucucuacuaaaaauacaaaaaauuagcugggugugcuggcggguagagcuacuagggaggcugaggcaggagaauggcgugaauccgggaggcggagcuuucagugagccgagaucgcgccauugcacuccagccugggcaacagagugagacuccgucucaaaaagaaaaaagaauuaaauggggucaggauggucucagaucuuauaacaagaaggcaaugaagcaaaaggcuccaaagguuugagaaaaagugccaggaauuuuauacuuugccaaaguugucuuaaaauacaaaggcuauagauguucucaaguuuguaagaacuuaaaaguacaaaucaugagucuuugggaaaaaaccgcccaauaaugaaauucaacuaaagaagaaaugaaucaaauuaaggacuuaggaacaaagaaucaguaaaaggaguguuaguaaacacuucagaaacuuuaaaaauaaugggcaaugauuauaaagucaaauauuaugaacacugucuauauagaaauaacaaguagagugugcugguuucaucuuucauagcaggaaucacguacagucucuaauuaaaacauacaugaagcuagaaaaaaaaauagccucuuuauuguuuuccguaauuguccuuaauuuuaggagaaaucuuacaagacuagauucucuuguggggaaaaaaguauuuaaauugagcauuuuuucaguuucacacuuguuuuccuuuuguuaauuugcuauauuuguuaaauauagcauaugagcccacuuuuguaaaacuauuuguauauaugugccuguaugauuaagagagaugguuggaaugaaugaaguucaccagauaguacuucuuucuggcugaugaacuuuugaaugauuuuuaaacuuuguacuuuuauauaaugcuuauuuucuuuuuacaauaaacauguuaauuucuacaaggaagaaa

SEQ ID NO: 37 - RNA, 4760 nt, Homo sapiens
  ref <308>: DATABASE ACCESSION NUMBER: NM_024097.4
  ref <309>: DATABASE ENTRY DATE: 2021-06-26
  ref <313>: RELEVANT RESIDUES IN SEQ ID NO: (1)..(4760)
gccuuuaugcgcaggcucuuccuacucgcacagcccagggaguggggaggauaaggcgcugucauggaggacgccgccgcgccggggcggaccgaggggguccuugaaaggcaaggagcgccgccagcugcaggccagggaggagcccugguggagcucaccccgacccccggcggccuggcccuggugagccccuaccacacccaccgggccggggaccccuuagaccucguggcgcucgcagagcaggugcagaaggcugaugaauucauccgagcaaaugccaccaacaagcugacagucauagcugagcaaauccaacauuugcaagaacaagccaggaagguacuggaagaugcucacagagaugccaaccugcaccauguagcuuguaauauagugaaaaaaccuggcaacauuuacuaucucuauaaacgggagaguggucagcaguauuuuuccaucauuucuccaaaggaaugggggacaaguuguccacaugacuuccuuggugccuacaaacuacagcaugacuuguccuggacuccguaugaggacauugagaagcaagaugcuaaaaucagcaugauggacacguugcuaagccagucaguggcccugccuccgugcacugaacccaacuuccagggacugacucacugagagugggcuuugacaaacagcucucacaggaccuggcugucaaccuccuuguugcccccacuguugccuugagaauugaagacauguaggugacucacaaacuucuuggaaagagacccugugugaauguaaaugcugucauuaugacuuuuaauugggaugggaauaaucauugagacagagucacugucuuucgggauccucuuuggaccacagauacccaagucagucaguuucagaguauuggccaguguacuuuccuucuuccucuccaucacuucuucccuuaaguuaaaguguuagagaagaccuucuucccauaaaaagguugguggaucguuuuccagagagaacuguuuucagucuuuuauauauauauauauauauauauaacugguaguauuuaacauugggguugaggcugucucacaguuggugcaacuccaguuuaaaauucaucugggaacgagaauguugaguaauggagggugauuuaaaaaacaacaacaacaacaaaaaaaaaaacacuuggcuuggcuggaccagagagugugugauucugggauuucugugcuguugaugagguuugucuugccccuuuugcucucucugccuugcuagagugacagaaucugaggcagcaaaggccagguuuggggagagucaacuuugaaacucacaaauuuuagacuuggaagagacauuggaugcugccacuaaucauuauagaguccacuguguagucucccaaguuggaaaucccaggcucagccaccugcagcagaacaaacccauuuaaauggcuacuuaacacuguggaagagaguaaggcaguagagugagaagcaagaaaaacagccaaauuguugacugugaguuaacucagauugaaauccauuuugaccagaauuauucaagaacuuaacaggguuuuggcaaaauuacuguaguucuuauucugacuaggcucuuguaggacucuaccuucuuuccuaucuguuugguggaauuagcuaaguuuauggauuauuucgcauggcuauaaucuauugcaauaugcuguaaaaggagggagagauguguacuugaacugacaauuggccaaauuucucucugggaaugauguuccugaugcugaucuguuugguaauacccuccccgggccgccccccgauucauggucuuuccucauaaacugcauuucuugugagcuguaagaggucuaccuaaggccuagcacaggauggggcuagaaucuaacaaacucacuccgaugauacuugguugcucgguucacaugggaaugcucuuuggacucagcugcagaggucuuuugcucaggugcugcagagccggauggaagagacauuagugguagaguaguggauucugacuacagaaaguuucccagguuuucagaagguucugaccugcaugaugaugacacauuuugaauaggugcuuuauuaguuacagauaaacaaauuacugcaaacuuaaugguuuaaagcaacaccuauuuauuucagagcuuacagguuaggaguucaggcaccauuucauugaguccucugcuucacaguuucucugaaacccuggaaggcugaaaucaaguuauuggucagggcuguucuuaucugaaggccugaguggggaaggaucugcuuccaagcucacuuaaauuguuagcagaauuucuuuucuuuccuuucuuuuuuuuugagacagagucucgcuguauugccaggccugaguacaguggcucaaucucagcuuaccgcaaccuccgccuccugguucaagugauucuccugccucagccuccucaguagcugagauuacaggcgcacgccaccaugcccagcuaauuuuuuuauuuuuaguagagacaggguuucaccauguuggccaggagggucucuaucucuugaccucaugauccaccugccucagccucccacagugcuggcguuugagccaccauaccuggccuguuggcagaauuucaauuccacaaggguugcuggcuuccgggguuccacuagcugacuguuggcuggagguugcccucacuuccuugccacacgagccucuauaacauggcagcuuucuuaagcaaaguaaacaaggauuagagucugcuagccagauggaaguuacagucucauguagucugaucacagaagugacauuucaucaccuuugccguaaucuuuugguuggaagcaagucacuaggcuagcccauauucaagggagggaguuauacaaaggcauguauaccagaaggcagacaucauugaaucauugaacagauccuagaaucuaucugccacagugcucaguugagaagaagaaaggaaaauucuagguaaaaaaggcaauauuacuaagugguuaagugugaugaugucaaagucacagaucugcacuugacugucaacucugacaagacagccucccagcuggcaucccugcuucccccucuguccccucuccaauccauucugcaugcagucaucagcuugaucauuugaaaaugccaaaucucccucacucuugacauuuccuaaccuaaagagugggagaguagugauugaaaagagagaaacugccuucuguucaguaagauguacucccugcacugaccaccaaggggugucauugcucuuuccugguuaagccaguuaccacacuugggaagggcuaguggcaaugugaggugauuuggggcaccucagaaugggaaacuaggacccagugauuauauacaguaaucacuuuucauuuacccauccaucuguucaccugauaauuucauuaguguuaagugccuucuagguuccagguucuggugaauaauggagcugaauaagaaaucacaauuuccagggagcucacaguauaguagggaaggaaacaaguuaacaauuacaagaugcucuaagaaggggugugauagagguacgcauggaauacuauggagcuccggggagggguuccuuacugagcuuggagaauuugggaagacacuuggaggagcugaugccugcuugguguugaaaaccauacaaguuagaacuuucuggaggcuccaauugcccuuucauaggaaguuugucuuuuggggguugggguaggguucauuuuuggauggaguuuauuugugaaugaagaaagugacaaccauuuaagccauaucaaccaaagcaguccuggcaaugaggagggguggagugagauauguccuggcuagaaaauccuuguguuuaaaagcuaggagauaguugaagacagggaggaaaaaacauuccagacaaaaaauaacauggaagggacauugaccagaauugacacgacauuuggggaaucgcagccaguuaaacaggagaauauggacuggggaauaugagagaagaacuaauggaaggauuuggggaggcugguaauauuggcagcagaguuuggguagagggaagacaggagauuccaucccuaaucaaggcaagggaugaugggagucugaauuaagcaguaguagaaugagagggccuguauuucaccaauuacaagguguacuucccccccccccccccccacauuuuggugucugucaaacuuggacacuucuaaaagucaauggcaugucacaguuuaaguggcauuuuuucuuucuuagagcuacauaaaagaaucauaacaagcugggcgcaguggcucacgccuguaaucccagcacuuugggcggcugaggcgggcggaucacgaggucaggagauugagaccauucuggcuaacauagugaaauccugucucuacuaaaaauacaaaaaauuagccgggcgugguggcgggcgccuguaaucccagcuacucgggaggcugaggcaggagaaugucgugaaccugggaggcgaagcuugcagugagccgagauugcaccacugcacuccaacuugggcaacagagcaagacuccaucucaaaaaaaaaaaaaaaagaaucauaacauagcaucuuacauuugaugaagcauggcacauugaaaguacaaucgauugccaucuuguuaauaaaaugucagagggauuagaggaaa

SEQ ID NO: 38 - RNA, 1668 nt, Homo sapiens
  ref <308>: DATABASE ACCESSION NUMBER: NM_004813.4
  ref <309>: DATABASE ENTRY DATE: 2022-06-09
  ref <313>: RELEVANT RESIDUES IN SEQ ID NO: (1)..(1668)
gcaggaagcagggcgccgcagccugucguacgguccuucugugggucugucggugccgagggcaggauggagaagcugcggcuccugggccuccgcuaccaggaguacgugacucgucacccggccgccacggcccagcuggagacagcagugcggggcuucaguuaccugcuggcaggucgauucgccgauucgcacgagcugucagagcugguguacucugccucuaaccugcuugugcugcucaaugacgggauccuacggaaggagcuucggaaaaaguugccugugucgcugucccagcagaagcugcugacauggcugagcgugcuggagugcguggagguguucauggagaugggagcugccaagguguggggugaagugggccgcuggcuugucaucgcccucguccagcuggccaaggcuguacugcggaugcuccugcugcucugguucaaggcuggccuccagacuucacccccuaucguuccacuggacagagagacccaggcacagcccccggauggugaccacagcccuggcaaccaugagcaguccuacguggggaagcggucaaaccggguggugcgaacccuccagaacacgccgucccugcacuccaggcacuggggagcuccccagcagcgggagggacggcagcagcagcaucacgaggagcugagugcgacccccaccccccuggggcugcaggagaccaucgcagaguuuuuguacauugcccggccgcugcugcacuugcucagccugggccuguggggucagaggucguggaaacccuggcucuuggcugguguuguggacgugaccagccugagccuccugagugacagaaagggccugacccggagggagcggcgggagcugcggcgccggaccauccugcugcucuacuaccugcugcgcucuccuuucuaugaccgcuucuccgaggccaggauccucuuccugcuccaguugcuggccgaccacgucccuggcguuggccuggucacaaggccgcucauggauuacuugcccaccuggcagaaaaucuacuucuacaguuggggcugacagaccucccggaaggaggguguggggagggguggggcagggagccccucuucccuaauaaaacugacuccggcagcgucccagcgugcggccucuccgugccuaccggccaggccccacacacagcccuggucgccaccagcguuccucccaggacacccuugacugcgcucuccugugacgaugccacugcagcccgcaccuugucacugcugggccaagaagccuucacuaggagugggauccaggcuccucucccacagaaagcggugacuucaccucauggagcccgggaagcugcucgccucggcagccauaggagcgaacacugcugcucucucgcuggccccuggugaggacaggaagccugaacccgggugauggcugaacgcugcccagcgugucuucuggcuggggcccuccgucugccccuucuccgcagggcccuguggcucuggcagccccaggccauggcguugccagccucccugugacagagccuggugaacagugagccuggcucccacgcaaguggcacuuuaagcccugcauccucgguugagaguaaaaggcuuuucucccuuagaaaaa

SEQ ID NO: 39 - RNA, 3311 nt, Homo sapiens
  ref <308>: DATABASE ACCESSION NUMBER: NM_004220.3
  ref <309>: DATABASE ENTRY DATE: 2022-04-17
  ref <313>: RELEVANT RESIDUES IN SEQ ID NO: (1)..(3311)
gaggacuuccggcgccgggagcucgcggcggaagugggaucuccugggccguagugggcguuguguguuucgggggcgggggcgggggcgggggccggggcggggacggggccucuggccgccuggcuccaacaucaagcaccgggcuccgaguggccgggaucagcgccccgaggcagaggccggagggcgcgcgcacugcuaggaagugcuggucccccgcgccgcucugccagcuuggucccccggcagacgccccuguacgaucgccgcucgccccgcgggcgaggcugcgguggacagcgcggggcuccggcuggcucgccuucccgccugccguguccugcugagcgacccuggaguacacauccagaugccagcccagcuaccacaggggaucccucugggagacugaaaguacagguucuggggcccagguugaagccgaccaacccugagccucaggccaggggaauggcagcccccuuggaggcccaggaccaggccccuggggagggagaagggcuucugauugugaaaguggaagauuccuccugggaacaggaaucugcccagcaugaggauggcagggauuccgaagccugccgccagcgcuuccggcaauucugcuacggggaugugcaugggccucaugaggccuucagccagcucugggagcucugcugccgcuggcugcggcccgagcugcguaccaaggagcagauccuggagcugcuggugcuggagcaguuccugacagugcugccaggggagauccagggcugggugcgugagcagcacccgggaagcggugaggaggcugucgccuugguggaggaccuacagaagcagccagugaaagccuggcgacaggaugugcccucggaggaggcggaacccgaggcugcaggccggggaucccaggccacggggccucccccgacggugggggcacggaggcggccgucuguuccccaggagcagcacagccauagcgcccagccuccugcucuucuuaaagagggucgucccggagagacgacggacaccugcuuugucucugggguccauggaccuguggcauugggagacaucccauucuauuucucccgggaagaauggggcacccuggacccugcucagcgggaucucuucugggacauaaagcgggagaacucccggaacaccacccuggguuuugggcucaaaggccaaagugagaagucccugcugcaggagauggugccgguggugccaggccagacaggcagcgacgugacuguguccuggagccccgaggaggcugaggccugggagagcgagaaccggccgagggcggcccugggcccaguggugggcgcgcgacgggggcggccacccacucgccggcgccaguuccgggaccuggcagccgagaagccgcacagcugcgggcaguguggaaagcgcuuccgcuggggcucggaccuggcgcggcaccagcgcacgcacacgggcgagaagccacacaagugcccugagugcgacaagagcuuccgcagcuccucggaccuggugcgccaccaaggcgugcacacgggcgagaagcccuucuccuguuccgagugcggcaagagcuucagccgcagcgccuaccuggccgaccaccagcgcauacacacgggcgagaagccuuucggcugcagcgacugcggcaagagcuucucgcugcgcuccuaccugcuggaccaucggcgugugcacaccggugagcggcccuucggcugcggagagugcgacaagagcuucaagcagcgcgcgcaccucaucgcgcaucagagccugcacgccaagauggcccagcccguggggugagcagcuggcuuggccggaaacccgggggaggcccagccacggcacauccugcuuuguucaccacugggacucuccuuccaucuguggccaccucccgggcuguccgagggaccccaggguaccucacacucggagcucgccugcccugcuuggcucugaggaccugcccagcgcucaaagggaacggaagccuuccccucccgcccccgaucuuguccucuuucccccuucugcgccuagcguuccucuuccccucuaguuuccuggagccccaacacauuccuggcagggacagcaggguggcaaggacucaggucuaggucccuucccagaagcccccgagccucauuugacuguguggcucuuuggcccccacccugugggguggguccaugggucaggccucugcccuaccaaccugugccuuucagugggcguggaggacuggccuuggccccccagggggcugcuggacuuugggagagacagcccacaccugugggaccgcgggucuuagucacggcggcaggggcuuucuggcccccucccacucccguuuccaggccaugaccacucugcccuguccuggccauacggacucggccugccuuugcccucggccuacuugcccuagcaugaggcucugagagccaccugcccaccaaucuggugaggauaaugguggcuccagcgacaggaggccaacccuggagaccaagaacagggcgccuggcugccaucuuuuccuccagagguggggcugcaccagacucagcacuagcacuccaucagcacuagcaccucacuccaucagcacuagcaccucacuccaucggccccggcacccugcuccaucggcacuggcgcccugcuccaucggcacuaaugcuccacucggcgccccacuccaucggccccgcuccaucggcacuaaugccccacucggcgccccacuccaucagcacuaaugcuccacuccauuggcacuaacgccccaacuccagcggcacuaaugacccgcuccuuugacauuggugccccacuccaucagcacuaacgcccugcuccaucggcacuggugucccacuccauugucacuaacguccggcuccaucggcacuaccaccccgcuccaucaucacuauguccagcuccgucggcacuaccacccugcuccaucaucacuacguccagcuccaacggcacuggugccccauuccaucggcacuaacgccccgcuccaccggcaccagugccucgcuccauuggcaccaacgcccagcuccaccgguacuggcucccugcuccaucggcacuaacgcccugcuucauuggcacuuugcugcugccuccugagcacugccuuccaugaacagggacagaccagaggcccugcaaggacucccccucagaccuccaaagggcaacagaagaguauuaauaaacgugaaaacuua

SEQ ID NO: 40 - RNA, 7177 nt, Homo sapiens
  ref <308>: DATABASE ACCESSION NUMBER: NM_015322.5
  ref <309>: DATABASE ENTRY DATE: 2021-11-25
  ref <313>: RELEVANT RESIDUES IN SEQ ID NO: (1)..(7177)
cucuccucccggcccugucugcgaaagcucgucuuccuccccgcccaaguuccggcgccgcucuugcgggagcguuccgcaucgccccgggggccccuacgcgaggaucuccggggccguuggcagcgccuggcacuucugagcucggcggacgagagccugcagguuuccuccagucagggacggcgaagggcggaaagcgcaggaggaggcaguguuaggccuuaggccgggugggccgggucaggagagacgcgcccaucuuucgccauccggggugcgcgagguccucucgggacccggccggcgacccguagcucgggcacgcgccugucgcaucccgcaggaaggagggguccggccugaggcccggggcggcguccgccauggagauccccucgguccagggccggcgccugggaccuggcgggcggcccugaccgccuuccucccugcgcgggcugggucgcggacgugcccuucgcggcacucggccuccucugcgucuccgccuucccugggccgcacugcugccugggcgcggcggcggcgacggcgcccuguugaaugggcugugagggcccagguuuaaagcgcuggcgaacgcggccuccgggggcgcacggcagcugcagcgguggcgaccaaacggguguuggaguuggcggcggccauggagggccuggcuggcuauguauacaaggcggccagcgagggcaaggugcugacucuggccgccuugcuucucaaccggucugaaagcgacauccgcuaucugcuuggcuaugucagccagcagggagggcagcgcuccacgccccucaucaucgcagcccgcaauggacacgcaaaggugguacgcuugcucuuagaacauuaccgggugcagacucagcagacuggcaccguccgcuucgacggguaugucauugauggugccacugcucuuuggugugcagcuggagcaggacauuuugaaguuguuaaacuucuagucagccauggagccaacgugaaccauaccacaguaacuaauucaaccccccugcgggcagcaugcuuugauggcagacuggacauugugaaauacuugguugaaaauaaugccaacaucagcauugccaacaaauaugacaacaccugccuaaugauugcggcauauaagggacacacugauguggucagauaccuuuuagaacaacgugcugaucccaaugccaaagcacauuguggagccacagcauugcacuuugcagcugaagcugggcacauagauauugugaaagagcugauaaaauggcgugcugcuauaguagugaauggccaugggaugacgccauugaaaguagcugccgaaagcuguaaagcugaugucguagaacuguuacucucucaugcugauugcgaccgaagaagucggauugaagcuuuggaacucuugggugccuccuuugcaaaugaccgugagaacuaugacaucauaaagacauaccacuaucuauauuuagccauguuagagagguuccaagauggugauaacauucucgaaaaagagguucuuccaccaauccaugcuuaugggaauagaacugaauguagaaauccucaggaacuggaguccauucggcaagacagagaugcucuucauauggaaggccuuauaguucgggaacggauuuuaggugcugacaauauugauguuucucaucccaucauuuacagaggagcuguuuaugcggauaauauggaauuugagcaguguaucaaguuguggcuucaugcccugcaccucagacaaaaagguaacaggaacacccacaaggaucuucuucgauuugcucaaguuuucucacaaaugauacauuugaaugaaacugugaaggccccagacauagaauguguuuugagaugcaguguuuuggaaauagaacaaaguaugaacagagugaaaaauauuucagaugcugauguccacaaugcuauggacaauuaugaauguaaucucuauaccuuucuguauuuagugugcaucucuaccaaaacacagugcagcgaagaagaucagugcaaaauuaacaagcagaucuacaaccugauucaccuugaucccagaacucgugaagguuucaccuugcugcaucuggcugucaauuccaauacuccaguugaugauuuccacaccaaugacgucugcagcuuuccaaaugcacuugucacaaagcuccugcuggacuguggugcugaggugaaugccguggacaaugagggaaacagugcccuucauauuaucguucaguacaacaggcccaucagugauuuuuugaccuugcacuccaucaucauuagccuaguugaagccggagcucacacugacaugacgaauaaacagaauaagacuccgcuagacaaaaguacaacugggguaucugaaauacugcuuaaaacucaaaugaagaugagucucaagugccuggcugcccgagcaguucgggcuaaugacauuaacuaccaagaccagauccccagaacucuugaagaguuuguuggauuucauuaagugacuggauauguaaagucguuuaauguggugcuaaaaaguaaaggacuuuuaaucacagacaguagaauuauguguucauaaauucugcuuuucuuuccacuacccuuccucccaucccauccuuccuuaguucuguauuuguuuuucuugccucaugguaauugauuucagacagacuuuaacaaaaccacauuguuuugguguaacuauaagguauuugcauauugguuaccuauuugucuuucuuuuuuuuaaaggaacagauauaaaauguuuuguuuauguaacaagggacauuuauaauuucaaguugauaauguuuuaaacagcugcuuacaaaaguauuucuguuaagccuaugucagcauguuauccaugcagcaguuuugaggauuuuaugaagaaaaagagcuaaaaaggaacauuaagaggaaugggauauccagguguucugcacaugccaaacugcuguagauaguuuacacucuuccauuauuuauacggagugaugcagcacauuuuagcauucaggaggauuuuuaaaaaauagcugcagauuaaucuggaaaaugugcuaauuuaauaauaguuacaaauuuauaaauuuaaauccauuugaaauuguugcauuaugcuggguaguauauacaaaaugguuaucaucuuaaaccaacuuuucagagaaucuugauggacucugccuuuaggcuugaauucuucaaagucuauuuuaaugaaauuuaucuaaauugcagcagucuauuugauucagcucauagacauguaaaaauuaugaaugcuguuuucuuaugaaacaaauugucacaguguaguuauacauucuauuuuuguccccuuuucccuuuuucuccuguaucuuuuaaaauuuggaaacuacuuuuccagaaggcauuauuuaugccucccuaauaagguauuuuacuuaugacagaugaaaaggaaccaggauauguuugaauuuuuucacuuucuuagucugugacaagaaguagaaauaucacuagugugguauaggaacuuacauguuuuuuaugaugaaaauaauucucaaugccacuugaaagguaauugugucugagagcugcaaauuuuucaaccacaaaaugucacuuauuccuacaggcuauacagaggucuuuaugguuuuuuuguuuuguuuuaauggcaacauuguaacugucaaacuaaaaggguauucugugauuaucuuuuaagcauuacagaaauucaagugaaaguuauaugcuuauuucuauugauguuaaaaaugauaaugaaagcaaaauuagcuguaucuguaauuuucucucuagugccaaaugaaugccuuagcuacucauagugcaugguacuguaagugaagaccuguagcuuuuuuuuuuucuuuaaugaaaagcauuauaaugauguagcagcaucagauauaaacuuaaaaaaaaagguuucaauuaacauuuuauauauggauaaugcuuuguaaaguguaagagaaagguugcaguuggaucaguauaaaacaaugaccaagccaaaaucagcacccuagggccuuaaauaaaauagagauaccccacaaaaugaaauauuuugaaggaugggaggggacagaaggggggacuaucccccaaggaugcaagauacuuuuuacaguugcuaucauuauacuuugucuucuugcuucaguuaggaagguuuugaugguaaauucugucaugguaauagauaccuauuuuccuaaccugagauuuuacaaggaagguuuuuagguuggcuuaaauggagguauuuauuugagugagcccuaauuugaaaauuaccagacuugauccuccugguaugaaucaaggaaauacuguaccuugcucuuagcaacucucaguggccugaaagagaaaaugcacuucuucaaggugagugaaucaugcuuuuuccacaguucagaucagacccuuuauuuuucaaacuacaaggccugcuuauuggacagucuaaggaauagcuuugauacuugguauucaguugguuccauuauuaauguauauuauuuuuauuuaaacaucaaaacuuaaaauaucagauaauguugcauguuuuaaaaacacauuucaucccuuuggcuacccaggaccagcacuauuuaagacacugcuuauuuauaaaaugagaauaaugauuauauguacauauucagacaucaaaauuuaaugacauucuauuuugagaagggaagacaaugcugaagaaaguaaaacuguugauugaaaugcugacaggguggagaacgaauuugaagacaguggaauaaacuaugaacuaugcuaaugguaauuagauuuuuuguuuguuuaguauagugguaaauuguguaguaucuugcugagaaucuaauucuaucuuguaaaaucaauccaaauguguauugugaaacaccuguauaaaaucauuuuugaaauaagugaucuacaugccuguuuuuuuaaaauguuuuugguagaauuguuugaaaauauguuuagccagacccuuccccaacuuaauuuuuuuuuaaaaaaggaaaauagguaagaaaaugauaguucuauaucucagcccucugugaacucuaggcugucuccauuuugcagccacuuuggucauuugauguuuacuauugguauacuuaugcuuugaauuguaaaucuucauugaugguauuacagauucaaccaaaacuucucuguccuguucaguaauuguuauauaaaucuggaugagcuagggaggcccagguggauguuuucauucgcaaaucaugagaaacuuaaguggguuuuaugcacuugauagaguuggcaaaauugaacuaugaaguuaacuauuuaacucaaggaaugggcggcaaacccauccccucgauugauaaagaaggggaacauuuuuacauuagaacugacacugaaaacauagcuuuuucaguccacccugguugcucuaguagcccacagcccaaucacguuaagguucuuugcugugggaauuuuaaauaaaccaaaccccaaagcagaccaucuguaagcuuuggucugcuuguuucugagaaggguuuuauuucauuauacuaauaguggacuaauaauugguaauugugagaacuuagguaugauaacauuguuugaaguaaaauaugauuugggggcagcagcuuucuaaauaccaacucuguuugacaaauguuuugaaaauuaaaauuucaagugagcaacacccuguuaagaguuuucacuauaguugaggcagcuacuuuaugaauaagaccacuuuggguuauuuaagcagaagcguuucuuuuuuuuuuuuggaauggggugggaugaggagugaguugccagaccuuugauuaguuugcugguuuagaaacagccaguggcugaauuagugaguaaaugaaugaaaguauaaaggacuuguuuuuuaugauagauuuucuguaagaaucuuaaauguuccuuuucaaauuagauguguugaugcacacaugacuauucuguuuuucucacugacuauauaacauuaaaaagggguuaaagaaaacaaaacucugccuuuugugcuaugaaauauuuuuaguccagagguuuuaagcuguguguccauuccuacucugaaaaugcauagcuuuguucuggaugucaucucuugaaaguagaaaacuccuauguguuuaucacauugcagggcuuucuuauguauuucuggcagacuugcccaaaucuuuagaugggcuggguuauacagcaugcccucccccaaauaagggaucugaaauaaauacuacacuauugauaguggagauauauuaauuuuuaaaacuguaaaguaaauguggucucuagguuugugguguguaccuuuguguuaauguguagggaagagacagugacuugaugguuauggggaguguaucuugauguguguauagggguaaguauugcuaaauuauuuacagcuuuuauucagggugagucaugugaugaauggccuaaucagaaaagugaaggagcgaagaugcaagcuugccaaaugaugaaaugaacaagauuuuguaucuauuuuuuaucagguguuguaaaauuugugcauggcuuuuuguuguuguugcuuaguaacugguagaggagaaaagaugaggaaagaaacucagcuuuccucacagucuuuucaaagguacacaguuggggaguaaaaucugacuggccuaaucgauggaaaagacccuguccuuuucaccccauccugcaauccuccgugcagaggaacuacacuguuguauucuaguaauucacugugauuuauaacaaaccggugaugucauucuauugugcacuuuugucaaaccauuuaugugacuuuaauaaacauaguaaacuugcugacugcaccagagguccauuagugauuuauauauugcaugacauuuucuauuugaguuugacauguagagucauuuuuaguuucauggcaauugacaguccuaauaacucagcuaauuugaaacuaacaaucuugcuguguaaaaggaaaaaaugguguuuguguucaguaaauguuugaaaaaaa

SEQ ID NO: 41 - RNA, 2306 nt, Homo sapiens
  ref <308>: DATABASE ACCESSION NUMBER: NM_000538.4
  ref <309>: DATABASE ENTRY DATE: 2022-06-09
  ref <313>: RELEVANT RESIDUES IN SEQ ID NO: (1)..(2306)
aggcgcagucggggcgccuucccgguauaggcgccuuuuaccccagcguguccugagucuuugguucgcgaagugccguuaggccaagcaggugcuaaaagcccggggucguggaccccggccaggucuuagcagcauggaggcgcaggguguagcggagggcgcggggccgggcgccgccagcggcgugccccaccccgcggcccuagccccggcugcggcucccaccuuggcgccagccucgguggcggccgcggccucucaauucacccugcuagugaugcaacccugugcugggcaggacgaggcugcggcccccgggggcagcguuggggcgggcaagcccguuagguaccugugcgaaggggccggggauggcgaagaggaggcuggggaggacgaggcggaccuguuagacacuucggacccuccggggggaggcgagagcgcggcuaguuuggaggaucuagaggacgaggagacucacucggggggcgagggcagcagcgggggcgcccggaggcggggcagcggugggggcagcaugagcaagaccugcaccuacgaaggcugcagcgagaccacgagccagguggccaagcagcgcaaaccguggaugugcaagaaacaccgcaacaagauguacaaggacaaguauaaaaagaagaagagcgaccaggcccugaacugcggugggacugccucgacuggcagcgcgggaaacgucaaacucgaggaaagugcagauaacauacucuccauuguuaaacaaagaacaggaucuuuuggggaucguccugcaagaccuacucuuuuagaacaaguguuaaaucaaaaaagacugucguuacuaagaaguccagaaguagugcaauuuuuacagaaacagcaacagcuauuaaaucagcaaguuuuggagcaaagacaacagcaguuuccaggaacaucaaugugagggaacuuaccaagaacaucuacaugguuuuuuaucuuauuguaauagaugagcauauuuuuuuaccagacauaaaugggguaauaaucuaugccuguagaacauaaacauuuuccuguaaauguaugugugcauuuggggauaaguaaguauugcacuuugugcaucuaaucuuucagauuacugugaguuugaagaagucagcuuaucuuuccaaauaacauuuaauuauaauguuuuuuaaaaaauauauuccucuucagucauuguuacugaagguaaugaagcaguuacuuucuguggaagucauaaaguuaauagauauuaaucuugacucaucuagcucagugguucucaucaagggucaauuugauugucauagugaccuugaaaaccacuggcuuuuagugaguggccaggaaugcuaaauguucugcagugucagggguagucccacauacuaaagauugucucacccgcagugccaauaacacuccuaagaaauguugauggcuauuuuguggugcuaacauguaguuggggcaccuauaauuggguucucuuaauaaccuuucuuugcaguuaagacugaagcugucaaagagguaagcacauuuuauauagacguaaggaaagugauuauuguuuaauaucugugaauuuaggaugugcaucucuuuucagagguguguuaguaaaaccugacggauuaacuaagcacacugggaugugucuccuacaguuggcuucucucuuugauguuaccuguuagugcugaucucuuaaagcagacauuucuuguuuguugaauuugugaacaguauagaucucagcccaccaaugccaagacaaaauuauuuuucuuauacuuauuuuuuauuaaacaaaaugaaaaagauccuuuucaaaaaggugauccugaaaauaaaacuaacacuccaguauuuugucauuguuuuucgcaauugagcuaucugaaaacuguuauuccuaaguaauguucaaaaaugauaaguaaucuggauaccuuuuucuuauacuuucuccuaggaaaacuuuaaaacuuuaaaaaggcaaaccuaccaauaggaauaacaaauuaaaugucaagagaguauauccaauauuaggauauaaauguaugugucucaaguuuaacucuacaaaaauuuguuacuuguuuuuuaaacucuauauauaaaguucgacuuaaucauggcuguucuaagaaguacuuauggagagcaagaacguuuuuguucauuucuuaauguguguguuuuuacuugcauaucuguucaaaacacuuuuaacaaaauuaauucauuaaaguccaguuguugaccuuuga

SEQ ID NO: 42 - RNA, 2318 nt, Homo sapiens
  ref <308>: DATABASE ACCESSION NUMBER: NM_005870.4
  ref <309>: DATABASE ENTRY DATE: 2021-04-10
  ref <313>: RELEVANT RESIDUES IN SEQ ID NO: (1)..(2318)
uaaagugucgagcuucuccucgcgagagacuuagugcucaugcucgcugcaggggucggaggucagggcgagcgucucgcaggccguaggaggaagauggcgguggagucgcgcguuacccaggaggaaauuaagaaggagccagagaaaccgaucgaccgcgagaagacaugcccacuguugcuacgggucuucaccaccaauaacggccgccaccaccgaauggacgaguucucccggggaaauguaccguccagcgaguugcagaucuacacuuggauggaugcaaccuugaaagaacugacaagcuuaguaaaagaagucuacccagaagcuagaaagaagggcacucacuucaauuuugcaaucguuuuuacagauguuaaaagaccuggcuaucgaguuaaggagauuggcagcaccaugucuggcagaaaggggacugaugauuccaugacccugcagucgcagaaguuccagauaggagauuacuuggacauagcaauuaccccuccaaaucgggcaccaccuccuucagggcgcaugagaccauauuaaauucuauuuacuauuuguugaauuuauuuuuccgucaguuauguaaaauaaacauacucuucuuccuccccugauuauugccauuaagccuuuaaauucuaaacaaauuauaaugcaucaucuauuuaggaguuagauuuggaugugcuauuguaugauuacgaauagucuguauguuucaagcccuucuguaaaauaugaagaaaagugcucuuagcauucuguguaaaacuguacuguuaaauauauguguguaaucagccugagugugaaaguuaauggaggccugggaacaggguuuauacuaaguaguggaggagcguaugguuaagcccaaaggggcagaacaagaaagccacgggauuguaugaaaucaaacaugaguuugugguguagacugcuguaugcaguuggaaaggacacgugagagugagcucaaguggcagcagaggcaguuuggaauaagcugcggcaaguugaagaagcuguguuggacgagauugccccgccuugaucaggguaccaugacauaaaagguuaaagaacaggcaacacaaugagcacuuaaguuuuuaacauguggggaauagggcauuuuaaaggcuggaaccaguucagaggaaacaaggguuuggguagagguagaaagguuuaauuaacccuggagaucugcaaauggagggaggggugaaggaggaaucuuaagaccgagggagaagagccaaggacaaggucagguugaaugaguaggagguguucacacauccucuugugugcuagguaguggcgugcaucugaucugagaggcaguaaagcagugaugaagacagugcagcagaacauugaaguccugauugcagcagugacucugcaaccuuggauaaauuauuucaaucuuuauuuuguaaaacggagauaacggccuauaggauuguuaagacugaauauuuuaaaagagcuuagaauggugccugacaaaugagaaacaauaaauacugauuauuggccauaaagugguauuaaaacuggaucucuuuucuuccauggcacuacucucauuuugaaguuuaugaacaguacgacaguguauuguaaauucccuuuaguuuguuacaguagccaauugaaaauguauaucccaaaauuuaaaaaaaaaaguaccagcagcaauaaaauugcacuaguuuuaucugguacauacaaaaacagccauuuaaaaucugaugauucuguggaauuggugucccuguuagcaauggcagagaccagccuggagccuagaucuggugcuucuucugugcugugguuuaccccaaaccuuuagguuguuuauucauucagauuagauagacuggagccauaaaguuaauuugcaccuagcuuuuuggagaauagccaugauuaacugcuauucguggugggggugggggggaacccuaugauuuacuaugcagaugaagaggguaggaacuaaauaaaggacuuuguaagccaaagugguugggcugcuuugucaaauuccacccuuugcuugguuccaacauggaauuucuccuuuaaagauuucaauaucccaucaaaaguacaguuagagaauaccuagugaauuuauugaauauaaugcuaagaaucaaaagacuuguacuaguuuaccucuggcuuugguaaguguggcuuugaguaauaacugagcuguuuccccgcauauaaaauagaaauaaauaaguagucaucaugacagcaaaaaaaaaaaaaaaaaa

SEQ ID NO: 43 - RNA, 4798 nt, Homo sapiens
  ref <308>: DATABASE ACCESSION NUMBER: NM_020745.4
  ref <309>: DATABASE ENTRY DATE: 2022-06-03
  ref <313>: RELEVANT RESIDUES IN SEQ ID NO: (1)..(4798)
guaaagucacugcccggagcuacgauggcagcgucaguggcagcugcagcccggaggcugcggcgggccauucgaaggucgcccgcauggcggggccucagccaucggccgcucucaucggagcccccugcagccaaggccucggccgugagggccgccuuucugaacuucuuucgggaccgccauggccaccggcuggugcccuccgcuuccgugcggccccgcggcgaccccaguuugcuuuuugucaaugcgggcaugaaccaguucaagccaaucuuucugggcaccguggauccacgaagcgagauggcaggcuuccgacguguggccaacagccagaaaugugugagagcuggaggacaccauaacgaccuggaagaugugggucgagaccuuucccaucauaccuucuuugaaaugcuuggcaauugggccuuugggggugaauauuuuaaggaggaggcuuguaacauggccugggaacugcugacucaggucuaugggaucccugaggaaaggcucuggaucuccuacuuugauggugaccccaaggcagggcuggacccagaccuggagaccagggacaucuggcugagcuuaggggugccugcuagccgugugcuuuccuuuggaccacaagagaacuucugggagaugggggauacuggcccuugugggcccuguacugagauccacuacgaccuugcugguggggugggagccccccagcugguagagcuuuggaaccuggucuucaugcaacacaacagagaggcagauggaagccugcagccccugccccagcggcauguggacacaggaaugggccuggaaaggcugguggcugugcugcaaggcaaacacuccaccuaugacacugaccucuuuuccccgcugcucaacgccauacagcagggcugcagggcacccccuuacuugggccgaguagggguggcagacgaggggcgcacagacacagcguaccgcgugguggcugaccacauccgcacacucagugucugcaucucugauggcaucuucccugggaugucaggucccccgcugguucuucgucggauccugcgucgagcugugcguuucuccauggagaucuuaaaggcaccaccuggcuuccuaggcagccugguaccuguagugguggagacacugggagaugcuuauccagaacugcaaaggaacucagcccagaucgccaaccuggugucagaggacgaggcagccuuccuggccucccuggagcgggguaggcggaucauugaucggacucugaggacccuggggccuucagauauguucccugcugaaguggccugguccuugucacuguguggagaccugggacuccccuuggacaugguagagcugaugcuggaggagaaagggguccagcuagacuccgcuggacuggagcgguuggcccaagaggaggcccagcaccgggcacggcaggcugagccaguucagaagcagggauuguggcuugauguccaugcgcuuggggagcugcagcgccaaggagugcccccaacugacgacagccccaaguacaacuacucccugcgacccagcggaaguuaugaguucggcaccugugaggcccagguguugcaacuguauacagaggacgggacagcaguggccuccguggggaaaggccagcgcuguggccuccucuuggacaggaccaacuucuacgcagaacaggggggccaggcuucagaccguggcuaccuggugcgggcagggcaagaggacgugcuguucccaguagcccgggcccaggucuguggagguuucauccugcaugaggcaguagccccugagugccugcgguuaggggaccaggugcagcugcauguggaugaggccuggcgucuaggcugcauggcgaagcauacggccacccaccugcugaacugggcacugaggcagacccugggcccuggcacagagcagcagggcucccaucucaauccugagcagcugcgcuuggaugugaccacccagaccccauugaccccagagcagcuccgggcaguggagaacacugugcaggaggccguggggcaggaugaggcuguguacauggaggaggugccccuggcgcucacugcccaggucccuggccugcgcucucuggaugagguuuacccagacccugugcgggugguaucagugggggugcccguggcccaugcauuggacccagccucccaagccgcacugcagaccucuguggagcuaugcugugggacgcaccuguuacguacuggggcuguaggggaccugguuaucaucggggaccgccagcuuuccaagggcacuacccgccugcuggccgucacuggggagcaggcccagcaggcccgagagcuaggccagagccuggcccaggaagugaaagcggccacugagcggcugagucuggggagccgggauguggcggaggcacugaggcuguccaaggacauaggacgacucauugaagcuguggaaacugcugugaugccccaguggcagcggcgggagcugcuggccacagugaagaugcugcagcggcgugccaacacugccauccguaagcugcaaaugggacaggcugcaaagaaaacucaggagcugcuggagcggcacucgaaggggccucugauuguggacacagucucugcugagucucucucagugcuggugaaggugguacggcagcugugugagcaggcccccagcacgucugugcuccuacucagcccccagcccauggggaaggugcugugugccugucagguggcccagggugccaugcccaccuucacagcagaggccugggcacuggcagugugcagccacauggggggcaaggcguggggcucacgagugguggcccaaggcaccggaagcacuacugaccuggaagcugcccucaguauagcccaaaccuaugcccucagccagcucugacccagguccgcccagggacccacauggacuaaaggcaaugccaggagcccugaaggagccagcagaaccuucccacaugcugaagcgaggacuagaggcuggaggccaagcagcugagccaaagaagaucaccugggccagggccuacgugacacaaggagacaugggcuagggaaggcagaacugggcccaaagacacacacccccuuggugaaagagaagacauggacaauuauggcugugacugcacauuagaaauuauuucacuggccggguguggugguucacaccuguaauccaagcacuuugggaggccgaggcagguagaucaugaggucaggagaucaaaaccauccuggcuaacauggugaaaccccaucucuacuaaaaauacaaaaaauuagccgggugugguggcacacgccuguagucccagcuacucgggaggcugaggcagaagaaucgcuugaaccugggagguggagguugcagugggccaagaucgcaccacugcacuccagccugggugacagagcgagacucugucucaaauaauuauuucacagaggauagcaagcuccagaagguucuauuaccuuuaaaaaagaguaguuuugccccucccagcaaccuaagaccagucugaacucacacugucugaaaggccacccaggauccaaaucgggccaucuucauugccaugaguguucggacaggcucugcgugccaggcaacagugggcagacaaagggcucccuggcuacugggaagauacagaaccauccugugaaaaucauuucagcacuggagaccuucuuugcuauuacuucuuucauuaacaaugaccucuaauuuaaauugggcccugcugguuuguugaugaauugagcaacugagaacguggcgggcacuaaacugacaguuccuuccuucauggagccugcuucauguggaggaaacaggaaauaaagcaaaagaagagggucaggcuugguggcucaugccuauaauccuggcauuuuaggaggcugagguuggagaaucgcuugaggccagaagugcaagaccagccugcgcaaacagcaagacccugucuacaaaaaaaaaaaaaaguaaucuuuaugccucagccucccagguagcugagacuacccacaccuuggucccagcuacuugggaggcugaggugggaaaaucacuuugcccaggaauucaaggccgcagugagcuaugauugcaccacugcacuccaggcaacagagugagacccugucuuaaaaaaaagaagggagaaagugucagauggugaugaggucugggggggaaauagagaauggggaucaggaguguggauggugauauucccucaccaagaggugacaugugagcagggagcugggaggugagggugugacccguguggaaaucagggaaaagcauuccagccugagggaacagccaaugcagaggccgugagguggccagugccacugagcagugagcugggauaggggcaggugaggaggcuggagagcggggucagacaaacaauaugcuuauuaaaacaaguuguacagcaccugccucaagccugagcugaagcugaaaauuggcacuucaaagcaggaggaagcagagauggaggaaagcccuccuucccuaccagaaugaguccaccucagacagccagacuuuggaccucugccuuuguuccuagaucucuguugcuagaugaaggaaagggccucaggggauccaucuucaaaucaagggcucccuggcuaccaaauucugucuaguaaaaauuauucgagca

SEQ ID NO: 44 - RNA, 4040 nt, Homo sapiens
  ref <308>: DATABASE ACCESSION NUMBER: NM_018715.4
  ref <309>: DATABASE ENTRY DATE: 2022-06-01
  ref <313>: RELEVANT RESIDUES IN SEQ ID NO: (1)..(4040)
guauuuacauuuuaaguguaucuggugagugggcuggagcccucgucugggccggaaaaaaaaaagcccuccgauccgucuuuuaguugcuucucuuccuuuuuucucuccgguuucucaucacuccaaccagccgcgaccaugcccaggaagaaggcggcggcggcggccugggaggagccgagcucgggcaacggcacugcccgcgccgggcccaggaaacgcggcggcccggcgggcaggaagcgcgagcggcccgagcgcugcaguagcagcagcggcggcggcagcagcggcgacgaggacggccuggagcucgacggggcccccggcgggggcaagcgcgcggcgcggccggcgacagcaggcaaggcgggcggcgcggccguggucaucaccgaacccgagcacaccaaggagcgcgucaaacuugaagggucaaagugcaaagggcagcuuuugauuuuuggggcaaccaacugggacuugauuggucgaaaagaagugccuaaacagcaagcugcuuaccgcaaucucggucagaauuugugggggccccacagauaugggugccuggcggggguccgggugcggacaguggucucgggcucgugugcugcacacagccuccucaucaccacggaagggaagcuguggagcuggggucgaaaugagaaggggcagcugggacauggugacaccaagagaguagaagccccuagacucaucgagggucuuagccacgaagugauugugucugcagcaugugggcggaaccacaccuuggccuugacggaaacgggcuccguguuugcguuuggggaaaacaagauggggcagcugggccuuggcaaccagacagacgcuguucccagccccgcgcagauaauguacaacggccagccaauuaccaaaauggccuguggggcugaauucaguaugauaauggacugcaaaggaaaccucuauuccuuugggugcccugaauauggucagcugggacacaacucagaugggaaguucaucgcccgggcacagcggauagaguacgacugugaacuaguuccccggcgaguggccaucuucauugagaagacgaaagauggacagauucugccuguaccaaacgugguuguacgagacguggccuguggcgcuaaccacacgcugguccuggacucccagaagcgagucuucuccuggggcuuugguggcuauggccggcugggccacgcagagcagaaggaugagauggucccccgccuggugaagcuguuugacuucccugggcguggggcuucccagaucuaugcugguuacaccugcuccuuugcugucagugaaguggguggucuguuuuucuggggggccaccaacaccucccgugaaucuaccauguacccaaaagcagugcaggaccucugcggcuggagaauccggagccuggcuugugggaagagcagcaucauuguggccgccgaugagagcaccaucagcugggguccgucaccgaccuuuggggaacugggcuacggggaccacaagcccaagucuuccacugcagcccaggagguaaagacucuggauggcauuuucucagagcaggucgccaugggcuacucacacuccuuggugauagcaagagaugaaagugagacugagaaagagaagaucaagaaacugccagaauacaacccccgaacccucugaugcucccggagacuccuccgacuccacaccucucgcggcagcugucauuuccaugugcacugggacgggaagucaaacgaggaauuuaaaaaagcaaaaguugaccgaaggugcauuuuuguuuagacucccugagguuccguuuuacacaugauccaacguuaacuaccuuuuuuucuguaugcuuuccaaaguccuuuuuuuucccuuaauguugaauuaaaauacuugcucauaguugauuuaccauuccuacaaaagaggcagaaacuuugagcaaucuagguuuuuuuuuuuuuuaaguuuuuucuuucuuccucuccugaauacacuccccaaaacaccccuuuccaguuacaauuagcaucgugauccaagcagaugccacauggaagaggaaucgccauuuacucagaaaaaaugucccuuacaggaaccggcagcagcuaggcagucaccggcccgccuccauccaaaaucacgcucgcgugcuucggaagcauccgggucacuccuucuccgcuuuuucuugcagaugggccuaggccggugucgguucuguuucuccccuuggcugccuguacgcccacagccuucuggcugcgacauuauagaaucggccguguccccccuggugggggauuggggaucuguguuuagccauuuauaucuacuuuagcuguuaaagagguccaaaugaaaaucaggugauuguggaaccauggggacuuggggguggggcagaggugggaacauuuguaucaguugagucagcuugguggcucccuguggagccagggcugagccuugucacgcgcacucgccaauuaagagauggaccagccagcagucaagugcauucuccaguccuugcaagaaggaucagcccuuucugugccagccucgaucgccuugugcuuuggucucuuuuucucccccccgccuggauccugccucgcgcgggccguccuguugcugagacucgggguaccguucugcugacccagcucccuuuagucacguuugcuuggcucugguaccaaauaguugggauuaccgaagaguccccuuccuugcgugucagcacggaugcugugacugccaccugcguccucgucaagugcccgagcucgccgccgugugugcugcgcugagugaguuaugaggugccuuucccggaacccuccucucgccuggacccaagagaggcgacagcuguggcuggggcucuugguuuccagagggucuggacugguuugggugcuuuaaaauagauauuuaguucaguggugcuuaugggggagaugggacuagaacuuaagugugagacuuggguggaugggaaaguuaaauauuggucucuucaaguuuuuuuuuucuuuugcuuuguuaccacuugucacugucuccauguuaaaaugccaaaaaugauguaguuguuguugcuuuuuucccuauuuuccaccccagucgcuccuuaccgugacuccugcccuuggagggcauguagcagugucuguccugccagucccaaggcccugugggaggagacuggccugcaucucucuaagacuuagucugacgccacgcgcaucucuuguucuguguucaaucaguaguccaggggagaagcuucugcuacuucagagcuuugcuaaacuaaccuaauuuguccaaaucaccccaaaaccaccaucucugacguaagcuuccaugcgacagccugauccguuucccuggacaggucucuuuccuggaaugcagcccaggcaccugugcuccuggcacccuugaggucucuccuuugagccguggucaccgagaggguugaggacgcagcacccgaggucccagccuuugcaggagccucccugggcuuagcuggacuuagaucuucgguggccucauguaaacguggcagccagccucuucuagaacccuagcccagggacuggagcaggaaagggaccuucaaagugaagacugccuugucccgcagcuccuucuggcuuagauugaaaaaugggcuuccuaauggguuaaauccuuuaaaacaaggaguugugggggaagggugucgugcacuccuagagaaagguacacaguugcccgguugggaaugugcuuggcgcugacccugcgggcaucugacuggucuuccagcucaggaaaaagaauuugaaagaggcuuagcgugaaggggaaucaaagaggagguugugauuuggucgaaggugccugguuuagugcuguaauugucuuauuauuuuuuuuauauauauauuucuuggaguaaacauuuuaaauaaacaacaacauugucuacuguc

SEQ ID NO: 45 - RNA, 3117 nt, Homo sapiens
  ref <308>: DATABASE ACCESSION NUMBER: NM_003680.3
  ref <309>: DATABASE ENTRY DATE: 2020-05-24
  ref <313>: RELEVANT RESIDUES IN SEQ ID NO: (1)..(3117)
gcaacagccucccgccgagccggccgggacagaggcggcccccacccccacugggcaggggacugcagacggcgcuccccgacucucaagacgaccagggagcuucccuuuuccucagcggggugaggggcaucugucgcccgccgacuuggagguggaggggcagggacauucccucgccaucaccucaggagcuagaacgcgcccuucucgcagcagggcgugggccucccaaucccuccaaggggcggacggcgugcgcaacccccuggagaagcaacaggcgcccccaucccccuccaagccggguaacgccgacccccucgucccucccgcucuucaccuccggaagccgggccaaagccugggcgaacgacugcgccucacuccgcccccugcgccauuuuaucgcccccuccccgaccuccccuaccccgagucagccgggccaacaccagggggagggcaaaccagcagugauuggcaggaaaccgucccgccucuaggaggguugugccccgcucaggcgccucagccccgcccuccgcaccgcccaccgugcccacuggcuccaccuuuucccgucucaucgaccugcucgacucucugcugcuccucacuuuuuccggccgcugccggagggguccaggccgaguaagcggagcgccgagcccagcugaugcaaccuggcuggacucgcgugacaguucccggcacgcggcggcgacggugacccaggaaggggcucuggugccgggcugagcgggggaagcagggguagcggagccaugggggacgcucccagcccugaagagaaacugcaccuuaucacccggaaccugcaggagguucugggggaagagaagcugaaggagauacugaaggagcgggaacuuaaaauuuacuggggaacggcaaccacgggcaaaccacauguggcuuacuuugugcccaugucaaagauugcagacuucuuaaaggcagggugugagguaacaauucuguuugcggaccuccacgcauaccuggauaacaugaaagccccaugggaacuucuagaacuccgagucaguuacuaugagaaugugaucaaagcaaugcuggagagcauuggugugcccuuggagaagcucaaguucaucaaaggcacugauuaccagcucagcaaagaguacacacuagauguguacagacucuccuccguggucacacagcacgauuccaagaaggcuggagcugaggugguaaagcagguggagcacccuuugcugaguggccucuuauaccccggacugcaggcuuuggaugaagaguauuuaaaaguagaugcccaauuuggaggcauugaucagagaaagauuuucaccuuugcagagaaguaccucccugcacuuggcuauucaaaacggguccaucugaugaauccuaugguuccaggauuaacaggcagcaaaaugagcucuucagaagaggaguccaagauugaucuccuugaucggaaggaggaugugaagaaaaaacugaagaaggccuucugugagccaggaaauguggagaacaaugggguucuguccuucaucaagcauguccuuuuuccccuuaaguccgaguuugugauccuacgagaugagaaaugggguggaaacaaaaccuacacagcuuacguggaccuggaaaaggacuuugcugcugagguuguacauccuggagaccugaagaauucuguugaagucgcacugaacaaguugcuggauccaauccgggaaaaguuuaauaccccugcccugaaaaaacuggccagcgcugccuacccagaucccucaaagcagaagccaauggccaaaggcccugccaagaauucagaaccagaggaggucaucccaucccggcuggauauccguguggggaaaaucaucacuguggagaagcacccagaugcagacagccuguauguagagaagauugacgugggggaagcugaaccacggacuguggugagcggccugguacaguucgugcccaaggaggaacugcaggacaggcugguaguggugcugugcaaccugaaaccccagaagaugagaggagucgagucccaaggcaugcuucugugugcuucuauagaagggauaaaccgccagguugaaccucuggacccuccggcaggcucugcuccuggugagcacguguuugugaagggcuaugaaaagggccaaccagaugaggagcucaagcccaagaagaaagucuucgagaaguugcaggcugacuucaaaauuucugaggagugcaucgcacaguggaagcaaaccaacuucaugaccaagcugggcuccauuuccuguaaaucgcugaaaggggggaacauuagcuagccagcccagcaucuuccccccuucuuccaccacugagucaucugcugucucuucagucugcuccauccaucacccauuuacccaucucucaggacacggaagcagcggguuuggacucuuuauucggugcagaacucggcaaggggcagcuuacccuccccagaacccaggaucauccugucuggcugcagugagagaccaaccccuaacaagggcugggccacagcagggaguccagcccuaccuucuucccuuggcagcuggagaaaucugguuucaauauaacucauuuaaaaauuuaugccacaguccuuauaauuggaaaaauacuggugcccagguuuucuuggaguuauccaagcagcugcgccccuagcugggaucugguaccuggacuaggcuaauuacagcuucuccccaacaggaaacugugggauuugaaaaggaaagggaagggaaaacagagaaccuaguggucuaccaagugguuggcaacuuucccaaugucugcuuacucugaggcuuggcacugggggccagggccugccccagggcuccuggaauuucccuugauccagcuaggcugggacacucccuaaaucagcugcguguuguuagcaucaggcagaaugaauggcagagagugauucugucuucauagagggugggguacuucuccauaaggcaucucagucaaauccccaucacugucauaaauucaaauaaaaugucugaacaaggg

SEQ ID NO: 46 - RNA, 3397 nt, Homo sapiens
  ref <308>: DATABASE ACCESSION NUMBER: NM_005676.5
  ref <309>: DATABASE ENTRY DATE: 2022-08-07
  ref <313>: RELEVANT RESIDUES IN SEQ ID NO: (1)..(3397)
aguagguggaugguggucggagcgccgacucccuucucgucgucgccauuuugagcuggugacuguggccggcugggaguaggcggcagugaguuucccugggagggcagcgcgcuuggcgcuucuccccuccccccgaucugccuccagucucggacuugguuguugcgcgcuccggcuccggcugagcugggagaguuggaggagguggcggcgggcagaggugaugucugggagcccuuccuugacagcccgggccgagaagagucccugcaggaagcaucacccaggcuggcagaucaugguagcagcagcggggguggcugggaagugaaacggagccagcggcugaggaggggccccagcagcccccgaaggcccuaucaggacauggaguaugaaagacgugguggucguggugacaggacuggccgcuauggagccacugaccgcucgcaggaugaugguggggagaaccgcagccgagaccacgacuaccgggacauggacuaccguucauauccucgcgaguauggcagccaggagggcaagcaugacuaugacgacucaucugaggagcagagugcggaggauuccuacgaggccuccccgggcuccgagacucagcguaggcggcggcggcggcacaggcacagccccaccggcccgccaggcuucccccgagacggcgacuaucgggaccaggacuaucggaccgagcaaggggaggaggaggaggaggaggaggaugaggaggaggaggagaaggccaguaacaucgucaugcugaggaugcugccacaggcagccacugaggaugacauccguggccagcugcagucgcacggcgugcaagcacgggagguucggcugaugcggaacaaaucuucaggucagagccggggcuucgccuucgucgaguuuagucacuugcaggacgcuacacgauggauggaagccaaucagcacucccucaacauccugggccagaaggugucgaugcacuacagugaccccaagcccaagaucaaugaggacuggcugugcaauaaguguggcguccagaacuucaaacgccgagagaagugcuucaaauguggcgugcccaagucagaggcagagcagaagcugccccucggcacgaggcuggaucagcagacacugccacuggguggccgggagcugagccagggccugcuuccccugccgcagcccuaccaggcccagggaguccuggccucccaagcccugucacagggcucggagccaagcucagagaacgccaaugacaccaucauuuugcgcaaccugaacccacacagcaccauggauuccauccugggggcccuggcacccuacgcggugcuguccuccuccaacgugcgcgucauaaaggacaagcagacccaacugaaccgcggcuuugccuucauccagcucuccaccaucguggaggcagcccagcugcugcagauccugcaggcccugcacccaccacucacuaucgacggcaagaccaucaauguugaguuugccaaggguucuaagagggacauggccuccaaugaaggcagucgcaucagugcugccucuguggccagcacugccauugcugcggcccagugggccaucucacaggccucccaagguggggaggguaccugggccaccuccgaggagccgccggucgacuacagcuacuaccaacaggaugagggcuauggcaacagccagggcacagagucuucccucuaugcccauggcuaccucaagggcaccaagggcccuggcaucacuggaaccaaaggggaucccacuggagcaggucccgaggccucccuagagccuggggccgacucugugucgaugcaggcuuucucucgcgcccagccuggugcugcuccuggcaucuaccaacaaucagccgaggcgagcaguagccagggcacugcugccaacagccagucguauaccaucaugucacccgcugugcucaaaucugagcuccagagcccuacccauccuaguucugcucucccaccggcuaccagccccacugcccaggaauccuacagccaguacccuguucccgacgucucuaccuaccaguacgaugagaccuccggcuacuacuaugacccccagaccggccucuacuaugaccccaacucccaguauuacuacaaugcucagagccagcaguaccuguacugggauggggagaggcggaccuauguucccgcccuggagcagucggccgacggacauaaggagacaggggcacccucgaaggagggcaaagagaagaaggagaagcacaagaccaagacagcucaacagauugccaaggacauggaacgcugggcccgcagucucaacaaacaaaaagaaaacuucaaaaauagcuuccagccuaucagcucccugcgagaugacgagaggcgggagucagccacugcagaugcuggcuaugccauccucgagaagaagggagcacuagccgagagacagcacaccagcauggaucucccgaaauuggccagugacgaccgcccaagcccuccgcgaggacugguggcagccuacagcggggagagugacagugaggaggagcaggagcgugggggcccugagcgggaggagaagcucaccgacuggcagaagcuggccugucugcucugccgacgccaguuccccagcaaagaggcgcucauccggcaccagcagcucucagggcuccacaagcaaaaccuugagauucaccggcgagcccacuugucagaaaacgagcuagaagcacuagagaagaaugacauggagcaaaugaaguaccgggaccgugcagcugaacgcagagaaaaguauggcauccccgagccgccagagcccaagaggaggaaguacggcggcauauccacagccucuguagacuucgagcagccuacucgggacgggcugggcagugacaacauuggcagucggaugcugcaggccaugggcuggaaagagggcagcggccugggccgcaagaagcagggcauuguaacgccuaucgaggcccaaacacgggugcggggcuccggccugggugcacggggcagcuccuacggggucaccucaaccgaguccuacaaggagacacugcacaagacaauggugacccgcuucaacgaggcccagugagcagcuucaagagcaacuucuccacauguuggguguccauccuggggcagggaaggacagaguguuggauggcugggacggggccuugcucuugucggccagcccacuccccagccagagagggcuugaccaaaucaaauugagguggugacuuuuguuggaaaauugggcugggaucacguccuguuuuguaauaaaagcugaaaagucugca

SEQ ID NO: 47 - RNA, 1028 nt, Homo sapiens
  ref <308>: DATABASE ACCESSION NUMBER: NM_000969.5
  ref <309>: DATABASE ENTRY DATE: 2022-08-11
  ref <313>: RELEVANT RESIDUES IN SEQ ID NO: (1)..(1028)
ccuuuucccacccccuagcgccgcugggccugcaggucucugucgagcagcggacgccggucucuguuccgcaggaugggguuuguuaaaguuguuaagaauaaggccuacuuuaagagauaccaagugaaauuuagaagacgacgagaggguaaaacugauuauuaugcucggaaacgcuuggugauacaagauaaaaauaaauacaacacacccaaauacaggaugauaguucgugugacaaacagagauaucauuugucagauugcuuaugcccguauagagggggauaugauagucugcgcagcguaugcacacgaacugccaaaauauggugugaagguuggccugacaaauuaugcugcagcauauuguacuggccugcugcuggcccgcaggcuucucaauagguuuggcauggacaagaucuaugaaggccaaguggaggugacuggugaugaauacaauguggaaagcauugauggucagccaggugccuucaccugcuauuuggaugcaggccuugccagaacuaccacuggcaauaaaguuuuuggugcccugaagggagcuguggauggaggcuugucuaucccucacaguaccaaacgauucccugguuaugauucugaaagcaaggaauuuaaugcagaaguacaucggaagcacaucaugggccagaauguugcagauuacaugcgcuacuuaauggaagaagaugaagaugcuuacaagaaacaguucucucaauacauaaagaacagcguaacuccagacaugauggaggagauguauaagaaagcucaugcugcuauacgagagaauccagucuaugaaaagaagcccaagaaagaaguuaaaaagaagagguggaaccgucccaaaaugucccuugcucagaagaaggaucggguagcucaaaagaaggcaagcuuccucagagcucaggagcgggcugcugagagcuaaacccagcaauuuucuaugauuuuuucagauauagauaauaaacuuaugaacagcaacua

SEQ ID NO: 48 - RNA, 1299 nt, Homo sapiens
  ref <308>: DATABASE ACCESSION NUMBER: NM_014205.4
  ref <309>: DATABASE ENTRY DATE: 2022-06-27
  ref <313>: RELEVANT RESIDUES IN SEQ ID NO: (1)..(1299)
gaacuaccaccggcgugcguucgcagguagaucgcggugccaguugccauggagccggccgggcccuguggcuucugcccggcgggggagguccagccagcgcguuacaccugcccucgcuguaaugcgcccuacugcucgcugcgcugcuaccggacgcauggcaccugcgcagaaaacuucuaccgugaccaggugcugggagagcuccgcgguugcagcgcuccucccagccgccuagcaagcgcccuacgccggcugcgucagcaacgcgagaccgaggacgagccuggggaagcaggccucagcuccggcccggcgcccggcggccuaucgggacucugggagcggcuggcgccgggcgaaaaagcugccuucgagcggcugcugagccgcggugaggccgggcggcugcugccuccauggcggccguggugguggaaccgcggagccggaccgcagcuucuggaggagcuggauaaugccccggguagugacgccgcggagcuggagcucgccccugcgaggaccccgccggauucugugaaagaugccuccgccgcggagcccgcggccgccgagcggguucuuggagaugucccgggggccugcacgcccgucguacccacccgcauccccgcgauagucagccugagccgcggcccagucucgccgcucgugcgcuuccagcugcccaaugugcuguucgccuacgcgcauacucucgcccuguaucacggcggugacgacgcgcugcucucugacuucugugccacacugcucggcguuuccggagcccugggugcccagcaagucuucgccucugcggaagaagcccugcaggcggcagcccacgugcuggaagcaggcgagcacccgccggggccccugggcacacgaggggcuaugcacgaggucgcccgcauccugcugggcgagggcccgaccaaccagaaaggcuacacgcuggcagcacugggggaccuggcacagacccugggccgugcccggaaacaggccguggcuagagaagagcgagaucaucuuuaccgggcccggaaaaagugccaguuccuccuggccuggaccaacgaaaaugaggcggcccucacaccccuggcccuagacugcgccagggcucaccaagcccaugcugugguagccgaggagguggccgcccucacuggggagcuggagcggcuuuggggaggccccgugccaccugccccaagaacucucauugaggagcucccuagcugaacggguuacccuggucauuaauaaagcugugacugguca

SEQ ID NO: 49 - RNA, 4230 nt, Homo sapiens
  ref <308>: DATABASE ACCESSION NUMBER: NM_001126111.3
  ref <309>: DATABASE ENTRY DATE: 2021-02-16
  ref <313>: RELEVANT RESIDUES IN SEQ ID NO: (1)..(4230)
cuccggcgccuggcucccugcaggcgaggagcggaagccgcuaucuggaggcggacuccggcgccacagagccggggcagccgcggcaacggcgaggcgcgaggcaggggcgcccggcagaccccgcgaccccgagccagcgggcgccccgagcgggcagccucgccgggggaggcggaggcggccacggcggccgcgcucgggcgccccucgcgcagcgcuccaugcccguguggugcugccgcugcucccuggccggucauuucagaaacuauagugacacugaaacugaaggagagauuuuuaauuccuuagugcaauacuuuggugacaacuuggggcgaaaaguuaaagcgaugccauuaguugaagaaacuucuuuacuggaagauucgucagugacuuuuccugugguaauaauaggaaauggacccucaggaauaugccuuucuuauauguuaucaggcuacagaccguauuuaucaucagaagcaauacacccaaauacaaucuuaaauaguaaauuagaagaagcaagacaucuuuccauuguugaucaggacuuagaauacuugucugagggccuugagggccgaucauccaauccaguugcaguacuuuucgauacacuucuucauccagaugcugacuuuggguaugauuauccauccguuuugcauuggaaauuagagcaacaucauuauaucccucacguaguucuugguaaagguccaccugguggggcuuggcauaauauggaaggcuccauguugacaaucagcuuuggaaguuggauggaacuaccuggacuuaaauuuaaggacuggguaucaaguaaacgaaggagccuaaaaggggaucgaguuaugccagaggaaauagcucgcuacuauaaacauuauguaaaagucaugggucuucagaagaauuucagagagaauacuuacauaacuuccguaucaagacucuacagagaucaagaugaugaugauauucaagacagagauauuucaacaaagcauuuacagauagagaagucaaacuuuaucaagagaaacugggaaauuagggguuaucagcgaauagcugaugguucucauguucccuucugccucuuugcugagaauguagcgcuggcaacuggaacgcuggauucuccugcccaucuggaaauugaaggggaagauuuuccuuuuguguuucauucaaugccugaauuuggagcugcuauaaacaaaggaaaguugcguggcaaaguggauccaguguuaauuguagguucugggcuuacugccgcugacgcaguacugugugcuuacaacaguaauaucccugugauucauguguuucgcagacgaguaacugauccaagcuuaauuuucaaacagcuucccaaaaagcuguauccugaauaucauaaagucuaucauaugauguguacucagucauauucuguagacucaaaucuuuuaucugauuauaccagcuuucccgagcaccgugugcuuuccuuuaagucggacaugaaauguguucuccaaagcguuucuggauugaagaaaauauuuaagcugucugcagcaguaguauugauagguucucauccuaaucugucuuuucugaaggaucaaggguguuaccuaggccauaagucaagccagccaaucacauguaaggguaauccuguggaaauagauacauauaccuaugaguguauuaaagaagccaaccuuuuugcauuggguccuuugguuggagacaauuuuguucgauuuuuaaagggaggggcgcuggguguuacacgcuguuuagcuacaagacagaagaaaaagcauuuguuuguugaaagaggaggaggagaugggauagcuuaaagcaaguuuacaaguaauuaaaauggacaguuugccauuaaagauuuuuaauagugguuuugcaguguacuggcuugaauuuucuggacuugaguuaacugaaggagagccucaaacuauaguaacuucauuuuuaaaaguuacuagaauuugguauccugauuuauauugcaguguuucaaaggugucacugucagacaaauagaaacacugccaacuugguguaacuuaagcuuucauuuaacuaaaacauucuuuucuugcaaaacuuauuuuucaugaucauuuuugguuauuuauuauacuugauuccaaaauaguacagccuugaaucuauaaaacugugcagucauuaugccagaaauuaucuuaaauauauaaugggucaccuugcuguucaaaggguggugcaagguccugcagcaucuuacaucuguagcuuguuagaaauguaaacucucaggccccacaacuuacuuccugcauuuuaacaagauccccaagggauauguaugcucauaaaaauuugagacacugguuuaaaugaaaauggauauaagguauguauaacuggggguggggugaggguaggaggcauuuacaacucagauuuuauuuauuuugaaauuaucaauuguauaaaucuaauuuauuaccaaauagggucuuuuaaaaaauauuuuuaucguugaaaccuugacagguacuucauauucuucuaauaauuuaaacaguccaauaaugugguauacacuuugacauccaagaacucaccaagauguuuuucagagauuuauucucgauuuaacuaucauagcauuuaaugaaucugauuuguaguucaauaaauuguggguugaacuacuuaucccugugugaacauugaauuacuuucugucacugaaacugagguauuugggugugguaaguacuucgaaaauuguaauacuguuugggcauugucuaaauuauuaaagguuaaaauagaaaauaaagucagaauuuuucuuuuccauuccaaagguguacuuagagaucucuauuaguauucauucgagaugacauagcagcucauaucaugguuguuuauuggauuuaucuguucuaauuauauaaguguguuuacugucuguguuuucacacaaacugcuagaauuuuuaauguuaagacgaaaacaucugaaguucuccauggcaaauugaauuuuucagucauuuucuuuucuuuuuuugguacaauuacuucaucuggaaugucuucauugaacucguuauucuauuuuucuuagaauuaaaaguggauuaauguggguuuuucuguucauuuuauugcaguauuaaaugcuuaagcuuauuaggaccauaauucacuuuaaauauaauuguauagaauauauuugcgucgaucaaauaauugcuucagaugaauucuuagacucuugauaauaucacaccuaauuuaacuugauuuuacaagcuguacaauccaguuuuaguuuucuauugugauaauaacuuuuuucaaaccaguuucacaucuuaaugaaauaacauucucugacugcacuugcuucaguacucucuugccugccuguuuuugaccucugcaugaguuggauuagauguuuuucuuacugucacuucuaaauagaaaaugacaguguuauaaaaaagggaaggauaaaaccuuugacauccccuugugucucaaaaguccacaguuauucaaacaauggcuuuuuuugugaugagaguauuuguuaaaaaaaaaaaaagacuucaagaaaaauaaaaguucaguggagcugcaaauaaaucuggugaauaauuucaucuuugguaaucucccauuuccugaguucuuccucaauccaagcuguccuguguaguauauaacauuugggcauuuucucugauauacuauacucucauguucuauaaauuucugucccguaauucuaacacuuuacauuuuuucuuugcuaucagcuauagcuauucauggaagggaagaaucacuaaauacuugucuaguuauagcaugaugugagcaucuccuccuuaucccucgaugccuggcuuggugucuggcaaacaguccauaauuagcagauguugaaagaccguuuacaaagcagaauuuggggauuuaaagugcaaugauacaacaaaaagauuuaauuacagcuuccaguguuuugacuaugugaaccauauccaacuacuuuuuugaaaaucuaguucuauguaauauauuucuguggcaucaaauuuuaguugauuguauuagucaauaggaagugguggaaaauuucuaaauaaauucaacuauuaaauaaaugcaaguuccacuaaa

SEQ ID NO: 50 - RNA, 2706 nt, Homo sapiens
  ref <308>: DATABASE ACCESSION NUMBER: NM_022073.4
  ref <309>: DATABASE ENTRY DATE: 2022-05-08
  ref <313>: RELEVANT RESIDUES IN SEQ ID NO: (1)..(2706)
cuggccgcagucgcggcagugguggcuucccauccccaaaaggcgcccuccgacuccuugcgccgcacugcucgccgggccaguccggaaacgggucguggagcuccgcaccacucccgcugguucccgaaggcagaucccuucucccgagaguugcgagaaacuuucccuuguccccgacgcugcagcggcucggguaccguggcagccgcagguuucugaaccccgggccacgcuccccgcgccucggcuucgcgcucguguagaucguucccucucugguugcacgcuggggaucccggaccucgauucugcgggcgagaugccccugggacacaucaugaggcuggaccuggagaaaauugcccuggaguacaucgugcccugucugcacgaggugggcuucugcuaccuggacaacuuccugggcgagguggugggcgacugcguccuggagcgcgucaagcagcugcacugcaccggggcccugcgggacggccagcuggcggggccgcgcgccggcgucuccaagcgacaccugcggggcgaccagaucacguggaucgggggcaacgaggagggcugcgaggccaucagcuuccuccugucccucaucgacaggcugguccucuacugcgggagccggcugggcaaauacuacgucaaggagaggucuaaggcaaugguggcuugcuauccgggaaauggaacagguuauguucgccacguggacaaccccaacggugauggucgcugcaucaccugcaucuacuaucugaacaagaauugggaugccaagcuacauggugggauccugcggauauuuccagaggggaaaucauucauagcagauguggagcccauuuuugacagacuccuguucuucuggucagaucguaggaacccacacgaagugcagcccucuuacgcaaccagauaugcuaugacugucugguacuuugaugcugaagaaagggcagaagccaaaaagaaauucaggaauuuaacuaggaaaacugaaucugcccucacugaagacugaccgugcucugaaaucugcuggccuuguucauuuuaguaacgguuccugaauucucuuaaauucuuugagauccaaagauggccucuucagugacaacaaucucccugcuacuucuugcauccuucacaucccugucuugugugugguacuucauguuuucuugccaagacuguguugaucuucagauacucucuuugccagaugaaguuacuugcuaacuccagaaauuccugcagacauccuacucggccagcgguuuaccugauagauucgguaauacuaucaagagaagagccuaggagcacagcgagggaaugaaccuuacuugcacuuuauguauacuuccugauuugaaaggaggagguuugaaaagaaaaaaauggaggugguagaugccacagagaggcaucacggaagccuuaacagcaggaaacagagaaauuugugucaucugaacaauuuccagauguucuuaauccagggcuguugggguuucuggagaauuaucacaaccuaaugacauuaauaccucuagaaagggcugcugucauagugaacaauuuauaagugucccauggggcagacacuccuuuuuucccaguccugcaaccuggauuuucugccucagccccauuuugcugaaaauaaugacuuucugaauaaagauggcaacacaauuuuuucuccauuuucaguucuuaccugggaaccuaauuccccagaagcuaaaaaacuagacauuaguuguuuugguugcuuuguuggaauggaauuuaaauuuaaaugaaaggaaaaauauaucccugguaguuuuguguuaaccacugauaacuguggaaagagcuaggucuacugauauacaauaaacaugugugcaucuugaacaauuugagaggggagguggaguuggaaauguggguguuccuguuuuuuuuuuuuuuuuuuuuuuaguuuuccuuuuuaaugagcucacccuuuaacacaaaaaaagcaaggugauguauuuuaaaaaaggaaguggaaauaaaaaaaucucaaagcuauuugaguucucgucugucccuagcagucuuucuucagcucacuuggcucucuagauccacugugguuggcaguaugaccagaaucauggaauuugcuagaacuguggaagcuucuacuccugcaguaagcacagaucgcacugccucaauaacuugguauugagcacguauuuugcaaaagcuacuuuuccuaguuuucaguauuacuuucauguuuuaaaaaucccuuuaauuucuugcuugaaaaucccaugaacauuaaagagccagaaauauuuuccuuuguuauguacggauauauauauauauagucuuccaagauagaaguuuacuuuuuccucuucugguuuuggaaaauuuccagauaagacaugucaccauuaauucucaacgacugcucuauuuuguuguacgguaauaguuaucaccuucuaaauuacuauguaauuuauucacuuauuauguuuauugucuuguauccuuucucuggaguguaagcacaaugaagacaggaauuuuguauauuuuuaaccaaugcaacauacucucagcaccuaaaauagugccgggaacauaguaagggcucaguaaauacuuguugaauaaacucagucuccuaca

SEQ ID NO: 51 - RNA, 968 nt, Homo sapiens
  ref <308>: DATABASE ACCESSION NUMBER: NM_001033678.4
  ref <309>: DATABASE ENTRY DATE: 2022-06-02
  ref <313>: RELEVANT RESIDUES IN SEQ ID NO: (1)..(968)
guccccgccgccccgacgcuuccgcccgguccgucggcacgucggcccugccucccggaccacccugcggggcgcaccagcgaucuggggugcggggcucggccucccugcgcuccuggcugaccgugugaccuugggucuuaaccaugaacuucucuggaggagggaggcaggaagcagcaggguccagggguagaagggcucccagaccccgagaacaggaccgagacgugcagcuguccaaggcucuguccuaugcccugcgccauggggccuugaagcuggggcuucccaugggagcugauggcuucgugccccugggcacccuccugcaguugccccaguuccgcggcuucucugcugaagaugugcagcgcgugguggacaccaauaggaagcagcgguucgcccugcagcugggggaucccagcacuggccuucucauccgggccaaccagggccauucccugcagguaccuaaguuggagcugaugccccuggagacaccgcaggcccugcccccgaugcuaguccaugguacauucuggaagcacuggccauccauccuacucaaaggccuguccugccagggaaggacgcacauucaccuggccccaggacugccuggagaccccgguaucaucaguggcaugcggucccauugugaaauagcuguguucaucgauggaccccuggcucuggcagauggaauacccuucuuccgcucugccaauggggugauucugacuccagggaauacugauggcuuccuccuucccaaguacuucaaggaggcccugcagcuacgcccuacccgaaagccccuuuccuuggcuggugaugaagagacagagugucagaguagccccaagcacagcuccagagaaaggaggaggauccaacaauaaaauauuaauuuauaaaaaagaaauuuuaaaaaguaacaagaaagaacucguuugaaacca

SEQ ID NO: 52 - RNA, 3873 nt, Homo sapiens
  ref <308>: DATABASE ACCESSION NUMBER: NM_207362.3
  ref <309>: DATABASE ENTRY DATE: 2022-06-01
  ref <313>: RELEVANT RESIDUES IN SEQ ID NO: (1)..(3873)
gagcggagccgagcgaagccggcgcgccugcagcugggagcugggagcugggagcgcgccgggcgcagggccgggccggccgcgggaggguagugucagcgcccgcguccagcucuucggccgcagacccgggcugucuggaggccgcgcggccaccugcugcgucgccccggcuuugggcuggggcuagacgcgcaccgcggcgggagcagccccggcucgccgcaccccgcggaaggugcccuucucugcgcgucucgccgagcccgggccacgcuccugucccagcgagcuugacaugauuuccacaagggugauggacauuaagcuucgggaggcugcagaaggccuuggggaggacagcacaggaaagaaaaaaucuaaauucaaaacuuuuaagaaguuuuuugggaagaagaagagaaaagaaucgccgucguccacaggaaguagcaccuggaaacaaagucagacgaggaaugaggucauugccaucgaauccgggccagugggcuacgacuccgaggaugagcuggaggagucgaggggcacgcugggcagccgggcccuuucucacgacaguauuuucauuccugaguccggacaggacgcuacucggccugugcggguguuuucccaagaaaaugugugugaucggauuaaagcucugcaguuaaaaauacaguguaaugugaaaauggggccaccaccuccuccaggggggcuuccugccaagcggggagaggaugccggcaugagcucugaggacgacgggcugcccaggagccccccagagaugucucugcugcacgacguggguccugguaccaccauaaaggucucugucgugucuccagaccacgugagcgacagcaccgucucugcccggaucucagacaacagccuggcaccaguggcugacuucaguuauccugcagaauccuccuccugccuggacaacucugcagcuaagcacaagcuccaggucaagccccgcaaccagcggucgaguaagaugaggcggcucucaucgcgcgcucagucugaaucccugagcgaccugacgugcaccccagaggaggaggaaaacgaggagaagccacuuuuggaagucagcccagaagagcgccccagcucugggcagcaggacguggcgccagacagaggcccugagccugggccaccggcgcccuugccgccacccggaggggcccgugccagacgcgcccgccugcagcacuccuccgcgcucacggccagcguggaggaggggggcguccccggggaggaccccucaagccgcccggccaccccggagcucgccgagcccgagucggcccccacucuccgcguggagcccccguccccgccggagggccccccgaaucccggucccgacggcggaaagcaggauggggaggcgccccccgcaggcccgugugccccggccacggacaaggcggaggagguggucugugcucccgaagacgucgcgagcccguuucccaccgccaucccugagggggacacgacuccccccgagacugaccccgccgccaccucagaggcgcccucugcucgcgacgggccagaacgcagugucccgaaggaagcggagccgacgccgcccgugcucccggaugaggagaaggggcccccagggccggcgccugagcccgagagagaagcggagacggagcccgagagaggagcggggaccgagcccgagagaauugggaccgagcccuccacggcgcccgccccgagcccgccggcgcccaagagcugccugaaacaccggcccgcggccgccagcgagggccccgccgcguccccgccgcuugccgcugcggagucucccccgguggagcccggccccgguucccucgacgcagaggccgccgccccggagcgccccaaggccgagcgagccgaggcgccaccggcgggcgccgagagggcggcgccagagcggaaggcggagaggggcggugccgagcugcgaggcgcgaagaaguucucgguguccucgugccgagcgcggccucguccgggcgucucccgcccgcuggaacgggccagcggccgccugccccucgcgaggagcggcccggucuggaggagcgaggcggcucuugacgaccuccagggucuccccgagccccagcacgcgaaaccuggcccucggaagcuggcggagcgcggcccucaggacucgggggacagggcggccagcccggccgggccgcgcaagagcccucaggaggcggccgccgcgcccggcacgagagagcccugcccagccgcccaggagccggccccgagugaggacagaaaccccuuccccgucaagcuccgguccaccucccucucgcucaaauacagggauggcgccucucaggaggugaagggugugaagagguacagugccgagguccgguuagaaaggucgcugaccgugcucccgaaggaggagaaguguccccucgggacggcccccgcccuucgaggcaccagggcccccagcgaccaaggaaaggggaaggcccggccccccgagccgcucagcuccaagccgccccugccccggaagccgcuucugcagagcuucacgcucccgcaccagcccgcgccccccgacgccggcccgggagagcgggaacccaggaaggagcccaggacggcggagaaaaggccgcugcgcaggggagcugaaaagagucugccgccugcagcaacagggccuggagcugaugggcagccugcgccacccuggaucaccgucacucggcagaagcggagggggaccuuggaccagccacccaaccaggaagacaagccuggggcacggacccugaagucugaaccaggaaagcaagccaaggugcccgagagaggccaggagccugugaagcaagcugacuuuguucgcagcaagucuuuccugauaaccccugugaagcccgcuguggaccggaagcagggggcaaagcucaacuucaaggaggggcugcaaagaggaaucucauugucccaucagaacuuggcucagucugcagugaugauggagaaggaacugcaucagcugaagagagccaguuaugccaguacagaucagccauccuggauggaacuugccagaaagaaaucucaagcuuggagugacaugccccagauuauaaaauaggugggcagugugcugauaaagcauguccacuacccugacaagccacuuaguuaaaaacugccaguguauuguggcaaacaaacugugaaacuuaaagauugauuuuaucaucaaguuaugacgaacuuggggaagaggaaggaaccaggcaaaacaaagaggauaaacacacagccauacuccugggccagagggcccagaaacaagaacaacauugaaaaaguccaggaaccaguuccugugcccaaagguacgaaugugucuuuucgaauucccuugauaaagaagccucagacaaggaagaaaauagaaaugaaauuggacacauaaauaggacacccagcaaaggccagaagagaagcccuucuaggagaagggacaucugugcuuccagcugagaucagcccaugucucuggucgcucucuugccuguggauccugucuucuuucauguuuacuacgaggcaugacauucauggcaaaacagggaugccguuuccaagagcagcuauaaaacaaucuguuuuccuaaagacagccacucugagaaggagaugaaggaacacggcaggguucaucaauucauagacuguuuccaaaaauaauuuuuaaacuuuuccacuaggcaucugugucuuuaaccacugcacaaugcauugccuguuuaauaaaagguuucaaacaug

SEQ ID NO: 53 - RNA, 1250 nt, Homo sapiens
  ref <308>: DATABASE ACCESSION NUMBER: NM_001747.4
  ref <309>: DATABASE ENTRY DATE: 2022-04-17
  ref <313>: RELEVANT RESIDUES IN SEQ ID NO: (1)..(1250)
gagaagccggguggggcaggcuggaaggaagacgaaccuacgaagcagagaucugaagacagcauguacacagccauuccccagaguggcucuccauucccaggcucagugcaggauccaggccugcauguguggcggguggagaagcugaagccggugccuguggcgcaagagaaccagggcgucuucuucucgggggacuccuaccuagugcugcacaauggcccagaagagguuucccaucugcaccuguggauaggccagcagucaucccgggaugagcagggggccugugccgugcuggcugugcaccucaacacgcugcugggagagcggccugugcagcaccgcgaggugcagggcaaugagucugaccucuucaugagcuacuucccacggggccucaaguaccaggaaggugguguggagucagcauuucacaagaccuccacaggagccccagcugccaucaagaaacucuaccaggugaaggggaagaagaacauccgugccaccgagcgggcacugaacugggacagcuucaacacuggggacugcuucauccuggaccugggccagaacaucuucgccuggugugguggaaaguccaacauccuggaacgcaacaaggcgagggaccuggcccuggccauccgggacagugagcgacagggcaaggcccagguggagauugucacugauggggaggagccugcugagaugauccagguccugggccccaagccugcucugaaggagggcaacccugaggaagaccucacagcugacaaggcaaaugcccaggccgcagcucuguauaaggucucugaugccacuggacagaugaaccugaccaagguggcugacuccagcccauuugcccuugaacugcugauaucugaugacugcuuugugcuggacaacgggcucuguggcaagaucuauaucuggaaggggcgaaaagcgaaugagaaggagcggcaggcagcccugcagguggccgagggcuucaucucgcgcaugcaguacgccccgaacacucagguggagauucugccucagggccaugagagucccaucuucaagcaauuuuucaaggacuggaaaugagggugggcgucuuccugccccaugcuccccugccccccaccaccugccugcuugcuucucuggcugccuggucagugcagaggugcccccugcagauguucaauaaaggagacaagugcuuucccagcucuuuuccugca

SEQ ID NO: 54 - RNA, 4801 nt, Homo sapiens
  ref <308>: DATABASE ACCESSION NUMBER: NM_014700.4
  ref <309>: DATABASE ENTRY DATE: 2021-06-26
  ref <313>: RELEVANT RESIDUES IN SEQ ID NO: (1)..(4801)
agccuccuuagucuccucauccugcuucacaggcuccgcggccuccggccuccucggcccccgucccccggccuccucggcccccgucccccgccauccgccgcccggauccucgccgcccucccuaggccgccccgccgccaugggccugcgcccgccgcgccgccgggccgagggcagcugaggcgcggugcgaagaugggcgaggacagagcagggcccgagcgccagccccagcagcccgggcgccccgcgcgcgcccgcccgcgccgccgaggggaugcccgcgcccgccgccgcgcccugagcgccuuugucugccgcccgcgcccuuccgcaccacuagccucucgggagcauggcgucggccccgccggccucgcccccgggcucggagccgccggggcccgacccggagccgggcgggccggacgggccgggggcggcacaacuggcuccgggcccugcggagcuacgccucggagcgcccgucggcggccccgacccgcaguccccgggccuggaugagccugcgcccggggccgcugcagauggcggggcgcguuggagcgccgggccggccccggggcuggagggaggcccgcgagaccccgggccguccgccccgccgccgcgcuccggcccgcgggggcagcuugcgagccccgacgccccgggcccagggccgcgcuccgaagcgccgcuuccagaacucgacccguuguucuccuggacugaggagcccgaggaguguggccccgcgagcugcccggagagcgcgccuuuccgcuugcagggguccagcagcagccaccgagcgcggggcgaggucgacgucuucucucccuuccccgcgcccacggcgggcgagcuggcgcuggagcaaggucccggguccccgccgcagcccucggaccucagccagacccacccccuuccgagcgagcccguggggagucaggaggacggcccccgccuccgagccguguucgaugcccuggacggggauggggacgguuucguccgcaucgaggacuucauccaguuugcuacggucuacggggcagagcaggugaaggacuuaacuaaguacuuggaucccagugggcucggcgugaucagcuuugaagacuucuaccaagggaucacagccaucagaaacggagauccugauggccagugcuacgguggugucgcuucugcccaagaugaggagccccuggccugcccggacgaguucgaugacuucgucaccuaugaggccaacgaggugacggacagcgcguacaugggcuccgagagcaccuacagugagugugagaccuucacggacgaggacaccagcacccuggugcacccugagcugcaaccugaaggggacgcagacagugccggcggcucggccgugcccucugagugccuggacgccauggaggagcccgaccauggugcccugcugcugcucccaggcaggccucacccccauggccagucugucaucacggugaucgggggcgaggagcacuuugaggacuacggugaaggcagugaggcggagcuguccccagagacccuaugcaacgggcagcugggcugcagugaccccgcuuuccucacgcccaguccgacaaagcggcucuccagcaagaagguggcaagguaccugcaccagucaggggcccugaccauggaggcccuggaggacccuucccccgagcucauggagggcccagaggaggacauugcugacaagguugucuuccuggaaaggcgugugcuggagcuggaaaaggacacggcagccaccggugagcaacacagccgccugaggcaggagaaccugcagcuggugcacagagcaaacgcccuggaggagcagcugaaggagcaggagcugagagccugcgagaugguccuggaagagacccggcgucagaaggagcuccugugcaagauggagagggagaagagcauugagaucgagaaccugcagaccaggcuacagcaacuggacgaggagaacagugaacuccgguccugcacgcccugucugaaggccaacauugagcgucuggaggaggagaagcagaagcuguuggaugagauagagucgcugacgcugcggcucagugaagagcaggagaacaagaggagaaugggggacaggcugagucacgagaggcaccaguuccagagggacaaggaggccacccaggagcugaucgaggaccuccgaaagcagcuggagcaccugcagcuccucaagcuggaggccgagcagcggcggggccgcagcagcagcaugggccugcaggaguaccacagccgcgcccgggagagcgagcuggagcaggagguccgcaggcugaagcaggacaaccgcaaccugaaggagcagaacgaggagcugaacgggcagaucauuacccucagcauccagggcgccaagagccucuucuccacagccuucucugagucccuggcugcagagaucagcuccgucucccgagaugagcucauggaggcgauucagaagcaggaggagaucaacuuccgccugcaggacuacaucgacaggaucaucguggccaucauggagaccaacccguccauccuggaggucaaguagaggcaggaagguccagccugagcuggauucgggacuccaacacccuggagugguuccgucagaccaugaggagccaagaccagcaggucccacagccgacagugcccagagcaugcagggaacccucgugcagcugagcuggggccgccaaagaccggggcugccaaaggggcagagggugguggagaggagagggagaaagggaagucccagggcccgggguccacagaggaugaggguuguggcagggccguccaucagcgcugaccuuccgggggcccagagcuucccagcccugagucaagcuggccaugaacgcguacacuucaguucagcaggaugggcuggagagccucucugugcagcgguguggggugagcccugcuguggccuccuugugguggucccucuucccacgugcagcccuguugggaagaaaggaagaaaacaggucccuccaggggugcugcugccuaagccacccacauaaguacgcuggugccgugucacccauguugagccgcuccugauggcugacgggcucccagacccucaccucggacaugguggugggggaaggacgggugggcaaggcuggugcguuccccagcucucccuacgcugcucgggccauugcccagccagauguggucaccucaguccagcucuggggccuccaggccauguggcuguucccacggcccaguccucgcugcaguaaccccugggggcucugaccaccuaugggggccgggcaggagccucuggggccuccacuccgacaucaggaccugagaugaccgcuguguggcgcucucucccugggcaggguggaugccacaggccccucuggcucccaggugcugcuucuccacaggugcggccuggcccggccuccuaaaggccacacccuccccacgcacuucccaggccagaauccaaacaucgggaacccuguuuucuucugggugugucucacuuagaaaucgugguucuuccccgagggugcauguugcaggagggagagggcagggaagacucacagcagagcaggagggggccugugcuucucggggucugcaccccaggcacagcggugucaccccgcaggaccgcgggccugccccaacccccagcauucccgggugggcccagaccccaucaccaagacuggccacccgcugcgugugugugcgcgcgcguguacguguggccccacauccgccgccuuccacgcuaggauguaagaggucgccuccuauuguacauuuggggaaagccuuggguguaaaucaguguaaacuuggaggagagauuuuucuaucauguagaguagguauuuuuuauagauugaagguugaucaauuuuuuaauacuuucaagagaaaacuguguauacacaugaaauauauauauauauauauauauauauauguauaauauauaaagacuggcacccugccucucugugcccaggcccagcccuggugacauggcaccacucagcagugcugucacuguaagcauggacucccaggagacagugugggaaacgcuccugcuuuaauuccccgagaaacggcucuuccugccuggaugcaggagggcaggggccaccacagauuaaagcuguuacugcacacgcagaggccggcuucuuccuccaggggggccacaggguggggugggagucuucugggccuggcccagcuucccucaaggcguccgaggccaucaguugucugagcucagaacccagcagaccugccuggcucugcagcuuggucagacuaagacccuuccaaggccguaggauugcaccuccaggcccaguggagccuggcagcuuuccagccagguucuggauccugacgagcugcugagacagcaucaagguggggcagguggggcggggcaaauugggcagcacagggcugugugggcaucagggcucugggucacacgccucuggggacguacgucccaugucgggagagccgucaaaacguccccgggauuaagagaagaguggggggcagggcgcagaggcucacgccauaaucucagcacuuugggaggccaaggcaggaggaucgcuugaacccgggaguucaagaccagccugggcaacauagcaaggccccaucucuaaaaaauaaaaaauuaaccagg

SEQ ID NO: 55 - RNA, 9787 nt, Homo sapiens
  ref <308>: DATABASE ACCESSION NUMBER: NM_153711.5
  ref <309>: DATABASE ENTRY DATE: 2021-09-06
  ref <313>: RELEVANT RESIDUES IN SEQ ID NO: (1)..(9787)
gccacagcugcucugccaauaacaaaggcacagcauuuucccucugugcaucuccaacauggaugcuuuucagggcauuuuaaaauucuuccuuaaucagaaaacuguuauuggcuacagcuucauggcucugcugaccgugggaagugagcgucucuuuucuguuguggcuuuuaagugccccugcagcacugagaauaugaccuaugggcugguuuuccucuuugcuccugccuggguguuacugauccugggauucuuucugaacaauaggucguggagacucuucacaggcugcugugugaaucccaggaaaaucuuucccagaggccacagcugccguuucuucuacguccucggccagaucacucugagcucauugguggcuccagugauguggcuuucuguggcucugcucaauggaacuuucuaugaaugugccaugagcgggacgagaaguucaggacuccuggaacugauuugcaaggguaagcccaaagagugcugggaagaacuucacaaaguaucuuguggcaaaacuagcaugcuaccuaccgucaaugaagaacugaaacucucccuucaggcccagucucagauucuaggauggugccugauuuguucagcgucuuucuucucucugcucaccacauguuaugcucgcugccgaucuaaaguuagcuaccuucagcugaguuuuuggaagacauaugcacaaaaggagaaggagcaguuggaaaauacauuucuggacuaugccaacaagcugagcgagaggaaccugaaauguuuuuuugaaaacaagaggccagauccuuuucccaugccuacguuugcugccugggaggcugcuucagagcugcauucuuuccaccaaagccagcaacacuauagcacccuccacagagugguggacaauggucugcaacuuagcccugaggaugaugagacgacaaugguccuuguggguacugcccacaauauguagcucauccaccaucaaugacucaugguguugaguggcaugcucauucugugauccuccuaacguaucaccagcaaccugugugucucuguauuuucuuacauuuggaguauguuuacaagacaauaacacaaaggaaacugcuuugaagcucucaaguggaacaucaggaugugcucaaauugaugcugaggggugacaaaggugcucuugcauugguggagaggggcucaguaggccuaaauguugcuccaagaucuaagauuuuaucauucaacagacagacucaugugagagggcucaucagucucuguaaauauugguugcagaauuuaggggcagaagcaauggcuugugagugagcaaguuacucuuaacauagguugcccuacacaguggcuccugcagugugaucagcaggagucucuguccuacaaaucuacacuuuuaaggauguaugaaauuguggaauucauucccucaggaucugaagaucucaguucucaaaaaucacaaguugcaagaauaugauaccuaaaauugaaaguuuuucacauguuuucacuguauacugaaaauacuccccuggaagcagggcugcaucauaauuuuugugggcuuuauguacuuuugccuuugcgaaccauuuccuacauucaaaauaguaguaacaaauauauauauauauauauauauauauauauauauauauauauauauauacacacacacagaaauauauauuuauaacugcauugauaaaaggugaauauauuagcauuuucuucaaccuaaaaguuuauguauuuucuucugauuuuaaaagaaauuaaaaacauuuuagugggcccuuacaaguauuaugguccuuaggcacagugcuuacuaggccguggguaugucuuagugcguuugggcuacuauaacaaaauaucauagauagguggcuuauacaaaauagaaauuuauuucucacaguucuggaggcugggaaguccgaggucaggcugccagcaugcucagguucuggugagagcaauuuuccagguugcaaacugcuugcugacuucucaguguccuuccaugguggaaagugcaaauaagcucccuugagcauuuuuauaaggcacuaaucccauuccuggaggcuccaccuucaugacccaauuacuucucaaggcucugcuucuuaguacuaucagcuuggggguuaagauuucaacauaugaaauugggggagacacaaacauucagacuauaguaggguaaauugaccuuggcuaaaggucuggggacacuguccaaagcaguacuggcauaaaacugcauugcagucuuacauuuaucagaaauaaugcaaaucuugcauucuacaauguacuauauccauguuaguauguacauauauauguguauguauauauguacauaaauucuccuuaaaucucugagaagaauugugcuuuaaaaaagaacuauguuuugugcuguggcucauaccuuacagugguagucuaugucucuuaguuugagaaagcugaauauuaacuucuuauuaacaugaaacaugauggcaaacugaauccuacaaguauaggacccagaacauaucuauguuauugucauuuacaagaagaaauauguauuuggucuuucuuccuauuccuggugcagagcuccuaaaaccuuugggaccucccaagugaugugucugguaguacgcgaaugagaugacugauaguuaggggcuucugaauggccucagaauagggucugguugccacgggaaccaaccauguguuuagaggguuggaacuuucagccccacucccagaccuuggggaaggaagaggggcugaagguugagucgaucacaaauggauaaugauuuaaucaaucaugccuaagaauaaagcuucccauaaaaaucuaaaggacaagguucagagagcuucugcauugcucaauacaugaggaguggugagcccaaaggggccguggaagcuccgugccccuucccccaaaccuguuccugugccuccaucuggcuguuucugaguuauauuauuuuauaauagacugaugauaguaaauaaagcccuuuccugaguucugugagcuacucuagcaaauggugaaaucugagaaggaaaucaugggaaccucugcuuuguagccacgucagauagaagaugugaguaacuuuggaaccuacugcuugugacuggugucugaaguugggggagaucuuaugggacugagcucuuaaccugcuggggaucugcacuuaaugccgauuaaugccagaauugaauuguaugauauccaguugguauuggagaauuggccagugugggagaaaacccacacaguugguggcaaggaguguucaguguugagugugaaugucaauaaaaaaagguuuguuuuuucuacguacaauaucuuugggaacuuggaggugcagggucagacgucuguguguguauguggcagggacagaacagggauuagggaugugaggggguagaguuuucagaauaaagggagagaaaauucagcaaagaguucuccuucuuaaucuuucuugacuuaaaguuugucugcccuuaagugucuucugagcuggaaaaagacauauaggaacuggccgaugccgaugccucuuuugcagugugugagaggcugaaacagaaguucuacagaaguagguuagcuguaaggagcugaggauugaggagucauaucugggggauauuccugccacccucaaguccagggacuuucugcacucaaaaaacuuacgcucaugacaaugacugccucauucaagaaaacccaagcugacuggcaagaagccagcagccuggguacuuguugcugcagcuuaguaaguagcagaagagcucauccuccuccucuggaagaagccacuuaacagguucucauccugacuccuaaauuccuuaacaguccuuuacucaggaauaaaacaaguccaagaaaacuuaucugucccacaaauaguccuggaggcaguggaguaagauagggagagcaaauuggugucugcucuugaguaucuugagggaauggguaucuagaggcagcaggaugaacuggcuuugauagcagagacuuuaaaauguguggaccauuaguugucuuuggaugugcucccuagaggcugcgccugacacagggagcuguguauacaugauuuauuaaaaggaaauccucuuaggagaaacauauuagcaggauaaacacaagaaagaaugcaguuacaaguggagucucaaucaaucccagaggaaauucugggacuucagccucagcuugaccuaugggggggcucuggaaugucagauuucuccugaucccaagugugcucuggggcuuccauugcagccaagagagagcauuccccagaaaacuccaggugaggagucuccaguggcccaagggugauccuccagaggagguuacacugccgaacugggacauuggcacccuggauuugaauaucuagagagggcacccaauggucauacuaauguuugccauaacagucucuucuaggucuagacuugaagcauauggaaggguccuugggcuagagccaacccaagcccaggcugacucccuuuuaauacuucuaauuuucccugaagaucuggucuucucccuaccaccacuacccuauuacauaagagaaaaggauuggagaaugcucuugaaaagaaugugaugcuuucccauacaaggaauauacucaaggaaaaauuucauggcacaaaaugcugauucuguccaauuuaucccaugaauaaauuguguacacauauauauaugauucuuuuguuuuuccugugcaguuccuuccuuuuuucaggcucaaccacuuaauucagcuuuaggauacauucagcuuauucauaucaaauauaaagugggagaaaaaaagguauguaacaaagauuacaaaaauaaggcauggcagacaaaucacuacauaauucuaugcauuauaccuuaaaguaauguauuguaaaauauauguguccaaagcaacuuuuaaaauuuuuguaugguuuucuacuugagaccuuuaaagcaucacuaaaaauguauacauuuaaugagaaaacgaugacauuauugauucaaauuaguuguuucaaagccugaugauccacugauuuacucuaaaaacugguuaaagcaauccagaagucacuggguagcagaaaacugcuaccgcuucucacaugauauauuucaaguacuucaacuauuaacuauauuauucuauuuagacuuuggugguuccgaauucucuaagguaauuaucuuaauuuucuaauauuucccucucauuuuucucucuuauuuuaaauauccccuguuguacaagccagaggauauuaauuuccuuuguggguagugcuuggacuucucugauuuuagaucccaaaaucauuaagaaaaagcuaagagguuggcaugagagguuggauggaaagcaucuaucuguguuugucaguagcucucgauguuggcugcaaauuggaaucaccugggggccuuugacaaucacaaugucuggacccauuuccugagaaccugauguaacuuuucuggguuagggccugaacauugagauuucuuuuaaaagcuuucuaagugauuauaaugugcagucauggugaagaacuacugauuuuauauggggagucagugaagguggugaggggcugugagguugaaauauuggaaauguuaucuggauuucaaacaguagaguaugauaaaaauuuaaaaacuggcucuccaggaaaaaagagacauccuaauaugauguauuugccaauauaccauggguuuucuugggcugggaugaacuuucagaaguuuuacagugagcauuuuuuuuuuuaugaauuuaaauugguucuagcuaagcaauuuuggaguaucuuugauguaccugggucucucauuuuaauaaacauaacagugguauguuagucaggguucuccagagaaacagaaccaacaguacaacaguauauauacauacauacauauauaugugugugcauguguguguguguaugcguguauauauauauaugcuacauauaugaaaggggauuucuugugggaauauauauuauauauaacaaaacaugacaaauauauaacaaaauauaugugcuauauauauuguguguguauauauauaugaaagaggauuuauuguggaaauuggcucaugugauuaugaaggcugagaaggcccaugacgugccaccuguaagcuggagaaucagggaaauguguaggguggcucaagucccagucugagggccugagaaccaggggaguugaugguacaaaucccagagccaaaggcaaaggaaccugaaguuuuuauuuccaagggccaauagauaaggagaauauuggugacccaacuccaggagacagagaaaagauuggugacccaacucgaguggagagagagggagagagagagagacagagagcaaauuugcccauccucucccuuuuuguccuacccaggcccucaauggauuguauggugccugcccagguuaggugagggcagauuuuccuuacucaguccacugauucaaaugccaaucuuuuuuggaaacaccccuacauacauacccagaaauaaugcuuuaauaacuaucuggcuaccccuuaauccaaucaaguugacaucuaaaauuaaccagcacaugugauaaauuaaauaugguucaauggcuccuucuggcagauguagucguguuauccucacuuugaaucugaccuuaugaaccaauaaaauauggcagacaugagguagauuguuaagcaguaucacaguuuaugccuuggucucuuacaauguugcaaugcucaacagucugguaaaagccacuauguaagaacugcaauuacucagggacuaccaagcucugaagaggcccaagcuagccagugggaggagagagagagagauaaaaagagagagagagagagaucccaauggcuccaauugucucagauauucuagcuuaggggucaggcaugugaaugaaggagccauuuuaaacauucaggccccaguguacacgacauggagaagaaccaggaaguccaaccaacagugagaauaaaaggccccagaucuauggcccuaaucaaaucauugcagccaucuccaaacauuugacccacuccaguggaggcugcaggcuuuguagagaaguccuugccauguccuauccaacuccaugaccccaaaaaucaugagcuuaaauaaauguuuguggcuuuaagccacugaauuuuggggcaauaugaagcgacagaaccugaacacuaacaugaaaaucagaauuuauaaaaagacaaaaauuacaggcaaauuguauuuauacauuuauacauucucccuaugcauagagagcauuuucuucaaucauguuuucuuuuucuuuagguuccaguuagcaguauagcaucuacagcaugauuugauagaugcugauaaguaggucucuaaaaucucuccaccaaagcuggagauucacuuucaauuauuuuugguuaauuuuuguuugaaaggaauacaaacauauuauugggaggaguaacacauuaaacuauuauuugucauuaauaucuacgaaaaaauagcaagugaggguuucagacuaggcuggccuucaguaagcuggaaagagauuguucuucuagucgucucucaaaauguuaucuucagacugucaacaucaguaucacgaugaagcuuguuagaaaugcagaaucagacaguaucuaaaaccuguugagucaagccugcaguuuaacgugauccuagcugauuccaguucaagcuaaauuugauaacacuaaggcagugguucucaaacuuuaguccauauaaaaaucaucuggagggcuuauuaaaauguggauugcugaacuccacccccagggugucugauucaguaggucuggggugaggcaagauaaucugcauuucuaacagguucucaggugauacugaugcugcuggucuagggacuacaucuugaaaauaacugcucuacagcacagagcaggcuauucuggggaaggcaggcuguaagaaacucagaauccuggccugcacuccagaucuacugaaucggucaucagcccguccaagauuuuggggugccuuuccauucauucaaggauaccuuucuauucauucaaggauauauuuuguccuuuaggaauauuuuaaaguuuacuucacauaaguuuuacauguuucuuguaaacucuauuuguagguguuuucuuuguuccuguuguaaaugguuucuuuuuuccacuauaucuuucuaagugauuguugucugaguacgugaaaagcuauugucuuaugcuuauuaauuuuaucucucuacuaauucuuuuauuguuuguagggcuuuucagaauuugaaaguaaacauuuugcaaccuuugaugacaaaauggaucuagacaauaaauaaugaauucuaaacuauuaggugaaagcagaauggggucagaccuaugcaauuugaagccacucaucuauauuagcaucacuaaaaaucaaagcagaugcuacgugccuuaugaugcaaggcaagaauaaguacauaguaucacaugugaaauauuguuuccuaagaagcuaaaccuaaucaaguauuuagacauuacugcuaguuuauaggaaacauaagcaacaaaaaacaaggcaauuagcaccacaaaaaaacaaccagguacaucuagaucuaacagaacauuccauuuuacagaugacucagucuccccacagauuaacaguauuaagaaaggagaauaaaggagauauaagugacuuaauaaccagauaugcuauauagaccuuauuuguauccuguuuccaacaaccaacuguaacaagauaucuuugagauauuuaggaaaauaugaaugaauauuagaugauuuuuagagaauuauuaauuuugcuaugugugguaauggugugccgauuuauguuuuuuaaaaaguucuucaguuagagauguauauuuaaauauuuaugugugaacugccaugaugucuagaaguuucuuuaaaauacucuagaaaaaagaguuuagggagaaagcugaaagaugaaauaagauuggcaaaauauggaaacuauugaagcuggaugguagguacacggaauucauuacauuauucucuuuacauuuguauauguuugaaaaauuucauaagguacacaacaaaauaaaugacauggucaccucaguuauuuugcugaaguuuugucaaucacccccugcuuagcuggaguuuaccuucuaguaucauucucagcaaaggcucacaggaaugcuauucucugaauucuuacauauucagccuguuggccuguucucgaauugaaggacagcuugucugguucauacuuucucuacaugaguguauuguaagcauuacuucauugucuuuugucauugaauauugcugagguggccacagccugauuucugaugccaucucuauuucuuucuuuauuacuaacaagauguuuuauccuauauucucauugguuucuuuaucuuucauguccaaaaauguuaaauaggccuucagguaggucuuuuuggaacgauuuuuccugcuacaaguuuacucuuucauuguggagaaucauuuucuuauauuucaauaaaaaaguuggaaaauuuucaa

SEQ ID NO: 56 - RNA, 9419 nt, Homo sapiens
  ref <308>: DATABASE ACCESSION NUMBER: NM_001714.4
  ref <309>: DATABASE ENTRY DATE: 2021-06-24
  ref <313>: RELEVANT RESIDUES IN SEQ ID NO: (1)..(9419)
aucauuccgcaccggcugcugcagggccagagggagcagguggagcgagagagcgagccgcgagccggagcgcgccagacccagggcgagacugcagugacgcggcccgggagacauggcggacgggcgucucugaauaagcagaauccggagccccucgcuacccgcggccgccgcagcccgggccaugccgcacggcugcugaccgcacgcaggggccggccccgaggacacaugcggcggccuuugccgccucgccccugacccucugcccuguucuccauguugcauuucucgucaguuucucgggcgguguagcugccgcugccaccagagccggcggggcaucgcgcugcucauucauccggccgcacuuucuuuuccguuuccacccaucccuucccauuuccuucucccuuuccccgccagcuucgcauccaucucccccaccccguaacccccuccugccuccauccaccggggcuauggccgcagaagagguauugcagacgguggaccauuauaagacugagauagagaggcuaaccaaggagcucacggagaccacccacgagaagauccaggcugccgaguacgggcugguggugcuggaggagaagcugacccucaaacagcaguaugaugaacuggaggcugaguacgacagccucaaacaggagcuggagcagcucaaagaggcauuugggcaguccuucuccauccaccggaagguugcugaagauggagagacucgggaggaaacgcuucugcaggagucagcaucgaaggaggcuuacuaucuggggaagaucuuggagaugcagaacgagcugaaacagagccgggcuguggucacuaauguacaggcagaaaacgagaggcucaccgcagucgugcaggaucugaaggagaacaaugagaugguggagcuacagagaauacggaugaaggaugaaauccgagaauauaaguuccgggaggcacggcuccuucaggacuauacugaauuggaagaagaaaauaucacauugcagaaacuaguguccacguugaagcagaaccagguugaauacgaaggcuuaaagcaugagauuaagcgauuugaggaggagacgguacugcugaacagccagcuggaagaugccauccgauugaaagagauugcugagcaccaacuggaagaagcccucgagacuuuaaaaaaugaaagagagcaaaagaacaaccugcggaaggagcucucccaguauaucagccucaaugauaaccauaucagcaucucaguagauggacucaaauuugccgaggaugggagugaaccaaacaaugaugacaaaaugaacggucauauccaugggccucuugugaaacugaauggagacuaucggacucccaccuuaaggaaaggagagucucugaacccugucucugacuuauucagugagcugaacauuucagaaauacagaaguugaagcagcagcuuaugcagguagagcgggaaaaggccauucuuuuggccaaccuacaggagucacagacacagcuggaacacaccaagggggcacugacggagcagcaugagcgggugcaccggcucacagagcacgucaaugccaugaggggccugcaaagcagcaaggagcucaaggcugagcuggacggggagaagggccgggacucaggggaggaggcccaugacuaugagguggacaucaaugguuuagagauccuugaaugcaaauacaggguggcaguaacugaggugauugaucugaaagcugaaauuaaggccuuaaaggagaaauauaauaaaucuguagaaaacuacacugaugagaaggccaaguaugagaguaaaauccagauguaugaugagcaggugacaagccuugagaagaccaccaaggagaguggugagaagauggcccacauggagaaggaguugcaaaagaugaccagcauagccaacgaaaaucacaguacccuuaauacggcccaggaugaguuagugacauucagugaggaguuagcucagcuuuaccaccaugugugucuauguaauaaugaaacucccaacagggucaugcuggauuacuauaggcagagcagagucacccgcaguggcagccugaaagggcccgaugaucccagaggacuuuuguccccacgauuagccaggcggggugugucauccccgguagaaacaaggaccucaucugaaccaguugcaaaagaaagcacagaggccagcaaagaaccaaguccaacuaagacccccacaaucucuccuguuauuacugccccaccgucaucuccaguauuggauacaagugacauccgcaaagagccaaugaauaucuacaaccuuaaugccauaauccgggaccaaaucaagcaucugcagaaagcuguggaccgguccuugcaacugucucgucaaagagcagcggcucgggagcuagcccccaugauugauaaagacaaggaagccuuaauggaagagauccucaagcuaaagucccugcugagcaccaaacgggagcagaucgccacauugagggcgguguugaaagccaacaagcagacagcugagguggcgcuagcuaaucucaagaacaaauaugaaaaugaaaaagcaauggugacugaaaccaugacgaagcuuagaaaugaacugaaggcuuugaaagaagaugcugcaaccuucucaucccugagagcaauguuugcaacaagaugugaugaauaugucacccaguuggaugagaugcagagacaguuagcagcugcagaggaugagaagaagacucugaacacuuuguuacgaauggcuauccagcaaaaacucgcccugacccagaggcuggaggacuuagaguuugaccaugagcagucccgacgcagcaaaggcaaacuuggaaagagcaagaucggcagcccuaaaguaaguggggaggcaucagucaccgugcccaccauagacacuuaccuccugcauagucagggcccacagacacccaacauucgggucagcaguggcacucagaggaaaagacaauuuucaccuucccuuugugaucagagccgucccaggacuucaggggcuuccuaccuacagaauuuauuaagaguucccccugaucccaccuccacagaaucauuucuucugaagggccccccuuccaugagugaauucauccaagggcaccggcucagcaaggaaaaaagguuaaccguggcuccaccagauugucagcagccugcugccuccguaccgccacagugcucacaacuagccgggaggcaagacugcccaacugucaguccugacacagcucucccugaggagcagccacauuccagcucccagugcgccccucuccacugucucuccaagccuccucaccccuagucuucaucuccuguggacgaacaucugggguggaaguuuuguagccacacacaggauacugcccaagauccagcggguguuuucuucucgguuguuagauguacaauuggauuaauguccaucguuuuggaagacgagagaaaguugagaagaacacgaagcacagacccugaugugauaaaacauuuugugguuucucugagucacagauaaacuucugccaucaaauggcuacaguucauuuaaauuuaaaaaaaagaaaaaagaaacagaaaacgugucucagauggcuggcuuuaccucgauagcauaagagagaccuaagacauguaaaauacguauauugcaguaucaucuuuccucacacuccaaauucagcuagggaaguugauuccaauauguuugucauugauauuuauuuuguacuuuauuugcuacaugauuuaugucuauacaaauaauuucucugaggugaauuuaauucauuuauuuucaaauaagcauaauuugcucaauuaaguaugaguuugaauuuaguuugaaaucuggaauuggccagacuguggucauuuuucuugcacaaaaugauaaaugaggugcaucagaauguuaacaauaacuguauuuugcugcuacgcugguauuguuuaggcacuuacuuucuaaucaguagcucauuaacugcuggguuuuguuuuuguauuuuuaacuagaguucuucacuggacauuacuaaguaaaucuaagaaagagacuaaguuaugauuuauugacuuauucaaguuuugauggcaucuuuacuuuuauuuuuucauugcaguugaguagauguacuggugcugcuccuuuguauguguguaugugugugauaacguuaaagaaggcucaauaacuugaagggaaaaaaaugguguguuuauuuaaugaccagaauuuuuuuuacuucucccaaaaucaggguccuacugagucuuuccugauaaaagguaucaaguacaaaugggaaaauauauacauauauagauguaagauagccuuuaauuaauuaauuuacccagagaguguucaaaguuugauuaaaauuguauguuuuguuguuguuaguuuuuuucauaacgaaucuuccuugccaaaaaaacaauagauaauaaaccuuagcucauugucuacuuuugacaaacacucaggugccuacaaacauucuauuaucuugagauaauacauguuccugguuaauuuacagauucugcuggguaacuuuuaugacuugauuuaaggcaguggauuuucauagcaaaauuuauuuugcacauaaucugacaaacaaggaaaacagcuaacugagcugaaaggucuaacacucucgggcuccuuaaccaucaagugcugcccacacaauugcuccuagcccugauuccuugucuuugucagguagccuuaacuuauuuaaaaccauaaggguuugaccuuuucauauaaagacaucuguaacuucucuucaaaacaaaaaugacccucacuguuaacacaaaggacaguuaccaguguagggcacugagaaaggagauccaggagccacucagcuuauugagcggacauggguggcacagaaauggagugguccaucagagaccaaaugguaagucguaaagcacauugcaggagcuuucguuagucauacgccuauuaaguaguggucgcuauuaccaaagcaaccaggacguuuuguguucuuuauaugagugcuauuuuccacagugucauuuauuaugcaacguggaguaaagaguugaaaucuucaccaaaauaaggguucagguuaaaguacagaaacagaaacaaaacgauucgacaugaaguucccaaccauagcagccuaaccuucugcucuuuucuaccugcauggccuugcuguuccuaaacauugucuucauuucacuggcguuguaucugccuaguaccaguauuagugagacuguggauuuuaucagaguucaaugaaagaaagcuuaccccugcccucacuuuguguuuuuuauuuuucccccuuucucaagcaccacguauuuggaccugagaaguggcauagcugcuaaguugacuuuuaauaaaaaacuguuugugccugagggaaauauaugccuuuuuaaaaaguaccucagaacauguucguagaucgucucaucgguuuuguuuggugggacugggaguucagcaggaaguauugucugugugugaugacggggcaguauugccagucggcaauguuguauuugcauucuuuaucccuaacucugaaucuaggacuccaugaaaagccgggucacccacaaaacauuucuuccaauacaaaguguauuauuacgucuauuauaaacuaguuucauuucaguuauuuaucuucauagagugggaaagucuuuuucugaaauacuaauucuaugaaaauaauaauuuuagugaaucuuugccaagaaguuauagaaacuuggaaauaaauuucucauuuucagcacaaaauuaagugauagucccuaagcaguaagagagaauaccauguauuuacacuguggcaucauuuaguauaguuuacacugagccauauuuauuuauaggcauuuaaagugaaucaguauguguaguguugauaaaaaucaguaucuguaaagauaacaucaggaauucaaaaggaaauugaaaaggggcagaacuagugagaaccaaaaagaaugaacuccaguaagacuaauugaagacauacuuggagaggaaaaauaaacagcagaaaugcacuaaaaaauacuggcuaauuauugcaaacagagugagcaaagauuaaugcuaacuaguaaaaguaguuagcaaaguaaacagguaguuuuuuaaaaguaagcacaauuacaauauugaauaguauggagaaaugcuuuucuccugaggauuuuccuuaaucaaaaaauaagaagaagcaauaaauugucuuuggccugagugcugaguggcauaaauaaaggagcuuggcuuaaucuuucugguccacacauaauccauuagaucccauucauauauccaacucuggauucuaauaaaauaauuuggagaacuuugagaaaaaggaaaacuguuaaauaagaaaagaccuauuuagaaauuauacuuuagucuaacgaagaggagguacaauaguagucuucaagaaaagaauauuuaacaaaugauagauaucagcuauagauauuguuacuaagucuagaauaaaaaaggcuucagugugaaggauguauaguaacucuaggaaagagauuuuuaacacugggaaauuaacguggaaauugauaaucauaaagaauauuuguuuuguaaaaauugaauaucauugguggucuaguaaguauuaaguggcaagauaggagaaugcauuucaguucugggagcucauaauccugugauucuuuuauuuuaaguuuuaaaauacauuaaaauuuuacugguuaaucauaaaauauuuuauauuugaaaauaggauggcaaucuuuauuuuuccaagauauuuguuggaaaaaaaugaucguugguauuauuuuuucuuucagcuauauuccaugguaucuuuaaaauuguucagaauauaggacuuuuacuaaaaaaauucaauugucagagucuaaaaaaacaauguccucaggaauacuucagaaauagaagguaugcaaucccuuaguggggggcaaacacauuucagauuucgucuuuauuuaaaaggaucuucagucuacaacauuugcuuuuuucgugcuuuggaaaauauccuugugcagauaaguucuucuguuugaaacaagcaugaaacauugguccaauaaccuaagaauguggagauuucuugauauucuguauuaauuaaaguucucuacaugucucaaauggagaucacaaaacaugaaaaggaagaaaugaaauaucuuuucuucugggaugagugucaauuuuaauguauaauugucauaaucaaucagccaaaaguuacaaucugucccucagagacauugggggaagcugauggauuuccaguaaggaacugugguggguucaaacucaggcugccacaaauacauugauaugguagguaggaaguaggacuaaauuugaaagaaacuuaugggauaaaguucacaauuuuaaaaagguugcccuccaguuuuuuuguuuguuuauaaacuagagugauuuuuaaguaacccuuauauuuaaggcugcaaaaaauguacaagaguauguuuucaaguaaagggaacauaauucuucaaaaagcuguugaacuggaguuaugauagguuaugacagagagcaugaucagugcugauacugacaaguacuuuuuuaccuuaaaaucaacuucuauggaacuacaagaucaaucuagcucccgagugacauuuuccauugucuguaauaaugcccucggaugaguugugucuaaaauuaaguucaucuuuauuuauaugcgaacuuaacugccauagucccuaauguauugcguuuguaaccugaucguauuauguuuacagcugaaagauuucaucuagacaugucuuucguccuuauuauucaaaguguaauugaaagagauauuuaguauuaagacauguuccccaauugagaauuuuccagaauauucuacuuaagaagaagaagagcaauuaacugccuuuaguguaagggcgagagugcauagaaauaugcaauguaaaauguuugcaugaauuauuucacaucauguaagcuuucccauauucauaagaugaacacuauagaagucucauuucucugugaucuucuguccauuaggaaaguaaggagauuguuaucuauaucuagucuccuuuccauauugaacugcauggcucuaauccucaguguauuuuuaucccuucucgaguuauuuaaaauuugcccuauuuaagcugaagccuggauaaacugcugagccagauuauuccugugauuggaguuuaauugcuguagaacacuuguugagaacacauuuuuuacuuuuuuucuuucagaauuauuccucaaucauuguucucuaaugaaaacaccgagaaguaaaucuggugcuucuuaguguaaauauguuuuucuuuuuaaaaagucauuuuaaaacaaagaugagagaacuauaaaguaaggggaaauaucauuuuauuuaaaaauaauuauucaaugauagauaauggagauacagaaugacucauuggguucauagguaugauuugugcagccagucuuagaggaaauacaucauuuuaacauuauucccacaaaauguuaaagcucaaugguuugacuaugagagucaaacaauuacuauuugaaaaucucuuaugacauuuuuauaaucuacucuguuuuuauuugcaugauuaugcuuaugugaugaaccauccaugauucugauauuguauuucuuuccaguauuaacauguguauuguguggccaacucugauuuguacaccguauuauuguuguaaugauucaacuguuccuacauuuaauugcaguuuaauuauuaugccuuuugggguuaaaugaugugaaguguuuccaccuacugauaaauaccauaugaaaacacgacuaaaagacugauuuaagaaacuuguuauuuuuaaaaauaugaauuucaaaagaaauguucucaaagacacaugcauuuauuuuaaauucaaaaaauucuuaugaauuuauuauugcuuguacuuuaacuuuuuuuauuguagacaauccuaagugaauaaugucucaugggagaacacaugauaugugcuuguauuuguguaaucugaucaugcacucaaugguuggaaaaggcacuccagugcuaagaaaauaaccagaagccaauauaucgauucuuauaucucgguuuauguuaccaacugaauaugguguaaugcauaacuuuuuccaguaaauaaacugguuauuuuuuguucauuuca

SEQ ID NO: 57 - RNA, 1899 nt, Homo sapiens
  ref <308>: DATABASE ACCESSION NUMBER: NM_012280.4
  ref <309>: DATABASE ENTRY DATE: 2022-04-17
  ref <313>: RELEVANT RESIDUES IN SEQ ID NO: (1)..(1899)
guucacaucaggucccggcccgccggaaccugggcgauccacgaugccgaguuugccacgcugcgacagcccauaggcuugcccccccgggcauucggguggacuacgaacacaaacugaagcccuaggacuugucgcccguuugcgcucucgccgaggcacaggcugcucgcggaccacccugcuccgaaaacuaagagguaggugguagcccauucaucugguuacugauacuggccggcaucaguggacugucggcagguccuugagcaacuggugugugaaaugggacggacgucaaaggacaagcgggaugucuacuaccgccuggccaaggagaauggcuggcgugcucgcagcgccuucaaacugcuacaacuggauaaggaauuccaacucuuccaaggcgugacacgggcaguugaccugugugcagccccaggcagcuggagccaggugcugagccagaagaucgggggccaaggguccggccacgugguggcuguggaccugcaggcuauggcuccacuaccaggugugguacagauccagggggacaucacccagcuguccacugccaaggagaucauccagcacuuuaagggcugcccugcggaccuaguggugugugacggggcuccugauguaaccggucuccaugauguugaugaguauaugcaggcccagcuccuccuagcugcucugaacauugcuacacauguccugaagccagggggcugcuuuguggccaagauauuccgaggccgggaugugacgcuccucuacagccagcugcaggucuucuucuccagcgugcugugugccaagcccaggagcagccggaacucuagcaucgaggccuucgcugucugucagggcuaugacccucccgagggcuucaucccggaccugagcaaaccccugcuggaccauucuuacgacccagauuucaaccagcuggauggucccacccgcaucauugugccuuuugugaccuguggggaccugagcuccuaugauucggaccgcaguuacccacuggaccuagagggcggcucagaguacaaguacacuccacccacacagccccccaucucgccaccauaccaggaggccugcacguugaagaggaaggggcagcuggccaaggagauccgcccccaggacugccccaucagcagaguggacacguuuccccagccccuggccgccccucagugccacacccugcuggccccugagauggaagacaaugaaaugaguuguucaccuuaacccauuacgcuggcaaacugauacaacucagaaacugcucaaugucaggaaaaccggaacuuguugaugaacuuguuuucaaaauaucaucucaucaacgaggccuggacaaacaagcccugaggagggaucugcaacaacccugaagacaacaaggaaagaaaccaugaaagucugucuguacugcagugggaauucuugagugaggucuuaccucuucuuuaaaccucuucaggaguguccugauuauguccagaauuuucccuaaaggcagggauucuuaaccuggauagaagccagggguaaccaugaacuugauggaagaaaauguuacaucuuuauuuucagcaaugaaacugaaauuuagccuuacucccaaguuauaaaugcuggcaacaaaucacaguaguaaaagcaguaccugggauuuucccaccaauacaaaccaguuacagcaacuguaauguaacauaaaaagggcaucuugaaguauuguucauauuugucaccucuuggaaugauagcagauccuguaaguugauguauuaauuaaaaagcccauauauuacugcaucucaauuuuguuucaauuuaauauauaguaauaauuguaauucaauguaa